(19) United States
(12) Patent Application Publication
(10) Pub. No.: US 2026/0265233 A1
(43) Pub. Date: Sep. 10, 2026

(54) BENZOTHIOPHENE DERIVATIVES AS RXFP1 AGONISTS

(71) Applicant: BRISTOL-MYERS SQUIBB COMPANY, Princeton, NJ (US)

(72) Inventors: Sreekantha Ratna Kumar, Bangalore (IN); Laxman Pasunoori, Warangal (IN); Pitani Veera Venkata Srinivas, West Godawri (IN); Srinivasan Kunchithaptham Duraisamy, Hosur (IN); Vikram Bhogadi, Prakasam (IN); Subramanya Hegde, Bangalore (IN); Michael J. Orwat, New Hope, PA (US); Durga Buchi Raju Barre, East Godavari District (IN); Donal J.P. Pinto, Churchville, PA (US); Leon M. Smith, II, Somerset, NJ (US)

(21) Appl. No.: 18/718,502

(22) PCT Filed: Dec. 14, 2022
(86) PCT No.: PCT/US2022/081521

§ 371 Date: Jun. 11, 2024

Related U.S. Application Data
(60) Provisional application No. 63/289,848, filed on Dec. 15, 2021.

(51) Int. Cl. C07D 409/12; A61K 31/381; A61K 31/397; A61K 31/4155; A61K 31/422; A61K 31/424; A61K 31/4245; A61K 31/4535; A61K 31/506; A61K 31/5377; A61K 31/541; A61K 31/55; A61P 9/04; C07D 333/70; C07D 409/14; C07D 413/12; C07D 417/12; C07D 498/04

(52) U.S. Cl. CPC C07D 409/12; A61K 31/381; A61K 31/397; A61K 31/4155; A61K 31/422; A61K 31/424; A61K 31/4245; A61K 31/4535; A61K 31/506; A61K 31/5377; A61K 31/541; A61K 31/55; A61P 9/04; C07D 333/70; C07D 409/14; C07D 413/12; C07D 417/12; C07D 498/04

(57) ABSTRACT

The disclosure relates to compounds of Formula (I), which are RXFP1 agonists, compositions containing them, and methods of using them, for example, in the treatment of heart failure, fibrotic diseases, and related diseases such as lung disease (e.g., idiopathic pulmonary fibrosis), kidney disease (e.g., chronic kidney disease), or hepatic disease (e.g., non-alcoholic steatohepatitis and portal hypertension). Variables X¹, X², R¹, R², and R⁴ are as defined in the disclosure.

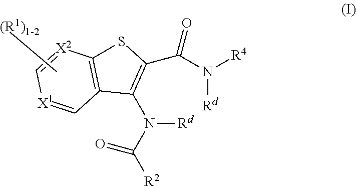

### CROSS-REFERENCE TO RELATED APPLICATIONS

This application claims the benefit of U.S. Provisional Application No. 63/289,848, filed Dec. 15, 2021, the disclosure of which is incorporated herein by reference in its entirety.

### BACKGROUND OF THE INVENTION

The present disclosure relates to novel compounds which are relaxin family peptide receptor 1 (RXFP1) agonists, compositions containing them, and methods of using them, for example in the treatment of heart failure, fibrotic diseases, and related diseases such as lung disease (e.g., idiopathic pulmonary fibrosis), kidney disease (e.g., chronic kidney disease), and hepatic disease (e.g., non-alcoholic steatohepatitis and portal hypertension).

The human relaxin hormone (also called relaxin or H2 relaxin) is a 6-kDa peptide composed of 53 amino acids whose activity was initially discovered when Frederick Hisaw in 1926 injected crude extracts from swine corpus luteum into virgin guinea pigs and observed a relaxation of the fibrocartilaginous pubic symphysis joint (Hisaw F L., Proc. Soc. Exp. Biol. Med., 1926, 23, 661-663). The relaxin receptor was previously known as Lgr7 but is now officially termed the relaxin family peptide receptor 1 (RXFP1) and was deorphanized as a receptor for relaxin in 2002 (Hsu S Y., et al., Science, 2002, 295, 671-674). RXFP1 is reasonably well conserved between mouse and human with 85% amino acid identity and is essentially ubiquitously expressed in humans and in other species (Halls M L., et al., Br. J Pharmacol., 2007, 150, 677-691). The cell signaling pathways for relaxin and RXFP1 are cell type dependent and quite complex (Halls M L., et al., Br. J Pharmacol., 2007, 150, 677-691; Halls M L., et al. Ann. N Y Acad. Sci., 2009, 1160, 108-111; Halls M L., Ann N Y Acad. Sci., 2007, 1160, 117-120). The best studied pathway is the relaxin-dependent increase in cellular levels of cAMP in which relaxin functions as an RXFP1 agonist to promote GaS coupling and activation of adenylate cyclase (Halls M L., et al., Mol. Pharmacol., 2006, 70, 214-226).

Since the initial discovery of relaxin much experimental work has focused on delineating the role relaxin has played in female reproductive biology and the physiological changes that occur during mammalian pregnancy (Sherwood O D., Endocr. Rev., 2004, 25, 205-234). During human gestation, in order to meet the nutritional demands imposed upon it by the fetus, the female body undergoes a significant ~30% decrease in systemic vascular resistance (SVR) and a concomitant ~50% increase in cardiac output (Jeyabalan A C., K. P., Reanl and Electolyte Disorders. 2010, 462-518), (Clapp J F. & Capeless E., Am. J. Cardio., 1997, 80, 1469-1473). Additional vascular adaptations include an ~30% increase in global arterial compliance that is important for maintaining efficient ventricular-arterial coupling, as well as an ~50% increase in both renal blood flow (RBF) and glomerular filtration rate (GFR), important for metabolic waste elimination (Jeyabalan A C., K. P., Reanl and Electolyte Disorders. 2010, 462-518), (Poppas A., et al., Circ., 1997, 95, 2407-2415). Both pre-clinical studies in rodents as well as clinical studies performed in a variety of patient settings, provide evidence that relaxin is involved, at least to some extent, in mediating these adaptive physiological changes (Conrad K P., Regul. Integr. Comp. Physiol., 2011, 301, R267-275), (Teichman S L., et al., Heart Fail. Rev., 2009, 14, 321-329). Importantly, many of these adaptive responses would likely be of benefit to HF patients in that excessive fibrosis, poor arterial compliance, and poor renal function are all characteristics common to heart failure patients (Mohammed S F., et al., Circ., 2015, 131, 550-559), (Wohlfahrt P., et al., Eur. J. Heart Fail., 2015, 17, 27-34), (Damman K., et al., Prog. Cardiovasc. Dis., 2011, 54, 144-153).

Heart failure (HF), defined hemodynamically as “systemic perfusion inadequate to meet the body's metabolic demands as a result of impaired cardiac pump function”, represents a tremendous burden on today's health care system with an estimated United States prevalence of 5.8 million and greater than 23 million worldwide (Roger V L., et al., Circ. Res., 2013, 113, 646-659). It is estimated that by 2030, an additional 3 million people in the United States alone will have HF, a 25% increase from 2010. The estimated direct costs (2008 dollars) associated with HF for 2010 was $25 billion, projected to grow to $78 B by 2030 (Heidenreich P A., et al., Circ., 2011, 123, 933-944). Astoundingly, in the United States, 1 in 9 deaths has HF mentioned on the death certificate (Roger V L., et al., Circ., 2012, 125, e2-220) and, while survival after HF diagnosis has improved over time (Matsushita K., et al., Diabetes, 2010, 59, 2020-2026), (Roger V L., et al., JAMA, 2004, 292, 344-350), the death rate remains high with ~50% of people with HF dying within 5 years of diagnosis (Roger V L., et al., Circ., 2012, 125, e2-220), (Roger V L., et al., JAMA, 2004, 292, 344-350).

The symptoms of HF are the result of inadequate cardiac output and can be quite debilitating depending upon the advanced stage of the disease. Major symptoms and signs of HF include: 1) dyspnea (difficulty in breathing) resulting from pulmonary edema due to ineffective forward flow from the left ventricle and increased pressure in the pulmonary capillary bed; 2) lower extremity edema occurs when the right ventricle is unable to accommodate systemic venous return; and 3) fatigue due to the failing heart's inability to sustain sufficient cardiac output (CO) to meet the body's metabolic needs (Kemp C D., & Conte J V., Cardiovasc. Pathol., 2011, 21, 365-371). Also, related to the severity of symptoms, HF patients are often described as “compensated” or “decompensated”. In compensated heart failure, symptoms are stable, and many overt features of fluid retention and pulmonary edema are absent. Decompensated heart failure refers to a deterioration, which may present as an acute episode of pulmonary edema, a reduction in exercise tolerance, and increasing breathlessness upon exertion (Millane T., et al., BMJ, 2000, 320, 559-562).

In contrast to the simplistic definition of poor cardiac performance not being able to meet metabolic demands, the large number of contributory diseases, multitude of risk factors, and the many pathological changes that ultimately lead to heart failure make this disease exceedingly complex (Jessup M. & Brozena S., N. Engl. J. Med., 2003, 348, 3007-2018). Injurious events thought to be involved in the pathophysiology of HF range from the very acute such as myocardial infarction to a more chronic insult such as life-long hypertension. Historically, HF was primarily described as “systolic HF” in which decreased left-ventricular (LV) contractile function limits the expulsion of blood and hence results in a reduced ejection fraction (EF is stroke volume/end diastolic volume), or “diastolic HF” in which active relaxation is decreased and passive stiffness is increased limiting LV filling during diastole, however overall EF is maintained (Borlaug B A. & Paulus W J., Eur Heart J, 2011, 32, 670-679). More recently, as it became understood that diastolic and systolic LV dysfunction was not uniquely specific to these two groups, new terminology was employed: “heart failure with reduced ejection fraction” (HFrEF), and “heart failure with preserved ejection fraction” (HFpEF) (Borlaug B A. & Paulus W J., Eur Heart J., 2011, 32, 670-679). Although these two patient populations have very similar signs and symptoms, whether HFrEF and HFpEF represent two distinct forms of HF or two extremes of a single spectrum sharing a common pathogenesis is currently under debate within the cardiovascular community (Borlaug B A. & Redfield M M., Circ., 2011, 123, 2006-2013), (De Keulenaer G W., & Brutsaert D L., Circ., 2011, 123, 1996-2004).

Serelaxin, an intravenous (IV) formulation of the recombinant human relaxin peptide with a relatively short first-phase pharmacokinetic half-life of 0.09 hours, is currently being developed for the treatment of HF (Novartis, 2014). Serelaxin has been given to normal healthy volunteers (NHV) and demonstrated to increase RBF (Smith M C., et al., J Am. Soc. Nephrol. 2006, 17, 3192-3197) and estimated GFR (Dahlke M., et al., J Clin. Pharmacol., 2015, 55, 415-422). Increases in RBF were also observed in stable compensated HF patients (Voors A A., et al., Cir. Heart Fail., 2014, 7, 994-1002). In large clinical studies, favorable changes in worsening renal function, worsening HF, as well as fewer deaths, were observed in acute decompensated HF (ADHF) patients in response to an in-hospital 48 hour IV infusion of serelaxin (Teerlink J R., et al., Lancet, 2013, 381, 29-39), (Ponikowski P., et al., Eur. Heart, 2014, 35, 431-441). Suggesting that chronic dosing of serelaxin could provide sustained benefit to HF patients, improvement in renal function based on serum creatinine levels was observed in scleroderma patients given serelaxin continuously for 6 months using a subcutaneous pump (Teichman S L., et al., Heart Fail. Rev., 2009, 14, 321-329). In addition to its potential as a therapeutic agent for the treatment of HF, continuous subcutaneous administration of relaxin has also been demonstrated to be efficacious in a variety of animal models of lung (Unemori E N., et al., J. Clin. Invet., 1996, 98, 2739-2745), kidney (Garber S L., et al., Kidney Int., 2001, 59, 876-882), and liver injury (Bennett R G., Liver Int., 2014, 34, 416-426).

In summary, a large body of evidence supports a role for relaxin-dependent agonism of RXFP1 mediating the adaptive changes that occur during mammalian pregnancy, and that these changes translate into favorable physiological effects and outcomes when relaxin is given to HF patients. Additional preclinical animal studies in various disease models of lung, kidney, and liver injury provide evidence that relaxin, when chronically administered, has the potential to provide therapeutic benefit for multiple indications in addition to HF. More specifically, chronic relaxin administration could be of benefit to patients suffering from lung disease (e.g., idiopathic pulmonary fibrosis), kidney disease (e.g., chronic kidney disease), or hepatic disease (e.g., non-alcoholic steatohepatitis and portal hypertension).

### SUMMARY OF THE INVENTION

The present invention provides novel benzothiophene analogs, including stereoisomers, tautomers, pharmaceutically acceptable salts, or solvates thereof, which are useful as RXFP1 receptor agonists.

The present invention also provides processes and intermediates for making the compounds of the present invention.

The present invention also provides pharmaceutical compositions comprising a pharmaceutically acceptable carrier and at least one of the compounds of the present invention or stereoisomers, tautomers, pharmaceutically acceptable salts, or solvates thereof.

The compounds of the invention may be used, for example, in the treatment and/or prophylaxis of heart failure, fibrotic diseases, and related diseases, such as; lung disease (e.g., idiopathic pulmonary fibrosis), kidney disease (e.g., chronic kidney disease), or hepatic disease (e.g., non-alcoholic steatohepatitis and portal hypertension).

The compounds of the present invention may be used in therapy.

The compounds of the present invention may be used for the manufacture of a medicament for the treatment and/or prophylaxis of heart failure.

The compounds of the invention can be used alone, in combination with other compounds of the present invention, or in combination with one or more, preferably one to two other agent(s).

These and other features of the invention will be set forth in expanded form as the disclosure continues.

### DESCRIPTION OF THE INVENTION

The invention encompasses compounds of Formula (I), which are RXFP1 receptor agonists, compositions containing them, and methods of using them.

In a first aspect, the present invention provides, inter alia, compounds of Formula (I):

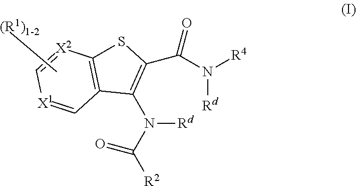

or pharmaceutically acceptable salts thereof, wherein:
X¹ and X² are each N or CR¹; provided X¹ and X² are not both N;
R¹ is H, halo, C₁₋₄ alkyl substituted with 0-5 halo, or C₃₋₆ cycloalkyl;
R² is phenyl substituted with 1-3 R³ and 1 R⁵, or a 5 to 6-membered heteroaryl comprising 1-4 heteroatoms selected from O, S(═O)ₚ, N, and NR^{2a} and substituted with 0-3 R³ and 0-1 R⁵;
R^{2a} is H or C₁₋₃ alkyl substituted with 0-2 halo or —OH;
R³ is halo, CN, OH, C₁₋₄ alkyl, or —OC₁₋₄ alkyl substituted with 0-5 halo, OH, —OC₁₋₄ alkyl, aryl, or heterocyclyl; R⁴ is C₁₋₆ alkyl substituted with 0-5 halo, CN, OH, or OC₁₋₃ alkyl, —(CR^{d}R^{d})₀₋₁—C₃₋₁₀-cycloalkyl substituted with 0-2 R^{4a} or 0-2 R^{4b}, phenyl substituted with 0-2 R^{4a} or 0-2 R^{4b}, —(CR^{d}R^{d})ₙ-3 to 12-membered heterocyclyl comprising 1-4 heteroatoms selected from O, S(═O)ₚ, N, NH, and NC₁₋₃ alkyl, and substituted with 0-2 R^{4a} or 0-2 R^{4b};
R^{4a} or R^{4b} is halo, CN, or C₁₋₄ alkyl substituted with 0-5 halo, OH, or —OC₁₋₄ alkyl substituted with 0-5 halo;
R⁵ is —NR^{5a}R^{5a}, —(CH₂)₁₋₂—NR^{5b}R^{5b}, —C(═O)NR^{5b}R^{5b}, —S(═O)ₚNR^{5b}R^{5b}, C₃₋₆ alkyl substituted with 0-2 OH, C₂-s alkenyl substituted with 0-3 R⁶ and 0-2 R⁷, C₂₋₈ alkynyl substituted with 0-3 R⁶ and 0-2 R⁷, C₃₋₁₂ carbocyclyl substituted with 0-3 R⁶ and 0-2 R⁷, or a 3- to 12-membered heterocyclyl comprising 1-4 heteroatoms selected from O, S(═O)ₚ, N and NR¹⁰ and substituted with 0-3 R⁶ and 0-1 R⁷;
R^{5a} and R^{5a} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R^{5b} is H or C₁₋₆ alkyl substituted with 0-3 R⁶ and 0-2 R⁷; or R^{5b} and R^{5b} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R⁶ is halo, CN, ═O, —OH, —OC₁₋₄ alkyl, or C₁₋₄ alkyl substituted with 0-2 halo or OH;
R⁷ is C₁₋₆ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, —C(═O)NR^{a}S(═O)ₚR^{c}, C₃₋₆ carbocyclyl substituted with 0-5 R^{e}, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NR^{a}R^{a}, —C(═O)NR^{a}OR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —NR^{a}S(═O)ₚNR^{a}R^{a}, —OC(═O)NR^{a}R^{a}, —OC(═O)NR^{a}OR^{b}, —S(═O)ₚNR^{a}R^{a}, —S(═O)ₚR^{c}, or —OP(═O)(OH)₂, —(CH₂)ₙ—C₃₋₆ carbocyclyl substituted with 0-3 R^{e},
   or —(CH₂)ₙ-heterocyclyl comprising 1-4 heteroatoms selected from O, S(═O)ₚ, and N, and substituted with 0-3 R^{e};
R¹⁰ is H, C₁₋₄ alkyl substituted with 0-2 R¹¹, —C(═O)R^{b}, —C(═O)OR^{b}, —C(═O)NR^{a}R^{a}, —C(═O)C(═O)OR^{b}, —S(═O)ₚR^{e}, C₃₋₆ carbocyclyl substituted with 0-5 R^{e}, or a 4- to 6-membered heterocyclyl comprising 1-4 heteroatoms selected from O, S(═O)ₚ, N and NR¹² and substituted with 0-5 R^{e};
R¹¹ is —OR, —C(═O)OR^{b}, —C(═O)NR^{a}R^{a}, —S(═O)ₚR^{e}, or aryl;
R¹² is H, C₁₋₄ alkyl, or aryl;
R^{a} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₆ alkyl substituted with 0-5 R^{e}, C₂₋₆ alkenyl substituted with 0-5 R^{e}, C₂₋₆ alkynyl substituted with 0-5 R^{e}, C₃₋₆ carbocyclyl, or heterocyclyl;
R^{d} is H or C₁₋₄ alkyl;
R^{e} is halo, CN, NO₂, ═O, C₁₋₆ alkyl substituted with 0-5 R⁹, C₂₋₆ alkenyl substituted with 0-5 R⁹, C₂₋₆ alkynyl substituted with 0-5 R^{g}, —(CH₂)ₙ-carbocyclyl substituted with 0-5 R^{g}, —(CH₂)ₙ-heterocyclyl substituted with 0-5 R⁹, —(CH₂)ₙOR^{f}, —C(═O)OR^{f}, —C(═O)NR^{f}R^{f}, —NR^{f}C(═O)R^{f}, —S(═O)ₚR^{f}, —S(═O)ₚNR^{f}R^{f}, —NR^{f}S(═O)ₚR^{f}, —NR^{f}C(═O)OR^{f}, —OC(═O)NR^{f}R^{f}, or —(CH₂)ₙNR^{f}R^{f};
R^{f} is H, C₁₋₆ alkyl substituted with 0-1 —OC₁₋₄ alkyl, C₃₋₆ cycloalkyl, aryl, or heterocyclyl; or R^{f} and R^{f} together with the nitrogen atom to which they are both attached form a heterocyclyl;
R^{g} is halo, CN, OH, S(═O)ₚC₁₋₃ alkyl, C₁₋₆ alkyl, C₃₋₆ cycloalkyl, or aryl;
n is zero, 1, 2, or 3; and
p is zero, 1, or 2.

In a second aspect within the scope of the first aspect, the present invention provides compounds of Formula (II):

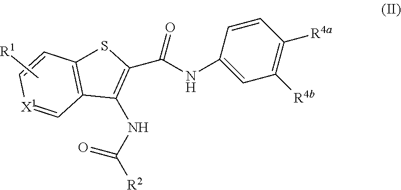

or pharmaceutically acceptable salts thereof, wherein:
X¹ is N or CR¹;
R¹ is H, halo or C₁₋₃ alkyl substituted with 0-4 halo;
R² is

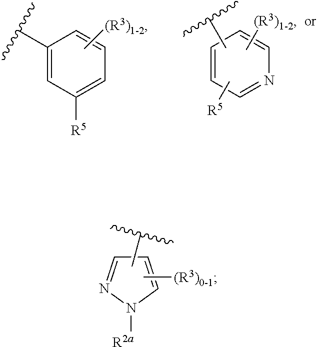

R^{2a} is C₁₋₃ alkyl substituted with 0-1 —OH;
R³ is halo, C₁₋₃ alkyl, or —OC₁₋₄ alkyl substituted with 0-4 halo;
R^{4a} is halo;
R^{4b} is C₁₋₄ alkyl substituted with 0-4 halo;
R⁵ is —NR^{5a}R^{5a}, —C(═O)NR^{5b}R^{5b}, C₂₋₆ alkenyl substituted with 0-3 R⁶ and 0-2 R⁷, C₂₋₆ alkynyl substituted with 0-3 R⁶ and 0-2 R⁷, C₃₋₆ cycloalkyl substituted with 0-3 R⁶ and 0-2 R⁷, phenyl substituted with 0-3 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R^{5a} and R^{5a} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R^{5b} is H or C₁₋₅ alkyl substituted with 0-1 R⁶ and 0-1 R⁷; or R^{5b} and R^{5b} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R⁶ is halo, CN, ═O, —OH, —OC₁₋₃ alkyl, or C₁₋₃ alkyl substituted with 0-2 halo or OH;
R⁷ is C₁₋₅ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(═O)ₚNR^{a}R^{a}, —OC(═O)NR^{a}R^{a}, —OC(═O)NR^{a}OR^{b}, —S(═O)ₚNR^{a}R^{a}, —S(═O)ₚR^{c}, or —OP(═O)(OH)₂;
R¹⁰ is H, C₁₋₄ alkyl substituted with 0-2 R¹¹, —C(═O)R^{b}, —C(═O)OR^{b}, —C(═O)NR^{a}R^{a}, —C(═O)C(═O)OR^{b}, —S(═O)R^{e} C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OR^{b}, —C(═O)NR^{a}R^{a}, or —S(═O)₂R^{c};
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₃ alkyl;
R^{e} is halo, CN, ═O, C₁₋₆ alkyl substituted with 0-5 R⁹, C₂₋₆ alkenyl substituted with 0-5 R⁹, C₂₋₆ alkynyl substituted with 0-5 R^{g}, —(CH₂)ₙ—C₃₋₆ cycloalkyl, —(CH₂)ₙ—C₆ aryl, —(CH₂)ₙ-heterocyclyl, —(CH₂)ₙOR^{f}, —C(═O)OR^{f}, or —S(═O)ₚR^{f};
R^{f} is H or C₁₋₃ alkyl substituted with 0-1 —OC₁₋₄ alkyl;
R^{g} is halo, CN, OH, C₁₋₆ alkyl, C₃₋₆ cycloalkyl, or aryl;
n is zero, 1, 2, or 3; and
p is zero, 1, or 2.

In a third aspect within the scope of the first and second aspects, the present invention provides compounds of Formula (III):

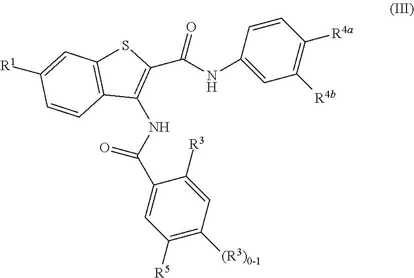

or pharmaceutically acceptable salts thereof, wherein:
R¹ is C₁₋₃ alkyl substituted with 0-3 halo;
R³ is halo C₁₋₂ alkyl, or —OC₁₋₄ alkyl;
R^{4a} is halo;
R^{4b} is C₁₋₃ alkyl substituted with 0-4 F;
R⁵ is —NR^{5a}R^{5a}, —C(═O)NR^{5b}R^{5b}, C₂₋₆ alkenyl substituted with 0-2 R⁶ and 0-2 R⁷, C₂₋₆ alkynyl substituted with 0-2 R⁶ and 0-2 R⁷, C₃₋₆ cycloalkyl substituted with 0-2 R⁶ and 0-2 R⁷, phenyl substituted with 0-2 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R^{5a} and R^{5a} together with the nitrogen atom to which they are both attached form a heterocyclyl comprising additional 0-5 heteroatoms selected from O, S(═O)ₚ, N, and NR¹⁰, and substituted with 0-2 R⁶ and 0-2 R⁷;
R^{5b} is H or C₁₋₅ alkyl substituted with 0-1 R⁶ and 0-1 R⁷; or R^{5b} and R^{5b} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R⁶ is halo, CN, ═O, —OH, —OC₁₋₃ alkyl, or C₁₋₃ alkyl;
R⁷ is C₁₋₅ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, —C(═O)NHOR^{b}, or C₁₋₃ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(═O)ₚNR^{a}R^{a}, —OC(═O)NR^{a}R^{a}, —OC(═O)NR^{a}OR^{b}, —S(═O)ₚNR^{a}R^{a}, or —S(═O)ₚR^{c};
R¹⁰ is H, C₁₋₄ alkyl substituted with 0-2 R¹¹, —C(═O)R^{b}, —C(═O)OR^{b}, —C(═O)C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OR^{b}, —C(═O)NR^{a}R^{a}, or —S(═O)₂C₁₋₄ alkyl;
R¹² is H or C₁₋₃ alkyl;
R^{a} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₄ alkyl substituted with 0-4 R^{e}, C₂₋₄ alkenyl substituted with 0-4 R^{e}, C₂₋₄ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}.
R^{c} is C₁₋₅ alkyl substituted with 0-4 R^{e} or C₃₋₆ carbocyclyl;
R^{d} is H or C₁₋₂ alkyl;
R^{e} is halo, CN, ═O, C₁₋₅ alkyl substituted with 0-5 R^{g}, —(CH₂)ₙ—C₃₋₆ cycloalkyl, —(CH₂)ₙ-heterocyclyl, —(CH₂)ₙ—C₆ aryl, —(CH₂)ₙ-heteroaryl, —(CH₂)ⁿOR^{f}, —C(═O)OR^{f}, or —S(═O)ₚR^{f};
R^{f} is H or C₁₋₃ alkyl; and
R^{g} is halo, CN, OH, C₁₋₅ alkyl, or C₃₋₆ cycloalkyl.

In a fourth aspect within the scope of the third aspect, the present invention provides compounds of Formula (III), or pharmaceutically acceptable salts thereof, wherein:
R¹ is C₁₋₂ alkyl substituted with 0-3 halo;
R³ is —OC₁₋₃ alkyl;
R^{4a} is halo;
R^{4b} is C₁₋₂ alkyl substituted with 0-4 F;
R⁵ is

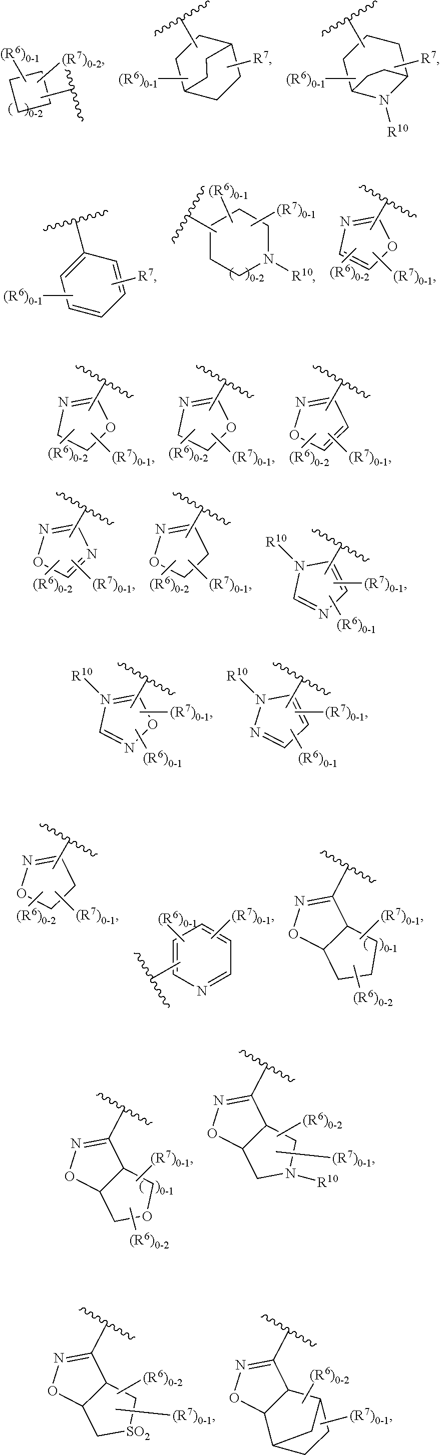

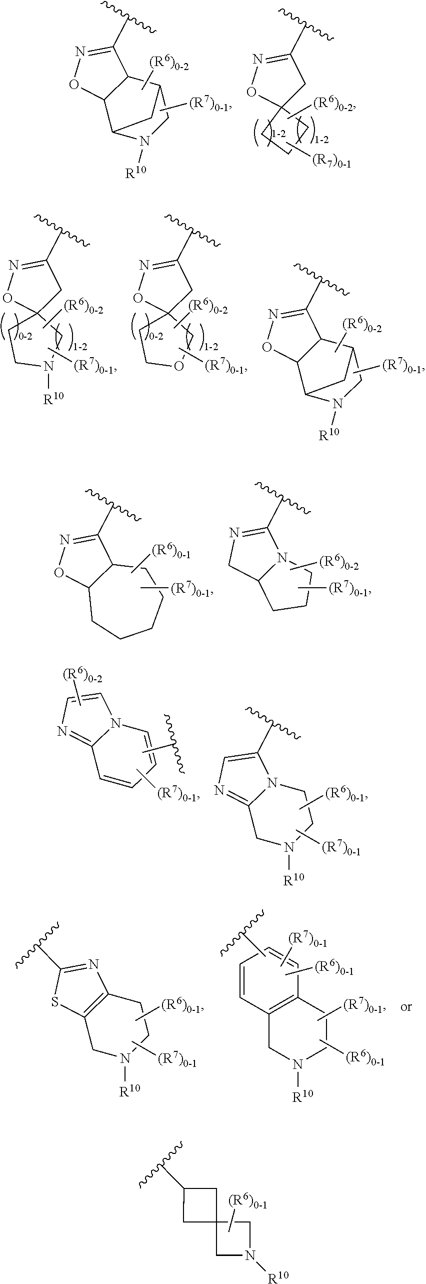

R⁶ is halo, ═O, —OH, or —OC₁₋₂ alkyl, or C₁₋₂ alkyl;
R⁷ is C₁₋₅ alkyl substituted with 0-1 R⁸ and 0-1 R⁹, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —S(═O)ₚR^{c}, —S(═O)ₚNR^{a}R^{a}, or C₃₋₆ cycloalkyl substituted with 0-2 R^{e};
R⁸ is halo, —C(═O)OR^{b} or C₁₋₃ alkyl substituted with 0-3 halo;
R⁹ is —OR^{b}, —NR^{a}R^{a}, —NHC(═O)R^{b}, —NHC(═O)OR^{b}, —NHS(═O)ₚR^{c}, —NHS(═O)ₚNR^{a}R^{a}, —OC(═O)NR^{a}R^{a}, —OC(═O)NR^{a}OR^{b}, —S(═O)ₚNR^{a}R^{a}, or —S(═O)ₚR^{c};
R¹⁰ is H, C₁₋₃ alkyl substituted with 0-2 R¹¹, —C(═O)R^{b}, —C(═O)OR^{b}, —C(═O)C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-4 R^{e};
R¹¹ is —OH, —C(═O)OH, —C(═O)OC₁₋₄ alkyl, or C(═O)NR^{a}R^{a};
R¹² is H or C₁₋₂ alkyl;
R^{a} is H, C₁₋₄ alkyl substituted with 0-4 R^{e}, —(CH₂)₀₋₁-phenyl substituted with 0-4 R^{e}, C₃₋₆ cycloalkyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₃ alkyl substituted with 0-4 R^{e}, C₂₋₃ alkenyl substituted with 0-4 R^{e}, C₂₋₃ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₅ alkyl;
R^{e} is halo, CN, ═O, C₁₋₅ alkyl substituted with 0-5 R^{g}, —(CH₂)ₙ—C₃₋₆ cycloalkyl, —(CH₂)ₙ-heterocyclyl, —(CH₂)ₙ—C₆ aryl, —(CH₂)ₙ-heteroaryl, —(CH₂)ₙOR^{f}, —C(═O)OR^{f}, or —S(═O)ₚR^{f};
R^{f} is H or C₁₋₂ alkyl; and
R^{g} is halo, CN, OH, or C₁₋₅ alkyl.

In a fifth aspect within the scope of the fourth aspect, the present invention provides compounds of Formula (IV):

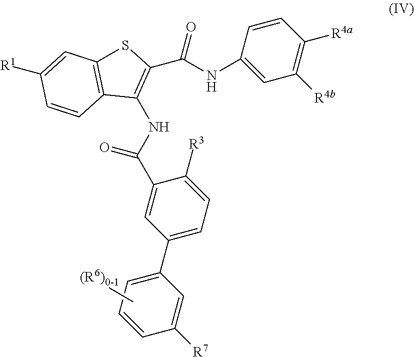

or pharmaceutically acceptable salts thereof, wherein:
R¹ is C₁₋₂ alkyl substituted with 0-3 halo;
R³ is —OC₁₋₃ alkyl;
R^{4a} is halo;
R^{4b} is C₁₋₂ alkyl substituted with 0-3 halo;
R⁶ is halo or C₁₋₂ alkyl;
R⁷ is C₁₋₂ alkyl substituted with 0-1 R⁸ and 0-1 R⁹, —NR^{a}C(═O)OR^{b}, —NR^{a}S(═O)ₚR^{c}, —C(═O)R^{b}, —C(═O)OR^{b}, —C(═O)NR^{a}R^{a}, —S(═O)ₚR^{c}, —S(═O)ₚNR^{a}R^{a}, or C₃₋₆ cycloalkyl substituted with 0-2 R^{e};
R⁸ is halo, —C(═O)OR^{b} or C₁₋₂ alkyl substituted with 0-3 halo;
R⁹ is —OR^{b}, —NR^{a}R^{a}, —NHC(═O)R^{b}, —NHC(═O)OR^{b}, —NHS(═O)ₚR^{c}, —NHS(═O)ₚNR^{a}R^{a}, —OC(═O)NR^{a}R^{a}, —S(═O)ₚNR^{a}R^{a}, or —S(═O)ₚR^{c};
R^{a} is H, C₁₋₃ alkyl substituted with 0-4 R^{e}, —(CH₂)₀₋₁-phenyl substituted with 0-4 R^{e}, C₃₋₆ cycloalkyl substituted with 0-4 R^{e} or heterocyclyl substituted with 0-4 R^{e}; or R^{a} and R^{a} together with the nitrogen atom to which they are both attached form a heterocyclyl substituted with 0-4 R^{e};
R^{b} is H, C₁₋₃ alkyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₄ alkyl;
R^{e} is halo, CN, ═O, C₁₋₄ alkyl substituted with 0-5 R^{g}, —(CH₂)ₙ—C₃₋₆ cycloalkyl, —(CH₂)ₙ-heterocyclyl, —(CH₂)ₙ—C₆ aryl, —(CH₂)ₙ-heteroaryl, —(CH₂)ₙOR^{f}, or —C(═O)OR^{f};
R^{f} is H or C₁₋₂ alkyl; and
R^{g} is halo, CN, OH, C₁₋₄ alkyl.

In a sixth aspect within the scope of the first aspect, the present invention provides compounds of Formula (V):

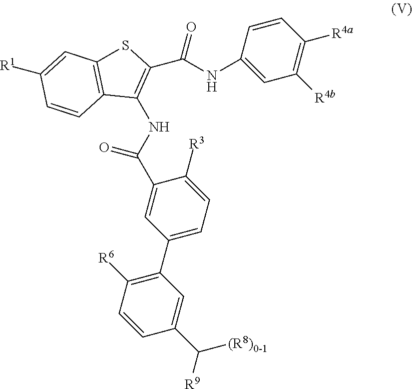

or pharmaceutically acceptable salts thereof, wherein:
R¹ is CF₃;
R³ is —OC₁₋₂ alkyl;
R^{4a} is F;
R^{4b} is CF₃;
R⁶ is halo;
R⁸ is —C(═O)OR^{b} or —CF₃;
R⁹ is —OR^{b}, —NR^{a}R^{a}, —NHC(═O)R^{b}, —NHS(═O)ₚR^{c}, —OC(═O)NR^{a}R^{a}, or —S(═O)₂R^{c};
R^{a} is H, C₁₋₃ alkyl, —(CH₂)₀₋₁-phenyl substituted with 0-2 R^{e}, or C₃₋₆ cycloalkyl; or R^{a} and R^{a} together with the nitrogen atom to which they are both attached form a heterocyclyl substituted with 0-3 R^{e};
R^{b} is H, C₁₋₃ alkyl substituted with 0-2 R^{e}, C₃₋₆ cycloalkyl, or heterocyclyl;
R^{c} is C₁₋₃ alkyl;
R^{e} is —OR^{f}; and
R^{f} is H or C₁₋₂ alkyl.

In a seventh aspect within the scope of the fourth aspect, the present invention provides compounds of Formula (III), or pharmaceutically acceptable salts thereof, wherein:
R¹ is CF₃;
R³ is —OCH₃;
R^{4a} is F;
R^{4b} is CF₃;
R⁵ is

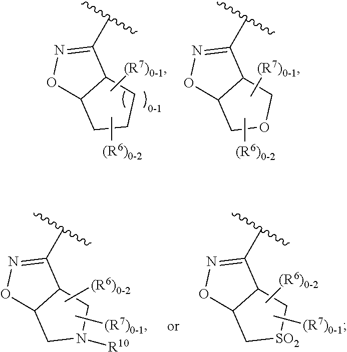

R⁶ is halo, —OH, —OC₁₋₂ alkyl, or C₁₋₂ alkyl;
R⁷ is C₁₋₅ alkyl substituted with 0-1 R⁸ and 0-1 R⁹, —NR^{a}R^{a}, or —NR^{a}C(═O)R^{b};
R⁸ is —C(═O)OR^{b};
R⁹ is OH;
R¹⁰ is H, C₁₋₃ alkyl substituted with 0-2 R¹¹, —C(═O)R^{b}, —C(═O)OR^{b}, —C(═O)NR^{a}R^{a}, or a 4- to 6-membered heterocyclyl comprising 1-4 heteroatoms selected from O, S(═O)ₚ, N and NR¹², and substituted with 0-4 R^{e}.
R¹¹ is —OH, —C(═O)OH, or C(═O)NR^{a}R^{a};
R¹² is H and C₁₋₂ alkyl;
R^{a} is H or C₁₋₃ alkyl;
R^{b} is H or C₁₋₃ alkyl substituted with 0-1 R^{e}; and
R^{e} is OH.

In an eighth aspect within the scope of the seventh aspect, the present invention provides compounds of Formula (V) or pharmaceutically acceptable salts thereof, wherein:
R⁵ is

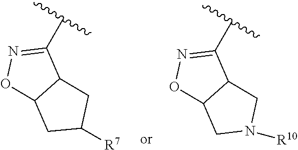

R¹⁰ is —C(═O)R^{b};
R^{b} is H or C₁₋₃ alkyl substituted with 0-1 R^{e}; and
R^{e} is OH.

In a ninth aspect within the scope of the fourth aspect, the present invention provides compounds of Formula (III) or pharmaceutically acceptable salts thereof, wherein:
R¹ is CF₃;
R³ is —OCH₃;
R^{4a} is F;
R^{4b} is CF₃;
R⁵ is

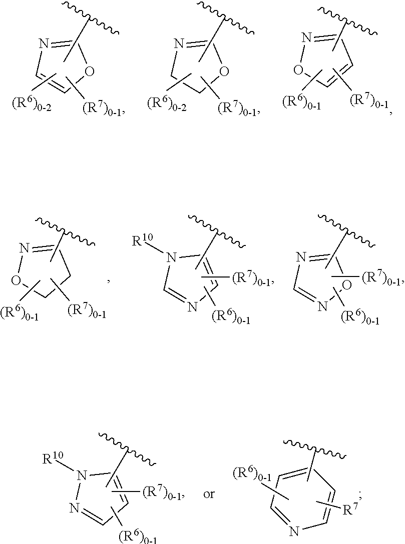

R⁶ is halo, —OH, or C₁₋₂ alkyl;
R⁷ is —NR^{a}R^{a}, —C(═O)NR^{a}R^{a}, or —S(═O)₂NR^{a}R^{a};
R¹⁰ is H, C₁₋₄ alkyl substituted with 0-1 R¹¹, or —C(═O)R^{b};
R¹¹ is —OH or —C(═O)OH;
R^{a} is H or C₁₋₃ alkyl; or R^{a} and R^{a} together with the nitrogen atom to which they are both attached form a heterocyclyl substituted with 0-3 R^{e};
R^{b} is H or C₁₋₃ alkyl;
R^{e} is C₁₋₃ alkyl or —(CH₂)₀₋₁OR^{f}; and
R^{f} is H or C₁₋₃ alkyl.

In a tenth aspect within the scope of the ninth aspect, the present invention provides compounds of Formula (III) or pharmaceutically acceptable salts thereof, wherein:
R⁵ is

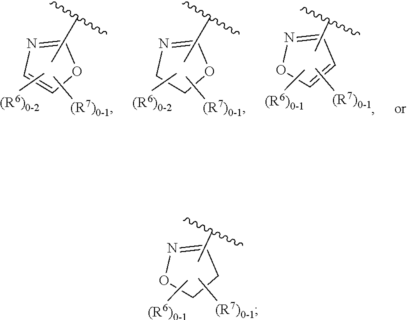

R⁶ is halo, —OH, or C₁₋₂ alkyl;
R⁷ is —NR^{a}R^{a}, —C(═O)NR^{a}R^{a}, or —S(═O)₂NR^{a}R^{a};
R^{a} is H or C₁₋₃ alkyl; or R^{a} and R^{a} together with the nitrogen atom to which they are both attached form a heterocyclyl substituted with 0-2 R^{e};
R^{e} is —(CH₂)₀₋₁OR^{f} and
R^{f} is H or C₁₋₂ alkyl.

In an eleventh aspect within the scope of the third aspect, the present invention provides compounds of Formula (III) or pharmaceutically acceptable salts thereof, wherein:
R¹ is CF₃;
R³ is —OCH₃;
R^{4a} is F;
R^{4b} is CF₃;
R⁵ is —C(═O)NR^{5b}R^{5b};
R^{5b} is H or C₁₋₅ alkyl substituted with 0-1 R⁶ and 0-1 R⁷; or R^{5b} and R^{5b} together with the nitrogen atom to which they are both attached form a heterocyclyl selected from

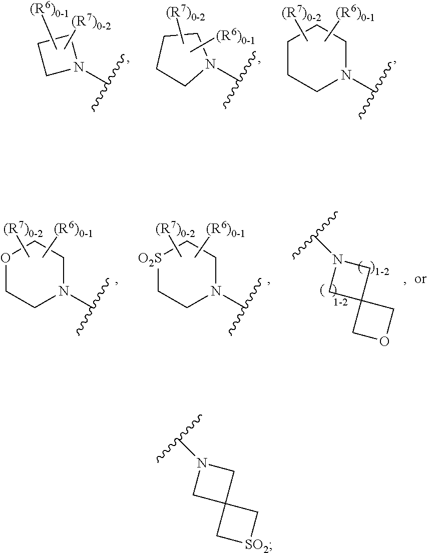

R⁶ is halo, —OH, or C₁₋₃ alkyl;
R⁷ is —S(═O)₂C₁₋₃ alkyl or C₃₋₆ cycloalkyl substituted with 0-2 R^{e};
R^{a} is H or C₁₋₃ alkyl;
R^{b} is H or C₁₋₃ alkyl; and
R^{e} is —S(═O)₂C₁₋₃ alkyl.

In a twelfth aspect within the scope of the eleventh aspect, the present invention provides compounds of Formula (III) or pharmaceutically acceptable salts thereof, wherein:
R^{5b} is H or C₁₋₄ alkyl substituted with 0-1 R⁶ and 0-1 R⁷;
R⁶ is halo, —OH, or C₁₋₄ alkyl substituted with 0-1 OH;
R⁷ is —S(═O)₂C₁₋₂ alkyl or C₃₋₆ cycloalkyl substituted with 0-2 R^{e}; and
R^{e} is —S(═O)₂C₁₋₃ alkyl.

In a thirteenth aspect within the scope of the eleventh aspect, the present invention provides compounds of Formula (III) or pharmaceutically acceptable salts thereof, wherein:
R^{5b} and R^{5b} together with the nitrogen atom to which they are both attached form a heterocyclyl selected from

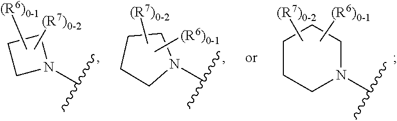

R⁶ is halo, —OH, or C₁₋₃ alkyl;
R⁷ is —S(═O)₂C₁₋₃ alkyl or C₃₋₆ cycloalkyl substituted with 0-2 R^{e};
R^{a} is H or C₁₋₃ alkyl;
R^{b} is H or C₁₋₃ alkyl; and
R^{e} is —S(═O)₂C₁₋₃ alkyl.

In a fourteenth aspect within the scope of the fourth aspect, the present invention provides compounds of Formula (III) or pharmaceutically acceptable salts thereof, wherein:
R¹ is CF₃;
R³ is —OCH₃;
R^{4a} is F;
R^{4b} is CF₃;
R⁵ is

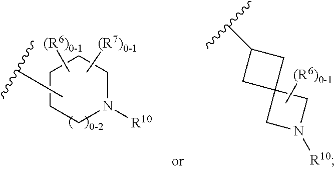

R⁶ is halo, ═O, —OH, —OC₁₋₂ alkyl, or C₁₋₂ alkyl;
R⁷ is C₁₋₂ alkyl substituted with 0-1 R⁸ and 0-1 R⁹, —NR^{a}R^{a}, —NR^{a}C(═O)R^{b}, —NR^{a}C(═O)OR, or —C(═O)OR;
R⁸ is —C(═O)OR^{b}, —C(═O)NHR^{a}, —C(═O)NHOR^{b}, or C₁₋₂ alkyl substituted with 0-3 halo or OH;
R⁹ is —NR^{a}C(═O)R^{b};
R¹⁰ is H, C₁₋₃ alkyl substituted with 0-2 R¹¹, —C(═O)R^{b}, —C(═O)OR^{b}, —C(═O)C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, —C(═O)OC₁₋₄ alkyl, or C(═O)NR^{a}R^{a};
R¹² is H or C₁₋₂ alkyl;
R^{a} is H or C₁₋₃ alkyl;
R^{b} is H, C₁₋₃ alkyl substituted with 0-2 R^{e}, C₃₋₆ cycloalkyl substituted with 0-2 R^{e}, or heterocyclyl substituted with 0-2 R^{e};
R^{e} is C₁₋₃ alkyl, OH, or —NR^{f}R^{f}; and
R^{f} is H or C₁₋₃ alkyl.

In a fifteenth aspect within the scope of the fourteenth aspect, the present invention provides compounds of Formula (III) or pharmaceutically acceptable salts thereof, wherein:
R⁵ is

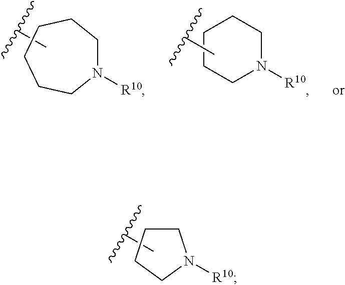

R¹⁰ is H, C₁₋₃ alkyl substituted with 0-2 R¹¹, —C(═O)R^{b}, —C(═O)OR^{b}, —C(═O)C(═O)OR^{b}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, —C(═O)OC₁₋₄ alkyl, or C(═O)NR^{a}R^{a};
R¹² is H or C₁₋₂ alkyl;
R^{a} is H or C₁₋₃ alkyl;
R^{b} is H, or C₁₋₃ alkyl substituted with 0-1 R^{e};
R^{e} is C₁₋₃ alkyl, OH, NR^{f}R^{f}; and
R^{f} is H or C₁₋₃ alkyl.

In a sixteenth aspect within the scope of the third aspect, the present invention provides compounds of Formula (III) or pharmaceutically acceptable salts thereof, wherein:
R¹ is CF₃;
R³ is —OCH₃;
R^{4a} is F;
R^{4b} is CF₃;
R⁵ is —NR^{5a}R^{5a};
R^{5a} and R^{5a} together with the nitrogen atom to which they are both attached form a heterocyclyl selected from

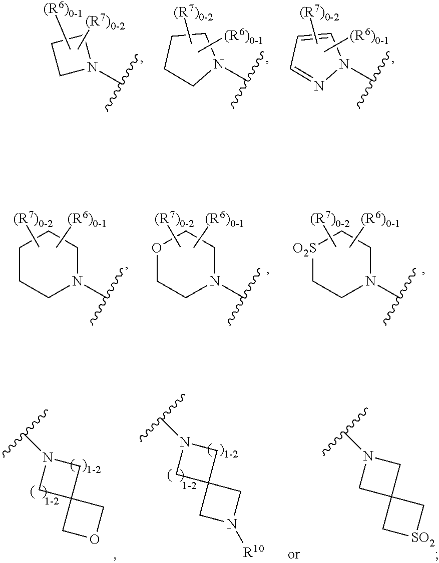

R⁶ is halo, —OH, or C₁₋₃ alkyl;
R⁷ is —S(═O)₂C₁₋₃ alkyl or C₃₋₆ cycloalkyl substituted with 0-2 R^{e};
R¹⁰ is H, C₁₋₄ alkyl substituted with 0-1R¹¹, —C(═O)R^{b}, or —S(═O)₂C₁₋₃ alkyl;
R¹¹ is —OH or —C(═O)OH;
R^{a} is H or C₁₋₃ alkyl;
R^{b} is H or C₁₋₃ alkyl; and
R^{e} is —S(═O)₂C₁₋₃ alkyl.

In a seventeenth aspect within the scope of the fourth aspect, the present invention provides compounds of Formula (III) or pharmaceutically acceptable salts thereof, wherein:

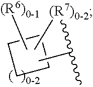

R⁵ is
R⁶ is —OH, or —OC₁₋₂ alkyl, or C₁₋₂ alkyl;
R⁷ is C₁₋₂ alkyl substituted with 0-1 R⁸ and 0-1 R⁹, —NR^{a}R^{a}, —C(═O)R^{b}, —C(═O)OR^{b}, or —C(═O)NR^{a}R^{a};
R⁸ is halo;
R⁹ is —OR^{b};
R^{a} is H, C₁₋₃ alkyl, C₃₋₆ cycloalkyl, or heterocyclyl; or R^{a} and R^{a} together with the nitrogen atom to which they are both attached form a heterocyclyl substituted with 0-3 R^{e};
R^{b} is H, C₁₋₃ alkyl substituted with 0-1 R^{e}, or heterocyclyl;
R^{e} is —OR^{f}; and
R^{f} is H or C₁₋₂ alkyl.

In an eighteenth aspect within the scope of the third aspect, the present invention provides compounds of Formula (III) or pharmaceutically acceptable salts thereof, wherein:
R¹ is CF₃;
R³ is —OC₁₋₂ alkyl;
R^{4a} is F;
R^{4b} is CF₃;
R⁵ is —C(═O)NR^{4b}R^{5b},

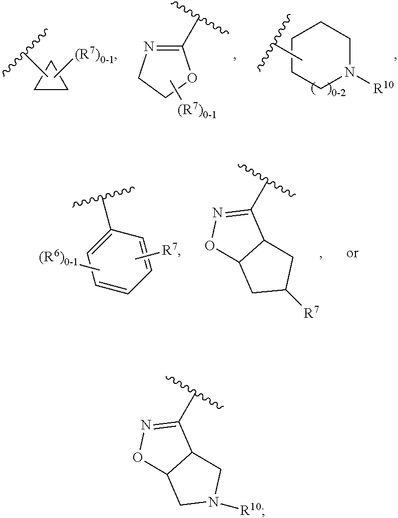

R^{5b} is H or C₁₋₄ alkyl substituted with 0-1 R⁶ and 0-1 R⁷; or R^{5b} and R^{5b} together with the nitrogen atom to which they are both attached form

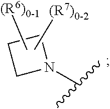

R⁶ is halo, —OH, or C₁₋₃ alkyl;
R⁷ is C₁₋₃ alkyl substituted with 0-1 R⁸ and 0-1 R⁹, —C(═O)NR^{a}R^{a}, —C(═O)OR^{b}, —NR^{a}C(═O)R^{b}, —S(═O)₂NR^{a}R^{a}, —S(═O)₂C₁₋₃ alkyl, or C₃₋₆ cycloalkyl substituted with 0-2 R^{e};
R⁸ is halo, —C(═O)OR^{b}, or C₁₋₃ alkyl substituted with 0-3 halo;
R⁹ is —OH, —NR^{a}R^{a}, —NR^{a}C(═O)R^{b}, NR^{a}S(═O)ₚC₁₋₄ alkyl, or —OC(═O)NR^{a}R^{a};
R¹⁰ is H, C₁₋₄ alkyl substituted with 0-2 R¹¹, —C(═O)R^{b}, —C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-4 R^{e};
R¹¹ is —OH, —C(═O)OH, —C(═O)NR^{a}R^{a}, or —S(═O)₂C₁₋₄ alkyl;
R¹² is H or C₁₋₃ alkyl;
R^{a} is H, C₁₋₃ alkyl, —(CH₂)₀₋₁—C₃₋₆ cycloalkyl, or —(CH₂)₀₋₁-heterocyclyl; or R^{a} and R^{a} together with the nitrogen atom to which they are both attached form a 5- or 6-membered heterocyclyl substituted with 0-2 R^{e};
R^{b} is H, C₁₋₃ alkyl substituted with 0-4 R^{e}, or heterocyclyl;
R^{e} is C₁₋₃ alkyl, —(CH₂)₀₋₁OR, or —S(═O)₂C₁₋₃ alkyl; and
R^{f} is H or C₁₋₃ alkyl.

In one embodiment, the present invention provides compounds of Formula (VI):

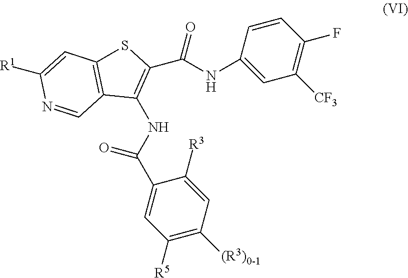

or pharmaceutically acceptable salts thereof, wherein:
R¹ is halo, C₁₋₃ alkyl substituted with 0-3 halo;
R³ is halo or —OC₁₋₄ alkyl;
R⁵ is —NR^{5a}R^{5a}, —C(═O)NR^{5b}R^{5b}, C₂₋₆ alkenyl substituted with 0-2 R⁶ and 0-2 R⁷, C₂₋₆ alkynyl substituted with 0-2 R⁶ and 0-2 R⁷, C₃₋₆ cycloalkyl substituted with 0-2 R⁶ and 0-2 R⁷, phenyl substituted with 0-2 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R^{5a} and R^{5a} together with the nitrogen atom to which they are both attached form a heterocyclyl comprising additional 0-5 heteroatoms selected from O, S(═O)ₚ, N, and NR¹⁰, and substituted with 0-2 R⁶ and 0-2 R⁷;
R^{5b} is H or C₁₋₅ alkyl substituted with 0-1 R⁶ and 0-1 R⁷; or R^{5b} and R^{5b} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R⁶ is halo, CN, ═O, —OH, —OC₁₋₃ alkyl, or C₁₋₃ alkyl;
R⁷ is C₁₋₅ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, —C(═O)NHOR^{b}, or C₁₋₃ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(═O)ₚNR^{a}R^{a}, —OC(═O)NR^{a}R^{a}, —OC(═O)NR^{a}OR^{b}, —S(═O)ₚNR^{a}R^{a}, or —S(═O)ₚR^{c};
R¹⁰ is H, C₁₋₄ alkyl substituted with 0-2 R¹¹, —C(═O)R^{b}, —C(═O)OR^{b}, —C(═O)C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OR^{b}, —C(═O)NR^{a}R^{a}, or —S(═O)₂C₁₋₄ alkyl;
R¹² is H or C₁₋₃ alkyl;
R^{a} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₄ alkyl substituted with 0-4 R^{e}, C₂₋₄ alkenyl substituted with 0-4 R^{e}, C₂₋₄ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}.
R^{c} is C₁₋₅ alkyl substituted with 0-4 R^{e} or C₃₋₆ carbocyclyl;
R^{d} is H or C₁₋₂ alkyl;
R^{e} is halo, CN, ═O, C₁₋₅ alkyl substituted with 0-5 R^{g}, —(CH₂)ₙ—C₃₋₆ cycloalkyl, —(CH₂)ₙ-heterocyclyl, —(CH₂)ₙ—C₆ aryl, —(CH₂)ₙ-heteroaryl, —(CH₂)ⁿOR^{f}, —C(═O)OR^{f}, or —S(═O)ₚR^{f};
R^{f} is H or C₁₋₃ alkyl; and
R^{g} is halo, CN, OH, C₁₋₅ alkyl, or C₃₋₆ cycloalkyl.

In another embodiment, the present invention provides compounds of Formula (VII):

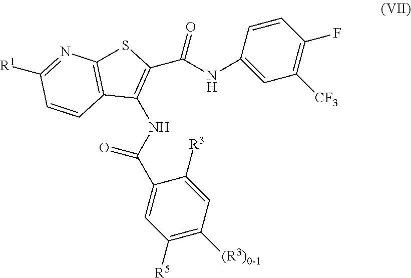

or pharmaceutically acceptable salts thereof, wherein:
R¹ is H, halo, C₁₋₃ alkyl substituted with 0-3 halo;
R³ is halo or —OC₁₋₄ alkyl;
R⁵ is —NR^{5a}R^{5a}, —C(═O)NR^{5b}R^{5b}, C₂₋₆ alkenyl substituted with 0-2 R⁶ and 0-2 R⁷, C₂₋₆ alkynyl substituted with 0-2 R⁶ and 0-2 R⁷, C₃₋₆ cycloalkyl substituted with 0-2 R⁶ and 0-2 R⁷, phenyl substituted with 0-2 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R^{5a} and R^{5a} together with the nitrogen atom to which they are both attached form a heterocyclyl comprising additional 0-5 heteroatoms selected from O, S(═O)ₚ, N, and NR¹⁰, and substituted with 0-2 R⁶ and 0-2 R⁷;
R^{5b} is H or C₁₋₅ alkyl substituted with 0-1 R⁶ and 0-1 R⁷; or R^{5b} and R^{5b} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R⁶ is halo, CN, ═O, —OH, —OC₁₋₃ alkyl, or C₁₋₃ alkyl;
R⁷ is C₁₋₅ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, —C(═O)NHOR^{b}, or C₁₋₃ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(═O)ₚNR^{a}R^{a}, —OC(═O)NR^{a}R^{a}, —OC(═O)NR^{a}OR^{b}, —S(═O)ₚNR^{a}R^{a}, or —S(═O)ₚR^{c};
R¹⁰ is H, C₁₋₄ alkyl substituted with 0-2 R¹¹, —C(═O)R^{b}, —C(═O)OR^{b}, —C(═O)C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OR^{b}, —C(═O)NR^{a}R^{a}, or —S(═O)₂C₁₋₄ alkyl;
R¹² is H or C₁₋₃ alkyl;
R^{a} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₄ alkyl substituted with 0-4 R^{e}, C₂₋₄ alkenyl substituted with 0-4 R^{e}, C₂₋₄ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{e} is C₁₋₅ alkyl substituted with 0-4 R^{e} or C₃₋₆ carbocyclyl;
R^{d} is H or C₁₋₂ alkyl;
R^{e} is halo, CN, ═O, C₁₋₅ alkyl substituted with 0-5 R^{g}, —(CH₂)ₙ—C₃₋₆ cycloalkyl, —(CH₂)ₙ-heterocyclyl, —(CH₂)ₙ—C₆ aryl, —(CH₂)ₙ-heteroaryl, —(CH₂)ₙOR^{f}, —C(═O)OR^{f}, or —S(═O)ₚR^{f};
R^{f} is H or C₁₋₃ alkyl; and
R^{g} is halo, CN, OH, C₁₋₅ alkyl, or C₃₋₆ cycloalkyl.

For a compound of Formula (I), the scope of any instance of a variable substituent, including R¹, R², R^{2a}, R³, R⁴, R^{4a}, R^{4b}, R⁵, R^{5a}, R^{5b}, R⁶, R⁷, R⁸, R⁹, R¹⁰, R¹¹, R¹², R^{a}, R^{b}, R⁰, R^{d}, R^{e}, R^{f}, and R^{g} can be used independently with the scope of any other instance of a variable substituent. As such, the invention includes combinations of the different aspects.

In one embodiment of Formula (I), X¹ and X² are CH; R¹ is CF₃.

In another embodiment of Formula (I), X¹ and X² are CH; R¹ is H.

In another embodiment of Formula (I), X¹ is CR¹; X² is CH; R¹ is CF₃.

In another embodiment of Formula (I), X¹ is N; X² is CH; R¹ is CH₃.

In another embodiment of Formula (I), X¹ is N; X² is CH; R¹ is C₃₋₆ cycloalkyl.

In another embodiment of Formula (I), X¹ is N; X² is CH; R¹ is halo.

In another embodiment of Formula (I), X¹ is N; X² is CH; R¹ is CH₃.

In another embodiment of Formula (I), X¹ is CH; X² is N; R¹ is H.

In another embodiment of Formula (I), X¹ is CH; X² is N; R¹ is CH₃.

In another embodiment of Formula (I), R^{4a} is F.

In another embodiment of Formula (I), R^{4b} is CF₃.

In another embodiment of Formula (III), X¹ is CH; R¹ is CF₃; R³ is F or —OCH₃; R^{4a} is F; R^{4b} is CF₃; R⁵ is —C(═O)NR^{5b}R^{5b}; R^{5b} is H or C₁₋₅ alkyl substituted with 0-1 R⁷; or R^{5b} and R^{5b} together with the nitrogen atom to which they are both attached form

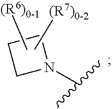

R⁶ is —OH or C₁₋₃ alkyl; R⁷ is C₁₋₃ alkyl substituted with 0-1 R⁹, —S(═O)₂C₁₋₃ alkyl, or C₃₋₆ cycloalkyl substituted with 0-2 R^{e}; R⁹ is —OH; and R^{e} is —S(═O)₂C₁₋₃ alkyl.

In another embodiment of Formula (III), X¹ is CH; R¹ is CF₃; R³ is F or —OCH₃; R^{4a} is F; R^{4b} is CF₃; R⁵ is

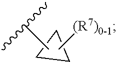

R⁷ is C₁₋₅ alkyl substituted with 0-1 R⁹ or —C(═O)NHR^{a}; R⁹ is —OH; R^{a} is H, C₁₋₃ alkyl, —(CH₂)₀₋₁—C₃₋₆ cycloalkyl, or —(CH₂)₀₋₁-heterocyclyl.

In another embodiment of Formula (III), X¹ is CH; R¹ is CF₃; R³ is F or —OCH₃; R^{4a} is F; R^{4b} is CF₃; R⁵ is

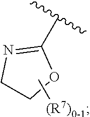

R⁷ is —C(═O)NR^{a}R^{a}; R^{a} and R^{a} together with the nitrogen atom to which they are both attached form

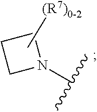

R^{e} is C₁₋₃ alkyl substituted with 0-2 R^{g}; R⁹ is —OH alkyl.

In another embodiment of Formula (III), X¹ is CH; R¹ is CF₃; R³ is F or —OCH₃; R^{4a} is F; R^{4b} is CF₃; R⁵ is

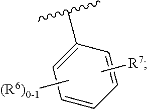

R⁶ is F; R⁷ is —S(═O)₂C₁₋₃ alkyl, —S(═O)₂NHR^{a}, or C₁₋₃ alkyl substituted with 0-1 R⁸ and 0-1 R⁹; R⁸ is —C(═O)OH, or CF₃; R⁹ is —NHR^{a}, —NHC(═O)R^{b}, —NHS(═O)ₚC₁₋₄ alkyl or —OC(═O)NHR^{a}; R^{a} is H, C₁₋₃ alkyl, —(CH₂)₀₋₁—C₃₋₆ cycloalkyl, or —(CH₂)₀₋₁-phenyl substituted with 0-2 R^{e}; R^{b} is H or heterocyclyl; R^{e} is C₁₋₃ alkyl, —(CH₂)₀₋₁OR; and R^{f} is H or C₁₋₃ alkyl.

In another embodiment of Formula (III), X¹ is CH; R¹ is CF₃; R³ is F or —OCH₃; R^{4a} is F; R^{4b} is CF₃; R⁵ is

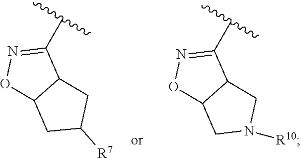

R⁷ is C₁₋₄ alkyl substituted with 0-1 R⁹; R⁹ is —OH; R¹⁰ is —C(═O)R^{b}; R^{b} is H or C₁₋₃ alkyl substituted with 0-4 R^{e}; R^{e} is —(CH₂)₀₋₁OR; and
R^{f} is H or C₁₋₃ alkyl.

Unless specified otherwise, these terms have the following meanings.

“Halo” includes fluoro, chloro, bromo, and iodo.

“Alkyl” or “alkylene” is intended to include both branched and straight-chain saturated aliphatic hydrocarbon groups having the specified number of carbon atoms. For example, “C₁ to C₁₀ alkyl” or “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁ to C₆ alkyl” or “C₁-C₆ alkyl” denotes alkyl having 1 to 6 carbon atoms. Alkyl group can be unsubstituted or substituted with at least one hydrogen being replaced by another chemical group. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl). When “C₀ alkyl” or “C₀ alkylene” is used, it is intended to denote a direct bond. “Alkyl” also includes deuteroalkyl such as CD₃.

“Alkenyl” or “alkenylene” is intended to include hydrocarbon chains of either straight or branched configuration having one or more, preferably one to three, carbon-carbon double bonds that may occur in any stable point along the chain. For example, “C₂ to C₆ alkenyl” or “C₂₋₆ alkenyl” (or alkenylene), is intended to include C₂, C₃, C₄, C₅, and C₆ alkenyl groups; such as ethenyl, propenyl, butenyl, pentenyl, and hexenyl.

“Alkynyl” or “alkynylene” is intended to include hydrocarbon chains of either straight or branched configuration having one or more, preferably one to three, carbon-carbon triple bonds that may occur in any stable point along the chain. For example, “C₂ to C₆ alkynyl” or “C₂₋₆ alkynyl” (or alkynylene), is intended to include C₂, C₃, C₄, C₅, and C₆ alkynyl groups; such as ethynyl, propynyl, butynyl, pentynyl, and hexynyl.

“Carbocycle”, “carbocyclyl”, or “carbocyclic residue” is intended to mean any stable 3-, 4-, 5-, 6-, 7-, or 8-membered monocyclic or bicyclic or 7-, 8-, 9-, 10-, 11-, 12-, or 13-membered bicyclic or tricyclic hydrocarbon ring, any of which may be saturated, partially unsaturated, unsaturated or aromatic. Examples of such carbocyclyls include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocyclyl (e.g., [2.2.2]bicyclooctane). A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. When the term “carbocyclyl” is used, it is intended to include “aryl,” “cycloalkyl,” and “spirocycloalkyl.” Preferred carbocyclyls, unless otherwise specified, are cyclopropyl, cyclobutyl, cyclopentyl, cyclohexyl, phenyl, and indanyl.

“Cycloalkyl” is intended to mean cyclized alkyl groups, including mono-, bi- or multicyclic ring systems. “C₃ to C₇ cycloalkyl” or “C₃₋₇ cycloalkyl” is intended to include C₃, C₄, C₅, C₆, and C₇ cycloalkyl groups. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl.

“Spirocycloalkyl” is intended to mean hydrocarbon bicyclic ring systems with both rings connected through a single atom. The ring can be different in size and nature, or identical in size and nature. Examples include spiropentane, spriohexane, spiroheptane, spirooctane, spirononane, or spirodecane.

“Bicyclic carbocyclyl” or “bicyclic carbocyclic group” is intended to mean a stable 9- or 10-membered carbocyclic ring system that contains two fused rings and consists of carbon atoms. Of the two fused rings, one ring is a benzo ring fused to a second ring; and the second ring is a 5- or 6-membered carbon ring which is saturated, partially unsaturated, or unsaturated. The bicyclic carbocyclic group may be attached to its pendant group at any carbon atom which results in a stable structure. The bicyclic carbocyclic group described herein may be substituted on any carbon if the resulting compound is stable. Examples of a bicyclic carbocyclic group are, but not limited to, naphthyl, 1,2-dihydronaphthyl, 1,2,3,4-tetrahydronaphthyl, and indanyl.

“Aryl” groups refer to monocyclic or polycyclic aromatic hydrocarbons, including, for example, phenyl, naphthyl, and phenanthranyl. Aryl moieties are well known and described, for example, in Lewis, R. J., ed., Hawley's Condensed Chemical Dictionary, 13th Edition, John Wiley & Sons, Inc., New York (1997).

“Benzyl” is intended to mean a methyl group on which one of the hydrogen atoms is replaced by a phenyl group, wherein said phenyl group may optionally be substituted with 1 to 5 groups, preferably 1 to 3 groups.

“Heterocycle”, “heterocyclyl” or “heterocyclic ring” is intended to mean a stable 3-, 4-, 5-, 6-, or 7-membered monocyclic or bicyclic or 7-, 8-, 9-, 10-, 11-, 12-, 13-, or 14-membered polycyclic heterocyclic ring that is saturated, partially unsaturated, or fully unsaturated, and that contains carbon atoms and 1, 2, 3 or 4 heteroatoms independently selected from the group consisting of N, O and S; and including any polycyclic group in which any of the above-defined heterocyclic rings is fused to a benzene ring. The nitrogen and sulfur heteroatoms may optionally be oxidized (i.e., N→O and S(O)ₚ, wherein p is 0, 1 or 2). The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocyclyl may optionally be quaternized. It is preferred that when the total number of S and O atoms in the heterocyclyl exceeds 1, then these heteroatoms are not adjacent to one another. It is preferred that the total number of S and O atoms in the heterocyclyl is not more than 1. Bridged rings are also included in the definition of heterocyclyl. When the term “heterocyclyl” is used, it is intended to include heteroaryl.

Examples of heterocyclyls include, but are not limited to, acridinyl, azetidinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, imidazolopyridinyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinylperimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridinyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocyclyls.

“Bicyclic heterocyclyl” “bicyclic heterocyclyl” or “bicyclic heterocyclic group” is intended to mean a stable 9- or 10-membered heterocyclic ring system which contains two fused rings and consists of carbon atoms and 1, 2, 3, or 4 heteroatoms independently selected from the group consisting of N, O and S. Of the two fused rings, one ring is a 5- or 6-membered monocyclic aromatic ring comprising a 5-membered heteroaryl ring, a 6-membered heteroaryl ring or a benzo ring, each fused to a second ring. The second ring is a 5- or 6-membered monocyclic ring which is saturated, partially unsaturated, or unsaturated, and comprises a 5-membered heterocyclyl, a 6-membered heterocyclyl or a carbocyclyl (provided the first ring is not benzo when the second ring is a carbocyclyl).

The bicyclic heterocyclic group may be attached to its pendant group at any heteroatom or carbon atom which results in a stable structure. The bicyclic heterocyclic group described herein may be substituted on carbon or on a nitrogen atom if the resulting compound is stable. It is preferred that when the total number of S and O atoms in the heterocyclyl exceeds 1, then these heteroatoms are not adjacent to one another. It is preferred that the total number of S and O atoms in the heterocyclyl is not more than 1.

Examples of a bicyclic heterocyclic group are, but not limited to, quinolinyl, isoquinolinyl, phthalazinyl, quinazolinyl, indolyl, isoindolyl, indolinyl, 1H-indazolyl, benzimidazolyl, 1,2,3,4-tetrahydroquinolinyl, 1,2,3,4-tetrahydroisoquinolinyl, 5,6,7,8-tetrahydroquinolinyl, 2,3-dihydrobenzofuranyl, chromanyl, 1,2,3,4-tetrahydroquinoxalinyl, and 1,2,3,4-tetrahydroquinazolinyl.

“Heteroaryl” is intended to mean stable monocyclic and polycyclic aromatic hydrocarbons that include at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Heteroaryl groups are substituted or unsubstituted. The nitrogen atom is substituted or unsubstituted (i.e., N or NR wherein R is H or another substituent, if defined). The nitrogen and sulfur heteroatoms may optionally be oxidized (i.e., N→O and S(O)ₚ, wherein p is 0, 1 or 2).

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring. Ring double bonds, as used herein, are double bonds that are formed between two adjacent ring atoms (e.g., C═C, C═N, or N═N).

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R groups, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The invention includes all tautomeric forms of the compounds, atropisomers and rotational isomers.

All processes used to prepare compounds of the present invention and intermediates made therein are considered to be part of the present invention.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

The term “chiral” refers to the structural characteristic of a molecule that makes it impossible to superimpose it on its mirror image. The term “homochiral” refers to a state of enantiomeric purity. The term “optical activity” refers to the degree to which a homochiral molecule or nonracemic mixture of chiral molecules rotates a plane of polarized light.

The invention is intended to include all isotopes of atoms occurring in the compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans- (or E- and Z-)geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt; a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

The term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

### Biological Methods

RXFP1 Cyclic Adenosine Monophosphate (cAMP) Assays. Human embryonic kidney cells 293 (HEK293) cells and HEK293 cells stably expressing human RXFP1, were cultured in MEM medium supplemented with 10% qualified FBS, and 300 μg/ml hygromycin (Life Technologies). Cells were dissociated and suspended in assay buffer. The assay buffer was HBSS buffer (with calcium and magnesium) containing 20 mM HEPES, 0.05% BSA, and 0.5 mM IBMX. Cells (3000 cells per well, except 1500 cell per well for HEK293 cells stably expressing human RXFP1) were added to 384-well Proxiplates (Perkin-Elmer). Cells were immediately treated with test compounds in DMSO (2% final) at final concentrations in the range of 0.010 nM to 50 μM. Cells were incubated for 30 min at rt. The level of intracellular cAMP was determined using the HTRF HiRange cAMP assay reagent kit (Cisbio) according to manufacturer's instructions. Solutions of cryptate conjugated anti-cAMP and d2 fluorophore-labelled cAMP were made in a supplied lysis buffer separately. Upon completion of the reaction, the cells were lysed with equal volume of the d2-cAMP solution and anti-cAMP solution. After a 1 h rt incubation, time-resolved fluorescence intensity was measured using the Envision (Perkin-Elmer) at 400 nm excitation and dual emission at 590 nm and 665 nm. A calibration curve was constructed with an external cAMP standard at concentrations ranging from 2.7 μM to 0.1 pM by plotting the fluorescent intensity ratio from 665 nm emission to the intensity from the 590 nm emission against cAMP concentrations. The potency and activity of a compound to inhibit cAMP production was then determined by fitting to a 4-parametric logistic equation from a plot of cAMP level versus compound concentrations.

The examples disclosed below were tested in the human RXFP1 (hRXFP1) HEK293 cAMP assay described above and found to have agonist activity.

TABLE 1
lists EC₅₀ values in the hRXFP1 HEK293
cAMP assay measured for the examples.

| | cAMP hRXFP1 HEK293 |
|---|---|
| Example | Assay EC₅₀ (nM) |
| 1 | 631 |
| 2 | 2010 |
| 3 | 350 |
| 4 | 375 |
| 5 | 150 |
| 6 | 349 |
| 7 | 661 |
| 8 | 2020 |
| 9 | 2380 |
| 10 | 111 |
| 11 | 2540 |
| 12 | 2130 |
| 13 | 87 |
| 14 | 135 |
| 15 | 1560 |
| 16 | 677 |
| 17 | 10.3 |
| 18 | 311 |
| 19 | 1010 |
| 20 | 954 |
| 21 | 363 |
| 22 | 0.5 |
| 23 | 3100 |
| 24 | 3360 |
| 25 | 3.8 |
| 26 | 153 |
| 27 | 1720 |
| 28 | 48.7 |
| 29 | 1340 |
| 30 | 2540 |
| 31 | 3280 |
| 32 | 718 |
| 33 | 216 |
| 34 | 850 |
| 35 | 794 |
| 36 | 244 |
| 37 | 158 |
| 38 | 194 |
| 39 | 2800 |
| 40 | 220 |
| 42 | 467 |
| 44 | 3740 |
| 45 | 975 |
| 46 | 295 |
| 47 | 1060 |
| 48 | 58.4 |
| 49 | 617 |
| 50 | 459 |
| 51 | 1380 |
| 52 | 166 |
| 53 | 71.0 |
| 54 | 306 |
| 55 | 320 |
| 56 | 9.5 |
| 57 | 636 |
| 58 | 612 |
| 59 | 216 |
| 60 | 743 |
| 61 | 599 |
| 62 | 1460 |
| 63 | 2060 |
| 64 | 293 |
| 65 | 662 |
| 66 | 897 |
| 67 | 1290 |
| 68 | 1080 |
| 69 | 760 |
| 70 | 46.8 |
| 71 | 2710 |
| 72 | 4310 |
| 73 | 1040 |
| 74 | 3370 |
| 75 | 420 |
| 76 | 498 |
| 77 | 469 |
| 78 | 835 |
| 79 | 390 |
| 80 | 237 |
| 81 | 203 |
| 82 | 978 |
| 83 | 608 |
| 84 | 226 |
| 85 | 1110 |
| 86 | 1150 |
| 87 | 1160 |
| 88 | 1540 |
| 89 | 2180 |
| 90 | 1580 |
| 91 | 95.8 |
| 92 | 165 |
| 93 | 48.4 |
| 94 | 245 |
| 95 | 870 |
| 96 | 324 |
| 97 | 607 |
| 98 | 186 |
| 99 | 1370 |
| 100 | 2010 |
| 101 | 4420 |
| 102 | 2230 |
| 103 | 690 |
| 104 | 277 |
| 105 | 149 |
| 106 | 248 |
| 107 | 918 |
| 108 | 1430 |
| 109 | 509 |
| 110 | 144 |
| 111 | 28.3 |
| 112 | 593 |
| 114 | 22.5 |
| 115 | 98.0 |
| 116 | 650 |
| 117 | 131 |
| 118 | 255 |
| 119 | 282 |
| 120 | 228 |
| 121 | 111 |
| 122 | 41.8 |
| 123 | 236 |
| 124 | 560 |
| 127 | 254 |
| 128 | 237 |
| 129 | 444 |
| 130 | 279 |
| 131 | 51.2 |
| 132 | 135 |
| 133 | 876 |
| 134 | 634 |
| 135 | 488 |
| 136 | 514 |
| 137 | 591 |
| 138 | 213 |
| 139 | 72.6 |
| 140 | 97.9 |
| 141 | 400 |
| 142 | 619 |
| 143 | 1540 |
| 144 | 1430 |
| 145 | 1,650 |
| 146 | 1,100 |
| 147 | 1,430 |
| 148 | 2,240 |
| 149 | 1,200 |
| 150 | 228 |
| 151 | 208 |
| 152 | 613 |
| 153 | 314 |
| 154 | 553 |
| 155 | 902 |
| 156 | 2,180 |
| 157 | 1,040 |
| 158 | 29.8 |
| 159 | 199 |
| 160 | 2,300 |
| 161 | 4,940 |
| 162 | 292 |
| 163 | 662 |
| 164 | 865 |
| 165 | 3,050 |
| 166 | 1,630 |
| 167 | 400 |
| 168 | 176 |
| 169 | 2,250 |
| 170 | 1,100 |
| 171 | 9.1 |
| 172 | 701 |
| 173 | 179 |
| 174 | 405 |
| 175 | 374 |
| 176 | 279 |
| 177 | 147 |
| 178 | 5.2 |
| 179 | 179 |
| 180 | 145 |
| 181 | 544 |
| 182 | 357 |
| 183 | 823 |
| 184 | 349 |
| 185 | 736 |
| 186 | 102 |
| 187 | 328 |
| 188 | 772 |
| 189 | 491 |
| 190 | 689 |
| 191 | 346 |
| 192 | 889 |
| 193 | 300 |
| 194 | 746 |
| 195 | 910 |
| 196 | 70.0 |
| 197 | 450 |
| 199 | 690 |
| 200 | 400 |
| 201 | 320 |
| 202 | 1010 |
| 203 | 36.1 |
| 204 | 1,170 |
| 205 | 1,100 |
| 206 | 2,080 |
| 207 | 1,460 |
| 208 | 1,480 |
| 209 | 1,480 |
| 210 | 1,930 |
| 211 | 2,770 |
| 212 | 3,330 |
| 213 | 3,360 |
| 214 | 2,150 |
| 215 | 1,430 |
| 216 | 3,490 |
| 217 | 2,290 |
| 218 | 1,130 |
| 219 | 1,140 |
| 220 | 2,530 |
| 221 | 1,100 |
| 222 | 1,610 |
| 223 | 1,250 |
| 224 | 3,580 |
| 225 | 1,060 |
| 226 | 1,760 |
| 227 | 1,280 |
| 228 | 1,540 |
| 229 | 2,570 |
| 230 | 1,400 |
| 231 | 1,680 |
| 232 | 4,840 |
| 233 | 4,650 |
| 234 | 1,340 |
| 235 | 1,500 |
| 236 | 1,390 |
| 237 | 3,770 |
| 238 | 1,400 |
| 239 | 2,100 |
| 240 | 3,300 |
| 241 | 3900 |
| 242 | 9.8 |
| 243 | 1050 |
| 244 | 360 |
| 245 | 463 |
| 246 | 724 |
| 247 | 531 |
| 248 | 545 |
| 249 | 3700 |
| 250 | 280 |
| 251 | 222 |
| 253 | 192 |
| 254 | 1020 |
| 255 | 498 |
| 256 | 239 |
| 257 | 166 |
| 258 | 673 |
| 259 | 364 |
| 260 | 1310 |
| 261 | 90.7 |
| 262 | 723 |
| 263 | 122 |
| 264 | 3140 |
| 265 | 1,140 |
| 266 | 2,410 |
| 267 | 3,130 |
| 268 | 4.9 |
| 269 | 737 |
| 270 | 532 |
| 271 | 379 |
| 272 | 900 |
| 273 | 857 |
| 274 | 278 |
| 275 | 440 |
| 276 | 204 |
| 277 | 398 |
| 278 | 408 |
| 279 | 582 |
| 280 | 44.1 |
| 281 | 95.1 |
| 282 | 9.6 |
| 283 | 1,120 |
| 284 | 1,300 |
| 285 | 1,860 |
| 286 | 5000 |
| 287 | 1,260 |
| 288 | 4,260 |
| 289 | 2,050 |
| 290 | 1,410 |

### Pharmaceutical Compositions and Methods of Use

The compounds of Formula (I) are RXFP1 receptor agonists and may find use in the treatment of medical indications such as heart failure, fibrotic diseases, and related diseases such as lung disease (e.g., idiopathic pulmonary fibrosis), kidney disease (e.g., chronic kidney disease), or hepatic disease (e.g., non-alcoholic steatohepatitis and portal hypertension).

Another aspect of the invention is a pharmaceutical composition comprising a compound of Formula (I) and a pharmaceutically acceptable carrier.

Another aspect of the invention is a pharmaceutical composition comprising a compound of Formula (I) for the treatment of a relaxin-associated disorder and a pharmaceutically acceptable carrier.

Another aspect of the invention is a method of treating a disease associated with relaxin comprising administering an effective amount of a compound of Formula (I).

Another aspect of the invention is a method of treating a cardiovascular disease comprising administering an effective amount of a compound of Formula (I) to a patient in need thereof.

Another aspect of the invention is a method of treating heart failure comprising administering an effective amount of a compound of Formula (I) to a patient in need thereof.

Another aspect of the invention is a method of treating fibrosis comprising administering a therapeutically effective amount of a compound of Formula (I) to a patient in need thereof.

Another aspect of the invention is a method of treating a disease associated with fibrosis comprising administering a therapeutically effective amount of a compound of Formula (I) to a patient in need thereof.

Another aspect of the invention is a method of treating or preventing kidney failure, comprising administering a therapeutically effective amount of a compound of Formula (I) to a patient in need thereof.

Another aspect of the invention is a method of improving, stabilizing or restoring renal function in a patient in need thereof, comprising administering a therapeutically effective amount of a compound of Formula (I) to the patient.

Unless otherwise specified, the following terms have the stated meanings.

The term “patient” or “subject” refers to any human or non-human organism that could potentially benefit from treatment with a RXFP1 agonist as understood by practioners in this field. Exemplary subjects include human beings of any age with risk factors for cardiovascular disease. Common risk factors include, but are not limited to, age, sex, weight, family history, sleep apnea, alcohol or tobacco use, physical inactivity, arrhythmia, or signs of insulin resistance such as acanthosis nigricans, hypertension, dyslipidemia, or polycystic ovary syndrome (PCOS).

“Treating” or “treatment” cover the treatment of a disease-state as understood by practitioners in this field and include the following: (a) inhibiting the disease-state, i.e., arresting it development; (b) relieving the disease-state, i.e., causing regression of the disease state; and/or (c) preventing the disease-state from occurring in a mammal, in particular, when such mammal is predisposed to the disease-state but has not yet been diagnosed as having it.

“Preventing” or “prevention” cover the preventive treatment (i.e., prophylaxis and/or risk reduction) of a subclinical disease-state aimed at reducing the probability of the occurrence of a clinical disease-state as understood by practitioners in this field. Patients are selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state. “Risk reduction” or “reducing risk” covers therapies that lower the incidence of development of a clinical disease state. As such, primary and secondary prevention therapies are examples of risk reduction.

“Therapeutically effective amount” is intended to include an amount of a compound of the present invention that is effective when administered alone or in combination with other agents to treat disorders as understood by practitioners in this field. When applied to a combination, the term refers to combined amounts of the active ingredients that result in the preventive or therapeutic effect, whether administered in combination, serially, or simultaneously.

“Disorders of the cardiovascular system” or “cardiovascular disorders” include for example the following disorders: hypertension (high blood pressure), peripheral and cardiac vascular disorders, coronary heart disease, stable and unstable angina pectoris, heart attack, myocardial insufficiency, abnormal heart rhythms (or arrhythmias), persistent ischemic dysfunction (“hibernating myocardium”), temporary postischemic dysfunction (“stunned myocardium”), heart failure, disturbances of peripheral blood flow, acute coronary syndrome, heart failure, heart muscle disease (cardiomyopathy), myocardial infarction and vascular disease (blood vessel disease).

“Heart failure” includes both acute and chronic manifestations of heart failure, as well as more specific or related types of disease, such as advanced heart failure, post-acute heart failure, cardio-renal syndrome, heart failure with impaired kidney function, chronic heart failure, chronic heart failure with mid-range ejection fraction (HFmEF), compensated heart failure, decompensated heart failure, right heart failure, left heart failure, global failure, ischemic cardiomyopathy, dilated cardiomyopathy, heart failure associated with congenital heart defects, heart valve defects, heart failure associated with heart valve defects, mitral stenosis, mitral insufficiency, aortic stenosis, aortic insufficiency, tricuspid stenosis, tricuspid insufficiency, pulmonary stenosis, pulmonary valve insufficiency, heart failure associated with combined heart valve defects, myocardial inflammation (myocarditis), chronic myocarditis, acute myocarditis, viral myocarditis, diabetic heart failure, alcoholic cardiomyopathy, heart failure associated with cardiac storage disorders, diastolic heart failure, systolic heart failure, acute phases of worsening heart failure, heart failure with preserved ejection fraction (HFpEF), heart failure with reduced ejection fraction (HFrEF), chronic heart failure with reduced ejection fraction (HFrEF), chronic heart failure with preserved ejection fraction (HFpEF), post myocardial remodeling, angina, hypertension, pulmonary hypertension and pulmonary artery hypertension.

“Fibrotic disorders” encompasses diseases and disorders characterized by fibrosis, including among others the following diseases and disorders: hepatic fibrosis, cirrhosis of the liver, NASH, pulmonary fibrosis or lung fibrosis, cardiac fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitreoretinopathy and disorders of the connective tissue (for example sarcoidosis).

Relaxin-associated disorders include but are not limited to disorders of the cardiovascular system and fibrotic disorders.

The compounds of this invention can be administered by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

“Pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, anti-bacterial agents, anti-fungal agents, lubricating agents and dispensing agents, depending on the nature of the mode of administration and dosage forms.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted.

Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V., Jr. et al., Remington: The Science and Practice of Pharmacy (2 Volumes), 22nd Edition, Pharmaceutical Press (2012).

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.01 to about 5000 mg per day, preferably between about 0.1 to about 1000 mg per day, and most preferably between about 0.1 to about 250 mg per day. Intravenously, the most preferred doses will range from about 0.01 to about 10 mg/kg/minute during a constant rate infusion. Compounds of this invention may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

Dosage forms (pharmaceutical compositions) suitable for administration may contain from about 1 milligram to about 2000 milligrams of active ingredient per dosage unit. In these pharmaceutical compositions the active ingredient will ordinarily be present in an amount of about 0.1-95% by weight based on the total weight of the composition. A typical capsule for oral administration contains at least one of the compounds of the present invention (250 mg), lactose (75 mg), and magnesium stearate (15 mg). The mixture is passed through a 60 mesh sieve and packed into a No. 1 gelatin capsule. A typical injectable preparation is produced by aseptically placing at least one of the compounds of the present invention (250 mg) into a vial, aseptically freeze-drying and sealing. For use, the contents of the vial are mixed with 2 mL of physiological saline, to produce an injectable preparation.

The compounds may be employed in combination with other suitable therapeutic agents useful in the treatment of diseases or disorders including: anti-atherosclerotic agents, anti-dyslipidemic agents, anti-diabetic agents, anti-hyperglycemic agents, anti-hyperinsulinemic agents, anti-thrombotic agents, anti-retinopathic agents, anti-neuropathic agents, anti-nephropathic agents, anti-ischemic agents, anti-hypertensive agents, anti-obesity agents, anti-hyperlipidemic agents, anti-hypertriglyceridemic agents, anti-hypercholesterolemic agents, anti-restenotic agents, anti-pancreatic agents, lipid lowering agents, anorectic agents, memory enhancing agents, anti-dementia agents, cognition promoting agents, appetite suppressants, agents for treating heart failure, agents for treating peripheral arterial disease, agents for treating malignant tumors, and anti-inflammatory agents.

The additional therapeutic agents may include ACE inhibitors, β-blockers, diuretics, mineralocorticoid receptor antagonists, ryanodine receptor modulators, SERCA2a activators, renin inhibitors, calcium channel blockers, adenosine A1 receptor agonists, partial adenosine A1 receptor, dopamine β-hydroxylase inhibitors, angiotensin II receptor antagonists, angiotensin II receptor antagonists with biased agonism for select cell signaling pathways, combinations of angiotensin II receptor antagonists and neprilysin enzyme inhibitors, neprilysin enzyme inhibitors, soluble guanylate cyclase activators, myosin ATPase activators, rho-kinase 1 inhibitors, rho-kinase 2 inhibitors, apelin receptor agonists, nitroxyl donating compounds, calcium-dependent kinase II inhibitors, antifibrotic agents, galectin-3 inhibitors, vasopressin receptor antagonists, FPR2 receptor modulators, natriuretic peptide receptor agonists, transient receptor potential vanilloid-4 channel blockers, anti-arrhythmic agents, I_{f} “funny current” channel blockers, nitrates, digitalis compounds, inotropic agents and β-receptor agonists, cell membrane resealing agents for example Poloxamer 188, anti-hyperlipidemic agents, plasma HDL-raising agents, anti-hypercholesterolemic agents, cholesterol biosynthesis inhibitors (such as HMG CoA reductase inhibitors), LXR agonist, FXR agonist, probucol, raloxifene, nicotinic acid, niacinamide, cholesterol absorption inhibitors, bile acid sequestrants, anion exchange resins, quaternary amines, cholestyramine, colestipol, low density lipoprotein receptor inducers, clofibrate, fenofibrate, bezafibrate, ciprofibrate, gemfibrizol, vitamin B6, vitamin B12, anti-oxidant vitamins, anti-diabetes agents, platelet aggregation inhibitors, fibrinogen receptor antagonists, aspirin and fibric acid derivatives, PCSK9 inhibitors, aspirin, and P2Y12 Inhibitors such as Clopidogrel.

The additional therapeutic agents may also include nintedanib, Pirfenidone, LPA1 antagonists, LPA1 receptor antagonists, GLP1 analogs, tralokinumab (IL-13, AstraZeneca), vismodegib (hedgehog antagonist, Roche), PRM-151 (pentraxin-2, TGF beta-1, Promedior), SAR-156597 (bispecific Mab IL-4&IL-13, Sanofi), simtuzumab ((anti-lysyl oxidase-like 2 (anti-LOXL2) antibody, Gilead), CKD-942, PTL-202 (PDE inh./pentoxifylline/NAC oral control. release, Pacific Ther.), omipalisib (oral PI3K/mTOR inhibitor, GSK), IW-001 (oral sol. bovine type V collagen mod., ImmuneWorks), STX-100 (integrin alpha V/beta-6 ant, Stromedix/Biogen), Actimmune (IFN gamma), PC-SOD (midismase; inhaled, LTT Bio-Pharma/CKD Pharm), lebrikizumab (anti-IL-13 SC humanized mAb, Roche), AQX-1125 (SHIP1 activator, Aquinox), CC-539 (JNK inhibitor, Celgene), FG-3019 (FibroGen), SAR-100842 (Sanofi), and obeticholic acid (OCA or INT-747, Intercept).

The above other therapeutic agents, when employed in combination with the compounds of the present invention may be used, for example, in those amounts indicated in the Physicians' Desk Reference, as in the patents set out above, or as otherwise determined by practitioners in the art.

Particularly when provided as a single dosage unit, the potential exists for a chemical interaction between the combined active ingredients. For this reason, when the compound of the present invention and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

The compounds of the present invention are also useful as standard or reference compounds, for example as a quality standard or control, in tests or assays involving RXFP1. Such compounds may be provided in a commercial kit, for example, for use in pharmaceutical research involving RXFP1. For example, a compound of the present invention could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, compounds according to the present invention could be used to test their effectiveness. The compounds of the present invention may also be used in diagnostic assays involving RXFP1.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises a first therapeutic agent, comprising a compound of the present invention or a pharmaceutically acceptable salt form thereof; and, (c) a package insert stating that the pharmaceutical composition can be used for the treatment of dyslipidemias and the sequelae thereof. In another embodiment, the package insert states that the pharmaceutical composition can be used in combination (as defined previously) with a second therapeutic agent for the treatment of dyslipidemias and the sequelae thereof. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

### Chemical Methods and Synthesis

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention.

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene, T. W. et al., Protecting Groups in Organic Synthesis, 4th Edition, Wiley (2007)).

Abbreviations are defined as follows: “1×” for once, “2×” for twice, “3×” for thrice, “° C.” for degrees Celsius, “aq” for aqueous, “eq” or “equiv.” for equivalent or equivalents, “g” for gram or grams, “mg” for milligram or milligrams, “L” for liter or liters, “mL” for milliliter or milliliters, “μL” for microliter or microliters, “N” for normal, “M” for molar, “nM” for nanomolar, “pM” for picomolar, “mol” for mole or moles, “mmol” for millimole or millimoles, “min” for minute or minutes, “h” for hour or hours, “rt” for room temperature, “RT” for retention time, “atm” for atmosphere, “psi” for pounds per square inch, “conc.” for concentrated, “aq” for “aqueous”, “sat.” for saturated, “MW” for molecular weight, “MS” or “Mass Spec” for mass spectrometry, “ESI” for electrospray ionization mass spectroscopy, “LC-MS” for liquid chromatography mass spectrometry, “HPLC” for high pressure liquid chromatography, “RP HPLC” for reverse phase HPLC, “NMR” for nuclear magnetic resonance spectroscopy, “SFC” for super critical fluid chromatography, “1H” for proton, “δ” for delta, “s” for singlet, “d” for doublet, “t” for triplet, “q” or quartet, “m” for multiplet, “br” for broad, “Hz” for hertz, “MHz” for megahertz, and “α”, “β”, “R”, “S”, “E”, and “Z” are stereochemical designations familiar to one skilled in the art.
ACN acetonitrile
AcOH acetic acid
BINAP 2,2′-bis(diphenylphosphino)-1,1′-binaphthyl
B2Pin2 Bis(pinacolato)diboron
Boc tert-butyloxycarbonyl
Boc2O Di-tert-butyl dicarbonate
BOP (Benzotriazol-1-yloxy)tris(dimethylamino)phosphonium hexafluorophosphate
BuLi butyl lithium
CDI 1,1′-Carbonyldiimidazole
COD 1,5-cyclooctadiene
DABAL-Me3 bis(trimethylaluminium)-1,4-diazabicyclo[2.2.2]octane adduct
DAST Diethylaminosulfur trifluoroide
DCE Dichloroethane
DCM Dichloromethane
DEA Diethylamine
DIEA diispropyl ethylamine
DIPA diispropyl amine
DMAP—dimethylamino pyridine
DMF Dimethylformamide
DMSO Dimethylsulfoxide
dppf 1,1′-Ferrocenediyl-bis(diphenylphosphine)
EDC N-(3-Dimethylaminopropyl)-N′-ethylcarbodiimide hydrochloride
Et2O diethyl ether
EtOAc Ethylacetate
EtOH Ethanol
HATU (1-[Bis(dimethylamino)methylene]-1H-1,2,3-triazolo[4,5-b]pyridinium 3-oxid hexafluorophosphate)
IPA isopropanol
i-Pr Isopropyl
LAH lithium aluminum hydride
LDA lithium diisopropyl amide
LiHMDS lithium bis(trimethylsilyl)amide
m-CPBA meta-Chloroperoxybenzoic acid
MeOH Methanol
Me Methyl
Ms Mesyl
NBS N-bromosuccinimide
NCS N-chlorosuccinimide
NMI 1-Methylimidazole
NIS N-iodosuccinimide
OAc acetate
OtBu tert-butoxide
OTf trifluoromethanesulfonate
Pd/C palladium on carbon
RuPhosPdG2 Chloro(2-dicyclohexylphosphino-2′,6′-diisopropoxy-1,1′-biphenyl)[2-(2′-amino-1,1′-biphenyl)]palladium(II)
selectfluor 1-Chloromethyl-4-fluoro-1,4-diazoniabicyclo[2.2.2]octane bis(tetrafluoroborate)
SFC Supercritical fluid chromatography
tBu tert-butyl
TCFH Chloro-N,N,N′,N′-tetramethylformamidinium hexafluorophosphate
TEA triethylamine
TFA trifluoro acetic acid
TFAA trifluoro acetic anhydride
Tf2O Trifluoromethanesulfonic anhydride
THF Tetrahydrofuran
Xantphos 4,5-Bis(diphenylphosphino)-9,9-dimethylxanthene

The following methods were used in the exemplified examples, except where noted otherwise. Purification of intermediates and final products was carried out via either normal or reverse phase chromatography. Normal phase chromatography was carried out using prepacked SiO2 cartridges eluting with either gradients of hexanes and EtOAc or DCM and MeOH or Pet ether and EtOAc unless otherwise indicated. Reverse phase preparative HPLC was carried out using C18 columns with UV 220 nm or prep LCMS detection eluting with gradients of Solvent A (90% water, 10% MeOH, 0.1% TFA) and Solvent B (10% water, 90% MeOH, 0.1% TFA) or with gradients of Solvent A (95% water, 5% ACN, 0.1% TFA) and Solvent B (5% water, 95% ACN, 0.1% TFA) or with gradients of Solvent A (95% water, 2% ACN, 0.1% HCOOH) and Solvent B (98% ACN, 2% water, 0.1% HCOOH) or with gradients of Solvent A (95% water, 5% ACN, 10 mM NH4OAc) and Solvent B (98% ACN, 2% water, 10 mM NH4OAc) or with gradients of Solvent A (98% water, 2% ACN, 0.1% NH4OH) and Solvent B (98% ACN, 2% water, 0.1% NH4OH).

LC/MS methods employed in characterization of examples are listed below.

Method A: Column: XBridge BEH XP C18 (50×2.1) mm, 2.5 μm: Mobile Phase A: 5:95 ACN:H2O with 10 mM NH4OAc; Mobile Phase B: 95:5 ACN:H2O with 10 mM NH4OAc; Temperature: 50° C.; Gradient: 0-100% B over 3 minutes; Flow: 1.1 mL/min; Detection: UV at 220 nm.

Method B: Column: XBridge BEH XP C18 (50×2.1) mm, 2.5 μm; Mobile Phase A: 5:95 ACN:H2O with 0.10% TFA; Mobile Phase B: 95:5 ACN:H2O with 0.10% TFA; Temperature: 50° C.; Gradient: 0-100% B over 3 minutes; Flow: 1.1 mL/min; Detection: UV at 220 nm.

Method C: Column: Kinetex XB-C18 (75×3) mm 2.6 μm; Mobile Phase A: 10 mM NH4COOH in H2O:ACN (98:2); Mobile Phase B: 10 mM NH4COOH in H2O:ACN (2:98); Temperature: 50° C.; Gradient: 20-100% B over 5 minutes; Flow rate: 1.1 mL/min; Detection: UV at 220 nm.

Method D: Column: Kinetix C18 (75×3) mm 2.6 μm; Mobile Phase A: 0.1% HCOOH in H2O; Mobile Phase B: ACN; Gradient: 20-100% B over 5 minutes; Detection: UV at 220 nm.

Method E: Acquity UPLC BEH C18 (50×3.0) mm 1.7 μm; Buffer: 10 mM NH4OAc in H2O; Mobile Phase A: Buffer: ACN (95:5); Mobile Phase B: Buffer: ACN (5:95); Gradient % B: 20-100% over 2.0 min, hold 100% 2.2 min; Flow Rate: 0.7 mL/min; Detection: UV at 220 nm.

Method F: Column: Acquity UPLC BEH C18 (50×3.0) mm 1.7 μm; Mobile Phase A: 5:95 ACN:H2O with 0.1% TFA; Mobile Phase B: 95:5 ACN:H2O with 0.1% TFA; Gradient: 2-98% B over 2 minutes; Flow: 0.8 mL/min; Column temp: 60° C.; Detection: UV at 220 nm.

Method G: Column: Luna 3.0 C18 (2) 100 Å LC column (20×4.0) mm, Mercury MS TM; Mobile Phase A: 10 mM NH4COOH in H2O:ACN (98:02); Mobile Phase B: 10 mM NH4COOH in H2O:ACN (02:98); Flow rate: 1.5 mL/min.

Method H: Column: Phenomenex Luna C18 (2) (30×2.0) mm, 3 m; Buffer: 10 mM NH4OAc in H2O; Mobile Phase A: Buffer: MeOH (90:10); Mobile Phase B: Buffer: MeOH (10:90); Gradient % B: 0-100% 2.0 min, hold 100% 3.0 min; Flow Rate: 1.0 mL/min; Detection: UV at 220 nm.

Preparative LCMS/HPLC/SFC conditions: Fractions containing the desired product were combined and dried via centrifugal evaporation.

Method 1: Column: Waters XBridge C18, (150×19) mm, 5 μm; Mobile Phase A: 5:95 ACN:H2O with 10 mM NH4OAc; Mobile Phase B: 95:5 ACN:H2O with 10 mM NH4OAc; Flow Rate: 15 mL/min; Column Temperature: 25° C.

Method 2: Column: Sunfire C18, (150×19) mm, 5 μm; Mobile Phase A: 5:95 ACN:H2O with 10 mM NH4OAc; Mobile Phase B: 95:5 ACN:H2O with 10 mM NH4OAc; Flow 19 mL/min.

Method 3: Column: Sunfire C18, (150×19) mm, 5 μm; Mobile Phase A: 10 mM NH4OAc in H2O, pH 4.5; Mobile Phase B: MeOH; Flow 19 mL/min.

Method 4: Column: Waters XBridge C18, (200×19) mm, 5 μm; Mobile Phase A: 5:95 ACN:H2O with 0.05% TFA; Mobile Phase B: 95:5 ACN:H2O with 0.05% TFA; Flow Rate: 20 mL/min; Column Temperature: 25° C.

Method 5: Column: YMC Triart EXRS C18 (250×20) mm 5 μm; Mobile Phase A: 10 mM NH4HCO3 in H2O, pH 9.5; Mobile Phase B: ACN; Flow: 19 mL/min.

Method 6: Column: Sunfire C18 (150×19) mm, 5 μm; Mobile Phase A: 0.1% TFA in H2O; Mobile Phase B: ACN; Flow 19 mL/min.

Method 7: Column: Xbridge Phenyl (250×19) mm, 5 μm; Mobile Phase A: 10 mM NH4HCO3 in H2O, pH 9.5; Mobile Phase B: ACN; Flow: 19 mL/min.

Method 8: Column: Chiralcel OD-H (250×30) mm, 5 μm; % CO2: 50%; Co-solvent: 50% 4M NH3 in MeOH; Total Flow: 80 g/min; Back Pressure: 100 bar; Temperature: 30° C.; UV: 240 nm.

Method 9: Column: ACE C18 PFP (250×21.2) mm, 5 μm; Mobile Phase A: 10 mM NH4OAc in H2O, pH 4.5; Mobile Phase B: ACN:IPA (70:30); Flow 19 mL/min; UV: 254 nm.

Method 10: Column: Chiralpak IC (250×30) mm, 5 μm; % CO2: 75%; Co-solvent: 25% of 4M NH3 in MeOH; Total Flow: 95 g/min; Back Pressure: 100 bar; Temperature: 40° C.; UV: 220 nm.

Method 11: Column: Gemini NX C-18 (250×21.2) mm, 5 μm; Mobile Phase A: 10 mM NH4HCO3 in Water, pH 9.5; Mobile Phase B: ACN; Flow: 19 mL/min; Gradient: 65-70%, 2 min, 70-85% 15 min, 85% 17 min, 85-100%, 18 min.

Method 12: Column Name: Luxcellulose C4 (250×21.5) mm, 5 μm; % CO2: 80%; Co-solvent: 20% of 4M NH3 in MeOH; Back Pressure: 100 bar; Temperature: 40° C.; UV: 246 nm.

Method 13: Analytical SFC conditions: Column Name: Luxcellulose C4 (250×4.6) mm, 5 μm; % CO2: 65%; Co-solvent: 35% of 4M NH3 in MeOH; Total Flow: 4.0 g/min; Back Pressure: 100 bar; Temperature: 30° C.; UV: 246 nm.

Method 14: Column: Cellulose-5 (250×19) mm, 5 μm; Mobile Phase: 10 mM NH4OAc in MeOH, Flow: 20 mL/min; UV: 220 nm.

Method 15: Column: C-5 (250×21.2 mm) 5 μm, DAD-1B; Mobile Phase: 0.1% DEA in ACN, Flow: 19 mL/min, UV: 254 nm.

Method 16: Column: Chiralpak IG (250×30) mm, 5 μm; % CO2: 50%; Co-solvent: 50% of 0.1% DEA IN IPA; Total Flow: 80 g/min; Back Pressure: 100 bar; Temperature: 35° C.; UV: 220 nm.

Method 17: Analytical SFC conditions: Column: Chiralpak IG (250×4.6) mm, 5 μm; % CO2: 60%; Co-solvent: 40% 0.2% DEA in IPA; Injected Volume: 10 μl; Outlet Pressure: 100 bar; Temperature: 35° C.; UV: 220 nm.

Method 18: Column: Chiralpak IC (250×21) mm, 5 μm; % CO2: 50%; Co-Solvent: 50% of 0.2% NH3 in MeOH; Total Flow: 90.0 g/min; Back Pressure: 100 bar; Temperature: 35° C.; UV: 265 nm.

Method 19: Analytical SFC conditions: Column: Chiralpak IC (250×4.6) mm, 5 μm; % CO2: 50%; Co-Solvent: 50% of 0.2% NH3 in MeOH; Total Flow: 4.0 g/min; Back Pressure: 100 bar; Temperature: 35° C.; UV: 265 nm.

Method 20: Column/dimensions: Chiralpak IG (250×30 mm), 5 μm; % CO2: 55%; % Co-solvent: 45% 0.2% DEA in MeOH:ACN (1:1); Total Flow: 170.0 g/min; Back Pressure: 100 bar; Temperature: 35° C.; UV: 220 nm.

Method 21: Analytical SFC conditions: Column: Chiralpak IG (250×4.6 mm), 5 μm; % CO2: 55%; % Co-solvent: 45% 0.2% DEA in MeOH:ACN (1:1); Total Flow: 4.0 g/min; Back Pressure: 100 bar; Temperature: 35° C.; UV: 220 nm.

Method 22: Column: Chiralpak IG (250×30) mm, 5 μm; % CO2: 70%; Co-solvent: 30% of 4M NH3 in MeOH; Flow Conditions: 150.0 g/min; Back Pressure: 100 Bar; Temperature: 30° C.; Detector Wavelength: 240 nm.

Method 23: Analytical SFC conditions: Column: Chiralpak IG (250×4.6) mm, 5 μm; % CO2: 70%; Co-solvent: 30% of 4M NH3 in MeOH; Flow Conditions: 4.0 g/min; Back Pressure: 100 Bar; Temperature: 30° C.; Detector Wavelength: 240 nm.

Method 24: Column: Chiralcel OD-H (250×21) mm, 5 μm; % CO2: 80%; Co-solvent: 20% 4M NH3 in MeOH; Total Flow: 95 g/min; Back Pressure: 100 bar; Temperature: 40° C.; UV: 220 nm.

Method 25: Analytical SFC conditions: Column: Chiralcel OD-H (250×4.6) mm, 5 μm; % CO2: 70%; Co-solvent: 30% 4M NH3 in MeOH; Total Flow: 4.0 g/min; Back Pressure: 100 bar; Temperature: 35° C.; UV: 220 nm.

Method 26: Column: Chiralpak AS-H (250×30) mm, 5 μm; % CO2: 80%; Co-solvent: 20% of 4M NH3 in MeOH; Total Flow: 140.0 g/min; Back Pressure: 130 bar; Temperature: 30° C.; UV: 225 nm.

Method 27: Analytical SFC conditions: Column: Chiralpak AS-H (250×4.6) mm, 5 μm; % CO2: 80%; Co-solvent: 20% of 4M NH3 in MeOH; Total Flow: 4.0 g/min; Back Pressure: 100 bar; Temperature: 30° C.; UV: 225 nm.

Method 28: Column: Chiralpak IC (250×30) mm, 5 μm; % CO2: 75%; Co-solvent: 25% of 0.2% NH3 in MeOH; Total Flow: 100.0 g/min; Back Pressure: 100 bar; Temperature: 35° C.; UV: 264 nm

Method 29: Analytical SFC conditions: Column: Chiralpak IC (250×4.6) mm, 5 μm; % CO2: 70%; Co-solvent: 30% of 0.2% NH3 in MeOH; Total Flow: 4.0 g/min; Back Pressure: 100 bar; Temperature: 35° C.; UV: 264 nm

Method 30: Column: Luxcellulose 4 (250×30) mm, 5 μm; % CO2: 50%; % Co-solvent: 50% of 4M NH3 in MeOH; Total Flow: 145.0 g/min; Back Pressure: 100 bar; Temperature: 40° C.; UV: 270 nm.

Method 31: Analytical SFC conditions: Column: Luxcellulose 4 (250×4.6) mm, 5 μm; % CO2: 50%; % Co-solvent: 50% of 4M NH3 in MeOH; Total Flow: 4.0 g/min; Back Pressure: 100 bar; Temperature: 30° C.; UV: 270 nm.

Method 32: Column: Chiralcel OJ-H (250×30) mm, 5 μm; % CO2: 80%; % Co-solvent: 20% of 0.2% DEA in IPA; Total Flow: 100.0 g/min; Back Pressure: 100 bar; Temperature: 35° C.; UV: 220 nm.

Method 33: Analytical SFC conditions: Column: Chiralcel OJ-H (250×4.6) mm, 5 μm; % CO2: 80%; % Co-solvent: 20% of 0.2% DEA in IPA; Total Flow: 4.0 g/min; Back Pressure: 100 bar; Temperature: 35° C.; UV: 220 nm.

Method 34: Column: Whelk(R,R) (250×21) mm, 5 μm; % CO2: 80%; % Co-solvent: 20% of 0.2% NH3 in MeOH; Total Flow: 85.0 g/min; Back Pressure: 100 bar; Temperature: 35° C.; UV: 250 nm.

Method 35: Analytical SFC conditions: Column: Whelk(R,R) (250×4.6) mm, 5 μm; % CO2: 70%; % Co-solvent: 30% of 0.2% NH3 in MeOH; Total Flow: 4.0 g/min; Back Pressure: 100 bar; Temperature: 30° C.; UV: 250 nm.

Method 36: Column: Chiralpak IA (250×30) mm, 5 μm; % CO2: 80%; % Co-solvent: 20% MeOH; Total Flow: 90 mL/min; Back Pressure: 150 bar; Temperature: 40° C.; UV: 220 nm.

Method 37: Analytical SFC conditions: Column: Chiralpak IA (100×4.6) mm, 5 μm; % CO2: 75%; % Co-solvent: 25% MeOH; Total Flow: 2.0 mL/min; Back Pressure: 150 bar; Temperature: 40° C.; UV: 220 nm.

Method 38: Column/dimensions: Chiralcel OD-H (250×50) mm, 5 μm; % CO2: 75%; Co-solvent: 25% of 0.2% NH3 in MeOH; Total Flow: 270.0 g/min; Back Pressure: 100 bar; Temperature: 30° C.; UV: 220 nm.

Method 39: Analytical SFC conditions: Column/dimensions: Chiralcel OD-H (250×4.6) mm, 5 μm; % CO2: 80%; Co-solvent: 20% of 0.2% NH3 in MeOH; Total Flow: 4.0 g/min; Back Pressure: 100 bar; Temperature: 35° C.; UV: 220 nm.

Method 40: Column: Chiralcel OJ-H (250×21.2) mm, 5 μm; % CO2: 75%; % Co-solvent: 25% of 0.2% NH3 in MeOH; Total Flow: 80.0 g/min; Back Pressure: 100 bar; Temperature: 40° C.; UV: 265 nm.

Method 41: Column: Chiralcel OJ-H (250×4.6) mm, 5 μm; % CO2: 85%; % Co-solvent: 15% of 0.2% NH3 in MeOH; Total Flow: 4.0 g/min; Back Pressure: 100 bar; Temperature: 40° C.; UV: 265 nm.

Method 42: Column: Chiralpak IC (250×50) mm, 5 μm; % CO2: 70%; Co-solvent: 30% of 0.2% NH3 in MeOH; Total Flow: 280 g/min; Back Pressure: 100 bar; Temperature: 30° C.; UV: 246 nm.

Method 43: Analytical SFC conditions: Column: Chiralpak IC (250×4.6) mm, 5 μm; % CO2: 70%; Co-solvent: 30% of 0.2% NH3 in MeOH; Total Flow: 4 g/min; Back Pressure: 100 bar; Temperature: 30° C.; UV: 246 nm.

Method 44: Column: Gemini NX, (250×21.2) mm, 5 μm; Mobile phase A: 10 mM NH4OAc, pH 4.5 in water, Mobile phase B: ACN; Flow Rate: 20 mL/min; Column Temperature: 25° C.

Method 45: Column: Chiralcel OD-H (250×30) mm, 5 μm; % CO2: 60%; Co-solvent: 40%; 4M NH3 in MeOH; Total Flow: 100 g/min; Back Pressure: 100 bar; Temperature: 35° C.; UV: 220 nm.

Methods 46 for the isolation of the 4 isomers of tert-butyl (3-(3-((2-((4-fluoro-3-(trifluoromethyl)phenyl)carbamoyl)-6-(trifluoromethyl)benzo[b]thiophen-3-yl)carbamoyl)-4-methoxyphenyl)-3a, 5,6,6a-tetrahydro-4H-cyclopenta[d]isoxazol-5-yl)carbamate: Column: Chiralcel OJ-H (250×21) mm, 5 μm; % CO2: 80%; % Co-solvent: 20% of 0.2% DEA in IPA; Total Flow: 80.0 g/min; Back Pressure: 100 bar; Temperature: 40° C.; UV: 265 nm) (Isomer 1 Prep SFC RT=10.6 min), (Isomer 2+3 a mixture) Prep SFC RT=13.9 min), (Isomer 4 Prep SFC RT=17.1 min). Analytical SFC conditions: (Column: Chiralcel OJ-H (250×4.6) mm, 5 μm; % CO2: 80%; % Co-solvent: 20% of 0.2% DEA in IPA; Total Flow: 4.0 g/min; Back Pressure: 100 bar; Temperature: 40° C.; UV: 265 nm). (Isomer 1: Analytical SFC RT=5.01 min, 100% ee) (Isomer 4: Analytical SFC RT=8.35 min, 100% ee). The mixture of isomer 2+3 were further purified by the following Prep SFC conditions: Column: Chiralpak AS-H (250×21) mm, 5 μm; % CO2: 80%; % Co-solvent: 20% of 0.2% NH3 in MeOH; Total Flow: 80.0 g/min; Back Pressure: 100 bar; Temperature: 30° C.; UV: 236 nm) (Isomer 2 Prep SFC RT=5.4 min, 100% ee) (Isomer 3 Prep SFC RT=7.5 min, 98% ee). Analytical SFC conditions: (Column: Chiralpak AS-H (250×4.6) mm, 5 μm; % CO2: 80%; % Co-solvent: 20% of 0.2% NH3 in MeOH; Total Flow: 3.0 g/min; Back Pressure: 100 bar; Temperature: 30° C.; UV: 236 nm).

Methods 47 for the isolation of the 4 isomers of methyl 5-(5-(hydroxymethyl)-3a, 5,6,6a-tetrahydro-4H-cyclopenta[d]isoxazol-3-yl)-2-methoxybenzoate: Column: Chiralpak AD-H (250×21) mm, 5 μm; % CO2: 85%; % Co-solvent: 15% MeOH; Total Flow: 45.0 mL/min; Back Pressure: 150 bar; Temperature: 40° C.; UV: 210 nm) to give isomer 1 (Prep SFC RT=4.07 min, >99% ee), isomer 2 (Prep SFC RT=4.55 min, >99% ee), isomer 3 (Prep SFC RT=5.66 min, >99% ee) and isomer 4 (Prep SFC RT=9.81 min, >99% ee). Analytical SFC conditions: (Column: Chiralpak AD-H, (4.6×100) mm, 3 micron; Mobile phase: 15% MeOH/85% CO2; Flow conditions: 2.0 mL/min, 150 Bar, 40° C.; Detector wavelength: 220 nm).

### Example 1

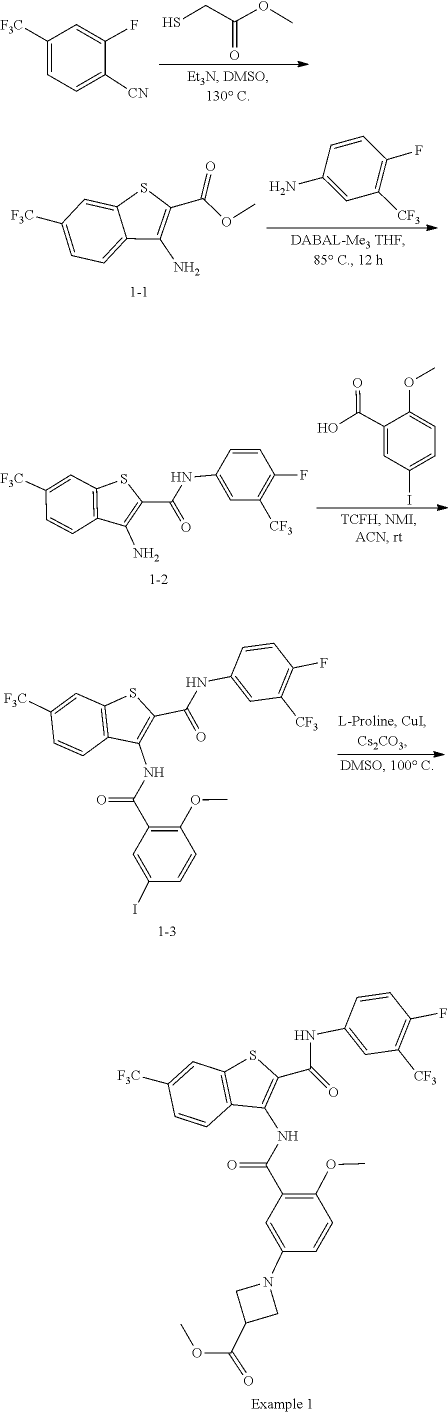

### Intermediate 1-1: Methyl 3-amino-6-(trifluoromethyl) benzo[b]thiophene-2-carboxylate

A mixture of 2-fluoro-4-(trifluoromethyl)benzonitrile (1.00 g, 5.29 mmol), methyl 2-mercaptoacetate (0.589 g, 5.55 mmol) and TEA (2.29 mL, 16.4 mmol) in dry DMSO (10 mL) was irradiated in a microwave reactor at 130° C. for 3 h. The cooled reaction mass was poured into ice-water and the solid precipitate was filtered, washed with water and dried under vacuum to give Intermediate 1-1 (1.00 g, 3.63 mmol, 69% yield). LC-MS RT: 1.91 min; MS (ESI) m/z 276.1 (M+H)⁺; Method E.

### Intermediate 1-2: 3-amino-N-(4-fluoro-3-(trifluoromethyl) phenyl)-6-(trifluoromethyl) benzo[b]thiophene-2-carboxamide

To a solution of Intermediate 1-1 (500 mg, 1.82 mmol) and 4-fluoro-3-(trifluoromethyl)aniline (260 mg, 1.45 mmol) in THF (15 mL) was added bis(trimethylaluminium)-1,4-diazabicyclo[2.2.2]octane adduct (466 mg, 1.82 mmol). The resulting reaction mixture was stirred for 12 h at 85° C. The reaction mass was quenched with aqueous sodium potassium tartrate solution (30 mL) and extracted with DCM (2×50 mL). The combined organic layers were dried over Na₂SO₄, filtered and concentrated under reduced pressure. The residue was purified by column chromatography to give Intermediate 1-2 (385 mg, 0.912 mmol, 50% yield). LC-MS RT: 1.12 min; MS (ESI) m/z 421.2 (M−H)⁻; Method E.

### Intermediate 1-3: N-(4-fluoro-3-(trifluoromethyl)phenyl)-3-(5-iodo-2-methoxybenzamido)-6-(trifluoromethyl)benzo[b]thiophene-2-carboxamide

To a solution of Intermediate 1-2 (1.20 g, 2.88 mmol) and 5-iodo-2-methoxybenzoic acid (1.00 g, 3.60 mmol) in ACN (11 mL) was added chloro-N,N,N′,N′-tetramethylformamidinium hexafluorophosphate (2.02 g, 7.19 mmol) followed by 1-Methylimidazole (1.48 g, 18.0 mmol). The resulting reaction mixture was stirred at rt for 16 h. The reaction mixture was quenched with ice water (50 mL). The solid precipitate was filtered and dried under vacuum to give Intermediate 1-3 (1.00 g, 1.47 mmol, 41% yield). LC-MS RT: 2.28 min; MS (ESI) m/z 683.0 (M+H)⁺; Method E.

### Example 1: Methyl 1-(3-((2-((4-fluoro-3-(trifluoromethyl)phenyl)carbamoyl)-6-(trifluoromethyl)benzo[b]thiophen-3-yl)carbamoyl)-4-methoxyphenyl)azetidine-3-carboxylate

To a solution of Intermediate 1-3 (300 mg, 0.44 mmol) in DMSO (2 mL) was added methyl azetidine-3-carboxylate (76 mg, 0.66 mmol), and the resulting solution was degassed with N₂ for 10 min. L-Proline (15 mg, 0.13 mmol), copper (I) iodide (17 mg, 0.088 mmol) and cesium carbonate (430 mg, 1.3 mmol) were added to the degassed reaction mixture and stirred at 100° C. for 16 h. The reaction solution was diluted with EtOAc (100 mL), washed with water (2×100 mL) and brine solution (50 mL). The separated organic layer was dried over Na₂SO₄, filtered and concentrated under reduced pressure. The residue was purified by prep LCMS (Method 1) to yield Example 1 (100 mg, 0.15 mmol, 34% yield). ¹H NMR (400 MHz, DMSO-d6) δ ppm 10.86 (s, 1H), 10.73 (s, 1H), 8.66 (s, 1H), 8.15 (dd, J=2.7, 6.6 Hz, 1H), 8.05 (d, J=8.8 Hz, 1H), 8.02-7.93 (m, 1H), 7.81 (dd, J=1.6, 8.7 Hz, 1H), 7.54 (t, J=9.8 Hz, 1H), 7.12 (d, J=8.8 Hz, 1H), 6.92 (d, J=3.2 Hz, 1H), 6.70 (dd, J=2.9, 8.8 Hz, 1H), 3.99 (t, J=7.7 Hz, 2H), 3.88 (s, 3H), 3.81 (t, J=6.6 Hz, 2H), 3.67 (s, 3H), 3.65-3.55 (m, 1H). LC-MS RT: 2.54 min; MS (ESI) m/z 670.2 (M+H)⁺; Method A.

### Example 2

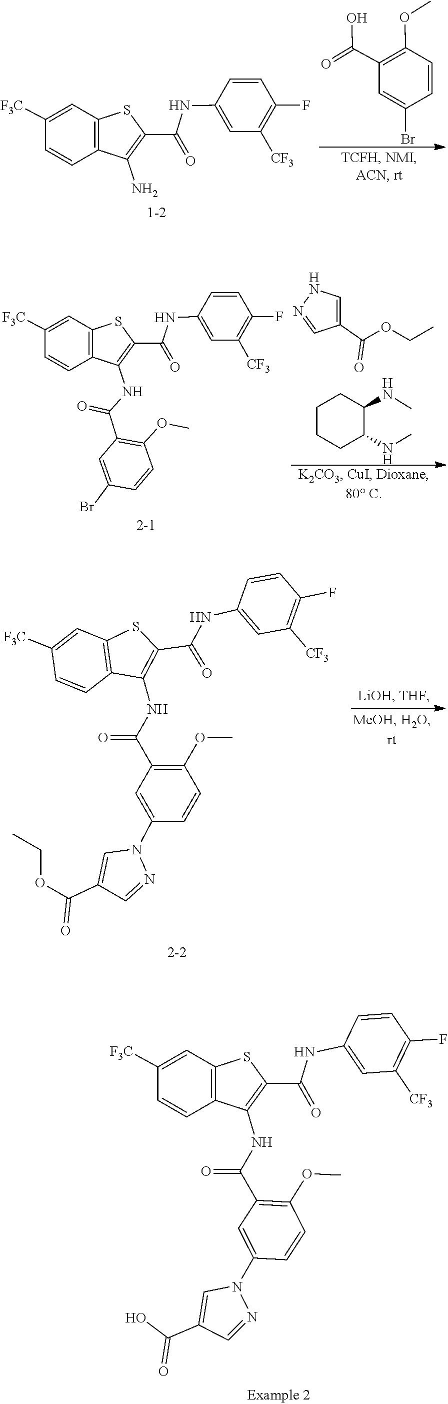

### Intermediate 2-1: 3-(5-bromo-2-methoxybenzamido)-N-(4-fluoro-3-(trifluoromethyl) phenyl)-6-(trifluoromethyl) benzo[b]thiophene-2-carboxamide

Intermediate 2-1 (0.48 g, 0.76 mmol, 80% yield) was prepared from Intermediate 1-2 (400 mg, 0.95 mmol) and 5-bromo-2-methoxybenzoic acid (220 mg, 0.95 mmol) in a similar way as Intermediate 1-3. LC-MS RT: 1.52 min; MS (ESI) m/z 635.0 (M+H)⁺; Method E.

### Intermediate 2-2: Ethyl 1-(3-((2-((4-fluoro-3-(trifluoromethyl)phenyl)carbamoyl)-6-(trifluoromethyl)benzo[b]thiophen-3-yl)carbamoyl)-4-methoxyphenyl)-1H-pyrazole-4-carboxylate

To a solution of Intermediate 2-1 (300 mg, 0.47 mmol) in 1,4-dioxane (2 mL) was added ethyl 1H-pyrazole-4-carboxylate (99 mg, 0.71 mmol), trans-N,N′-dimethylcyclohexane-1,2-diamine (27 mg, 0.094 mmol) followed by potassium carbonate (200 mg, 1.4 mmol) and copper(I) iodide (18 mg, 0.094 mmol). The resulting reaction mixture was stirred at 80° C. for 16 h. The reaction solution was diluted with EtOAc (100 mL), washed with water (2×100 mL) and brine solution (100 mL). The separated organic layer was dried over Na₂SO₄, filtered and concentrated under reduced pressure. The residue was purified by column chromatography to afford Intermediate 2-2 (210 mg, 0.30 mmol, 64% yield). LC-MS RT: 2.18 min; MS (ESI) m/z 695.2 (M+H)⁺; Method E.

### Example 2: 1-(3-((2-((4-fluoro-3-(trifluoromethyl) phenyl) carbamoyl)-6-(trifluoromethyl) benzo[b]thiophen-3-yl) carbamoyl)-4-methoxyphenyl)-1H-pyrazole-4-carboxylic acid

To a solution of Intermediate 2-2 (80 mg, 0.12 mmol) in THF (5 mL), MeOH (2 mL) and Water (1 mL) was added LiOH (5.5 mg, 0.23 mmol). The resulting reaction mixture was stirred at rt for 3 h. The reaction mass was concentrated under reduced pressure, the aqueous layer was acidified to pH 4-5, and the resulting precipitate was filtered and dried in vacuo. The precipitate was purified by prep LCMS (Method 1) to provide Example 2 (41 mg, 0.060 mmol, 53% yield). ¹H NMR (400 MHz, DMSO-d6) δ ppm 11.04-10.70 (m, 2H), 8.97 (s, 1H), 8.67 (s, 1H), 8.32 (d, J=2.7 Hz, 2H), 8.20-8.14 (m, 1H), 8.13-7.96 (m, 2H), 7.82 (dd, J=1.8, 8.7 Hz, 2H), 7.53 (t, J=9.8 Hz, 1H), 7.37 (d, J=9.0 Hz, 1H), 3.95 (s, 3H). LC-MS RT: 2.30 min; MS (ESI) m/z 667.1 (M+H)⁺; Method B.

### Example 3: N-(4-fluoro-3-(trifluoromethyl) phenyl)-3-(5-(4-(2-hydroxypropan-2-yl)-1H-pyrazol-1-yl)-2-methoxybenzamido)-6-(trifluoromethyl) benzo[b]thiophene-2-carboxamide

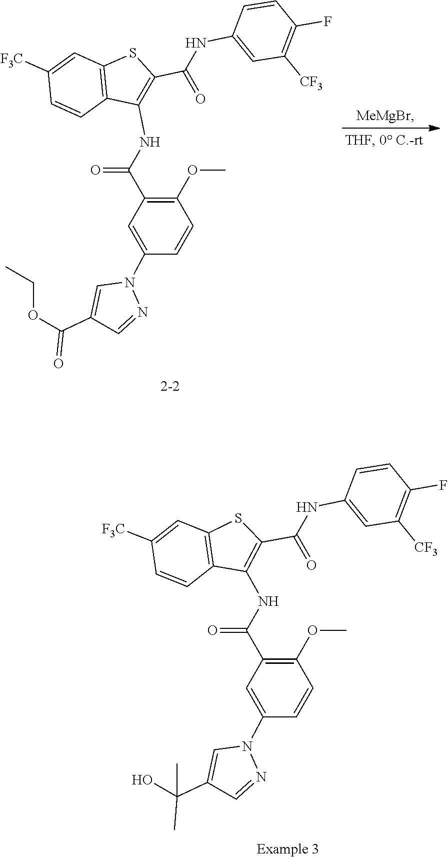 [SMILES: CCOC(=O)c1cnn(-c2ccc(OC)c(C(=O)Nc3c(C(=O)Nc4ccc(F)c(C(F)(F)F)c4)sc4cc(C(F)(F)F)ccc34)c2)c1 ; COc1ccc(-n2cc(C(C)(C)O)cn2)cc1C(=O)Nc1c(C(=O)Nc2ccc(F)c(C(F)(F)F)c2)sc2cc(C(F)(F)F)ccc12]

A solution of Intermediate 2-2 (80 mg, 0.12 mmol) in THF (10 mL) was cooled to 0° C. Methyl magnesium bromide solution (0.19 mL, 0.58 mmol) was added and the reaction solution was stirred at rt for 3 h. The reaction mass was quenched with water (50 mL) and extracted with EtOAc (2×50 mL). The separated organic layer was dried over Na₂SO₄, filtered and concentrated under reduced pressure. The residue was purified by prep LCMS (Method 1) to give Example 3 (9.3 mg, 0.031 mmol, 12% yield). ¹H NMR (400 MHz, DMSO-d6) δ ppm 10.97-10.88 (m, 1H), 10.82-10.70 (m, 1H), 8.68 (s, 1H), 8.28 (s, 1H), 8.24 (d, J=2.9 Hz, 1H), 8.18 (dd, J=2.7, 6.4 Hz, 1H), 8.10 (d, J=8.6 Hz, 1H), 8.05-7.93 (m, 2H), 7.82 (dd, J=1.6, 8.7 Hz, 1H), 7.65 (s, 1H), 7.54 (t, J=9.8 Hz, 1H), 7.35 (d, J=9.0 Hz, 1H), 4.95 (s, 1H), 3.97 (s, 3H), 1.46 (s, 6H). LC-MS RT: 2.39 min; MS (ESI) m/z 681.2 (M+H)⁺; Method A.

### Example 4 and 5

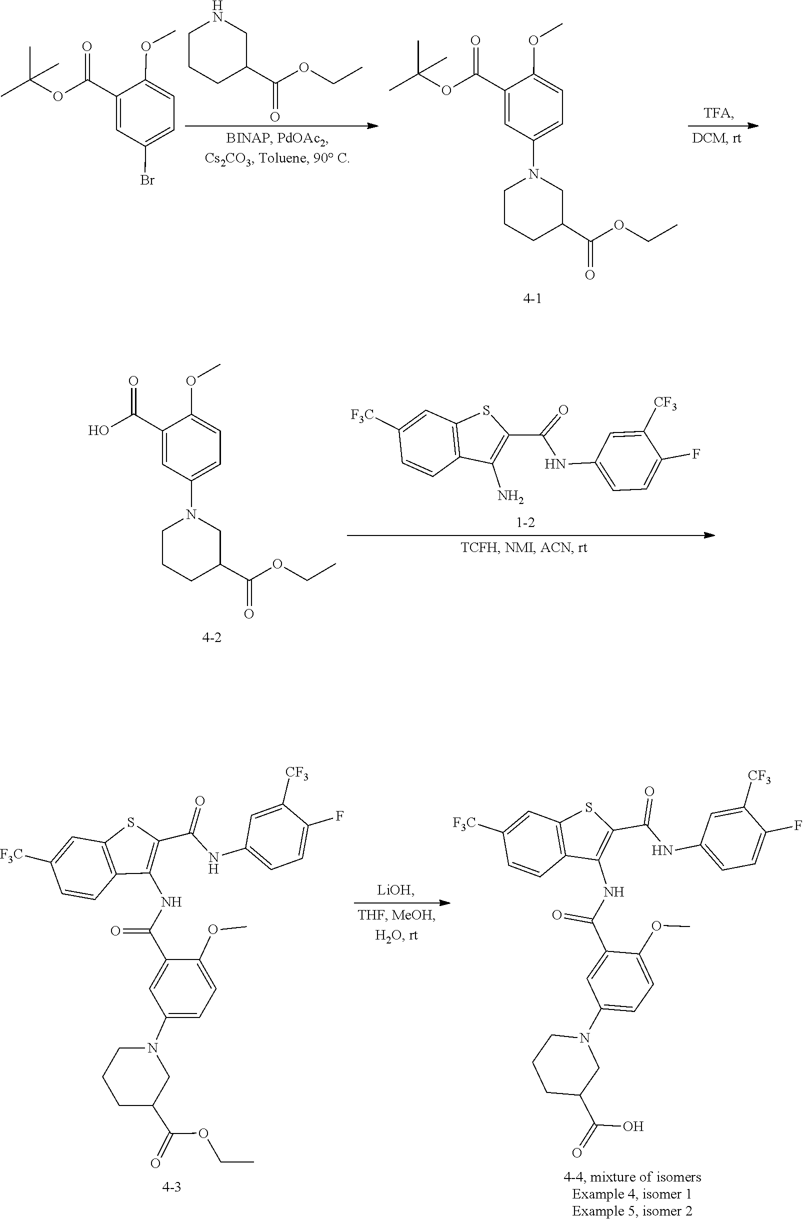

### Intermediate 4-1: Ethyl 1-(3-(tert-butoxycarbonyl)-4-methoxyphenyl) piperidine-3-carboxylate

To a solution of tert-butyl 5-bromo-2-methoxybenzoate (1.20 g, 4.18 mmol) and ethyl piperidine-3-carboxylate (854 mg, 5.43 mmol) in toluene (30 mL) was added cesium carbonate (4.08 g, 12.5 mmol). The resulting solution was degassed with N₂ for 10 min, Pd(OAc)₂ (94.0 mg, 0.418 mmol) and BINAP (520 mg, 0.836 mmol) were added, and the solution was degassed again for 5 min. The resulting reaction mixture was heated at 90° C. for 12 h. The reaction mass was concentrated under reduced pressure, the residue was diluted with EtOAc and washed with water. The organic layer was dried over Na₂SO₄, filtered and concentrated under reduced pressure. The residue was purified by column chromatography to give Intermediate 4-1 (350 mg, 0.963 mmol, 23% yield). LC-MS RT: 1.06 min; MS (ESI) m/z 364.2 (M+H)⁺; Method E.

### Intermediate 4-2: 5-(3-(ethoxycarbonyl) piperidin-1-yl)-2-methoxybenzoic acid

To a solution of Intermediate 4-1 (50 mg, 0.14 mmol) in DCM (2 mL) was added TFA (0.50 mL). The resulting reaction mixture was stirred at rt for 12 h. The reaction mass was concentrated under vacuum to give Intermediate 4-2 (40 mg, 0.13 mmol, 95% yield). LC-MS RT: 0.35 min; MS (ESI) m/z 307.8 (M+H)⁺; Method E.

### Intermediate 4-3: Ethyl 1-(3-((2-((4-fluoro-3-(trifluoromethyl) phenyl) carbamoyl)-6-(trifluoromethyl) benzo[b]thiophen-3-yl) carbamoyl)-4-methoxyphenyl) piperidine-3-carboxylate

Intermediate 4-3 (80 mg, 0.11 mmol, 48% yield) was prepared from Intermediate 4-2 (150 mg, 0.47 mmol and Intermediate 1-2 (100 mg, 0.24 mmol) in a similar way as Intermediate 1-3. LC-MS RT: 1.46 min; MS (ESI) m/z 712.3 (M+H)⁺; Method E.

### Example 4 and 5: 1-(3-((2-((4-fluoro-3-(trifluoromethyl) phenyl) carbamoyl)-6-(trifluoromethyl) benzo[b]thiophen-3-yl) carbamoyl)-4-methoxyphenyl) piperidine-3-carboxylic acid

Intermediate 4-4 was prepared by the general procedure described for Example 2, followed by purification by prep LCMS (Method 1). The isomers were separated by prep SFC purification (Column: Chiralpak IG (250×30) mm, 5 μm; % CO2: 50%; Co-solvent: 50% of 0.1% DEA IN IPA; Total Flow: 80 g/min; Back Pressure: 100 bar; Temperature: 35° C.; UV: 220 nm) to give Example 4 (Prep SFC RT=9.47 min, 98% ee) and Example 5 (Prep SFC RT=12.36 min, 98% ee).

Example 4: (29 mg, 0.042 mmol, 20% yield). ¹H NMR (400 MHz, DMSO-d6) δ ppm 10.87 (s, 2H), 8.64 (s, 1H), 8.20-8.12 (m, 1H), 8.08 (d, J=9.5 Hz, 1H), 7.98 (dddd, J=1.2, 2.7, 5.9, 7.5 Hz, 1H), 7.80 (d, J=8.3 Hz, 2H), 7.63-7.44 (m, 1H), 7.41-7.32 (m, 2H), 7.12 (s, 1H), 3.87 (s, 3H), 3.49 (br s, 2H), 3.44-3.39 (m, 2H), 2.87-2.63 (m, 2H), 2.61-2.53 (m, 1H), 1.98-1.82 (m, 1H), 1.58-1.44 (m, 1H). LC-MS RT: 3.38 min; MS (ESI) m/z 684.2 (M+H)⁺; Method C.

Example 5: (3.0 mg, 4.5 μmol, 2% yield). ¹H NMR (400 MHz, DMSO-d6) δ ppm 10.87 (s, 2H), 10.74 (s, 1H), 8.64 (s, 1H), 8.16 (dd, J=2.2, 6.4 Hz, 1H), 8.07 (d, J=8.8 Hz, 1H), 7.99 (td, J=3.7, 8.6 Hz, 1H), 7.80 (d, J=9.3 Hz, 1H), 7.52 (t, J=9.7 Hz, 1H), 7.38 (br s, 1H), 7.20-7.04 (m, 2H), 3.91-3.84 (m, 3H), 3.51 (br d, J=1.2 Hz, 1H), 2.86-2.75 (m, 2H), 2.70-2.62 (m, 1H), 1.94-1.84 (m, 1H), 1.79-1.68 (m, 1H), 1.62-1.45 (m, 2H), 1.10 (t, J=7.1 Hz, 1H). LC-MS RT: 2.05 min; MS (ESI) m/z 684.2 (M+H)⁺; Method A.

### Example 6: 1-(3-((2-((4-fluoro-3-(trifluoromethyl) phenyl) carbamoyl)-6-(trifluoromethyl) benzo[b]thiophen-3-yl) carbamoyl)-4-methoxyphenyl)-N-(methylsulfonyl) piperidine-3-carboxamide

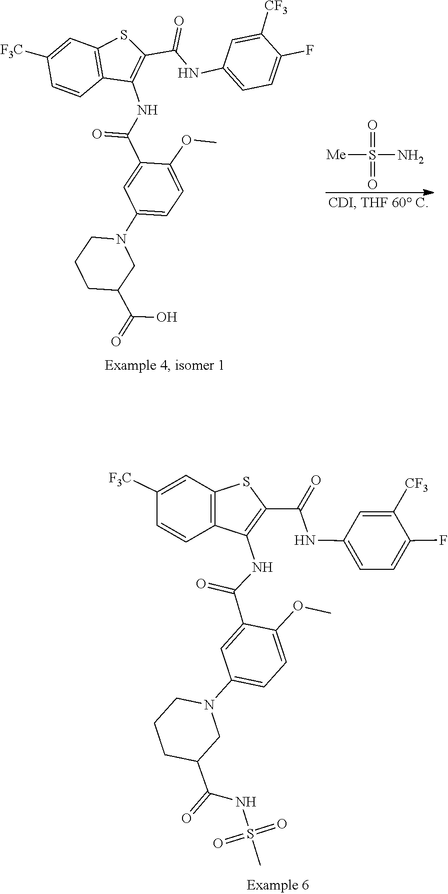 [SMILES: COc1ccc(N2CCCC(C(=O)O)C2)cc1C(=O)Nc1c(C(=O)Nc2ccc(F)c(C(F)(F)F)c2)sc2cc(C(F)(F)F)ccc12 ; CS(N)(=O)=O ; COc1ccc(N2CCCC(C(=O)NS(C)(=O)=O)C2)cc1C(=O)Nc1c(C(=O)Nc2ccc(F)c(C(F)(F)F)c2)sc2cc(C(F)(F)F)ccc12]

To a solution of Example 4 (10 mg, 0.015 mmol) in THF (3 mL) was added CDI (30 mg, 0.19 mmol) and the reaction mixture was heated at 60° C. for 2 h. Methanesulfonamide (50 mg, 0.53 mmol) was then added and the reaction mixture was continued heating for another 4 h. The reaction mass was diluted with EtOAc (10 mL), washed with water (2×10 mL). The separated organic layer was dried over Na₂SO₄, filtered and concentrated under reduced pressure. The residue was purified by prep LCMS (Method 1) to give Example 6 (1.2 mg, 1.6 mol, 10% yield). ¹H NMR (400 MHz, DMSO-d6) δ ppm 11.79 (br d, J=2.4 Hz, 1H), 10.87 (s, 1H), 10.74 (s, 1H), 8.66 (s, 1H), 8.16 (dd, J=2.7, 6.6 Hz, 1H), 8.06 (d, J=8.3 Hz, 1H), 8.02-7.94 (m, 1H), 7.81 (dd, J=1.3, 8.7 Hz, 1H), 7.54 (t, J=9.8 Hz, 1H), 7.45 (d, J=3.2 Hz, 1H), 7.25-7.19 (m, 1H), 7.17-7.08 (m, 1H), 3.91 (s, 3H), 3.69-3.61 (m, 1H), 3.45-3.38 (m, 2H), 3.22 (s, 3H), 2.67 (br d, J=2.0 Hz, 2H), 1.98-1.71 (m, 2H), 1.61-1.43 (m, 2H). LC-MS RT: 2.05 min; MS (ESI) m/z 761.2 (M+H)⁺; Method A.

### Example 7 and 8: 1-(3-((2-((4-fluoro-3-(trifluoromethyl) phenyl) carbamoyl)-6-(trifluoromethyl) benzo[b]thiophen-3-yl) carbamoyl)-4-methoxyphenyl)-N-(2-hydroxy-2-methylpropyl) piperidine-3-carboxamide

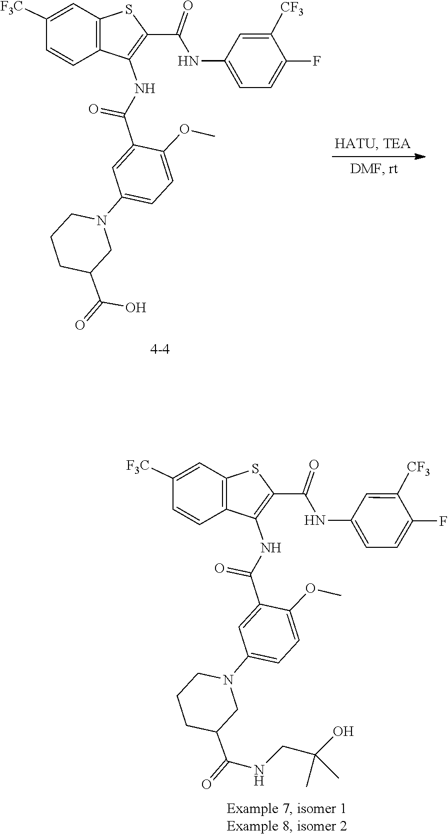 [SMILES: COc1ccc(N2CCCC(C(=O)O)C2)cc1C(=O)Nc1c(C(=O)Nc2ccc(F)c(C(F)(F)F)c2)sc2cc(C(F)(F)F)ccc12 ; COc1ccc(N2CCCC(C(=O)NCC(C)(C)O)C2)cc1C(=O)Nc1c(C(=O)Nc2ccc(F)c(C(F)(F)F)c2)sc2cc(C(F)(F)F)ccc12]

To a solution of Intermediate 4-4 (50 mg, 0.073 mmol), 1-amino-2-methylpropan-2-ol (6.5 mg, 0.073 mmol) in DMF (2 mL) was added TEA (0.031 mL, 0.22 mmol), followed by HATU (42 mg, 0.11 mmol). The resulting solution was stirred at rt for 12 h. The reaction mass was concentrated under vacuum. The residue was purified by prep HPLC (Method 2), followed by purification via prep SFC (Method 8) to give Example 7 (Prep SFC RT=2.9 min, 99% ee) and Example 8 (Prep SFC RT=4.4 min, 99% ee). Analytical SFC conditions: Column: Chiralcel OD-H (250×4.6) mm, 5 μm; % CO2: 60%; Co-solvent: 40% 4M NH3 in MeOH; Total Flow: 3.0 g/min; Back Pressure: 100 bar; Temperature: 35° C.; UV: 240 nm.

Example 7: (2.6 mg, 3.4 μmol, 5% yield). ¹H NMR (400 MHz, DMSO-d6) δ ppm 10.85 (s, 1H), 10.73 (s, 1H), 8.66 (s, 1H), 8.17 (dd, J=2.7, 6.6 Hz, 1H), 8.08-7.96 (m, 2H), 7.83-7.73 (m, 2H), 7.54 (t, J=9.8 Hz, 1H), 7.42 (d, J=2.9 Hz, 1H), 7.22-7.12 (m, 2H), 4.42 (s, 1H), 3.90 (s, 3H), 3.32 (s, 1H), 3.32-3.17 (m, 1H), 3.13-2.90 (m, 2H), 2.74-2.66 (m, 1H), 2.50 (td, J=1.8, 3.7 Hz, 2H), 1.87-1.69 (m, 1H), 1.65-1.40 (m, 2H), 1.04 (s, 6H). LC-MS RT: 2.34 min; MS (ESI) m/z 755.2 (M+H)⁺; Method B.

Example 8: (2.4 mg, 3.1 μmol, 4% yield)¹H NMR (400 MHz, DMSO-d6) δ ppm 10.85 (s, 1H), 10.73 (s, 1H), 8.66 (s, 1H), 8.16 (dd, J=2.6, 6.5 Hz, 1H), 8.08-7.96 (m, 2H), 7.83-7.73 (m, 2H), 7.54 (t, J=9.8 Hz, 1H), 7.42 (d, J=2.9 Hz, 1H), 7.22-7.12 (m, 2H), 4.42 (s, 1H), 3.90 (s, 3H), 3.43 (br d, J=12.2 Hz, 1H), 3.32 (s, 1H), 3.17 (s, 1H), 3.14-2.91 (m, 2H), 2.50 (td, J=1.9, 3.5 Hz, 2H), 2.33 (br d, J=1.7 Hz, 1H), 1.87-1.68 (m, 1H), 1.65-1.43 (m, 2H), 1.04 (s, 6H). LC-MS RT: 2.34 min; MS (ESI) m/z 755.2 (M+H)⁺; Method B.

### Example 9

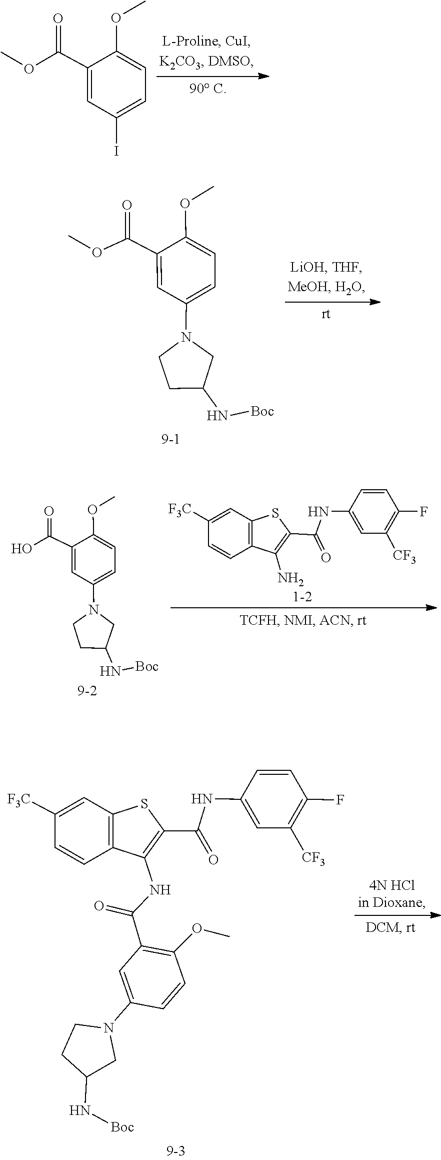 [SMILES: COC(=O)c1cc(I)ccc1OC ; COC(=O)c1cc(N2CCC(NC(=O)OC(C)(C)C)C2)ccc1OC ; COc1ccc(N2CCC(NC(=O)OC(C)(C)C)C2)cc1C(=O)O ; Nc1c(C(=O)Nc2ccc(F)c(C(F)(F)F)c2)sc2cc(C(F)(F)F)ccc12 ; COc1ccc(N2CCC(NC(=O)OC(C)(C)C)C2)cc1C(=O)Nc1c(C(=O)Nc2ccc(F)c(C(F)(F)F)c2)sc2cc(C(F)(F)F)ccc12]

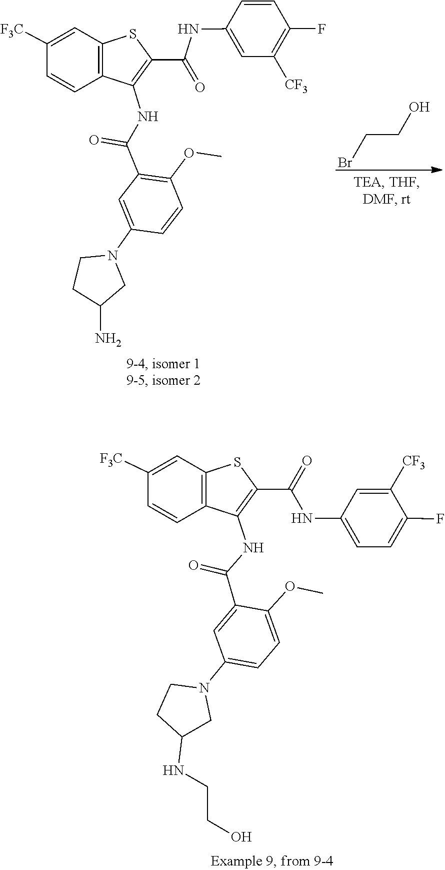

### Intermediate 9-1: Methyl 5-(3-((tert-butoxycarbonyl) amino) pyrrolidin-1-yl)-2-methoxybenzoate

Intermediate 9-1 (350 mg, 0.999 mmol, 58% yield) was prepared from methyl 5-iodo-2-methoxybenzoate (500 mg, 1.71 mmol) and tert-butyl pyrrolidin-3-ylcarbamate (319 mg, 1.71 mmol) according to the general method outlined for Example 1, except using K₂CO₃ as a base rather than Cs₂CO₃. LC-MS RT: 2.45 min; MS (ESI) m/z 351.4 (M+H)⁺; Method A.

### Intermediate 9-2: 5-(3-((tert-butoxycarbonyl) amino) pyrrolidin-1-yl)-2-methoxybenzoic acid

Intermediate 9-2 (400 mg, 1.19 mmol, 83% yield) was prepared from Intermediate 9-1 (500 mg, 1.43 mmol) by the general procedure described in Example 2. LC-MS RT: 1.65 min; MS (ESI) m/z 337.2 (M+H)⁺; Method C.

### Intermediate 9-3: Tert-butyl (1-(3-((2-((4-fluoro-3-(trifluoromethyl) phenyl) carbamoyl)-6-(trifluoromethyl) benzo[b]thiophen-3-yl) carbamoyl)-4-methoxyphenyl) pyrrolidin-3-yl) carbamate

Example 9-3 (250 mg, 0.337 mmol, 71% yield) was prepared from Intermediate 1-2 (200 mg, 0.474 mmol) and Intermediate 9-2 (159 mg, 0.474 mmol) in a similar way as Intermediate 1-3. LC-MS RT: 3.24 min; MS (ESI) m/z 739.3 (M−H)⁻; Method C.

### Intermediate 9-4 and 9-5: 3-(5-(3-aminopyrrolidin-1-yl)-2-methoxybenzamido)-N-(4-fluoro-3-(trifluoromethyl) phenyl)-6-(trifluoromethyl) benzo[b]thiophene-2-carboxamide

A solution of Intermediate 9-3 (100 mg, 0.135 mmol) in 4M HCl in Dioxane (5 mL) was stirred at rt for 12 h. The reaction mass was concentrated under reduced pressure. The residue was purified by prep LCMS (Method 1), followed by prep SFC (Method 10) to give Intermediate 9-4 (Prep SFC RT=7.25 min, 99% ee) and Intermediate 9-5 (Prep SFC RT=9.36 min, 99% ee). Analytical SFC conditions: Column: Chiralpak IC (250×4.6) mm, 5 μm; % CO₂: 75%; Co-solvent: 25% of 4M NH3 in MeOH; Total Flow: 4.0 g/min; Back Pressure: 100 bar; Temperature: 35° C.; UV: 220 nm.

Intermediate 9-4: (2.7 mg, 4.2 μmol, 3% yield) LC-MS RT: 1.98 min; MS (ESI) m/z 641.3 (M+H)⁺; Method B.

Intermediate 9-5: (3.7 mg, 5.5 μmol, 4% yield)¹H NMR (400 MHz, DMSO-d6) δ ppm 10.99-10.68 (m, 2H), 8.65 (s, 1H), 8.14 (dd, J=2.6, 6.5 Hz, 1H), 8.02 (d, J=7.8 Hz, 1H), 8.00-7.91 (m, 1H), 7.80 (dd, J=1.5, 8.3 Hz, 1H), 7.53 (t, J=9.9 Hz, 1H), 7.12 (d, J=2.9 Hz, 1H), 7.05 (d, J=9.3 Hz, 1H), 6.79 (dd, J=3.3, 8.9 Hz, 1H), 5.65-5.53 (s, 2H), 3.90-3.77 (m, 2H), 3.87 (s, 3H), 3.11-3.06 (m, 2H), 3.01-2.97 (m, 1H), 1.89 (s, 2H). LC-MS RT: 2.00 min; MS (ESI) m/z 641.3 (M+H)⁺; Method B.

### Example 9: N-(4-fluoro-3-(trifluoromethyl) phenyl)-3-(5-(3-((2-hydroxyethyl) amino) pyrrolidin-1-yl)-2-methoxybenzamido)-6-(trifluoromethyl) benzo[b]thiophene-2-carboxamide

To a solution of Intermediate 9-4 (20 mg, 0.031 mmol) and 2-bromoethan-1-ol (3.9 mg, 0.031 mmol) in THF (2 mL) and DMF (1 mL) was added TEA (4.4 μl, 0.031 mmol). The resulting reaction mixture was stirred at rt for 12 h. The reaction mass was diluted with EtOAc (20 mL), washed with water (2×20 mL), dried over Na₂SO₄, and concentrated under reduced pressure. The residue was purified by prep LCMS (Method 1) to give Example 9 (1.0 mg, 1.4 μmol, 5% yield). ¹H NMR (400 MHz, DMSO-d6) δ ppm 10.85 (br d, J=2.2 Hz, 1H), 10.73 (br s, 1H), 8.66 (s, 1H), 8.16 (dd, J=2.4, 6.6 Hz, 1H), 8.07-7.93 (m, 2H), 7.81 (dd, J=1.3, 8.7 Hz, 1H), 7.54 (t, J=9.9 Hz, 1H), 7.10 (d, J=2.7 Hz, 1H), 7.04 (d, J=8.8 Hz, 1H), 6.79 (dd, J=3.1, 9.2 Hz, 1H), 5.66-5.54 (m, 1H), 4.57-4.35 (m, 1H), 3.95-3.75 (m, 5H), 3.49 (br d, J=4.9 Hz, 3H), 2.92-2.82 (m, 1H), 2.68 (br s, 1H), 2.18-2.09 (m, 1H), 1.60-1.48 (m, 1H). LC-MS RT: 1.99 min; MS (ESI) m/z 685.3 (M+H)⁺; Method B.

### Example 10

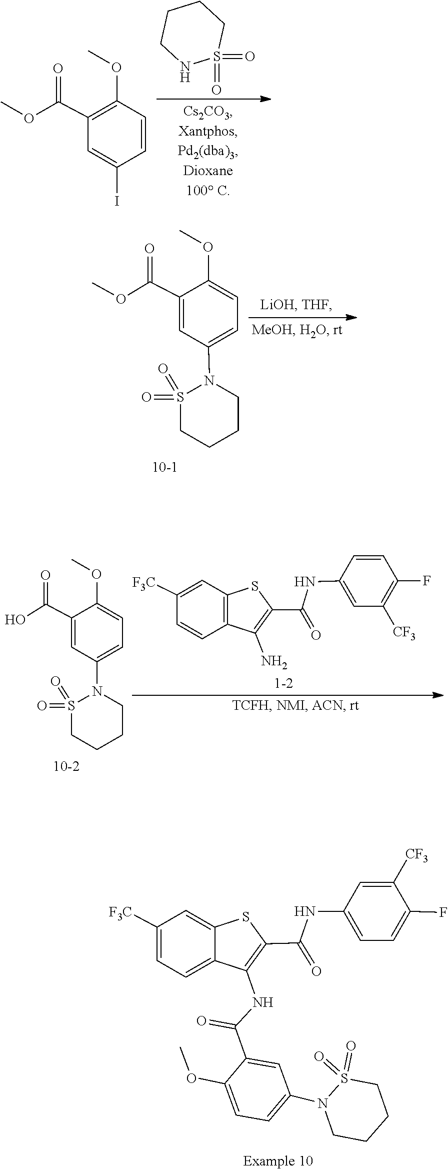

### Intermediate 10-1: methyl 5-(1,1-dioxido-1,2-thiazinan-2-yl)-2-methoxybenzoate

A solution of 1,2-thiazinane 1,1-dioxide (54 mg, 0.40 mmol), methyl 5-iodo-2-methoxybenzoate (120 mg, 0.40 mmol), Xantphos (23 mg, 0.040 mmol), cesium carbonate (260 mg, 0.79 mmol) in dioxane (2.1 mL) was purged with N₂ for 10 min, then Pd₂(dba)₃ (18 mg, 0.020 mmol) was added and the resulting reaction mixture was heated at 100° C. for 15 h. The reaction mixture was partitioned with water (10 mL) and EtOAc (30 mL). The aqueous layer was extracted with EtOAc (2×20 mL). The combined organic layers were washed with brine (15 mL), dried over MgSO₄, and concentrated under reduced pressure to give Intermediate 10-1 (120 mg, 0.40 mmol), which was used without further purification. LC-MS RT: 0.98 min; MS (ESI) m/z 300.2 (M+H)⁺; Method F.

Intermediate 10-2: 5-(1,1-dioxido-1,2-thiazinan-2-yl)-2-methoxybenzoic acid Intermediate 10-2 (61 mg, 0.21 mmol, 54% yield) was prepared from Intermediate 10-1 (120 mg, 0.40 mmol) by the general procedure described for Example 2. ¹H NMR (400 MHz, CDCl₃) δ ppm 8.12 (d, J=2.9 Hz, 1H), 7.69-7.63 (m, 1H), 7.13-7.09 (m, 1H), 4.11-4.10 (m, 4H), 3.28-3.18 (m, 2H), 2.44-2.34 (m, 2H), 1.72-1.58 (m, 4H). LC-MS RT: 0.67 min; MS (ESI) m/z 286.3 (M+H)⁺; Method F.

### Example 10: 3-(5-(1,1-dioxido-1,2-thiazinan-2-yl)-2-methoxybenzamido)-N-(4-fluoro-3-(trifluoromethyl)phenyl)-6-(trifluoromethyl)benzo[b]thiophene-2-carboxamide

Example 10 (1.6 mg, 2.3 μmol, 9% yield) was prepared from Intermediate 1-2 (10 mg, 0.025 mmol) and Intermediate 10-2 (7.0 mg, 0.025 mmol) in a similar way as Intermediate 1-3. ¹H NMR (500 MHz, DMSO-d6) δ ppm 10.94-10.71 (m, 1H), 8.66 (s, 1H), 8.18 (br d, J=3.5 Hz, 1H), 8.06 (br d, J=8.4 Hz, 1H), 8.01-7.93 (m, 1H), 7.83 (br d, J=8.6 Hz, 1H), 7.73 (br d, J=1.7 Hz, 1H), 7.60-7.45 (m, 2H), 7.26 (d, J=9.0 Hz, 1H), 3.97 (s, 3H), 3.62-3.54 (m, 2H), 3.45-3.35 (m, 2H), 3.31-3.22 (m, 1H), 2.21-2.07 (m, 2H), 1.81 (br d, J=3.9 Hz, 2H). LC-MS RT: 2.58 min; MS (ESI) m/z 690.2 (M+H)⁺; Method A.

### Example 11

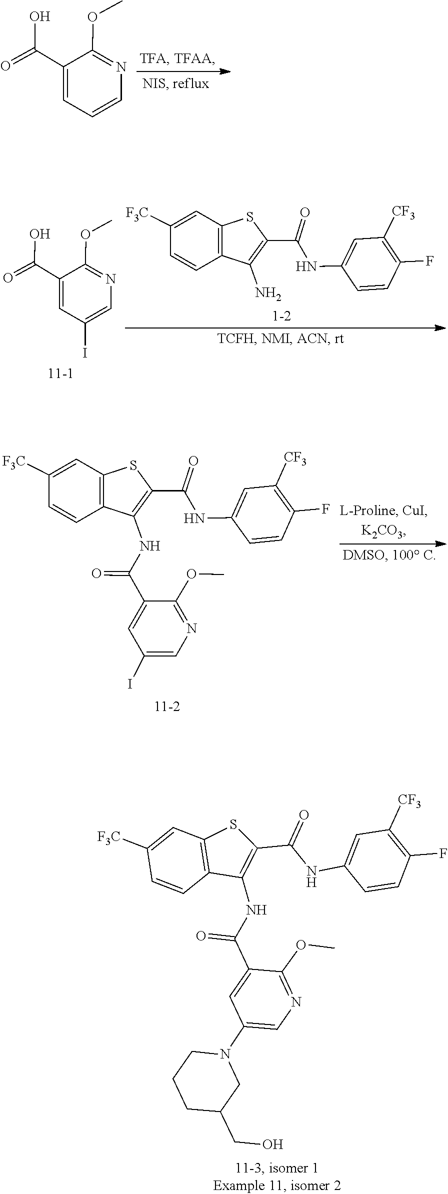

### Intermediate 11-1: 5-iodo-2-methoxynicotinic acid

To a solution of 2-methoxynicotinic acid (2.00 g, 13.1 mmol) in mixture of TFA (30.0 ml, 389 mmol) and TFAA (8.00 ml, 56.6 mmol) was added NIS (4.41 g, 19.6 mmol). The resulting reaction mixture was heated to reflux for 3 h. The reaction mass was quenched with aqueous sodium thiosulphate solution (100 mL) and extracted with EtOAc (2×100 mL). The separated organic layer was dried over Na₂SO₄, filtered and concentrated under reduced pressure. The residue was purified by column chromatography to give Intermediate 11-1 (2.40 g, 8.60 mmol, 66% yield). LC-MS RT: 0.25 min; MS (ESI) m/z 280.0 (M+H)⁺; Method G.

### Intermediate 11-2: N-(2-((4-fluoro-3-(trifluoromethyl) phenyl) carbamoyl)-6-(trifluoromethyl) benzo[b]thiophen-3-yl)-5-iodo-2-methoxynicotinamide

Intermediate 11-2 (900 mg, 1.32 mmol, 74% yield) was prepared from Intermediate 1-2 (378 mg, 0.896 mmol) and Intermediate 11-1 (500 mg, 1.79 mmol) in a similar way as Intermediate 1-3. LC-MS RT: 1.68 min; MS (ESI) m/z 682.1 (M−H)⁻; Method E.

### Intermediate 11-3 and Example 11: N-(2-((4-fluoro-3-(trifluoromethyl) phenyl) carbamoyl)-6-(trifluoromethyl) benzo[b]thiophen-3-yl)-5-(3-(hydroxymethyl) piperidin-1-yl)-2-methoxynicotinamide

Intermediate 11-3 and Example 11 were prepared from Intermediate 11-2 (60 mg, 0.088 mmol) and piperidin-3-ylmethanol (10 mg, 0.088 mmol) according to the general method outlined for Intermediate 9-1. The mixture of Intermediate 11-3 and Example 11 was purified by prep HPLC (Method 1), and the isomers were separated by prep SFC (Column: Chiralpak IC (250×30) mm, 5 μm; % CO₂: 85%; % Co-solvent: 15% of 5 mM NH4OAc in MeOH:ACN (1:1); Total Flow: 100.0 g/min; Back Pressure: 100 bar; Temperature: 35° C.; UV: 250 nm) to give Intermediate 11-3 (Prep SFC RT=12.9 min) and Example 11 (Prep SFC RT=14.4 min). Analytical SFC conditions: (Column: Chiralpak IC (250×4.6) mm, 5 μm; % CO₂: 85%; % Co-solvent: 15% of 5 mM NH4OAc in MeOH:ACN (1:1); Total Flow: 4.0 g/min; Back Pressure: 100 bar; Temperature: 35° C.; UV: 250 nm).

Intermediate 11-3: (2.6 mg, 3.8 μmol, 4% yield). LC-MS RT: 2.48 min; MS (ESI) m/z 671.2 (M+H)⁺; Method A.

Example 11: (3.0 mg, 4.5 μmol, 5% yield). LC-MS RT: 2.48 min; MS (ESI) m/z 671.2 (M+H)⁺; Method A.

### Example 12

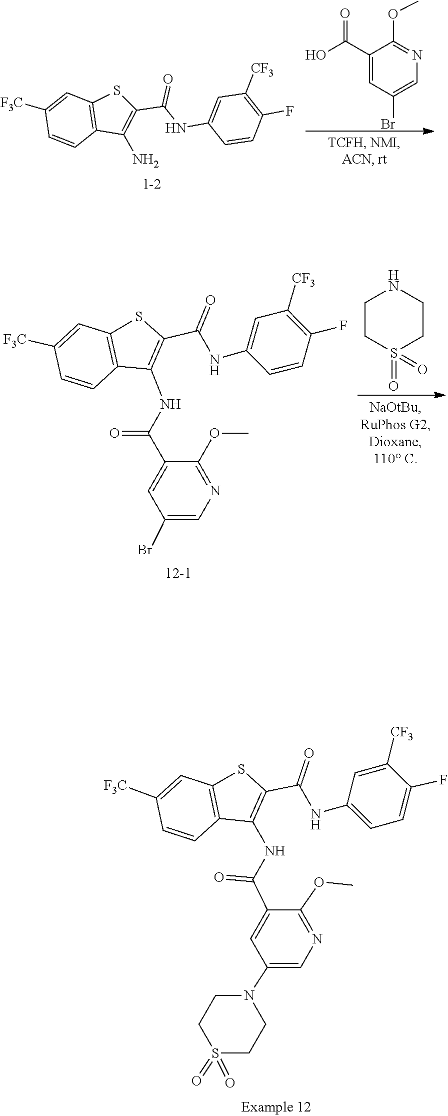

### Intermediate 12-1: 5-bromo-N-(2-((4-fluoro-3-(trifluoromethyl) phenyl) carbamoyl)-6-(trifluoromethyl) benzo[b]thiophen-3-yl)-2-methoxynicotinamide

Intermediate 12-1 (0.38 g, 0.48 mmol, 37% yield) was prepared from Intermediate 1-2 (0.27 g, 0.65 mmol) and 5-bromo-2-methoxynicotinic acid (0.30 g, 1.3 mmol) in a similar way as Intermediate 1-3. LC-MS RT: 1.51 min; MS (ESI) m/z 634.1 (M−H)⁻; Method E.

### Example 12: 5-(1, 1-dioxidothiomorpholino)-N-(2-((4-fluoro-3-(trifluoromethyl) phenyl) carbamoyl)-6-(trifluoromethyl) benzo[b]thiophen-3-yl)-2-methoxynicotinamide

To a solution of Intermediate 12-1 (25 mg, 0.039 mmol) and thiomorpholine 1,1-dioxide (11 mg, 0.079 mmol) in 1,4-dioxane (2.0 mL) was added sodium tert-butoxide (15 mg, 0.16 mmol). The resulting solution was degassed with N₂ for 10 min, followed by addition of RuPhosPdG2 (6.1 mg, 7.9 μmol). The resulting mixture was heated at 110° C. for 12 h. The reaction mass was diluted with EtOAc (50 mL) and washed with water (2×50 mL). The separated organic layer was dried over Na₂SO₄, filtered and concentrated under reduced pressure. The residue was purified by prep LCMS (Method 1) to afford Example 12 (6.0 mg, 8.7 μmol, 22% yield). ¹H NMR (400 MHz, DMSO-d6) δ ppm 10.88 (br s, 1H), 10.76 (br s, 1H), 8.67 (s, 1H), 8.49-8.32 (m, 1H), 8.21-8.12 (m, 1H), 8.08 (d, J=8.1 Hz, 1H), 7.97 (brd, J=2.9 Hz, 1H), 7.81 (dd, J=1.3, 8.7 Hz, 1H), 7.55 (t, J=9.7 Hz, 1H), 7.45 (s, 1H), 3.95 (s, 3H), 3.79-3.60 (m, 4H), 3.23-3.12 (m, 4H). LC-MS RT: 2.38 min; MS (ESI) m/z 691.2 (M+H)⁺; Method A.

### Example 13

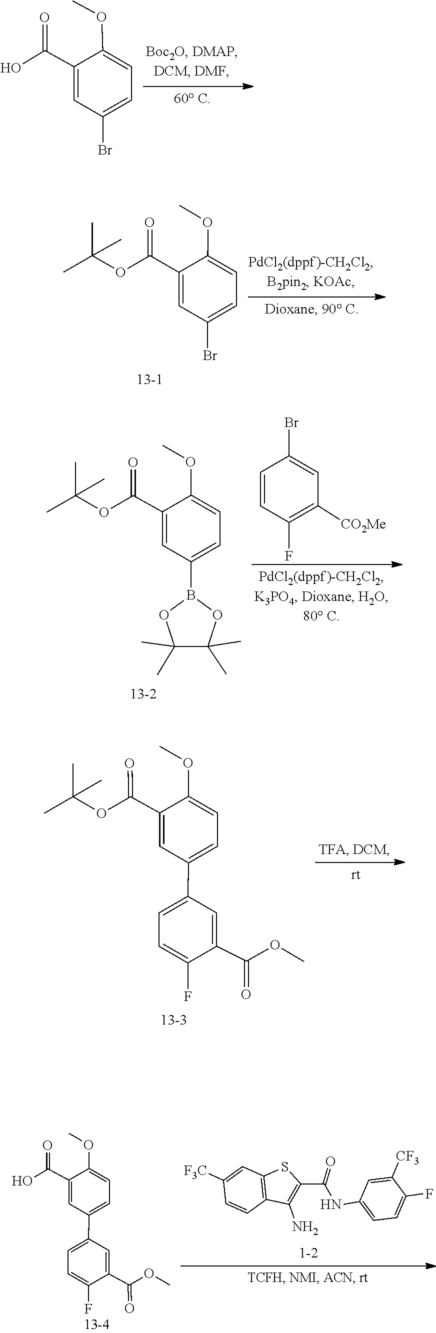

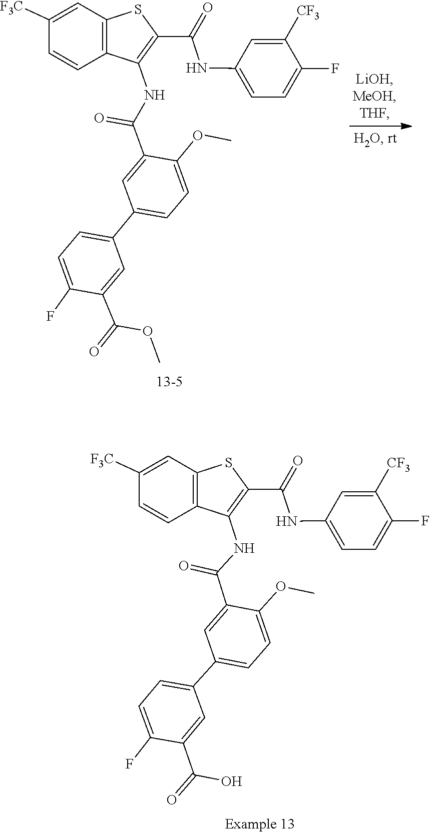 [SMILES: COC(=O)c1cc(-c2ccc(OC)c(C(=O)Nc3c(C(=O)Nc4ccc(F)c(C(F)(F)F)c4)sc4cc(C(F)(F)F)ccc34)c2)ccc1F ; COc1ccc(-c2ccc(F)c(C(=O)O)c2)cc1C(=O)Nc1c(C(=O)Nc2ccc(F)c(C(F)(F)F)c2)sc2cc(C(F)(F)F)ccc12]

### Intermediate 13-1: Tert-butyl 5-bromo-2-methoxybenzoate

To a solution of 5-bromo-2-methoxybenzoic acid (2.00 g, 8.66 mmol) in DCM (20 mL) and DMF (2 mL) was added DMAP (212 mg, 1.73 mmol) followed by Boc2O (2.01 mL, 8.66 mmol). The resulting reaction mixture was heated at 60° C. for 12 h. The reaction mass was diluted with water and extracted with DCM (2×50 mL). The separated organic layer was dried over Na₂SO₄, filtered and concentrated under reduced pressure. The residue was purified by column chromatography to afford Intermediate 13-1 (1.00 g, 3.50 mmol, 40% yield). ¹H NMR (400 MHz, DMSO-d6) δ ppm 7.78-7.75 (m, 2H), 7.12 (d, J=8.5 Hz, 1H), 3.81 (s, 3H), 1.46 (s, 9H).

### Intermediate 13-2: Tert-butyl 2-methoxy-5-(4, 4, 5, 5-tetramethyl-1, 3, 2-dioxaborolan-2-yl) benzoate

To a solution of Intermediate 13-1 (2.00 g, 6.96 mmol) in dioxane (20 mL) was added bis(pinacolato)diboron (2.12 g, 8.36 mmol) and potassium acetate (2.05 g, 20.9 mmol). The resulting solution was degassed with N₂ for 10 min, followed by addition of PdCl₂(dppf)-CH₂Cl₂ adduct (569 mg, 0.696 mmol) and additional degassing for 5 min. The reaction mixture was heated at 90° C. for 12 h, allowed to cool, diluted with EtOAc (50 mL), washed with water (2×20 mL) and brine solution (10 mL). The separated organic layer was dried over Na₂SO₄, filtered and concentrated under reduced pressure. The residue was purified by column chromatography to afford Intermediate 13-2 (1.20 g, 3.59 mmol, 52% yield). ¹H NMR (400 MHz, DMSO-d6) δ ppm 7.79 (s, 1H), 7.78-7.75 (m, 1H), 7.12 (d, J=8.5 Hz, 1H), 3.81 (s, 3H), 1.51 (s, 9H), 1.29 (s, 12H).

### Intermediate 13-3: 3-(tert-butyl) 3′-methyl 4′-fluoro-4-methoxy-[1, 1′-biphenyl]-3, 3′-dicarboxylate

To a solution of methyl 5-bromo-2-fluorobenzoate (400 mg, 1.72 mmol) and Intermediate 13-2 (574 mg, 1.72 mmol) in dioxane (10 mL) and water (2 mL) was added dibasic potassium phosphate (299 mg, 1.72 mmol). The reaction mass was degassed with Ar for 10 min, then PdCl₂(dppf)-CH₂Cl₂ adduct (1.40 g, 1.72 mmol) was added and the mixture was heated at 80° C. for 12 h. The cooled reaction mass was diluted with EtOAc (50 mL) and was washed with water (2×50 mL), followed by brine (50 mL). The separated organic layer was dried over Na₂SO₄, filtered and concentrated under reduced pressure. The residue was purified by column chromatography to afford Intermediate 13-3 (380 mg, 1.05 mmol, 61% yield). ¹H NMR (400 MHz, CDCl₃-d) δ ppm 8.05 (dd, J=2.5, 6.5 Hz, 1H), 7.84 (d, J=2.5 Hz, 1H), 7.65-7.47 (m, 1H), 7.21 (s, 1H), 7.15 (dd, J=8.8, 10.3 Hz, 1H), 6.98 (d, J=8.5 Hz, 1H), 3.92 (s, 3H), 3.88 (s, 3H), 1.57 (s, 9H).

### Intermediate 13-4: 4′-fluoro-4-methoxy-3′-(methoxycarbonyl)-[1, 1′-biphenyl]-3-carboxylic acid

Intermediate 13-4 (310 mg, 1.02 mmol, 97% yield) was prepared from Intermediate 13-3 (380 mg, 1.05 mmol) in a similar way as Intermediate 4-2. LC-MS RT: 2.23 min; MS (ESI) m/z 305.2 (M+H)⁺; Method D.

### Intermediate 13-5: Methyl 4-fluoro-3′-((2-((4-fluoro-3-(trifluoromethyl) phenyl) carbamoyl)-6-(trifluoromethyl) benzo[b]thiophen-3-yl) carbamoyl)-4′-methoxy-[1, 1′-biphenyl]-3-carboxylate

Intermediate 13-5 (145 mg, 0.186 mmol, 45% yield) was prepared from Intermediate 1-2 (175 mg, 0.414 mmol) and Intermediate 13-4 (126 mg, 0.414 mmol) in a similar way as Intermediate 1-3. LC-MS RT: 1.64 min; MS (ESI) m/z 707.2 (M+H)⁺; Method C.

### Example 13: 4-fluoro-3′-((2-((4-fluoro-3-(trifluoromethyl) phenyl) carbamoyl)-6-(trifluoromethyl) benzo[b]thiophen-3-yl) carbamoyl)-4′-methoxy-[1, 1′-biphenyl]-3-carboxylic acid

Example 13 (15 mg, 0.022 mmol, 10% yield) was prepared from Intermediate 13-5 (150 mg, 0.21 mmol) by the general procedure described in Example 2, followed by purification by prep LCMS (method 1). ¹H NMR (400 MHz, DMSO-d6) δ ppm 11.00-10.89 (m, 1H), 10.81 (br dd, J=3.2, 5.4 Hz, 1H), 8.67 (s, 2H), 8.21-7.94 (m, 5H), 7.92-7.77 (m, 3H), 7.52 (t, J=9.8 Hz, 1H), 7.42-7.25 (m, 1H), 3.96 (s, 3H). LC-MS RT: 2.00 min; MS (ESI) m/z 695.0 (M+H)⁺; Method A.

### Example 14

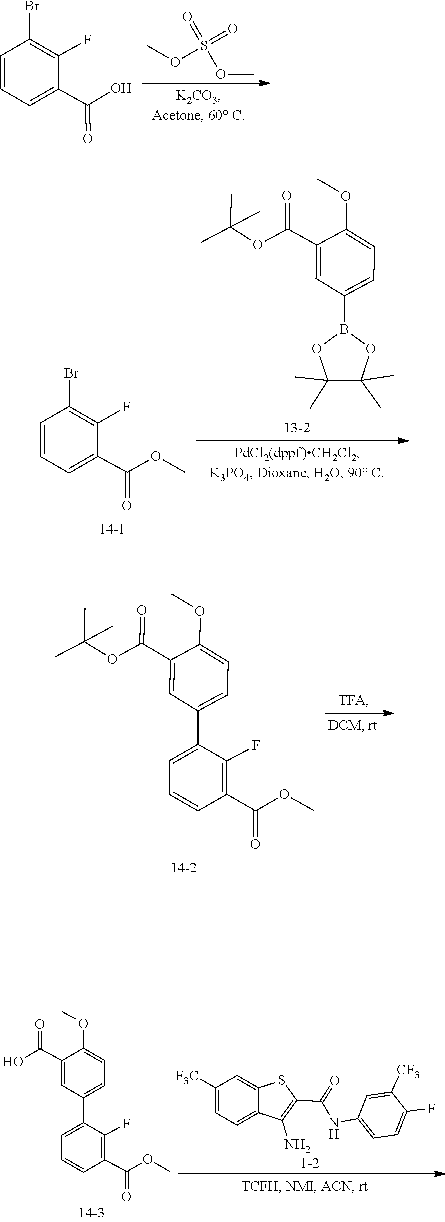

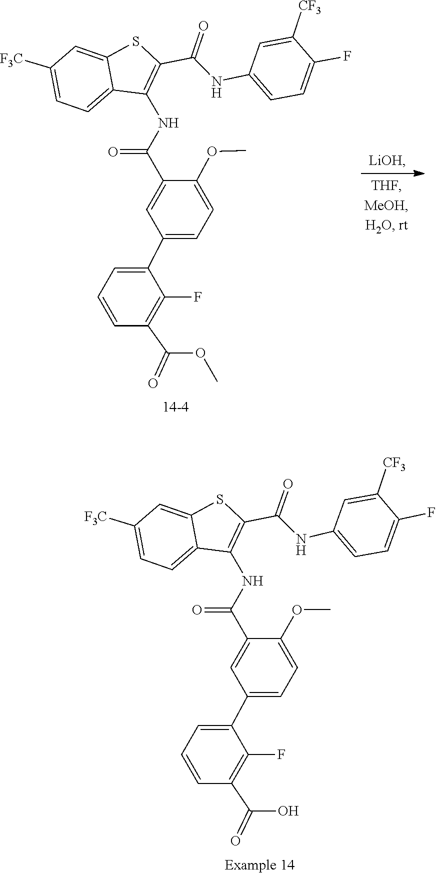 [SMILES: COC(=O)c1cccc(-c2ccc(OC)c(C(=O)Nc3c(C(=O)Nc4ccc(F)c(C(F)(F)F)c4)sc4cc(C(F)(F)F)ccc34)c2)c1F ; COc1ccc(-c2cccc(C(=O)O)c2F)cc1C(=O)Nc1c(C(=O)Nc2ccc(F)c(C(F)(F)F)c2)sc2cc(C(F)(F)F)ccc12]

### Intermediate 14-1: Methyl 3-bromo-2-fluorobenzoate

To a solution of 3-bromo-2-fluorobenzoic acid (500 mg, 2.28 mmol) in acetone (10 mL) was added K₂CO₃ (947 mg, 6.85 mmol) followed by dimethyl sulfate (0.431 mL, 4.57 mmol). The resulting reaction mixture was heated at 60° C. for 2 h. The reaction mass was concentrated under reduced pressure. The residue was diluted in water (100 mL) and extracted with EtOAc (3×100 mL). The separated organic layer was dried over Na₂SO₄, filtered and concentrated under reduced pressure to give Intermediate 14-1 (500 mg, 2.15 mmol, 94% yield). ¹H NMR (400 MHz, CDCl₃) δ ppm 7.91-7.87 (m, 1H), 7.77-7.74 (m, 1H), 7.14-7.10 (m, 1H), 3.98 (s, 3H).

### Intermediate 14-2: 3′-(tert-butyl) 3-methyl-2-fluoro-4′-methoxy-[1,1′-biphenyl]-3,3′-dicarboxylate

Intermediate 14-2 (320 mg, 0.888 mmol, 59% yield) was prepared from Intermediate 13-2 (500 mg, 1.50 mmol) and Intermediate 14-1 (383 mg, 1.65 mmol) in a similar way as Intermediate 13-3. LC-MS RT: 3.33 min; MS (ESI) m/z 305.2 (M+H-tBu)⁺; Method C.

### Intermediate 14-3: 2′-fluoro-4-methoxy-3′-(methoxycarbonyl)-[1,1′-biphenyl]-3-carboxylic acid

Intermediate 14-3 (240 mg, 0.789 mmol, 89% yield) was prepared from Intermediate 14-2 (320 mg, 0.888 mmol) in a similar way as Intermediate 4-2. LC-MS RT: 0.97 min; MS (ESI) m/z 305.2 (M+H)⁺; Method C.

### Intermediate 14-4: Methyl 2-fluoro-3′-((2-((4-fluoro-3-(trifluoromethyl) phenyl) carbamoyl)-6-(trifluoromethyl) benzo[b]thiophen-3-yl) carbamoyl)-4′-methoxy-[1,1′-biphenyl]-3-carboxylate

Intermediate 14-4 (12 mg, 0.017 mmol, 12% yield) was prepared from Intermediate 1-2 (60 mg, 0.14 mmol) and Intermediate 14-3 (86 mg, 0.28 mmol) in a similar way as Intermediate 1-3, followed by purification by prep LCMS (method 3). LC-MS RT: 1.50 min; MS (ESI) m/z 707.2 (M−H)⁻; Method E.

### Example 14: 2-fluoro-3′-((2-((4-fluoro-3-(trifluoromethyl) phenyl) carbamoyl)-6-(trifluoromethyl) benzo[b]thiophen-3-yl) carbamoyl)-4′-methoxy-[1,1′-biphenyl]-3-carboxylic acid

Example 14 (3.3 mg, 4.8 μmol, 34% yield) was prepared from Intermediate 14-4 (10 mg, 0.014 mmol) by the general procedure described in Example 2, followed by purification by prep LCMS (Method 4). ¹H NMR (400 MHz, DMSO-d6) δ ppm 10.90 (br dd, J=1.0, 2.0 Hz, 1H), 10.78 (br d, J=1.7 Hz, 1H), 8.67 (s, 1H), 8.17 (dd, J=2.3, 6.7 Hz, 1H), 8.10 (d, J=8.8 Hz, 1H), 8.04-7.94 (m, 2H), 7.87-7.79 (m, 2H), 7.79-7.72 (m, 1H), 7.70-7.60 (m, 1H), 7.53 (t, J=9.8 Hz, 1H), 7.42-7.28 (m, 2H), 3.99 (s, 3H). LC-MS RT: 1.99 min; MS (ESI) m/z 695.2 (M+H)⁺; Method A.

### Example 15

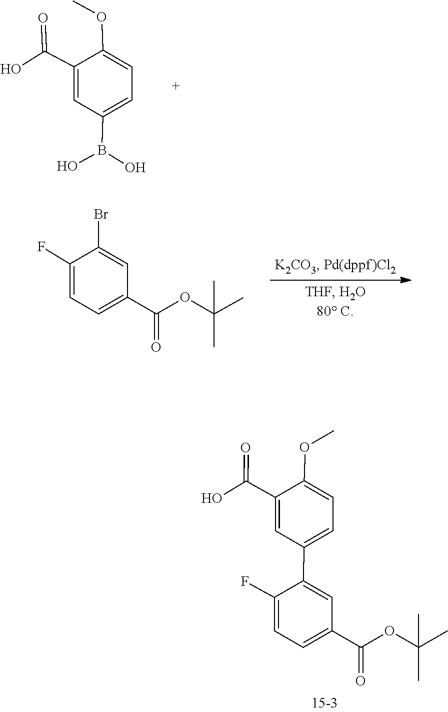 [SMILES: COc1ccc(B(O)O)cc1C(=O)O ; CC(C)(C)OC(=O)c1ccc(F)c(Br)c1 ; COc1ccc(-c2cc(C(=O)OC(C)(C)C)ccc2F)cc1C(=O)O]

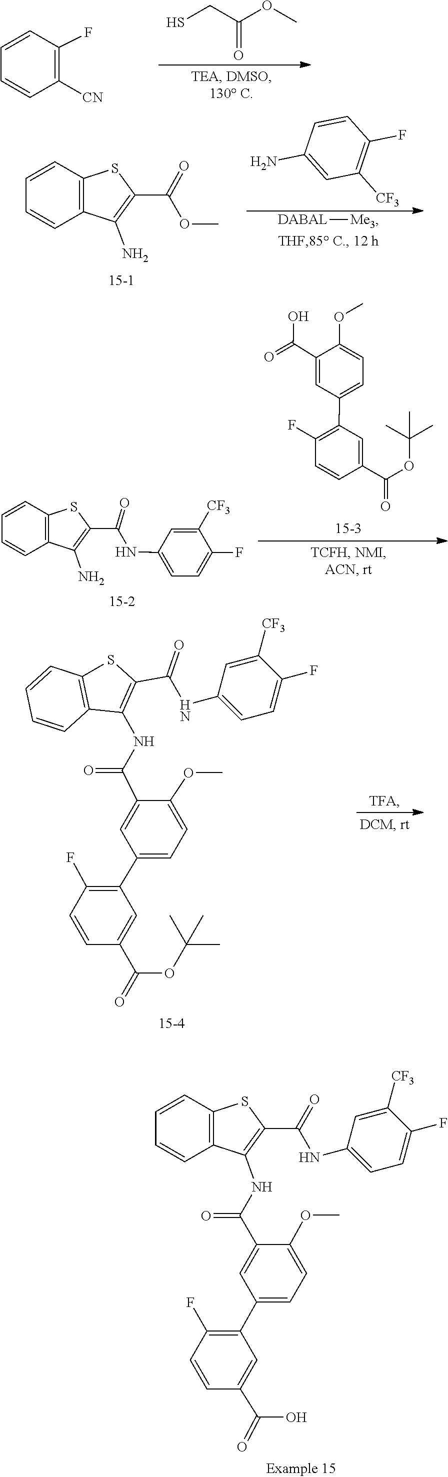

### Intermediate 15-1: Methyl 3-aminobenzo[b]thiophene-2-carboxylate

Intermediate 15-1 (0.97 g, 4.7 mmol, 57% yield) was prepared from 2-fluorobenzonitrile (1.0 g, 8.3 mmol) and methyl 2-mercaptoacetate (0.78 mL, 8.7 mmol) in a similar way as Intermediate 1-1. LC-MS RT: 1.72 min; MS (ESI) m/z 208.2 (M+H)⁺; Method E.

### Intermediate 15-2: 3-amino-N-(4-fluoro-3-(trifluoromethyl) phenyl) benzo[b]thiophene-2-carboxamide

Intermediate 15-2 (0.71 g, 2.0 mmol, 94% yield) was prepared from 4-fluoro-3-(trifluoromethyl)aniline (420 mg, 2.3 mmol) and Intermediate 15-1 (440 mg, 2.1 mmol) in a similar way as Intermediate 1-2. LC-MS RT: 0.90 min; MS (ESI) m/z 353.2 (M−H)⁻; Method E.

### Intermediate 15-3: 5′-(tert-butoxycarbonyl)-2′-fluoro-4-methoxy-[1,1′-biphenyl]-3-carboxylic acid

To a solution of 5-borono-2-methoxybenzoic acid (2.00 g, 10.2 mmol), tert-butyl 3-bromo-4-fluorobenzoate (3.37 g, 12.3 mmol) and K₂CO₃ (7.05 g, 51.0 mmol) in water (14.3 mL) and THF (100 mL) was added PdCl₂(dppf)-CH₂Cl₂ adduct (1.25 g, 1.53 mmol) and the solution was degassed with N₂ for 5 min. The reaction was heated at 80° C. for 16 h. The cooled reaction solution was diluted with EtOAc (300 mL), washed with a 1:1 solution of saturated brine and water (2×15 mL), dried over Na₂SO₄, filtered through celite and evaporated to dryness in vacuo to give Intermediate 15-3 (2.80 g, 8.06 mmol, 79% yield). LC-MS RT: 2.03 min; MS (ESI) m/z 347.1 (M+H)⁺; Method H.

### Intermediate 15-4: Tert-butyl 6-fluoro-3′-((2-((4-fluoro-3-(trifluoromethyl) phenyl) carbamoyl) benzo[b]thiophen-3-yl) carbamoyl)-4′-methoxy-[1,1′-biphenyl]-3-carboxylate

Intermediate 15-4 (150 mg, 0.220 mmol, 52% yield) was prepared from Intermediate 15-2 (150 mg, 0.423 mmol) and Intermediate 15-3 (147 mg, 0.423 mmol) in a similar way as Intermediate 1-3. LC-MS RT: 1.62 min; MS (ESI) m/z 683.2 (M+H)⁺; Method C.

### Example 15: 6-fluoro-3′-((2-((4-fluoro-3-(trifluoromethyl) phenyl) carbamoyl) benzo[b]thiophen-3-yl) carbamoyl)-4′-methoxy-[1, 1′-biphenyl]-3-carboxylic acid

Example 15 (15 mg, 0.023 mmol, 16% yield) was prepared from Intermediate 15-4 (100 mg, 0.146 mmol) in a similar way as Intermediate 4-2, followed by purification by prep HPLC (Method 9). ¹H NMR (400 MHz, DMSO-d6) δ ppm 11.9-11.8 (m, 1H), 10.78-10.84 (m, 1H) 10.61-10.66 (m, 1H) 8.13-8.18 (m, 1H) 8.10-8.12 (m, 1H) 8.06-8.10 (m, 2H) 7.96-8.03 (m, 2H) 7.92-7.96 (m, 1H) 7.77-7.83 (m, 1H) 7.47-7.60 (m, 1H) 7.36-7.47 (m, 1H) 7.34-7.59 (m, 2H) 3.99 (s, 3H). LC-MS RT: 2.75 min; MS (ESI) m/z 627.0 (M+H)⁺; Method C.

### Example 16 and 17

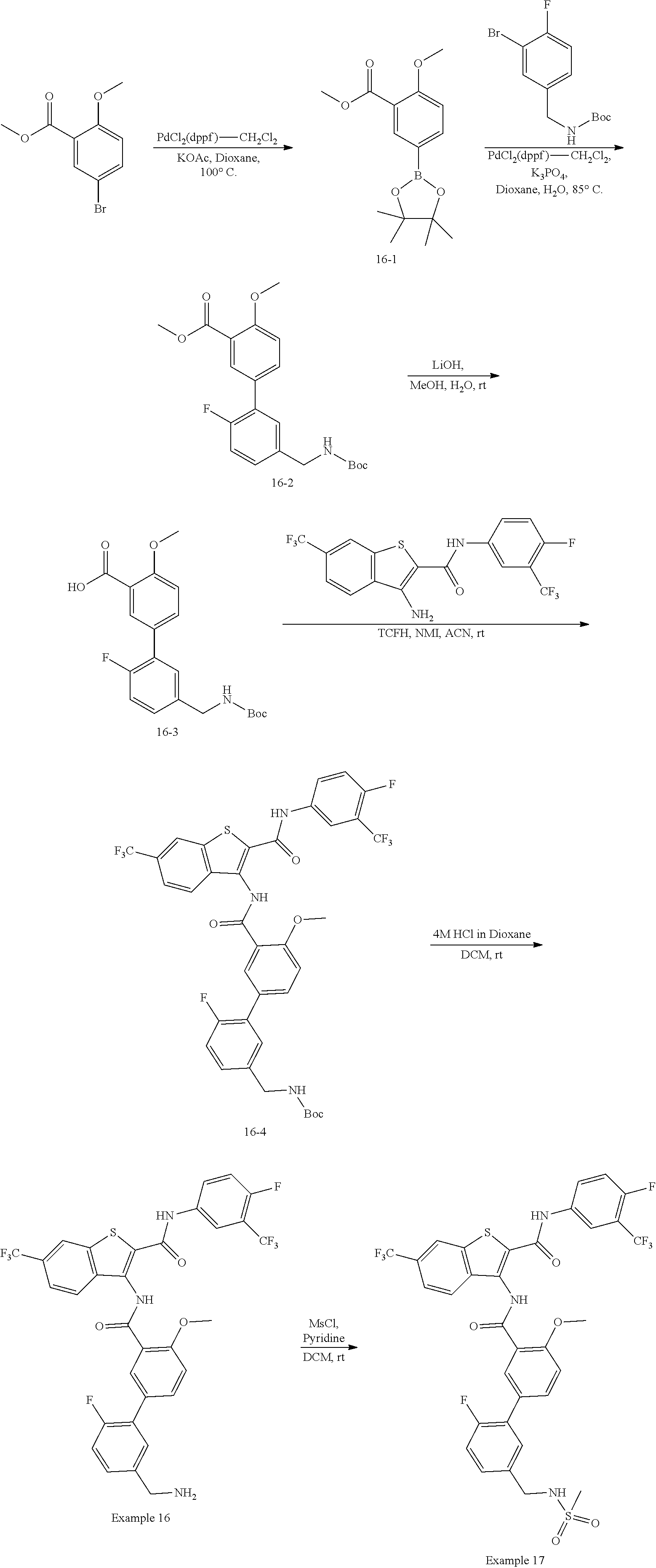

### Intermediate 16-1: Methyl 2-methoxy-5-(4,4,5,5-tetramethyl-1,3,2-dioxaborolan-2-yl) benzoate

Intermediate 16-1 (1.2 g, 4.1 mmol, 67% yield) was prepared from methyl 5-bromo-2-methoxybenzoate (1.5 g, 6.1 mmol) in a similar way as Intermediate 13-2. LC-MS RT: 0.82 min; MS (ESI) m/z 293.2 (M+H)⁺; Method E.

### Intermediate 16-2: methyl 5′-(((tert-butoxycarbonyl) amino) methyl)-2′-fluoro-4-methoxy-[1, 1′-biphenyl]-3-carboxylate

Intermediate 16-2 (600 mg, 1.54 mmol, 90% yield) was prepared from Intermediate 16-1 (500 mg, 1.71 mmol) and tert-butyl (3-bromo-4-fluorobenzyl)carbamate (573 mg, 1.88 mmol) in a similar way as Intermediate 13-3. LC-MS RT: 0.77 min; MS (ESI) m/z 334.2 (M+H-tBu)⁺; Method E.

### Intermediate 16-3: 5′-(((tert-butoxycarbonyl)amino)methyl)-2′-fluoro-4-methoxy-[1,1′-biphenyl]-3-carboxylic acid

Intermediate 16-3 (500 mg, 1.33 mmol, 80% yield) was prepared from Intermediate 16-2 (650 mg, 1.66 mmol) by the procedure described in Example 2. LC-MS RT: 1.03 min; MS (ESI) m/z 320.2 (M+H-tBu)⁺; Method E.

### Intermediate 16-4: Tert-butyl ((6-fluoro-3′-((2-((4-fluoro-3-(trifluoromethyl) phenyl) carbamoyl)-6-(trifluoromethyl) benzo[b]thiophen-3-yl) carbamoyl)-4′-methoxy-[1,1′-biphenyl]-3-yl) methyl) carbamate

Intermediate 16-4 (310 mg, 0.398 mmol, 84% yield) was prepared from Intermediate 1-2 (200 mg, 0.474 mmol) and Intermediate 16-3 (178 mg, 0.474 mmol) in a similar way as Intermediate 1-3. LC-MS RT: 1.55 min; MS (ESI) m/z 778.2 (M−H)⁻; Method E.

### Example 16: 3-(5′-(aminomethyl)-2′-fluoro-4-methoxy-[1,1′-biphenyl]-3-carboxamido)-N-(4-fluoro-3-(trifluoromethyl)phenyl)-6-(trifluoromethyl)benzo[b]thiophene-2-carboxamide

Example 16 (180 mg, 0.252 mmol, 78% yield) was prepared from Intermediate 16-4 (250 mg, 0.321 mmol) according to the general conditions outlined for Intermediates 9-4 and 9-5, followed by purification by prep LCMS (Method 1). ¹H NMR (400 MHz, DMSO-d6) δ ppm 11.00-10.60 (m, 2H), 8.63 (s, 1H), 8.42 (br s, 2H), 8.17-8.09 (m, 2H), 8.00-7.91 (m, 2H), 7.83-7.69 (m, 2H), 7.58-7.45 (m, 2H), 7.41-7.31 (m, 2H), 7.25 (dd, J=8.4, 10.9 Hz, 1H), 3.97 (s, 3H), 3.82 (br s, 2H). LC-MS RT: 2.23 min; MS (ESI) m/z 680.2 (M+H)⁺; Method A.

### Example 17: N-(4-fluoro-3-(trifluoromethyl)phenyl)-3-(2′-fluoro-4-methoxy-5′-(methylsulfonamidomethyl)-[1,1′-biphenyl]-3-carboxamido)-6-(trifluoromethyl)benzo[b]thiophene-2-carboxamide

To a cooled solution of Example 16 (40 mg, 0.056 mmol) in DCM (2 mL) was added pyridine (0.023 mL, 0.28 mmol) followed by MsCl (6.5 μl, 0.084 mmol). The reaction mixture was stirred at rt for 2 h, diluted with EtOAc (20 mL) and washed with water (2×20 mL). The separated organic layer was dried over Na₂SO₄, filtered and concentrated under reduced pressure. The residue was purified by prep LCMS (Method 2) to afford Example 17 (6.8 mg, 9.0 μmol, 16% yield). ¹H NMR (400 MHz, DMSO-d6) δ ppm 11.00-10.60 (m, 2H), 8.67 (s, 1H), 8.16 (dd, J=2.2, 6.1 Hz, 1H), 8.10 (d, J=8.3 Hz, 1H), 8.05-7.93 (m, 2H), 7.83 (dd, J=1.2, 8.8 Hz, 1H), 7.76 (br d, J=8.6 Hz, 1H), 7.66-7.59 (m, 1H), 7.57-7.44 (m, 2H), 7.43-7.35 (m, 2H), 7.33-7.24 (m, 1H), 4.21 (br d, J=5.9 Hz, 3H), 4.00 (s, 3H), 2.89 (s, 3H). LC-MS RT: 2.49 min; MS (ESI) m/z 756.1 (M−H)⁻; Method A.

### Example 18: 3-(5′-(cyclobutanecarboxamidomethyl)-2′-fluoro-4-methoxy-[1,1′-biphenyl]-3-carboxamido)-N-(4-fluoro-3-(trifluoromethyl)phenyl)-6-(trifluoromethyl)benzo[b]thiophene-2-carboxamide

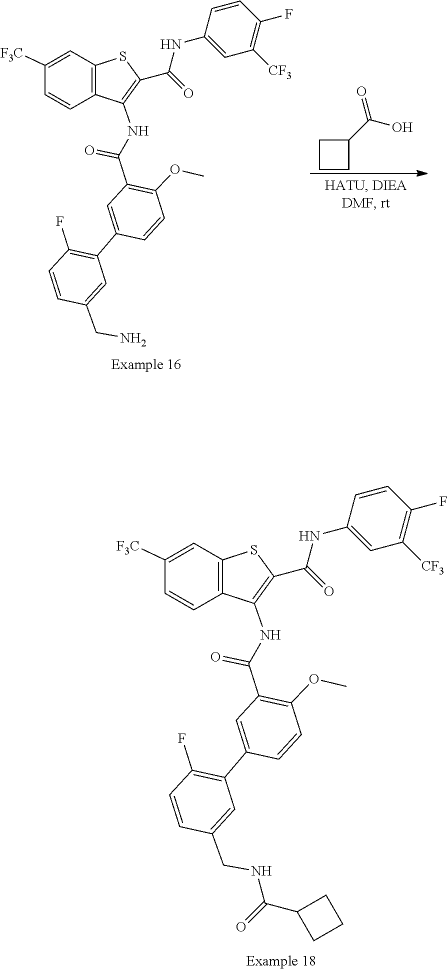 [SMILES: COc1ccc(-c2cc(CN)ccc2F)cc1C(=O)Nc1c(C(=O)Nc2ccc(F)c(C(F)(F)F)c2)sc2cc(C(F)(F)F)ccc12 ; O=C(O)C1CCC1 ; COc1ccc(-c2cc(CNC(=O)C3CCC3)ccc2F)cc1C(=O)Nc1c(C(=O)Nc2ccc(F)c(C(F)(F)F)c2)sc2cc(C(F)(F)F)ccc12]

Example 18 (6.4 mg, 8.4 μmol, 14% yield) was prepared from Example 16 (40 mg, 0.059 mmol) and cyclobutanecarboxylic acid (12 mg, 0.12 mmol) according to the general procedures outlined for Examples 7 and 8, except using DIEA as a base rather than TEA. The residue was purified by prep LCMS (Method 1). ¹H NMR (400 MHz, DMSO-d6) δ ppm 10.89 (s, 1H), 10.76 (s, 1H), 8.68 (s, 1H), 8.22 (t, J=5.7 Hz, 1H), 8.16 (dd, J=2.4, 6.6 Hz, 1H), 8.09 (d, J=8.6 Hz, 1H), 8.03-7.92 (m, 2H), 7.82 (dd, J=1.3, 8.7 Hz, 1H), 7.73 (br d, J=8.8 Hz, 1H), 7.54 (t, J=9.8 Hz, 1H), 7.37 (d, J=8.8 Hz, 2H), 7.25 (d, J=8.6 Hz, 2H), 4.28 (d, J=5.9 Hz, 2H), 3.99 (s, 3H), 3.09-3.00 (m, 1H), 2.20-2.08 (m, 2H), 2.06-1.95 (m, 2H), 1.92-1.82 (m, 1H), 1.79-1.67 (m, 2H). LC-MS RT: 2.61 min; MS (ESI) m/z 760.2 (M−H)⁻; Method A.

### Example 19: N-(4-fluoro-3-(trifluoromethyl) phenyl)-3-(2′-fluoro-5′-((isobutylamino) methyl)-4-methoxy-[1,1′-biphenyl]-3-carboxamido)-6-(trifluoromethyl) benzo[b]thiophene-2-carboxamide

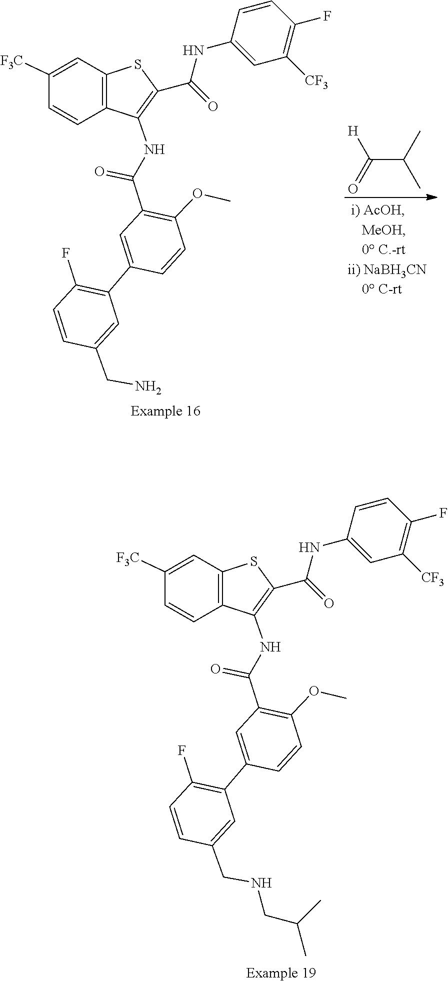

To a solution of Example 16 (40 mg, 0.059 mmol) in MeOH (2 mL) was added isobutyraldehyde (8.5 mg, 0.12 mmol) followed by AcOH (0.67 μl, 0.012 mmol) dropwise at 0° C. The resulting solution was stirred at rt for 12 h followed by addition of sodium cyanoborohydride (7.4 mg, 0.12 mmol) at 0° C. and the reaction mixture was stirred for 2 h at rt. The reaction mixture was concentrated under reduced pressure, the residue was dissolved in water (5 mL) and then extracted with EtOAc (3×10 mL). The separated organic layer was dried over Na₂SO₄, filtered and concentrated under reduced pressure. The residue was purified by prep LCMS (Method 1) to afford Example 19 (5.5 mg, 7.5 μmol, 13% yield). H NMR (400 MHz, DMSO-d6) ppm 11.07-10.47 (m, 2H), 8.66 (s, 1H), 8.19-8.05 (m, 2H), 8.04-7.92 (m, 1H), 7.81 (dd, J=1.6, 8.9 Hz, 1H), 7.75 (br d, J=8.8 Hz, 1H), 7.59-7.43 (m, 2H), 7.39-7.28 (m, 2H), 7.23 (dd, J=8.4, 10.9 Hz, 2H), 4.88 (br s, 1H), 3.99 (br s, 3H), 3.73 (s, 2H), 2.32 (d, J=6.8 Hz, 2H), 1.68 (td, J=6.6, 13.2 Hz, 1H), 0.89-0.80 (m, 6H). LC-MS RT: 2.65 min; MS (ESI) m/z 736.3 (M+H)⁺; Method A.

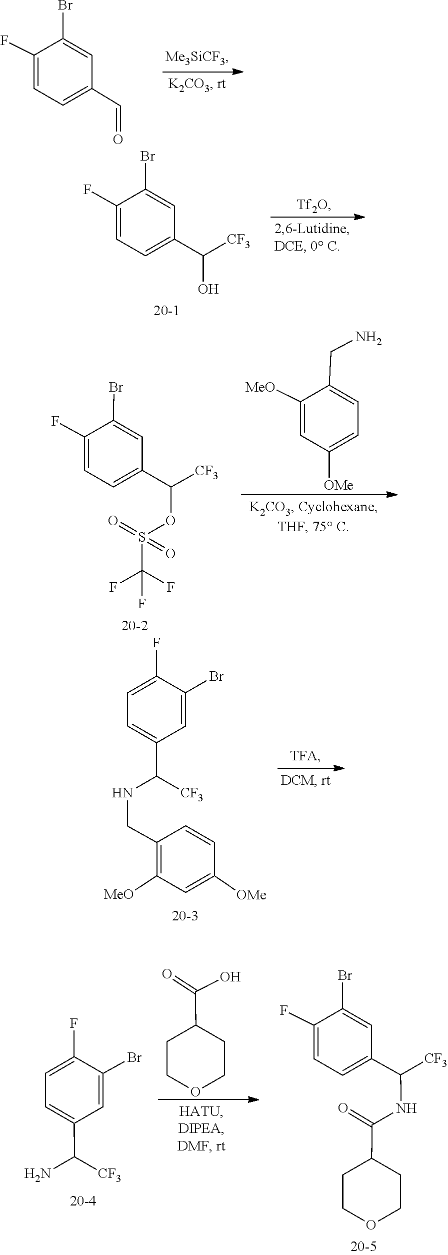

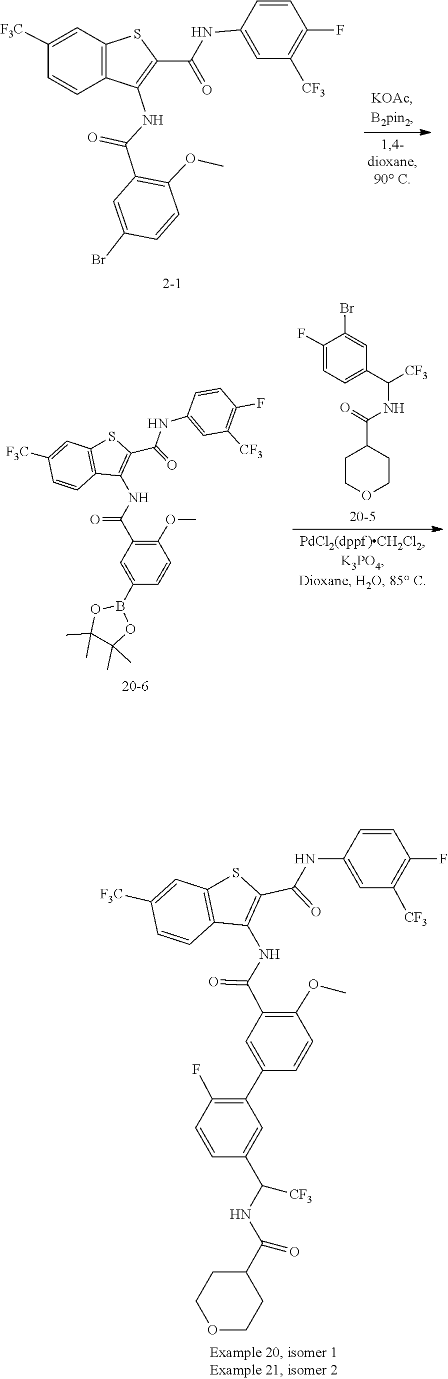 [SMILES: COc1ccc(Br)cc1C(=O)Nc1c(C(=O)Nc2ccc(F)c(C(F)(F)F)c2)sc2cc(C(F)(F)F)ccc12 ; COc1ccc(B2OC(C)(C)C(C)(C)O2)cc1C(=O)Nc1c(C(=O)Nc2ccc(F)c(C(F)(F)F)c2)sc2cc(C(F)(F)F)ccc12 ; O=C(NC(c1ccc(F)c(Br)c1)C(F)(F)F)C1CCOCC1 ; COc1ccc(-c2cc(C(NC(=O)C3CCOCC3)C(F)(F)F)ccc2F)cc1C(=O)Nc1c(C(=O)Nc2ccc(F)c(C(F)(F)F)c2)sc2cc(C(F)(F)F)ccc12]

### Intermediate 20-1: 1-(3-bromo-4-fluorophenyl)-2, 2, 2-trifluoroethan-1-ol

To 3-bromo-4-fluorobenzaldehyde (2.00 g, 9.85 mmol) and K₂CO₃ (0.136 g, 0.985 mmol) was added (trifluoromethyl) trimethylsilane (2.90 mL, 19.7 mmol). The resulting reaction mixture was stirred at rt for 12 h. The reaction mass was partitioned between water and EtOAc (2×50 mL), the combined organic layers were washed with brine solution, dried over Na₂SO₄ and concentrated under reduced pressure and the residue subsequently treated with 2N HCl for 1 h at 0° C. The reaction mass was then extracted with EtOAc (2×50 mL), then the combined organic layers were washed with brine solution, dried over Na₂SO₄ and concentrated under reduced pressure to give Intermediate 20-1 (2.08 g, 7.62 mmol, 77% yield). LC-MS RT: 0.66 min; MS (ESI) m/z 271.0 (M−H)⁻; Method E.

### Intermediate 20-2: 1-(3-bromo-4-fluorophenyl)-2, 2,2-trifluoroethyl trifluoromethanesulfonate

To a solution of Intermediate 20-1 (300 mg, 1.10 mmol) in DCE (10 mL) at −10° C. was added 2,6-lutidine (0.384 mL, 3.30 mmol) followed by a solution of triflic anhydride (0.278 mL, 1.65 mmol) in DCE (1 mL) dropwise. The reaction mixture was allowed to stir at 0° C. for 30 min. The reaction mass was partitioned between water (50 mL) and Et2O (50 mL). The separated organic layers were washed with brine solution, dried over Na₂SO₄, filtered and concentrated under reduced pressure to give Intermediate 20-2 (370 mg, 0.913 mmol, 83% yield). ¹H NMR (400 MHz, DMSO-d6) δ ppm 7.80-7.73 (m, 1H), 7.64-7.59 (m, 1H), 7.49-7.40 (m, 1H), 5.29-5.24 (m, 1H).

### Intermediate 20-3: 1-(3-bromo-4-fluorophenyl)-N-(2, 4-dimethoxybenzyl)-2,2,2-trifluoroethan-1-amine

To a solution of Intermediate 20-2 (500 mg, 1.23 mmol) and (2, 4-dimethoxyphenyl) methanamine (0.223 mL, 1.48 mmol) in mixture of cyclohexane (6 mL) and THF (3 mL) was added K₂CO₃ (512 mg, 3.70 mmol). The resulting reaction mixture was stirred at 75° C. for 24 h. The reaction mass was diluted with EtOAc (50 mL) and washed with water (2×50 mL). The separated organic layer was dried over Na₂SO₄, filtered and concentrated under reduced pressure. The residue was purified by column chromatography to afford Intermediate 20-3 (420 mg, 0.995 mmol, 81% yield). ¹H NMR (400 MHz, DMSO-d6) δ ppm 7.82-7.85 (m, 1H), 7.51-7.56 (m, 1H), 7.37-7.43 (m, 1H), 7.16-7.21 (m, 2H), 6.45-6.48 (m, 1H), 3.71 (d, J=14.56 Hz, 2H), 3.42-3.59 (m, 1H), 3.25-3.38 (m, 6H).

### Intermediate 20-4: 1-(3-bromo-4-fluorophenyl)-2, 2, 2-trifluoroethan-1-amine

To a solution of Intermediate 20-3 (420 mg, 0.995 mmol) in DCM (8 mL) was added TFA (0.230 mL, 2.98 mmol) and the reaction solution was stirred for 18 h at rt. The reaction mixture was concentrated under high vacuum to afford Intermediate 20-4 (260 mg, 0.956 mmol, 96% yield). ¹H NMR (400 MHz, DMSO-d6) δ ppm 7.98-8.01 (m, 1H), 7.54-7.70 (m, 1H), 6.45-6.51 (m, 1H), 3.74 (br s, 2H), 3.59 (br d, J=13.55 Hz, 1H).

### Intermediate 20-5: N-(1-(3-bromo-4-fluorophenyl)-2,2,2-trifluoroethyl)tetrahydro-2H-pyran-4-carboxamide

Intermediate 20-5 (610 mg, 0.60 mmol, 39% yield) was prepared from Intermediate 20-4 (502 mg, 1.84 mmol) and tetrahydro-2H-pyran-4-carboxylic acid (200 mg, 1.54 mmol) by the general procedure described in Example 18. LC-MS RT: 0.60 min; MS (ESI) m/z 382.1 (M−H)⁻; Method E.

### Intermediate 20-6: N-(4-fluoro-3-(trifluoromethyl) phenyl)-3-(2-methoxy-5-(4, 4, 5, 5-tetramethyl-1, 3, 2-dioxaborolan-2-yl) benzamido)-6-(trifluoromethyl) benzo[b]thiophene-2-carboxamide

Intermediate 20-6 (80 mg, 0.12 mmol, 50% yield) was prepared from Intermediate 2-1 (150 mg, 0.236 mmol) according to the general procedures described for Intermediate 13-2. LC-MS RT: 1.52 min; MS (ESI) m/z 683.2 (M+H)⁺; Method E.

### Example 20 and 21: N-(2,2,2-trifluoro-1-(6-fluoro-3′-((2-((4-fluoro-3-(trifluoromethyl)phenyl)carbamoyl)-6-(trifluoromethyl)benzo[b]thiophen-3-yl)carbamoyl)-4′-methoxy-[1,1′-biphenyl]-3-yl)ethyl)tetrahydro-2H-pyran-4-carboxamide

Example 20 and 21 were prepared from Intermediate 20-6 (120 mg, 0.176 mmol) and Intermediate 20-5 (186 mg, 0.193 mmol) in a similar way as Intermediate 13-3, followed by purification by prep HPLC (Method 2). The isomers were separated by prep SFC (Column/dimensions: Chiralcel OD-H (250×21) mm, 5 μm; % CO₂: 60%; % Co-solvent: 40% 0.2% DEA in IPA; Total Flow: 80.0 g/min; Back Pressure: 100 bar; Temperature: 35° C.; UV: 248 nm) to give Example 20 (Prep SFC RT=10.37 min) and Example 21 (Prep SFC RT=2.82 min). Analytical SFC conditions: (Column/dimensions: Chiralcel OD-H (250×4.6) mm, 5 μm; % CO₂: 70%; % Co-solvent: 30% 0.2% DEA in IPA; Total Flow: 4.0 g/min; Back Pressure: 100 bar; Temperature: 35° C.; UV: 248 nm).

Example 20 (4.0 mg, 4.4 μmol, 2% yield): ¹H NMR (400 MHz, DMSO-d6) δ ppm 10.62-11.01 (m, 2H), 9.02-9.26 (m, 2H), 8.48-8.80 (m, 1H), 8.12-81.8 (m, 2H), 7.91-8.20 (m, 2H), 7.61-7.89 (m, 2H), 7.31-7.56 (m, 1H), 7.23-7.25 (m, 2H), 5.77-6.01 (m, 1H), 3.98-4.06 (m, 1H), 4.00 (s, 3H), 3.22-3.31 (m, 2H), 2.60 (br s, 1H), 2.44-2.47 (m, 1H), 2.34-2.41 (m, 1H), 1.51-1.68 (m, 2H), 1.26 (br d, J=19.58 Hz, 2H). LC-MS RT: 1.21 min; MS (ESI) m/z 858.2 (M−H)⁻; Method C.

Example 21 (4.0 mg, 4.4 μmol, 2% yield): ¹H NMR (400 MHz, DMSO-d6) δ ppm 10.67-10.98 (m, 2H), 8.92-9.22 (m, 2H), 8.54-8.78 (m, 1H), 8.09-8.18 (m, 2H), 7.90-8.23 (m, 2H), 7.67-7.89 (m, 2H), 7.44-7.68 (m, 1H), 7.31-7.44 (m, 2H), 5.80-5.97 (m, 1H), 4.01 (s, 3H), 3.44 (s, 1H), 3.26-3.29 (m, 2H), 2.58-2.67 (m, 1H), 2.39-2.45 (m, 2H), 1.48-1.59 (m, 2H), 1.18 (m, 2H). LC-MS RT: 1.23 min; MS (ESI) m/z 860.2 (M+H)⁺; Method C.

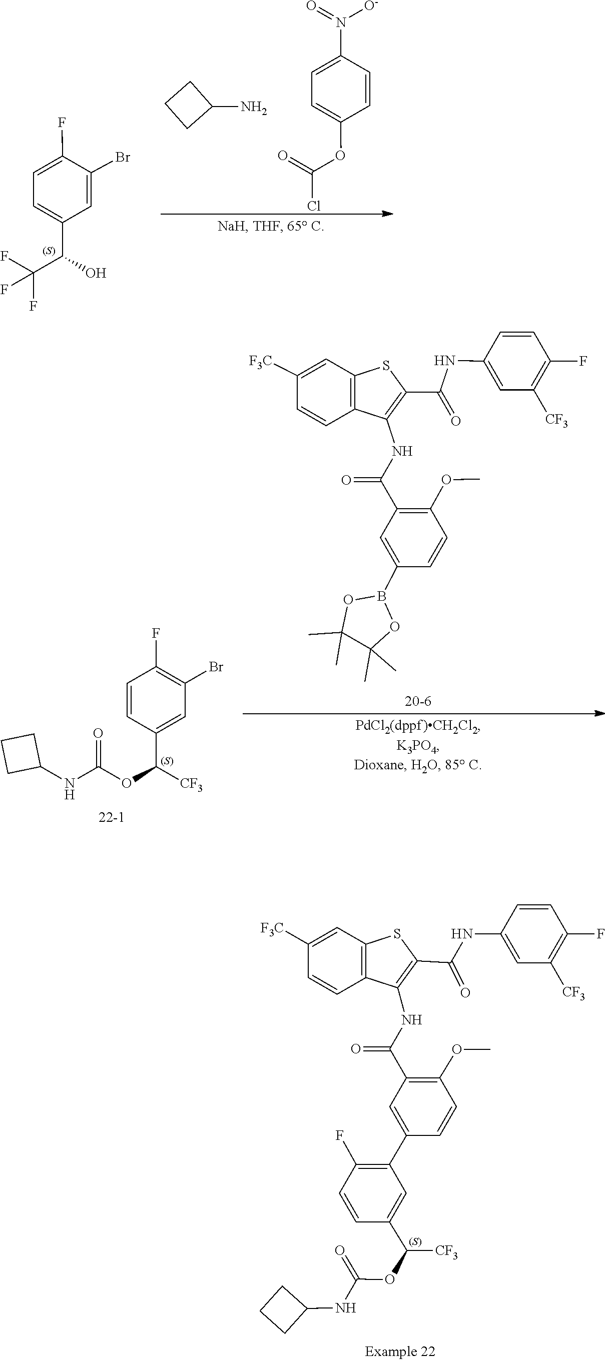

### Intermediate 22-1: (S)-1-(3-bromo-4-fluorophenyl)-2, 2,2-trifluoroethyl cyclobutylcarbamate

To a solution of (S)-1-(3-bromo-4-fluorophenyl)-2, 2, 2-trifluoroethan-1-ol (300 mg, 1.10 mmol) in THF (6 mL) at 0° C. was added NaH (96.0 mg, 2.20 mmol), and the reaction mixture was stirred at rt for 20 min. A solution of 4-nitrophenyl carbonochloridate (244 mg, 1.21 mmol) in THF (1 mL) was added and the reaction mixture was continued to stir for another 2 h. A solution of cyclobutanamine (156 mg, 2.20 mmol) in THF (4 mL) was added to the reaction mass and stirred for 6 h at 65° C. The reaction mass was quenched with aqueous NH4Cl solution (50 mL) and extracted with EtOAc (2×50 mL). The separated organic layer was dried over Na₂SO₄, filtered and concentrated under reduced pressure. The residue was purified by column chromatography to afford Intermediate 22-1 (150 mg, 0.405 mmol, 37% yield). LC-MS RT: 0.96 min; MS (ESI) m/z 368.1 (M−H)⁻; Method E.

### Example 22: (S)-2, 2, 2-trifluoro-1-(6-fluoro-3′-((2-((4-fluoro-3-(trifluoromethyl) phenyl) carbamoyl)-6-(trifluoromethyl) benzo[b]thiophen-3-yl) carbamoyl)-4′-methoxy-[1, 1′-biphenyl]-3-yl) ethyl cyclobutylcarbamate

Example 22 (25 mg, 0.027 mmol, 23% yield) was prepared from Intermediate 20-6 (80 mg, 0.12 mmol) and Intermediate 22-1 (43 mg, 0.12 mmol) in a similar way as Intermediate 13-3, followed by purification by prep HPLC (Method 3). ¹H NMR (400 MHz, DMSO-d6) δ ppm 10.71-11.00 (m, 2H), 8.61-8.74 (m, 1H), 8.10-8.19 (m, 1H), 7.98-8.04 (m, 1H), 7.93-7.97 (m, 1H), 7.80-7.86 (m, 1H), 7.74-7.79 (m, 1H), 7.67-7.72 (m, 1H), 7.61-7.65 (m, 1H), 7.53-7.59 (m, 1H), 7.46-7.48 (m, 1H), 7.43-7.45 (m, 1H), 7.38-7.40 (m, 1H), 6.36-6.40 (m, 1H), 3.92-3.94 (m, 1H), 3.88-3.91 (m, 3H), 3.34 (s, 2H), 2.66-2.69 (m, 1H), 2.32-2.35 (m, 2H), 1.82-1.97 (m, 1H). LC-MS RT: 2.02 min; MS (ESI) m/z 846.0 (M+H)⁺; Method C.

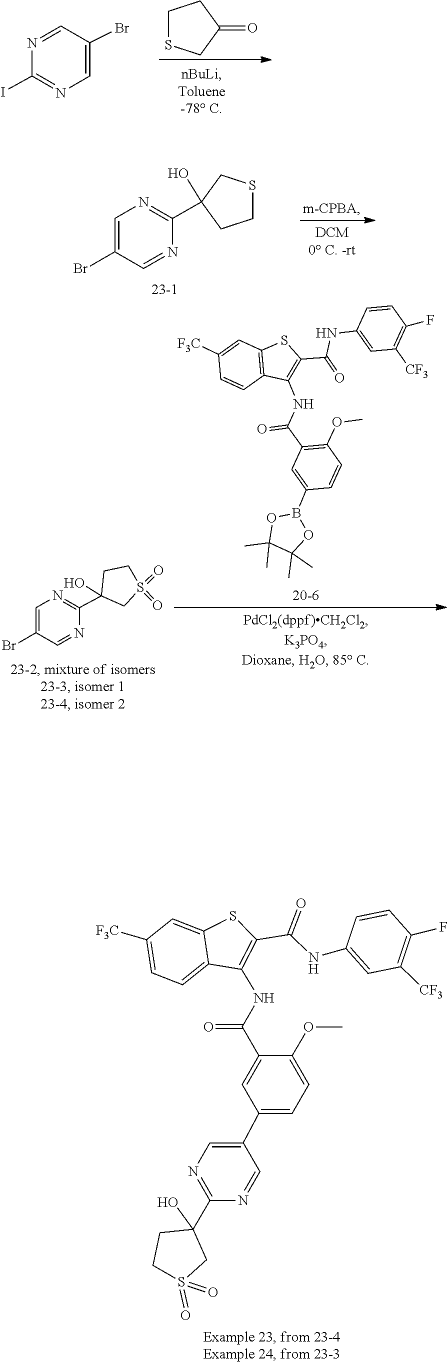 [SMILES: Brc1cnc(I)nc1 ; O=C1CCSC1 ; OC1(c2ncc(Br)cn2)CCSC1 ; O=S1(=O)CCC(O)(c2ncc(Br)cn2)C1 ; COc1ccc(B2OC(C)(C)C(C)(C)O2)cc1C(=O)Nc1c(C(=O)Nc2ccc(F)c(C(F)(F)F)c2)sc2cc(C(F)(F)F)ccc12 ; COc1ccc(-c2cnc(C3(O)CCS(=O)(=O)C3)nc2)cc1C(=O)Nc1c(C(=O)Nc2ccc(F)c(C(F)(F)F)c2)sc2cc(C(F)(F)F)ccc12]

### Intermediate 23-1: 3-(5-bromopyrimidin-2-yl)tetrahydrothiophen-3-ol

To a stirred solution of 5-bromo-2-iodopyrimidine (220 g, 772 mmol) and dihydrothiophen-3(2H)-one (165 mL, 1930 mmol) in toluene (5 L) at −78° C. was added nBuLi (804 mL, 1930 mmol) dropwise under N₂ and the reaction mixture was allowed to stir for 1.5 h-78° C. The reaction mixture was quenched with 150 mL of 10% aq NH4Cl solution at 0° C., and the resulting solution was extracted with EtOAc (2×1 L). The combined organic layer was washed with 500 mL of brine solution, dried over Na₂SO₄, filtered and concentrated under reduced pressure. The residue was purified by column chromatography. The resulting solid was precipitated from 200 mL pet ether and 20 mL IPA and dried over high vacuum to give Intermediate 23-1 (48.0 g, 185 mmol, 24% yield). LC-MS RT: 1.36 min; MS (ESI) m/z 261.0 (M+H)⁺; Method C.

### Intermediate 23-2, 23-3 and 23-4: 3-(5-bromopyrimidin-2-yl)-3-hydroxytetrahydrothiophene 1,1-dioxide

To the solution of Intermediate 23-1 (48.0 g, 184 mmol) in DCM (1 L) at 0° C., was added m-CPBA (113 g, 460 mmol) portionwise. The reaction mixture was stirred for 5 h at rt, then diluted with 500 mL DCM and cooled to 5° C. A solution of 1N NaOH was added to the reaction mixture until it turned clear, and the resulting solution was stirred for 10 min. The layers were separated and the aqueous layer was extracted with DCM (2×500 mL). The combined organic layer was washed with 250 mL of brine solution, dried over Na₂SO₄, filtered and concentrated under reduced pressure. The residue was precipitated from 200 mL of EtOH and dried over high vacuum to give Intermediate 23-2 (41.0 g, 140 mmol, 76% yield). LC-MS RT: 0.76 min; MS (ESI) m/z 293.0 (M+H)⁺; Method C.

Intermediate 23-2 was purified by prep SFC (Column: Chiralpak AD-H (250×50) mm, 5 μm; % CO₂: 50%; % Co-solvent: 50% of 0.2% NH3 in MeOH:ACN (1:1); Total Flow: 220.0 mL/min; Back Pressure: 100 bar; Temperature: 30° C., UV: 220 nm) to give Intermediate 23-3 (Analytical SFC RT=5.95 min, 100% ee; LC-MS RT: 0.76 min; MS (ESI) m/z 293.0 (M+H)⁺; Method C) and Intermediate 23-4 (Analytical SFC RT=9.26 min, 100% ee; LC-MS RT: 0.73 min; MS (ESI) m/z 293.0 (M+H)⁺; Method C) (Analytical SFC: Column: Chiralpak AD-H (250×4.6) mm, 5 μm; % CO₂: 55%; % Co-solvent: 45% of 0.2% NH3 in MeOH:ACN (1:1); Total Flow: 4.0 mL/min; Back Pressure: 100 bar; Temperature: 35° C., UV: 220 nm).

### Example 23: N-(4-fluoro-3-(trifluoromethyl)phenyl)-3-(5-(2-(3-hydroxy-1,1-dioxidotetrahydrothiophen-3-yl)pyrimidin-5-yl)-2-methoxybenzamido)-6-(trifluoromethyl)benzo[b]thiophene-2-carboxamide

Example 23 (3.0 mg, 3.9 mol, 8% yield) was prepared from Intermediate 23-4 (13 mg, 0.044 mmol) and Intermediate 20-6 (30 mg, 0.044 mmol) in a similar way as Intermediate 13-3, followed by purification by prep HPLC (Method 2). ¹H NMR (400 MHz, DMSO-d6) δ ppm 10.87 (br s, 2H), 10.73 (br s, 1H), 9.18 (s, 1H), 8.68 (s, 1H), 8.21 (s, 1H), 8.14 (br d, J=8.03 Hz, 1H), 8.06 (s, 1H), 8.04 (br s, 1H), 7.83 (br d, J=8.03 Hz, 1H), 7.54 (br t, J=9.54 Hz, 1H), 7.42 (br d, J=8.53 Hz, 1H), 6.33 (s, 2H), 3.98 (s, 3H), 3.79 (d, J=13.55 Hz, 1H), 3.36-3.52 (m, 3H), 2.49-2.55 (m, 2H). LC-MS RT: 2.28 min; MS (ESI) m/z 769.1 (M+H)⁺; Method B.

### Example 24: N-(4-fluoro-3-(trifluoromethyl)phenyl)-3-(5-(2-(3-hydroxy-1,1-dioxidotetrahydrothiophen-3-yl)pyrimidin-5-yl)-2-methoxybenzamido)-6-(trifluoromethyl)benzo[b]thiophene-2-carboxamide

Example 24 (4.0 mg, 5.2 mol, 11% yield) was prepared from Intermediate 23-3 (13 mg, 0.044 mmol) and Intermediate 20-6 (30 mg, 0.044 mmol) in a similar way as Intermediate 13-3, followed by purification by prep HPLC (Method 2). ¹H NMR (400 MHz, DMSO-d6) δ ppm 10.88 (br s, 1H), 10.75 (br s, 1H), 9.18 (s, 1H), 8.69 (s, 1H), 8.21 (d, J=1.7 Hz, 1H), 8.14 (br d, J=8.3 Hz, 1H), 8.09-7.97 (m, 1H), 7.89-7.80 (m, 1H), 7.65-7.47 (m, 2H), 7.47-7.24 (m, 1H), 6.35 (s, 2H), 3.97 (s, 3H), 3.79 (br d, J=13.4 Hz, 1H), 3.49-3.41 (m, 3H), 2.59 (br s, 2H). LC-MS RT: 2.28 min; MS (ESI) m/z 769.0 (M+H)⁺; Method A.

### Example 25 and 26

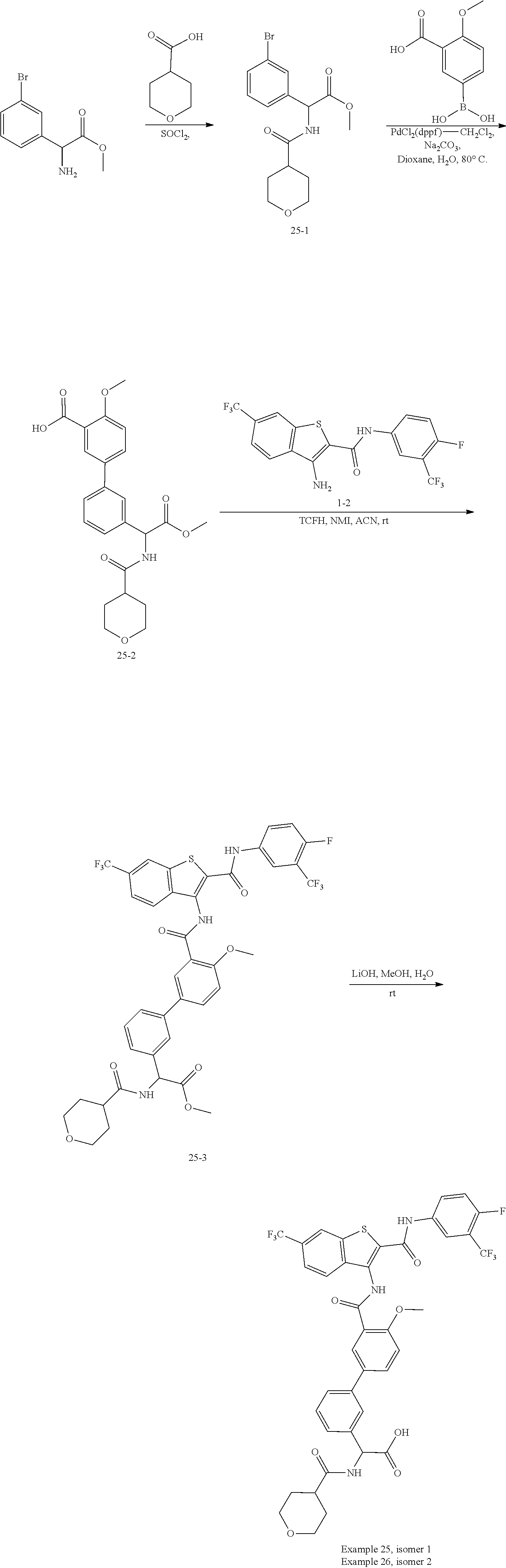

### Intermediate 25-1: Methyl 2-(3-bromophenyl)-2-(tetrahydro-2H-pyran-4-carboxamido)acetate

To a solution of tetrahydro-2H-pyran-4-carboxylic acid (400 mg, 3.07 mmol) in toluene (5 mL) was added thionyl chloride (0.381 mL, 5.23 mmol). The resulting reaction mixture was heated at 80° C. for 1.5 h. The reaction mass was concentrated under high vacuum and dissolved in dry DCM (2 mL). The solution was added dropwise to a solution of methyl 2-amino-2-(3-bromophenyl)acetate (750 mg, 3.07 mmol) and TEA (2.14 mL, 15.4 mmol) in DCM (5 mL) which had been stirred for 5 min at rt prior to the addition. This reaction solution was stirred at rt for 12 h. The reaction mass was partitioned between water (50 mL) and DCM (50 mL). The organic layer was washed with water (2×50 mL), the separated organic layer was dried over Na₂SO₄, filtered and concentrated under reduced pressure. The residue was purified by column chromatography to afford Intermediate 25-1 (300 mg, 0.842 mmol, 27% yield). LC-MS RT: 1.18 min; MS (ESI) m/z 355.9 (M+H)⁺; Method E.

### Intermediate 25-2: 4-methoxy-3′-(2-methoxy-2-oxo-1-(tetrahydro-2H-pyran-4-carboxamido)ethyl)-[1,1′-biphenyl]-3-carboxylic acid

Intermediate 25-2 (110 mg, 0.257 mmol, 51% yield) was prepared from Intermediate 25-1 (180 mg, 0.505 mmol) and 5-borono-2-methoxybenzoic acid (99.0 mg, 0.505 mmol) in a similar manner as Intermediate 13-3, except with Na₂CO₃ (161 mg, 1.52 mmol) as a base, rather than K₃PO₄. LC-MS RT: 0.57 min; MS (ESI) m/z 428.2 (M+H)⁺; Method E.

### Intermediate 25-3: Methyl 2-(3′-((2-((4-fluoro-3-(trifluoromethyl)phenyl)carbamoyl)-6-(trifluoromethyl)benzo[b]thiophen-3-yl)carbamoyl)-4′-methoxy-[1,1′-biphenyl]-3-yl)-2-(tetrahydro-2H-pyran-4-carboxamido)acetate

Intermediate 25-3 (80 mg, 0.096 mmol, 41% yield) was prepared from Intermediate 1-2 (99 mg, 0.23 mmol) and Intermediate 25-2 (100 mg, 0.23 mmol) in a similar manner as Intermediate 1-3. LC-MS RT: 1.16 min; MS (ESI) m/z 830.4 (M−H)⁻; Method E.

### Example 25 and 26: 2-(3′-((2-((4-fluoro-3-(trifluoromethyl) phenyl) carbamoyl)-6-(trifluoromethyl) benzo[b]thiophen-3-yl) carbamoyl)-4′-methoxy-[1, 1′-biphenyl]-3-yl)-2-(tetrahydro-2H-pyran-4-carboxamido) acetic acid

Example 25 and Example 26 was prepared from Example 25-3 (80 mg, 0.096 mmol) by the procedure described in Example 2. The isomers were separated by prep SFC (Column: Chiralpak IC (250×21) mm, 5 μm; % CO₂: 65%; Co-solvent: 35% 0.2% DEA in ACN:MeOH (1:1); OutletPressure: 100 bar; Flow Rate: 70 g/min; Temperature: 30° C. UV: 220 nm.) to give Example 25 (Analytical SFC RT=11.3 min, 100% ee) and Example 26 (Analytical SFC RT=8.38 min, 95% ee). [Analytical SFC: Column: Chiralpak IC (250×4.6) mm, 5 μm; % CO₂: 65%; Co-solvent: 35% 0.2% DEA in ACN:MeOH (1:1); Injected Volume: 40 μl; Outlet Pressure: 100 bar; Flow Rate: 4 mL/min; Temperature: 30° C.]

Example 25: (12 mg, 0.014 mmol, 15% yield)¹H NMR (400 MHz, DMSO-d6) δ ppm 12.25-13.12 (s, 1H), 10.80-11.31 (m, 2H), 8.66 (s, 1H), 8.22-8.29 (m, 2H), 8.07 (br d, J=6.02 Hz, 1H), 7.89-8.15 (m, 4H), 7.79 (br s, 2H), 7.54-7.63 (m, 1H), 7.31 (s, 1H), 7.29 (br s, 1H), 6.78 (s, 1H), 3.91 (s, 4H), 3.83 (br d, J=11.55 Hz, 1H), 3.27 (br s, 2H), 2.86 (q, J=7.19 Hz, 2H), 2.68-2.70 (m, 2H), 1.24 (s, 2H). LC-MS RT: 2.57 min; MS (ESI) m/z 816.0 (M+H)⁺; Method C.

Example 26: (12 mg, 0.014 mmol, 15% yield) ¹H NMR (400 MHz, DMSO-d6) δ ppm 12.84-13.42 (s, 1H), 10.75-10.91 (m, 1H), 10.48-10.65 (m, 1H), 8.14 (br d, J=4.02 Hz, 2H), 8.07 (br d, J=6.02 Hz, 2H), 8.00 (br s, 4H), 7.77-7.92 (m, 2H), 7.62-7.75 (m, 1H), 7.29-7.54 (m, 1H), 7.02-7.19 (m, 1H), 6.57 (s, 1H), 4.01 (s, 5H), 3.44 (br s, 2H), 2.44-2.49 (m, 4H), 1.22-1.30 (m, 2H). LC-MS RT: 2.57 min; MS (ESI) m/z 816.0 (M+H)⁺; Method C.

### Example 27

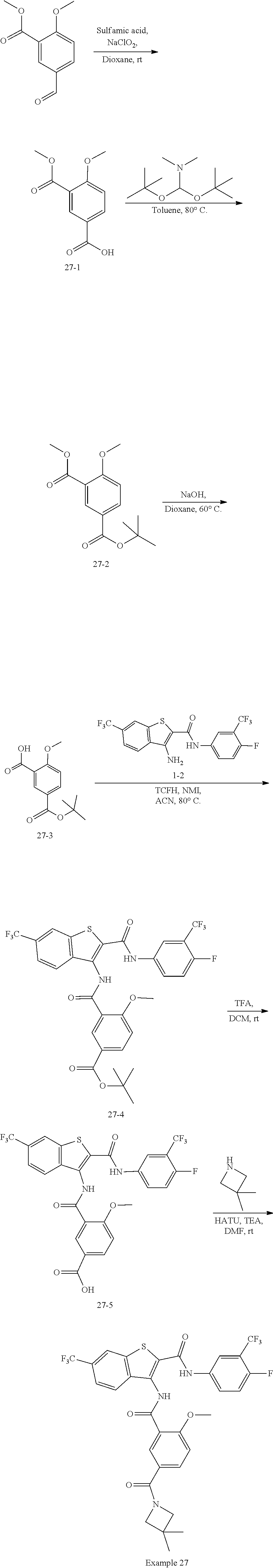

### Intermediate 27-1: 4-methoxy-3-(methoxycarbonyl) benzoic acid

To a solution of methyl 5-formyl-2-methoxybenzoate (1.50 g, 7.72 mmol) in dioxane (35 mL) was added sulfamic acid (4.50 g, 46.3 mmol) in 20 mL H2O and sodium chlorite (1.75 g, 15.5 mmol) in 10 mL H2O, respectively at 0° C., then the reaction mixture was stirred at rt for 2 h. The reaction mass was diluted with EtOAc, washed with water, brine, dried over Na₂SO₄, filtered and concentrated under reduced pressure to give Intermediate 27-1 (1.60 g, 7.61 mmol, 99% yield). LC-MS RT: 0.35 min; MS (ESI) m/z 209.2 (M−H)⁻; Method E.

### Intermediate 27-2: 1-(tert-butyl) 3-methyl 4-methoxyisophthalate

To a solution of Intermediate 27-1 (1.47 g, 7.01 mmol) in toluene (70 mL) was added N,N-dimethylformamide di-tert-butyl acetal (6.72 mL, 28.0 mmol) at 80° C. and the reaction solution was stirred at the same temperature for 16 h. The reaction mass was concentrated under reduced pressure, extracted with EtOAc, washed with 10% NaHCO₃, dried over Na₂SO₄, filtered and concentrated under reduced pressure. The residue was purified via column chromatography to afford Intermediate 27-2 (1.62 g, 6.08 mmol, 87% yield). LC-MS RT: 1.68 min; MS (ESI) m/z 267.2 (M+H)⁺; Method E.

### Intermediate 27-3: 5-(tert-butoxycarbonyl)-2-methoxybenzoic acid

To a solution of Intermediate 27-2 (500 mg, 1.88 mmol) in dioxane (10 mL) was added 1M NaOH solution (4.69 mL, 4.69 mmol) and the resulting reaction mixture was stirred at 60° C. for 2 h. The reaction mass was diluted with EtOAc (100 mL), washed with water (2×50 mL). The separated organic layer was dried over Na₂SO₄, filtered and concentrated under reduced pressure. The residue was dissolved in water (2 mL), acidified using cold 0.5 N HCl at 0° C., and the solid precipitate was filtered, washed with water and dried under vacuum to provide Intermediate 27-3 (320 mg, 1.27 mmol, 68% yield). LC-MS RT: 0.74 min; MS (ESI) m/z 251.1 (M−H)⁻; Method E.

### Intermediate 27-4: Tert-butyl 3-((2-((4-fluoro-3-(trifluoromethyl)phenyl)carbamoyl)-6-(trifluoromethyl)benzo[b]thiophen-3-yl)carbamoyl)-4-methoxybenzoate

Intermediate 27-4 (10 mg, 0.015 mmol, 21% yield) was prepared from Intermediate 1-2 (30 mg, 0.071 mmol) and Intermediate 27-3 (27 mg, 0.11 mmol) by the methods described to prepare Intermediate 1-3, followed by purification by prep LCMS (Method 1). ¹H NMR (400 MHz, DMSO-d6) δ ppm 10.96-10.67 (m, 2H), 8.66 (s, 1H), 8.27 (br d, J=1.2 Hz, 1H), 8.17-8.02 (m, 3H), 8.02-7.91 (m, 1H), 7.82 (dd, J=1.7, 8.8 Hz, 1H), 7.53 (t, J=9.7 Hz, 1H), 7.32 (br d, J=8.3 Hz, 1H), 3.99 (s, 3H), 1.52 (s, 9H). LC-MS RT: 2.77 min; MS (ESI) m/z 655.1 (M−H)⁻; Method A.

### Intermediate 27-5: 3-((2-((4-fluoro-3-(trifluoromethyl)phenyl)carbamoyl)-6-(trifluoromethyl)benzo[b]thiophen-3-yl)carbamoyl)-4-methoxybenzoic acid

Intermediate 27-5 (180 mg, 0.30 mmol, 98% yield) was prepared from Intermediate 27-4 (200 mg, 0.31 mmol) in a similar way as Intermediate 4-2, followed by purification by prep LCMS (Method 1). ¹H NMR (400 MHz, DMSO-d6) δ ppm 10.85 (br d, J=3.9 Hz, 2H), 10.77 (br s, 1H), 8.67 (d, J=0.7 Hz, 1H), 8.37 (d, J=2.4 Hz, 1H), 8.20-8.05 (m, 2H), 8.0 (td, J=3.8, 8.4 Hz, 1H), 7.82 (dd, J=1.7, 8.8 Hz, 1H), 7.54 (t, J=9.8 Hz, 1H), 7.34 (d, J=8.8 Hz, 1H), 4.00 (s, 3H). LC-MS RT: 1.83 min; MS (ESI) m/z 601.0 (M+H)⁺; Method A.

Example 27: 3-(5-(3, 3-dimethylazetidine-1-carbonyl)-2-methoxybenzamido)-N-(4-fluoro-3-(trifluoromethyl) phenyl)-6-(trifluoromethyl) benzo[b]thiophene-2-carboxamide Example 27 (4.8 mg, 7.2 μmol, 29% yield) was prepared from Intermediate 27-5 (15 mg, 0.025 mmol) and 3,3-dimethylazetidine, TFA (25 mg, 0.13 mmol) by the procedure described for Examples 7 and 8, followed by purification by prep LCMS (Method 1). ¹H NMR (400 MHz, DMSO-d6) δ ppm 10.81 (s, 1H), 10.74 (s, 1H), 8.67 (s, 1H), 8.14 (dd, J=2.2, 6.4 Hz, 1H), 8.08 (d, J=8.3 Hz, 1H), 8.04 (d, J=2.2 Hz, 1H), 8.02-7.91 (m, 1H), 7.88-7.77 (m, 2H), 7.54 (t, J=9.7 Hz, 1H), 7.28 (d, J=8.8 Hz, 1H), 4.03-3.87 (m, 5H), 3.72 (br d, J=1.0 Hz, 2H), 1.22 (s, 6H). LC-MS RT: 2.48 min; MS (ESI) m/z 668.2 (M+H)⁺; Method B.

### Example 28

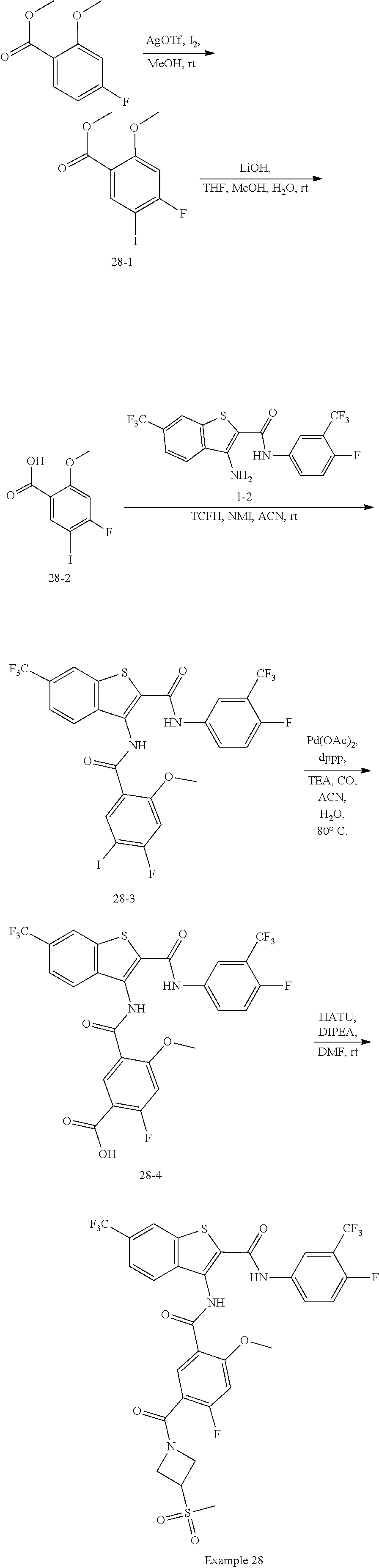

### Intermediate 28-1: Methyl 4-fluoro-5-iodo-2-methoxybenzoate

To a solution of methyl 4-fluoro-2-methoxybenzoate (1.00 g, 5.43 mmol) in MeOH (20 mL) was added silver trifluoromethanesulfonate (2.79 g, 10.9 mmol) followed by iodine (2.76 g, 10.9 mmol). The resulting reaction mixture was stirred at rt for 16 h. The reaction mass was filtered and the filtrate was concentrated under vacuum. The residue was purified by column chromatography to afford Intermediate 28-1 (850 mg, 2.74 mmol, 51% yield). LC-MS RT: 2.08 min; MS (ESI) m/z 311.2 (M+H)⁺; Method C.

### Intermediate 28-2: 4-fluoro-5-iodo-2-methoxybenzoic acid

Intermediate 28-2 (120 mg, 0.405 mmol, 63% yield) was prepared from Intermediate 28-1 (200 mg, 0.645 mmol) by the procedure described in Example 2. LC-MS RT: 0.59 min; MS (ESI) m/z 295.0 (M−H)⁻; Method C.

### Intermediate 28-3: N-(4-fluoro-3-(trifluoromethyl) phenyl)-3-(5-fluoro-4-iodo-2-methoxybenzamido)-6-(trifluoromethyl) benzo[b]thiophene-2-carboxamide

Intermediate 28-3 (165 mg, 0.236 mmol, 42% yield) was prepared from Intermediate 1-2 (235 mg, 0.557 mmol) and Intermediate 28-2 (165 mg, 0.557 mmol) in a similar way as Intermediate 1-3. LC-MS RT: 1.53 min; MS (ESI) m/z 701.2 (M+H)⁺; Method E.

### Intermediate 28-4: 2-fluoro-5-((2-((4-fluoro-3-(trifluoromethyl) phenyl) carbamoyl)-6-(trifluoromethyl) benzo[b]thiophen-3-yl) carbamoyl)-4-methoxybenzoic acid

To a solution of Intermediate 28-3 (500 mg, 0.71 mmol) in ACN (30 mL) and water (10 mL) was added 1,3-bis(diphenylphosphino)propane (29 mg, 0.071 mmol) and palladium(II) acetate (16 mg, 0.071 mmol). The solution was degassed with N₂ for 5 min, then TEA (3.0 mL, 22 mmol) was added. The resulting reaction mixture was heated in autoclave at 80° C. under 5 kg/cm² pressure in presence of CO gas for 3 h. The reaction mass was diluted with EtOAc (100 mL) and filtered. The filtrate was washed with water (2×50 mL). The combined aqueous layers were acidified with 12M HCl to pH 1 and extracted with EtOAc (2×100 mL). The combined organic layer was dried over Na₂SO₄, filtered and concentrated under reduced pressure. The residue was purified by prep LCMS (Method 4) to give Intermediate 28-4 (380 mg, 0.34 mmol, 48% yield). ¹H NMR (400 MHz, DMSO-d6) δ ppm 13.31-13.02 (m, 1H), 10.77 (d, J=2.7 Hz, 2H), 8.67 (s, 1H), 8.38 (d, J=8.8 Hz, 1H), 8.15 (dd, J=2.2, 6.1 Hz, 1H), 8.06 (d, J=8.6 Hz, 1H), 8.04-7.90 (m, 1H), 7.81 (d, J=9.0 Hz, 1H), 7.54 (t, J=9.7 Hz, 1H), 7.25 (d, J=13.0 Hz, 1H), 4.00 (s, 3H). LC-MS RT: 2.42 min; MS (ESI) m/z 619.0 (M+H)⁺; Method C.

### Example 28: 3-(4-fluoro-2-methoxy-5-(3-(methylsulfonyl)azetidine-1-carbonyl)benzamido)-N-(4-fluoro-3-(trifluoromethyl)phenyl)-6-(trifluoromethyl)benzo[b]thiophene-2-carboxamide

Example 28 (2.2 mg, 2.9 μmol, 12% yield) was prepared from Intermediate 28-4 (15 mg, 0.024 mmol) and 3-(methylsulfonyl)azetidine hydrochloride (4.2 mg, 0.024 mmol) in a similar way as Example 18, followed by purification by prep LCMS (Method 1). ¹H NMR (400 MHz, DMSO-d6) δ ppm 10.88-10.66 (m, 2H), 8.66 (s, 1H), 8.18 (dd, J=4.0, 2.1 Hz, 1H), 8.11-7.89 (m, 3H), 7.81 (d, J=8.6 Hz, 1H), 7.54 (t, J=9.7 Hz, 1H), 7.28 (d, J=12.2 Hz, 1H), 4.46-4.27 (m, 2H), 4.27-4.15 (m, 2H), 3.98 (s, 3H), 3.90 (s, 1H), 3.04 (s, 3H). LC-MS RT: 2.21 min; MS (ESI) m/z 736.0 (M+H)⁺; Method A.

### Example 29

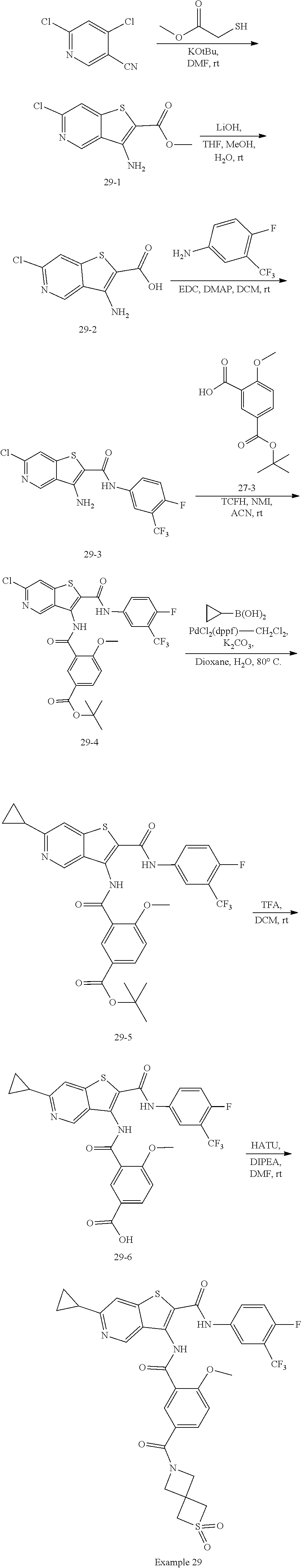

### Intermediate 29-1: Methyl 3-amino-6-chlorothieno[3,2-c]pyridine-2-carboxylate

To a solution of 4, 6-dichloronicotinonitrile (5.00 g, 28.9 mmol) in DMF (90 mL) was added methyl 2-mercaptoacetate (3.35 mL, 34.7 mmol) followed by potassium tert-butoxide (34.0 mL, 57.8 mmol). The resulting reaction mixture was stirred at rt for 12 h. The reaction mass was quenched with saturated NH4Cl solution (50 mL) at 5° C., and stirred at rt for 20 min. The solid precipitate was filtered and dried under vacuum to give Intermediate 29-1 (5.00 g, 20.6 mmol, 71% yield). LC-MS RT: 0.57 min; MS (ESI) m/z 243.0 (M+H)⁺; Method E.

### Intermediate 29-2: 3-amino-6-chlorothieno[3,2-c]pyridine-2-carboxylic acid

Intermediate 29-2 (175 mg, 0.765 mmol, 74% yield) was prepared from Intermediate 29-1 (250 mg, 1.03 mmol) by the general procedure described for Example 2. LC-MS RT: 0.31 min; MS (ESI) m/z 229.0 (M+H)⁺; Method E.

### Intermediate 29-3: 3-amino-6-chloro-N-(4-fluoro-3-(trifluoromethyl)phenyl)thieno[3,2-c]pyridine-2-carboxamide

To a solution of Intermediate 29-2 (250 mg, 1.09 mmol) in DCM (15 mL) was added 4-fluoro-3-(trifluoromethyl)aniline (294 mg, 1.64 mmol) followed by EDC (419 mg, 2.19 mmol) and DMAP (134 mg, 1.09 mmol). The resulting reaction mixture was stirred at rt for 16 h. The reaction mixture was diluted with EtOAc (50 mL), washed with water (2×50 mL), dried over Na₂SO₄, and concentrated under reduced pressure. The residue was purified via column chromatography to afford Intermediate 29-3 (250 mg, 0.641 mmol, 59% yield). LC-MS RT: 0.85 min; MS (ESI) m/z 388.0 (M−H)⁻ Method E.

### Intermediate 29-4: Tert-butyl 3-((6-chloro-2-((4-fluoro-3-(trifluoromethyl)phenyl)carbamoyl)thieno[3,2-c]pyridin-3-yl)carbamoyl)-4-methoxybenzoate

Intermediate 29-4 (200 mg, 0.179 mmol, 39% yield) was prepared from Intermediate 29-3 (180 mg, 0.462 mmol) and Intermediate 27-3 (233 mg, 0.924 mmol) in a similar way as Intermediate 1-3. ¹H NMR (400 MHz, DMSO-d6) δ ppm 11.09 (br d, J=2.7 Hz, 1H), 10.79 (br d, J=1.5 Hz, 1H), 9.00 (s, 1H), 8.39 (d, J=0.7 Hz, 1H), 8.34 (d, J=2.2 Hz, 1H), 8.15 (dd, J=2.2, 6.4 Hz, 1H), 8.07 (dd, J=2.3, 8.7 Hz, 1H), 8.02-7.93 (m, 1H), 7.54 (t, J=9.8 Hz, 1H), 7.33 (d, J=8.8 Hz, 1H), 3.99 (s, 3H), 1.53 (s, 9H). LC-MS RT: 2.61 min; MS (ESI) m/z 625.2 (M+H)⁺; Method B.

### Intermediate 29-5: Tert-butyl 3-((6-cyclopropyl-2-((4-fluoro-3-(trifluoromethyl)phenyl)carbamoyl)thieno[3,2-c]pyridin-3-yl)carbamoyl)-4-methoxybenzoate

Intermediate 29-5 (56 mg, 0.089 mmol, 79% yield) was prepared from Intermediate 29-4 (70 mg, 0.11 mmol) and cyclopropylboronic acid (70 mg, 0.82 mmol) in a similar way as Intermediate 13-3, except K₂CO₃ (47 mg, 0.34 mmol) was used as a base instead of K₃PO₄. LC-MS RT: 1.34 min; MS (ESI) m/z 630.3 (M+H)⁺; Method E.

### Intermediate 29-6: 3-((6-cyclopropyl-2-((4-fluoro-3-(trifluoromethyl)phenyl)carbamoyl)thieno[3,2-c]pyridin-3-yl)carbamoyl)-4-methoxybenzoic acid

Intermediate 29-6 (60 mg, 0.11 mmol, 82% yield) was prepared from Intermediate 29-5 (80 mg, 0.13 mmol) in a similar way as Intermediate 13-4. LC-MS RT: 0.43 min; MS (ESI) m/z 574.2 (M+H)⁺; Method E.

### Example 29: 6-cyclopropyl-3-(5-(2, 2-dioxido-2-thia-6-azaspiro [3.3]heptane-6-carbonyl)-2-methoxybenzamido)-N-(4-fluoro-3-(trifluoromethyl) phenyl) thieno [3, 2-c]pyridine-2-carboxamide

Example 29 (2.0 mg, 2.9 μmol, 11% yield) was prepared from Intermediate 29-6 (15 mg, 0.026 mmol) and 2-thia-6-azaspiro[3.3]heptane 2,2-dioxide (5.8 mg, 0.039 mmol) by the general procedures described for Example 18, followed by purification by Prep LCMS (Method 1). ¹H NMR (400 MHz, DMSO-d6) δ 11.14 (s, 1H), 10.66 (s, 1H), 9.00 (s, 1H), 8.22-8.16 (m, 1H), 8.06-8.03 (m, 1H), 8.03-7.98 (m, 1H), 7.89-7.83 (m, 1H), 7.59-7.53 (m, 1H), 7.33 (d, J=8.8 Hz, 1H), 7.21 (s, 1H), 4.60 (br dd, J=4.3, 2.8 Hz, 1H), 4.49 (br dd, J=3.8, 2.1 Hz, 4H), 4.29 (m, 4H), 1.25 (s, 3H), 1.06-1.01 (m, 4H). LC-MS RT: 2.04 min; MS (ESI) m/z 703.2 (M+H)⁺; Method A.

### Example 30 and 31

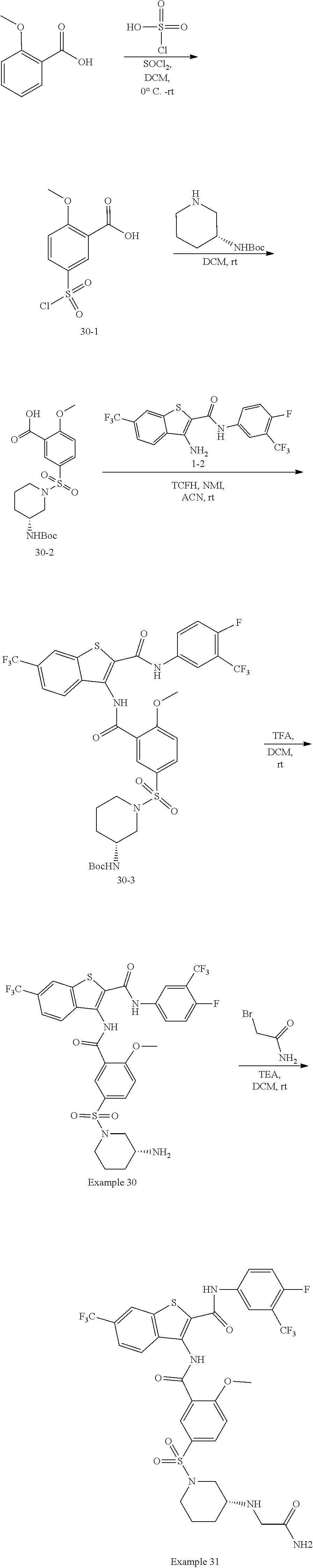

### Intermediate 30-1: 5-(chlorosulfonyl)-2-methoxybenzoic acid

To an ice cooled solution of chlorosulfonic acid (1.10 mL, 16.4 mmol) in DCM (5.0 mL) was added 2-methoxybenzoic acid (500 mg, 3.29 mmol) over a period of 15 min. Thionyl chloride (0.240 mL, 3.29 mmol) was added to the reaction mixture which was then stirred for 12 h at rt. The reaction mixture was poured into crushed ice to get white precipitate which was filtered, washed with pet ether, and dried under vacuum to give Intermediate 30-1 (800 mg, 3.20 mmol, 97% yield). LC-MS RT: 0.46 min; MS (ESI) m/z 251.0 (M+H)⁺; Method E.

### Intermediate 30-2: 5-(3-((tert-butoxycarbonyl) amino) piperidin-1-yl) sulfonyl)-2-methoxybenzoic acid

A mixture of Intermediate 30-1 (500 mg, 2.00 mmol) and tert-butyl (R)-piperidin-3-ylcarbamate (400 mg, 2.00 mmol) in DCM (10 mL) was stirred for 12 h at rt. The reaction mass was concentrated under reduced pressure. The residue was purified by column chromatography to give Intermediate 30-2 (290 mg, 0.700 mmol, 35% yield). LC-MS RT: 1.66 min; MS (ESI) m/z 315.1 (M+H-Boc)⁺; Method E.

### Intermediate 30-3: Tert-butyl (R)-(1-((3-((2-((4-fluoro-3-(trifluoromethyl) phenyl) carbamoyl)-6-(trifluoromethyl) benzo[b]thiophen-3-yl) carbamoyl)-4-methoxyphenyl) sulfonyl) piperidin-3-yl) carbamate

Intermediate 30-3 (250 mg, 0.305 mmol, 65% yield) was prepared from Intermediate 1-2 (200 mg, 0.474 mmol) and Intermediate 30-2 (294 mg, 0.710 mmol) by a similar method as Intermediate 1-3. LC-MS RT: 2.60 min; MS (ESI) m/z 819.2 (M+H)⁺; Method E.

### Example 30: (R)-3-(5-((3-aminopiperidin-1-yl) sulfonyl)-2-methoxybenzamido)-N-(4-fluoro-3-(trifluoromethyl) phenyl)-6-(trifluoromethyl) benzo[b]thiophene-2-carboxamide

To a mixture of Intermediate 30-3 (200 mg, 0.244 mmol) in DCM (15 mL) at 0° C. was added TFA (56.0 μL, 0.733 mmol). The resulting reaction mixture was stirred for 5 h at rt. The reaction mass was concentrated under vacuum and the residue was purified by prep LCMS (Method 1) to give Example 30 (150 mg, 0.209 mmol, 85% yield). ¹H NMR (400 MHz, DMSO-d6) δ ppm 10.88-10.83 (m, 1H), 10.78 (br d, J=1.2 Hz, 1H), 8.63 (s, 1H), 8.17 (dd, J=2.2, 6.4 Hz, 1H), 8.11 (d, J=8.6 Hz, 1H), 8.03 (d, J=2.2 Hz, 1H), 7.96-7.89 (m, 1H), 7.86 (dd, J=2.3, 8.9 Hz, 1H), 7.80 (d, J=8.8 Hz, 1H), 7.53 (t, J=9.7 Hz, 1H), 7.45 (d, J=8.8 Hz, 1H), 3.99 (s, 3H), 3.47-3.44 (m, 1H), 2.82-2.72 (m, 1H), 2.29-2.18 (m, 1H), 2.01 (br t, J=10.0 Hz, 1H), 1.76-1.61 (m, 2H), 1.52-1.39 (m, 1H), 1.02-0.87 (m, 2H). LC-MS RT: 2.17 min; MS (ESI) m/z 719.2 (M+H)⁺; Method A.

### Example 31: (R)—N-(4-fluoro-3-(trifluoromethyl) phenyl)-3-(5-((3-(3-hydroxypropanamido) piperidin-1-yl) sulfonyl)-2-methoxybenzamido)-6-(trifluoromethyl) benzo[b]thiophene-2-carboxamide

Example 31 (6.0 mg, 7.8 mol, 55% yield) was prepared from Example 30 (10 mg, 0.014 mmol) and 2-bromoacetamide (2.3 mg, 0.017 mmol) in a similar way as Example 9 except using DCM as a solvent, followed by purification by prep HPLC (Method 2). ¹H NMR (400 MHz, DMSO-d6) δ ppm 10.94-10.86 (m, 1H), 10.80 (br d, J=2.0 Hz, 1H), 8.67 (s, 1H), 8.23-8.18 (m, 1H), 8.11 (d, J=8.8 Hz, 1H), 8.07 (d, J=2.7 Hz, 1H), 8.00-7.94 (m, 1H), 7.90 (dd, J=2.2, 8.8 Hz, 1H), 7.85-7.78 (m, 1H), 7.60-7.50 (m, 1H), 7.46 (d, J=9.3 Hz, 1H), 7.26 (dd, J=1.7, 2.4 Hz, 1H), 7.07-6.98 (m, 2H), 4.01 (s, 4H), 3.48 (br d, J=2.0 Hz, 1H), 3.07 (s, 2H), 2.32-2.28 (m, 1H), 2.13-2.06 (m, 2H), 1.77-1.63 (m, 2H), 1.48-1.40 (m, 1H), 0.97 (br d, J=2.4 Hz, 1H). LC-MS RT: 2.17 min; MS (ESI) m/z 776.1 (M+H)⁺; Method A.

### Example 32 and 33

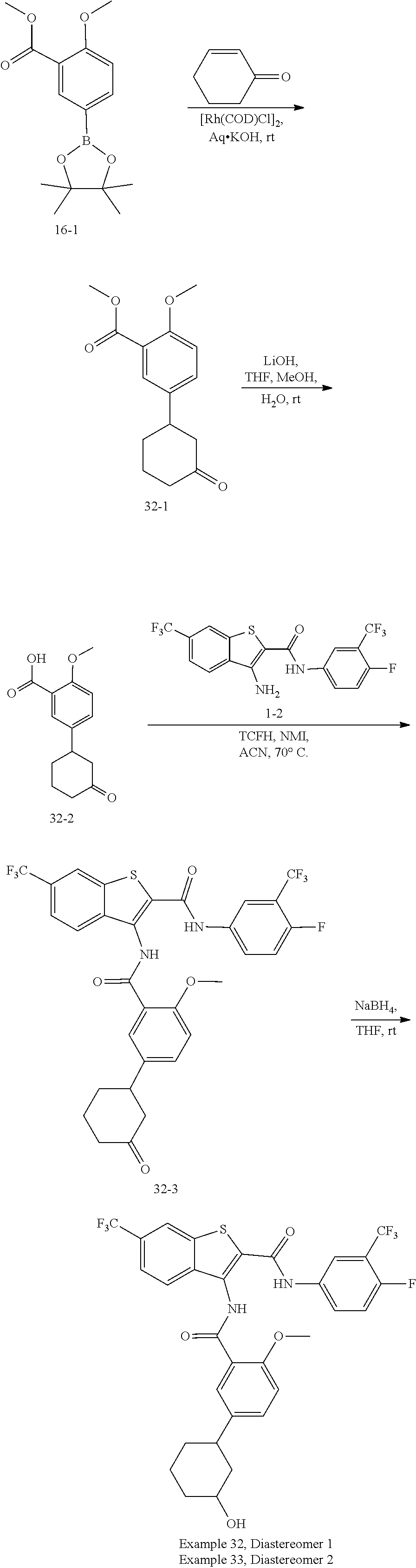

### Intermediate 32-1: Methyl 2-methoxy-5-(3-oxocyclohexyl) benzoate

To a solution of chloro(1,5-cyclooctadiene)rhodium(I)dimer (16 mg, 0.033 mmol) in 1,4-dioxane (15 mL) was added a previously degassed solution of KOH (370 mg, 6.5 mmol) in H2O (1 mL) and the reaction mixture was stirred for 15 min at rt. Intermediate 16-1 (3.8 g, 13 mmol) and 2-cyclohexen-1-one (1.3 mL, 13 mmol) were successively added to the reaction solution, and was stirred for 30 min at rt. The reaction mass was diluted with water and extracted with DCM (2×50 mL). The separated organic layer was dried over Na₂SO₄, filtered and concentrated under reduced pressure to afford Intermediate 32-1 (1.2 g, 4.6 mmol, 35% yield). ¹H NMR (400 MHz, DMSO-d6) δ ppm 7.47-7.42 (m, 1H), 7.46-7.40 (m, 1H), 7.07-7.05 (m, 1H), 3.93 (m, 6H), 2.99 (m, 1H), 2.45-2.40 (m, 2H), 2.01-1.99 (m, 2H), 1.07 (m, 4H).

### Intermediate 32-2: 2-methoxy-5-(3-oxocyclohexyl) benzoic acid

Intermediate 32-2 (4.0 g, 16 mmol, 85% yield) was prepared from Intermediate 32-1 (5.0 g, 19 mmol) by the procedure described in Example 2. LC-MS RT: 0.28 min; MS (ESI) m/z 249.2 (M+H)⁺; Method E.

### Intermediate 32-3: N-(4-fluoro-3-(trifluoromethyl) phenyl)-3-(2-methoxy-5-(3-oxocyclohexyl) benzamido)-6-(trifluoromethyl) benzo[b]thiophene-2-carboxamide

Intermediate 32-3 (2.0 g, 3.1 mmol, 32% yield) was prepared from Intermediate 1-2 (4.0 g, 9.5 mmol) and Intermediate 32-2 (2.4 g, 9.5 mmol) following the conditions described for Intermediate 1-3. LC-MS RT: 1.24 min; MS (ESI) m/z 651.2 (M−H)⁻; Method E.

### Example 32 and 33: N-(4-fluoro-3-(trifluoromethyl) phenyl)-3-(5-(3-hydroxycyclohexyl)-2-methoxybenzamido)-6-(trifluoromethyl)benzo[b]thiophene-2-carboxamide

To a solution of Intermediate 32-3 (120 mg, 0.184 mmol) in THF (2 mL) was added sodium borohydride (6.96 mg, 0.184 mmol) and the reaction mixture was stirred for 2 h at rt. The reaction mass was quenched with ice flakes and extracted with EtOAc (2×50 mL). The combined organic layers were dried over Na₂SO₄, filtered and concentrated under reduced pressure. The residue was purified by prep LCMS (Method 1), to give Example 32 (Prep LCMS RT=2.50 min) and Example 33 (Prep LCMS RT=2.49 min).

Example 32: (11.4 mg, 17.0 mol, 10% yield)¹H NMR (400 MHz, DMSO-d6) δ ppm 10.86-10.66 (m, 2H), 8.66 (s, 1H), 8.18-8.11 (m, 1H), 8.07 (d, J=8.6 Hz, 1H), 8.02-7.92 (m, 1H), 7.82 (d, J=8.8 Hz, 1H), 7.63 (s, 1H), 7.54 (t, J=10.0 Hz, 1H), 7.46-7.36 (m, 1H), 7.16 (d, J=8.3 Hz, 1H), 4.62 (d, J=4.4 Hz, 1H), 3.93 (s, 3H), 3.55-3.43 (m, 1H), 2.54 (br s, 1H), 1.97-1.82 (m, 2H), 1.80-1.71 (m, 1H), 1.68-1.59 (m, 1H), 1.43-1.06 (m, 4H). LC-MS RT: 2.50 min; MS (ESI) m/z 655.2 (M+H)⁺; Method B.

Example 33: (12.8 mg, 19.0 mol, 11% yield)¹H NMR (400 MHz, DMSO-d6) δ ppm 10.86-10.65 (m, 2H), 8.66 (s, 1H), 8.15 (dd, J=2.6, 6.2 Hz, 1H), 8.07 (d, J=8.3 Hz, 1H), 8.03-7.93 (m, 1H), 7.82 (d, J=8.6 Hz, 1H), 7.63 (s, 1H), 7.55 (t, J=10.0 Hz, 1H), 7.45-7.37 (m, 1H), 7.16 (d, J=8.8 Hz, 1H), 4.62 (d, J=4.2 Hz, 1H), 3.93 (s, 3H), 3.55-3.46 (m, 1H), 2.54 (br s, 1H), 1.98-1.82 (m, 2H), 1.79-1.71 (m, 1H), 1.69-1.59 (m, 1H), 1.41-1.01 (m, 4H). LC-MS RT: 2.49 min; MS (ESI) m/z 655.2 (M+H)⁺; Method A.

### Intermediate 34-1, Examples 34, 35, and 36: N-(4-fluoro-3-(trifluoromethyl)phenyl)-3-(5-(3-hydroxy-3-methylcyclohexyl)-2-methoxybenzamido)-6-(trifluoromethyl)benzo[b]thiophene-2-carboxamide

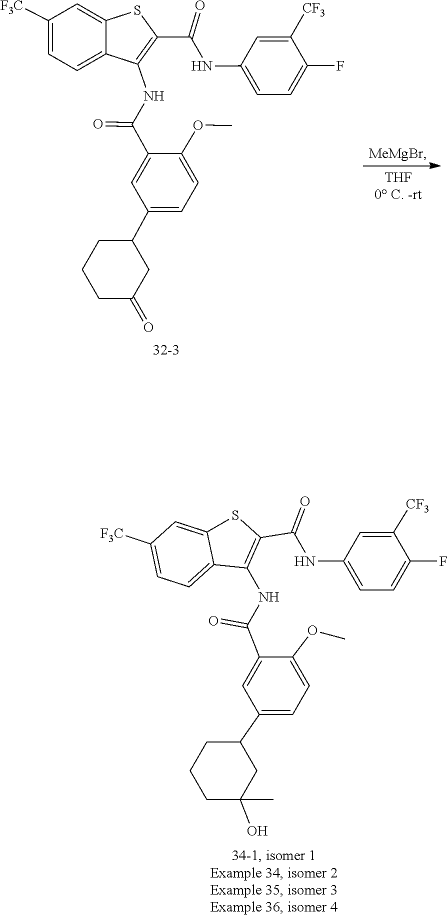 [SMILES: COc1ccc(C2CCCC(=O)C2)cc1C(=O)Nc1c(C(=O)Nc2ccc(F)c(C(F)(F)F)c2)sc2cc(C(F)(F)F)ccc12 ; COc1ccc(C2CCCC(C)(O)C2)cc1C(=O)Nc1c(C(=O)Nc2ccc(F)c(C(F)(F)F)c2)sc2cc(C(F)(F)F)ccc12]

To a solution of Intermediate 32-3 (160 mg, 0.245 mmol) in THF (2 mL) was added methylmagnesium bromide (0.123 mL, 0.245 mmol) at 0° C. The reaction mixture was stirred at rt for 12 h. The reaction mixture was diluted with EtOAc (50 mL) and washed with water (50 mL). The separated organic layer was dried over Na₂SO₄, filtered and concentrated under reduced pressure. The diastereomers were separated by prep HPLC (Column: YMC EXRS C18 (250×21.2) mm 5 μm; Mobile Phase A: 10 mM NH4OAc in H2O Mobile Phase B: ACN; Flow: 20 mL/min, Gradient: 80% 2 min, 80-93% 14 mins; diastereomer 1: RT=11.906 mins, diastereomer 2: RT=13.650 mins). The first eluting diastereomer from the prep HPLC was purified by prep SFC (Column: Whelk(R,R) (250×21) mm, 5 μm; % CO₂: 75%; % Co-solvent: 25% of 0.2% TFA in MeOH; Total Flow: 90.0 g/min; Back Pressure: 100 bar; Temperature: 40° C.; UV: 220 nm) to give Example 35 (Prep SFC RT=4.08 min) and Example 36 (Prep SFC RT=3.03 min, 99% de). Analytical SFC conditions: (Column: Whelk(R,R) (250×4.6) mm, 5 μm; % CO₂: 60%; % Co-solvent: 40% of 0.2% TFA in MeOH; Total Flow: 4.0 g/min; Back Pressure: 100 bar; Temperature: 30° C.; UV: 220 nm). The second eluting diastereomer from the prep HPLC was purified by prep SFC (Column: Chiralpak AD-H (250×21) mm, 5 μm; % CO₂: 50%; % Co-solvent: 50% of 0.2% TFA in MeOH; Total Flow: 90.0 g/min; Back Pressure: 100 bar; Temperature: 40° C., UV: 248 nm) to give Example 34 (Prep SFC RT=11.2 min) and Intermediate 34-1 (Prep SFC RT=6.3 min, 95% de). Analytical SFC conditions: (Column: Chiralpak AD-H (250×4.6) mm, 5 μm; % CO₂: 50%; % Co-solvent: 50% of 0.2% TFA in MeOH; Total Flow: 4.0 g/min; Back Pressure: 100 bar; Temperature: 40° C., UV: 248 nm).

Intermediate 34-1: (4.3 mg, 6.4 mol, 2% yield)¹H NMR (400 MHz, DMSO-d6) δ ppm 10.80-10.85 (m, 1H), 10.72-10.79 (m, 1H), 8.68-8.73 (m, 1H), 8.15-8.22 (m, 1H), 8.07-8.14 (m, 1H), 7.97-8.06 (m, 1H), 7.81-7.89 (m, 1H), 7.63-7.68 (m, 1H), 7.54-7.62 (m, 1H), 7.40-7.46 (m, 1H), 7.10-7.25 (m, 1H), 4.12-4.17 (m, 1H), 3.95-4.00 (m, 3H), 3.46-3.49 (m, 1H), 3.27-3.42 (m, 3H), 2.52-2.59 (m, 2H), 1.69-1.79 (m, 2H), 1.53-1.66 (m, 1H), 1.26-1.29 (m, 3H). LC-MS RT: 1.51 min; MS (ESI) m/z 669.2 (M+H)⁺; Method C.

Example 34: (15 mg, 22 mol, 9% yield). ¹H NMR (400 MHz, DMSO-d6) δ ppm 10.80 (s, 1H), 10.74 (s, 1H), 8.67 (s, 1H), 8.12-8.19 (m, 1H), 8.07 (d, J=8.53 Hz, 2H), 7.83 (d, J=8.30 Hz, 1H), 7.61 (d, J=2.01 Hz, 1H), 7.55 (t, J=9.78 Hz, 1H), 7.39 (d, J=7.86 Hz, 1H), 7.08-7.18 (m, 1H), 4.12 (m, 2H), 3.94 (s, 3H), 3.45 (s, 3H), 1.54 (br s, 3H), 1.33 (s, 2H), 1.24 (s, 3H). LC-MS RT: 1.51 min; MS (ESI) m/z 669.2 (M+H)⁺; Method C.

Example 35: (3.4 mg, 5.1 mol, 2% yield). ¹H NMR (400 MHz, DMSO-d6) δ ppm 10.78-10.85 (m, 1H), 10.69-10.75 (m, 1H), 8.63-8.70 (m, 1H), 8.13-8.18 (m, 1H), 8.05-8.10 (m, 2H), 8.06 (s, 1H), 7.79-7.85 (m, 1H), 7.69-7.75 (m, 1H), 7.55 (s, 1H), 7.46-7.60 (m, 1H), 7.16-7.24 (m, 1H), 4.52-4.12 (m, 1H), 3.94 (s, 3H), 3.36-3.45 (m, 1H), 3.08-3.29 (m, 1H), 1.77-1.89 (m, 1H), 1.61-1.77 (m, 1H), 1.39-1.43 (m, 3H), 1.24 (br s, 3H), 1.12 (m, 2H). LC-MS RT: 3.56 min; MS (ESI) m/z 651.2 (M-OH)⁺; Method C.

Example 36: (4.6 mg, 6.9 mol, 3% yield). ¹H NMR (400 MHz, DMSO-d6) δ ppm 10.65-10.92 (m, 2H), 8.63-8.69 (m, 1H), 8.14-8.19 (m, 1H), 8.05-8.11 (m, 1H), 7.94-8.03 (m, 1H), 7.80-7.85 (m, 1H), 7.64-7.66 (m, 1H), 7.52-7.59 (m, 1H), 7.38-7.45 (m, 1H), 7.13-7.20 (m, 1H), 4.38-4.41 (m, 1H), 3.91-3.96 (m, 3H), 3.27-3.37 (m, 2H), 1.57-1.74 (m, 2H), 1.54-1.79 (m, 1H), 1.43-1.53 (m, 3H), 1.31-1.42 (m, 1H), 1.16-1.26 (m, 3H). LC-MS RT: 3.96 min; MS (ESI) m/z 667.3 (M−H)⁻; Method C.

### Example 37 and 38

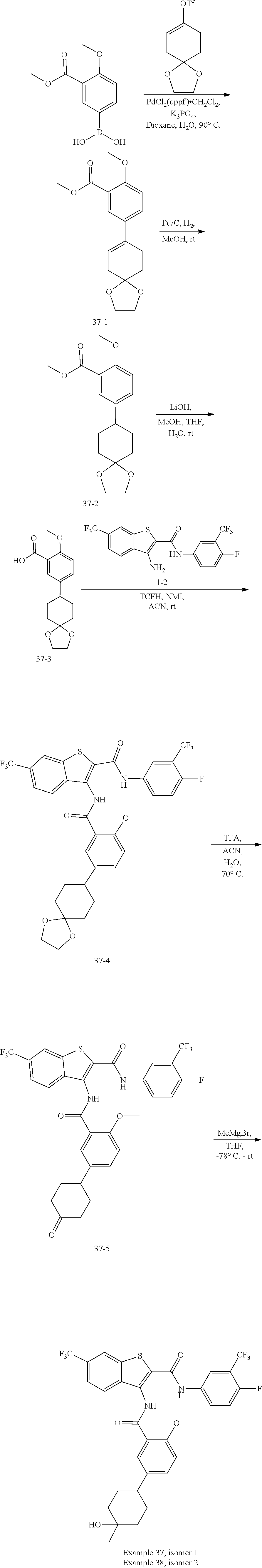

### Intermediate 37-1: Methyl 2-methoxy-5-(1, 4-dioxaspiro [4.5]dec-7-en-8-yl)benzoate

Intermediate 37-1 (4.80 g, 15.7 mmol, 66% yield) was prepared from (4-methoxy-3-(methoxycarbonyl)phenyl)boronic acid (5.00 g, 23.8 mmol) and 1,4-dioxaspiro[4.5]dec-7-en-8-yl trifluoromethanesulfonate (6.86 g, 23.8 mmol) in a similar way as Intermediate 13-3. LC-MS RT: 2.10 min; MS (ESI) m/z 305.2 (M+H)⁺; Method C.

### Intermediate 37-2: Methyl 2-methoxy-5-(1,4-dioxaspiro[4.5]decan-8-yl)benzoate

A solution of Intermediate 37-1 (5.00 g, 16.4 mmol) in MeOH (50 mL) was purged with N₂ gas, then Pd—C(1.75 g, 16.4 mmol) was added and the reaction was evacuated and purged with H2 gas via a balloon connected to the mixture. The reaction mixture was stirred at rt for 8 h. The suspension was filtered through a celite bed, the filtrate was collected and concentrated under reduced pressure to give Intermediate 37-2 (4.00 g, 13.1 mmol, 79% yield). LC-MS RT: 0.87 min; MS (ESI) m/z 307.1 (M+H)⁺; Method F.

### Intermediate 37-3: 2-methoxy-4~(1, 4-dioxaspiro [4.5]decan-8-yl)benzoic acid

Intermediate 37-3 (320 mg, 1.10 nmol, 75% yield) was prepared from Intermediate 37-2 (500 mg, 1.63 mmol) by the procedure described in Example 2. ¹H NMR (400 MHz, DMSO-d6) δ ppm 12.46-12.72 (m, 1H), 7.30-7.54 (m, 2H), 7.10 (dd, J=10.29, 8.78 Hz, 1H), 5.81 (s, 1H), 3.81-3.96 (m, 3H), 3.40 (br s, 2H), 2.53-2.67 (m, 6H), 2.31 (br d, J=14.06 Hz, 1H), 1.76-1.96 (m, 2H), 1.58-1.76 (m, 1H).

### Intermediate 37-4: N-(4-fluoro-3-(trifluoromethyl) phenyl)-3-(2-methoxy-5-(1,4-dioxaspiro[4.5]decan-8-yl)benzamido)-6-(trifluoromethyl)benzo[b]thiophene-2-carboxamide

Intermediate 37-4 (1.50 g, 2.15 mmol, 61% yield) was prepared from Intermediate 1-2 (1.50 g, 3.55 mmol) and Intermediate 37-3 (1.25 g, 4.26 mmol) in a similar way as Intermediate 1-3. LC-MS RT: 1.39 min; MS (ESI) m/z 695.3 (M−H)⁻; Method C.

### Intermediate 37-5: N-(4-fluoro-3-(trifluoromethyl) phenyl)-3-(2-methoxy-5-(4-oxocyclohexyl) benzamido)-6-(trifluoromethyl) benzo[b]thiophene-2-carboxamide

To a solution of Intermediate 37-4 (1.20 g, 1.72 mmol) in ACN (20 mL) and H2O (1 mL) was added TFA (1.33 mL, 17.2 mmol). The reaction mixture was heated at 70° C. for 30 min. The cooled reaction mixture was diluted with EtOAc (100 mL) and washed with saturated NaHCO₃ solution (50 mL). The separated organic layer was dried over Na₂SO₄, filtered and concentrated under reduced pressure. The residue was purified by column chromatography to give Intermediate 37-5 (1.00 g, 1.53 mmol, 89% yield). LC-MS RT: 1.26 min; MS (ESI) m/z 653.2 (M+H)⁺; Method E.

### Example 37 and 38: N-(4-fluoro-3-(trifluoromethyl) phenyl)-3-(5-(4-hydroxy-4-methylcyclohexyl)-2-methoxybenzamido)-6-(trifluoromethyl)benzo[b]thiophene-2-carboxamide

Example 37 and 38 were prepared from Intermediate 37-5 (120 mg, 0.184 mmol) in the same general manner as described for Example 34, 35, and 36, followed by purification by prep HPLC (Column: Gemini NX C-18 (250×21.2) mm, 5 μm; Mobile Phase A: 10 mM NH4HCO3 in Water, pH 9.5; Mobile Phase B: ACN; Flow: 19 mL/min; Gradient: 65-80%, 15 min, 80% 20 min, 80-100% 21 min) to give Example 37 (Prep HPLC RT=15.49 min, Analytical HPLC RT=6.84 mins) and Example 38 (Prep HPLC RT=18.96 min, Analytical HPLC RT=7.54 mins). [Analytical HPLC: Column: Kinetex BIPHENYL (4.6×100) mm, 2.6 μm; Buffer: 0.05% TFA in water; Mobile Phase A: Buffer:ACN (95:5); Mobile Phase B: ACN:Buffer (95:5).]

Example 37: (17 mg, 25 mol, 13% yield)¹H NMR (400 MHz, DMSO-d6) δ ppm 10.66-10.95 (m, 2H), 8.53-8.77 (m, 1H), 8.06-8.14 (m, 1H), 7.94-8.02 (m, 1H), 7.79-7.86 (m, 1H), 7.60-7.64 (m, 1H), 7.50-7.57 (m, 2H), 7.41-7.46 (m, 1H), 7.12-7.17 (m, 1H), 4.32-4.36 (m, 1H), 3.90-3.95 (m, 3H), 3.36-3.39 (m, 1H), 3.31-3.37 (m, 2H), 1.66-1.72 (m, 1H), 1.59-1.64 (m, 1H), 1.41-1.56 (m, 1H), 1.22-1.26 (m, 1H), 1.14-1.19 (m, 1H). LC-MS RT: 4.07 min; MS (ESI) m/z 667.3 (M−H)⁻; Method C.

Example 38: (4.9 mg, 7.3 mol, 4% yield)¹H NMR (400 MHz, DMSO-d6) δ ppm 10.64-10.93 (m, 2H), 8.63-8.68 (m, 1H), 8.08-8.16 (m, 1H), 7.92-8.02 (m, 1H), 7.79-7.85 (m, 1H), 7.60-7.65 (m, 1H), 7.48-7.56 (m, 2H), 7.40-7.47 (m, 1H), 7.11-7.17 (m, 1H), 4.31-4.36 (m, 1H), 3.90-3.95 (m, 3H), 3.31-3.37 (m, 2H), 1.59-1.73 (m, 3H), 1.42-1.56 (m, 2H), 1.22-1.27 (m, 3H), 1.13-1.20 (m, 2H). LC-MS RT: 3.86 min; MS (ESI) m/z 667.2 (M−H)⁻; Method C.

### Example 39

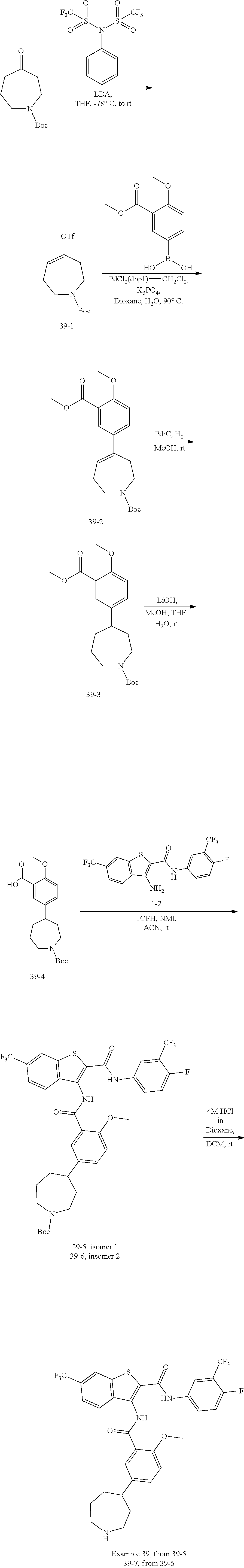

### Intermediate 39-1: Tert-butyl 4-(((trifluoromethyl) sulfonyl)oxy)-2,3,6,7-tetrahydro-1H-azepine-1-carboxylate

To a solution of tert-butyl 4-oxoazepane-1-carboxylate (5.00 g, 23.4 mmol) in THF (50 mL) was added LDA (29.3 mL, 58.6 mmol) dropwise at −78° C., and the reaction was continued to stir for 1 h. N-phenyl-bis(trifluoromethanesulfonimide) (10.1 g, 28.1 mmol) dissolved in THF (50 mL) was added at −78° C., and the reaction solution was allowed to warm to rt and stirred for 12 h. The reaction mass was quenched with saturated NH4Cl solution (100 mL) and extracted with EtOAc (3×100 mL). The combined organic layers were dried over Na₂SO₄, filtered and concentrated under reduced pressure. The residue was purified by column chromatography to give Intermediate 39-1 (2.50 g, 7.24 mmol, 31% yield). ¹H NMR (400 MHz, DMSO-d6) δ ppm 5.31 (m, 1H), 3.90 (br s, 1H), 3.38-3.56 (m, 2H), 3.33 (br s, 2H), 2.47-2.54 (m, 2H), 1.81-1.91 (m, 1H), 1.35-1.43 (m, 9H).

### Intermediate 39-2: Tert-butyl 4-(4-methoxy-3-(methoxycarbonyl)phenyl)-2,3,6,7-tetrahydro-1H-azepine-1-carboxylate

Intermediate 39-2 (400 mg, 1.11 mmol, 47% yield) was prepared from (4-methoxy-3-(methoxycarbonyl)phenyl)boronic acid (500 mg, 2.38 mmol) and Intermediate 39-1 (987 mg, 2.86 mmol) in a similar way as Intermediate 13-3. ¹H NMR (400 MHz, CDCl₃-d) δ ppm 7.97-8.02 (m, 1H), 7.36 (s, 1H), 6.97-7.09 (m, 1H), 4.02-4.22 (m, 1H), 3.85-3.98 (m, 6H), 2.04 (s, 4H), 1.60 (s, 2H), 1.47-1.56 (m, 9H), 1.21-1.31 (m, 2H).

### Intermediate 39-3: Tert-butyl 4-(4-methoxy-3-(methoxycarbonyl)phenyl)azepane-1-carboxylate

Intermediate 39-3 (350 mg, 0.963 mmol, 77% yield) was prepared from Intermediate 39-2 (450 mg, 1.25 mmol) in a similar way as Intermediate 37-2. ¹H NMR (400 MHz, CDCl₃-d) δ ppm 7.22-7.28 (m, 2H), 7.02 (dd, J=8.53, 2.51 Hz, 1H), 3.87 (m, 3H), 3.66-3.78 (m, 3H), 3.56-3.74 (m, 1H), 3.42-3.55 (m, 1H), 3.17-3.40 (m, 2H), 2.60 (s, 1H), 1.84-2.06 (m, 2H), 1.80 (br s, 2H), 1.57-1.76 (m, 2H), 1.44-1.49 (m, 9H).

Intermediate 39-4: 5-(1-(tert-butoxycarbonyl) azepan-4-yl)-2-methoxybenzoic acid Intermediate 39-4 (280 mg, 0.801 mmol, 83% yield) was prepared from Intermediate 39-3 (350 mg, 0.963 mmol) by hydrolyis conditions similar to that used for Example 2. ¹H NMR (400 MHz, DMSO-d6) δ ppm 12.54 (br s, 1H), 7.39-7.48 (m, 1H), 7.25-7.34 (m, 1H), 7.03 (dd, J=8.53, 2.51 Hz, 1H), 3.78 (s, 3H), 3.48-3.64 (m, 1H), 3.38-3.47 (m, 2H), 3.07-3.29 (m, 1H), 2.61 (br t, J=9.29 Hz, 1H), 1.79-1.89 (m, 2H), 1.57-1.77 (m, 4H), 1.42 (d, J=3.01 Hz, 9H).

### Intermediate 39-5 and 39-6: Tert-butyl 4-(3-((2-((4-fluoro-3-(trifluoromethyl)phenyl)carbamoyl)-6-(trifluoromethyl)benzo[b]thiophen-3-yl)carbamoyl)-4-methoxyphenyl)azepane-1-carboxylate

Intermediate 39-5 and 39-6 were prepared from Intermediate 1-2 (800 mg, 1.89 mmol) and Intermediate 39-4 (993 mg, 2.84 mmol) in a similar way as Intermediate 1-3. The isomers were separated by prep SFC purification (Method: Column/dimensions: Whelk(R,R) (250×30) mm, 5 μm; % CO₂: 75%; % Co-solvent: 25% of 0.2% DEA in IPA; Total Flow: 140.0 g/min; Back Pressure: 100 bar; Temperature: 30° C.; UV: 220 nm) to give Intermediate 39-5 (Prep SFC RT=12.76 min, 99% ee) and Intermediate 39-6, (Prep SFC RT=14.62 min, 99% ee). Analytical SFC conditions: (Method: Column/dimensions: Whelk(R,R) (250×4.6) mm, 5 μm; % CO₂: 75%; % Co-solvent: 25% of 0.2% DEA in IPA; Total Flow: 4.0 g/min; Back Pressure: 100 bar; Temperature: 35° C.; UV: 220 nm).

Intermediate 39-5: (400 mg, 0.531 mmol, 28% yield)¹H NMR (300 MHz, CDCl₃-d) δ ppm 10.94 (s, 1H), 9.72 (br d, J=6.8 Hz, 1H), 8.15 (s, 1H), 7.96-7.74 (m, 4H), 7.39 (dd, J=2.3, 8.7 Hz, 1H), 7.28 (br d, J=8.7 Hz, 1H), 7.10-7.00 (m, 2H), 4.07 (s, 3H), 3.70 (br d, J=14.0 Hz, 1H), 3.56-3.44 (m, 1H), 3.41 (s, 1H), 3.36-3.17 (m, 1H), 2.68 (br d, J=8.7 Hz, 1H), 2.20-1.87 (m, 4H), 1.87-1.74 (m, 1H), 1.67 (br s, 1H), 1.43 (d, J=2.3 Hz, 9H). LC-MS RT: 2.71 min; MS (ESI) m/z 752.2 (M−H)⁻; Method C.

Intermediate 39-6: (400 mg, 0.531 mmol, 28% yield)¹H NMR (300 MHz, CDCl₃-d) δ ppm 10.75 (br s, 1H), 9.81 (br d, J=10.2 Hz, 1H), 8.14 (s, 1H), 7.98 (s, 1H), 7.90-7.77 (m, 2H), 7.47-7.36 (m, 2H), 7.19 (s, 1H), 7.12-7.00 (m, 2H), 4.07 (s, 3H), 3.83-3.54 (m, 1H), 3.48 (br s, 1H), 3.40-3.16 (m, 1H), 2.77-2.65 (m, 2H), 2.54 (s, 1H), 2.01-1.82 (m, 4H), 1.80-1.62 (m, 1H), 1.43 (d, J=3.0 Hz, 9H). LC-MS RT: 2.04 min; MS (ESI) m/z 712.2 (M+H)⁺; Method A.

### Example 39 and Intermediate 39-7: 3-(5-(azepan-4-yl)-2-methoxybenzamido)-N-(4-fluoro-3-(trifluoromethyl)phenyl)-6-(trifluoromethyl)benzo[b]thiophene-2-carboxamide

Intermediate 39-7 (3.7 mg, 5.6 μmol, 4% yield) was prepared from Intermediate 39-6 (100 mg, 0.13 mmol) in a similar way as Intermediate 9-4 and 9-5, followed by purification by prep LCMS (Method 1). ¹H NMR (400 MHz, DMSO-d6) δ ppm 10.86-10.65 (m, 2H), 8.61 (s, 1H), 8.13-8.04 (m, 2H), 7.97-7.88 (m, 1H), 7.78 (dd, J=1.8, 8.7 Hz, 1H), 7.62 (d, J=2.4 Hz, 1H), 7.50 (t, J=9.7 Hz, 1H), 7.38 (dd, J=2.3, 8.4 Hz, 1H), 7.13 (d, J=8.6 Hz, 1H), 3.89 (s, 3H), 3.03-2.95 (m, 5H), 2.89-2.73 (m, 3H), 1.88 (s, 4H). LC-MS RT: 2.14 min; MS (ESI) m/z 654.2 (M+H)⁺; Method A.

Example 39 (3.6 mg, 5.5 mol, 4% yield) was prepared from Intermediate 39-5 (100 mg, 0.13 mmol) in a similar way as Intermediate 9-4 and 9-5, followed by purification by prep LCMS (Method 1). ¹H NMR (400 MHz, DMSO-d6) δ=10.82-10.63 (m, 2H), 8.61 (s, 1H), 8.14-8.04 (m, 2H), 7.97-7.88 (m, 1H), 7.78 (dd, J=1.5, 8.3 Hz, 1H), 7.63 (d, J=2.4 Hz, 1H), 7.50 (t, J=9.4 Hz, 1H), 7.38 (dd, J=2.3, 8.4 Hz, 1H), 7.13 (d, J=8.6 Hz, 1H), 3.90 (s, 3H), 3.02-2.94 (m, 2H), 2.90-2.76 (m, 2H), 1.88 (s, 3H), 1.83-1.50 (m, 4H). LC-MS RT: 2.15 min; MS (ESI) m/z 654.2 (M+H)⁺; Method A.

### Example 40

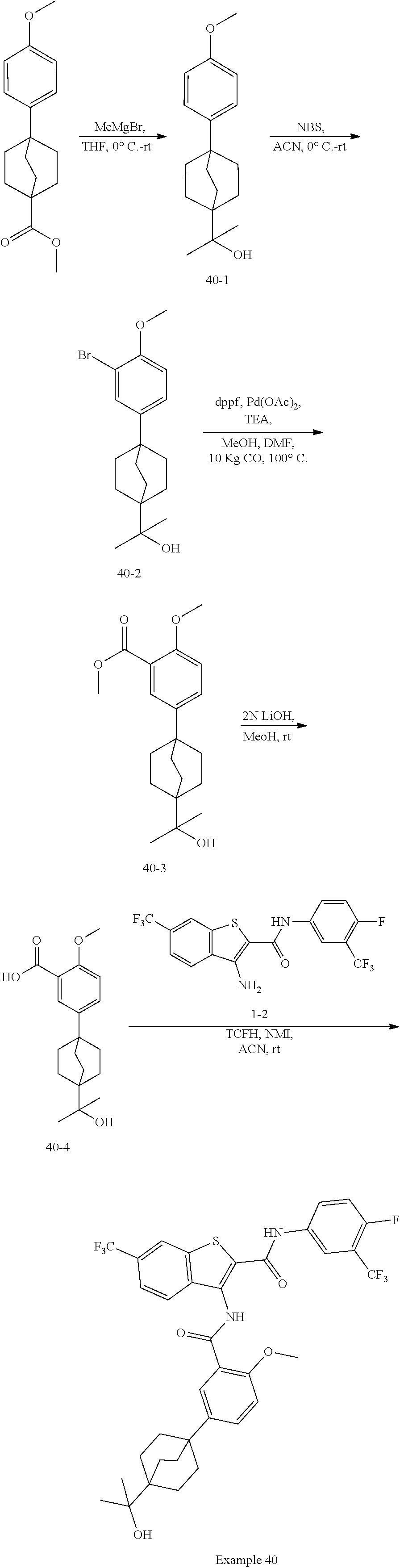

Intermediate 40-1: 2-(4-(4-methoxyphenyl) bicyclo [2.2.2]octan-1-yl) propan-2-ol Intermediate 40-1 (70 mg, 0.26 mmol, 70% yield) was prepared from methyl 4-(4-methoxyphenyl)bicyclo[2.2.2]octane-1-carboxylate (100 mg, 0.36 mmol) in a similar way as Example 3. ¹H NMR (400 MHz, DMSO-d6) δ ppm 7.21 (d, J=9.04 Hz, 2H) 6.82 (d, J=8.53 Hz, 2H) 3.85 (s, 1H) 3.70 (s, 3H) 1.61-1.74 (m, 6H) 1.47-1.61 (m, 6H) 1.01 (s, 6H).

### Intermediate 40-2: 2-(4-(3-bromo-4-methoxyphenyl) bicyclo [2.2.2]octan-1-yl) propan-2-ol

To a solution of Intermediate 40-1 (60 mg, 0.22 mmol) in ACN (3 mL) at 0° C. was added a solution of NBS (39 mg, 0.22 mmol) in ACN (1 mL). The resulting reaction mixture was stirred at rt for 12 h. The reaction mass was concentrated under reduced pressure and 3 mL of water was added to the residue. The solid precipitate was filtered and dried under vacuum to give Intermediate 40-2 (65 mg, 0.18 mmol, 84% yield). ¹H NMR (400 MHz, DMSO-d6) δ ppm 7.43-7.29 (s, 1H), 7.27-7.20 (d, J=8.53 Hz, 1H), 7.02-6.99 (d, J=9.04 Hz, 1H), 3.85 (s, 1H), 3.70 (s, 3H), 1.61-1.74 (m, 6H), 1.47-1.61 (m, 6H), 1.01 (s, 6H).

### Intermediate 40-3: Methyl 5-(4-(2-hydroxypropan-2-yl) bicyclo [2.2.2]octan-1-yl)-2-methoxybenzoate

To a solution of Intermediate 40-2 (250 mg, 0.71 mmol) in a mixture of DMF (6 mL) and MeOH (6 mL) was added dppf (59 mg, 0.11 mmol), Pd(OAc)₂ (16 mg, 0.071 mmol) and TEA (0.49 mL, 3.5 mmol) under a N₂ atmosphere. The reaction mixture was heated under 10 kg/cm² CO gas pressure at 100° C. for 12 h. The reaction mixture was filtered through celite and concentrated under reduced pressure. The residue was purified by column chromatography to afford Intermediate 40-3 (130 mg, 0.39 mmol, 55% yield). LC-MS RT: 0.74 min; MS (ESI) m/z 333.2 (M+H)⁺; Method E.

### Intermediate 40-4: 5-(4-(2-hydroxypropan-2-yl) bicyclo [2.2.2]octan-1-yl)-2-methoxybenzoic acid

Intermediate 40-4 (85 mg, 0.27 mmol, 68% yield) was prepared from Intermediate 40-3 (130 mg, 0.39 mmol) by the procedure described in Example 2. LC-MS RT: 0.83 min; MS (ESI) m/z 319.2 (M+H)⁺; Method E.

### Example 40: N-(4-fluoro-3-(trifluoromethyl) phenyl)-3-(5-(4-(2-hydroxypropan-2-yl) bicyclo [2.2.2]octan-1-yl)-2-methoxybenzamido)-6-(trifluoromethyl) benzo[b]thiophene-2-carboxamide

Example 40 (22 mg, 0.028 mmol, 22% yield) was prepared from Intermediate 1-2 (27 mg, 0.063 mmol) and Intermediate 40-4 (40 mg, 0.13 mmol) in a similar way as Intermediate 1-3 followed by prep LCMS purification (Method 1). ¹H NMR (400 MHz, DMSO-d6) δ ppm 10.74 (br d, J=12.2 Hz, 2H), 8.66 (s, 1H), 8.13 (dd, J=2.9, 6.6 Hz, 1H), 8.08 (d, J=8.6 Hz, 1H), 8.01-7.94 (m, 1H), 7.83 (dd, J=1.2, 8.3 Hz, 1H), 7.64 (d, J=2.4 Hz, 1H), 7.58-7.44 (m, 2H), 7.13 (d, J=8.6 Hz, 1H), 3.92 (s, 3H), 3.88 (s, 1H), 1.70-1.62 (m, 6H), 1.57-1.51 (m, 6H), 1.01 (s, 6H). LC-MS RT: 2.79 min; MS (ESI) m/z 723.2 (M+H)⁺; Method A.

### Example 42

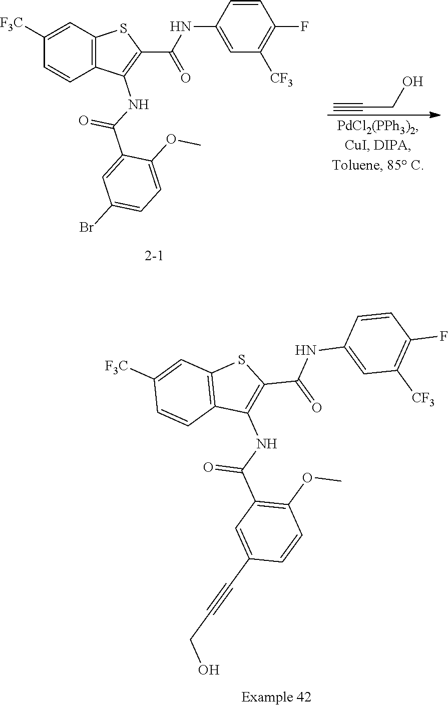

### Example 42: N-(4-fluoro-3-(trifluoromethyl)phenyl)-3-(5-(3-hydroxyprop-1-yn-1-yl)-2-methoxybenzamido)-6-(trifluoromethyl)benzo[b]thiophene-2-carboxamide

To a solution of Intermediate 2-1 (50 mg, 0.079 mmol) in toluene (2 mL) and diisopropylamine (0.056 mL, 0.39 mmol) were added copper(I) iodide (3.0 mg, 0.016 mmol) and bis(triphenylphosphine)palladium(II)dichloride (5.5 mg, 7.9 μmol) and the resulting solution was degassed with N₂ for 10 min before adding prop-2-yn-1-ol (22 mg, 0.39 mmol). The reaction mixture was heated at 85° C. for 5 h. The reaction mass was diluted with EtOAc (25 mL), washed with water (2×20 mL). The separated organic layer was dried over Na₂SO₄, filtered and concentrated under reduced pressure. The residue was purified by prep HPLC (Method 2) to give Example 42 (12 mg, 0.019 mmol, 24% yield). ¹H NMR (400 MHz, DMSO-d6) δ ppm 10.66-10.95 (m, 2H), 8.49-8.77 (m, 1H), 8.14-8.19 (m, 1H), 8.07-8.11 (m, 1H), 7.97-8.03 (m, 1H), 7.79-7.84 (m, 2H), 7.53-7.64 (m, 1H), 7.23-7.27 (m, 2H), 5.28-5.33 (m, 1H), 3.92-3.96 (s, 3H), 3.37-3.39 (m, 2H). LC-MS RT: 3.61 min; MS (ESI) m/z 611.2 (M+H)⁺; Method C.

### Example 44

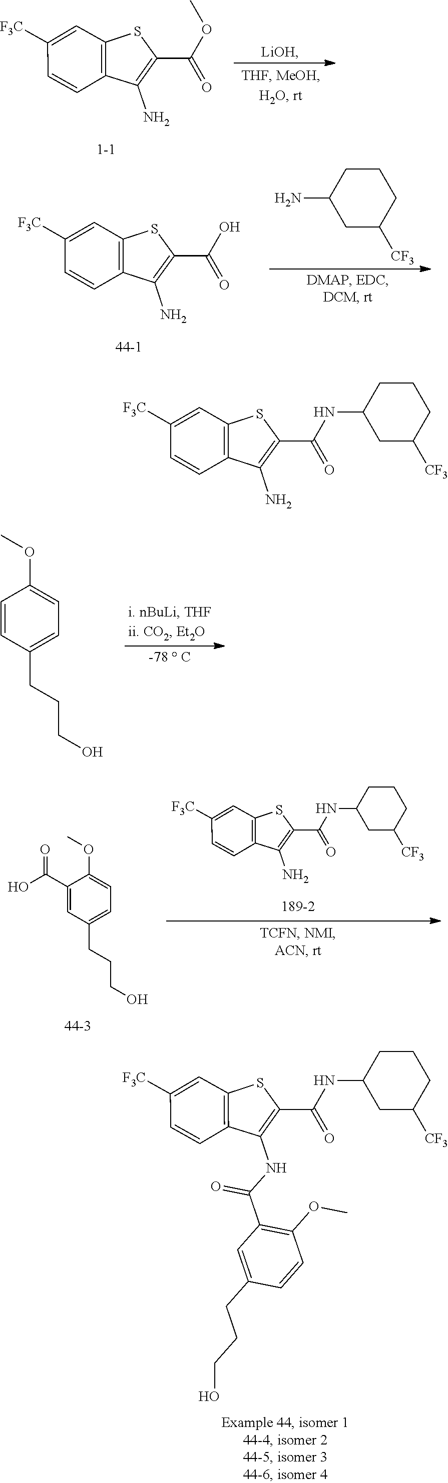

### Intermediate 44-1: 3-amino-6-(trifluoromethyl)benzo[b]thiophene-2-carboxylic acid

Intermediate 44-1 (184 mg, 0.704 mmol, 65% yield) was prepared from Intermediate 1-1 (300 by the general procedure described for Example 2. LC-MS RT: 0.83 min; MS (ESI) m/z 260.2 (M−H)⁻; Method E.

### Intermediate 44-2: 3-amino-6-(trifluoromethyl)-N-(3-(trifluoromethyl) cyclohexyl) benzo[b]thiophene-2-carboxamide

Intermediate 44-2 (50 mg, 0.11 mmol, 41% yield) was prepared from Intermediate 44-1 (70 mg, 0.27 mmol) and 3-(trifluoromethyl) cyclohexan-1-amine (90 mg, 0.54 mmol) in a similar way as Intermediate 29-3. LC-MS RT: 1.34 min; MS (ESI) m/z 410.0 (M−H)⁻; Method E.

### Intermediate 44-3: 5-(3-hydroxypropyl)-2-methoxybenzoic acid

To a solution of 3-(4-methoxyphenyl)propan-1-ol (1.00 g, 6.02 mmol) in THF (20 mL) at −78° C., was added n-butyl lithium (8.27 mL, 13.2 mmol) and the reaction mixture was allowed to warm to rt and stirred for 1 h. The reaction mixture was cooled to 0° C. and was slowly added to a suspension of carbon dioxide (dry ice) (6.62 g, 150 mmol) in Et20 (10 mL) and the reaction mixture was stirred at 0° C. for 4 h. The reaction mixture was quenched with water and concentrated under reduced pressure. The residue was diluted with ice water (20 mL) and extracted with ether (1× and organic layer discarded). The aqueous layer was then acidified to pH 6 with conc HCl and stirred for 10 min. The resulting precipitate was collected by filtration, washed with pet ether and dried under vacuum to give Intermediate 44-3 (700 mg, 3.33 mmol, 55% yield). LC-MS RT: 0.34 min; MS (ESI) m/z 209.2 (M−H)⁻; Method C.

### Example 44: 3-(5-(3-hydroxypropyl)-2-methoxybenzamido)-6-(trifluoromethyl)-N-(3-(trifluoromethyl) cyclohexyl) benzo[b]thiophene-2-carboxamide

Example 44 was prepared from Intermediate 44-2 (180 mg, 0.439 mmol) and Intermediate 44-3 (184 mg, 0.877 mmol) in a similar way as Intermediate 1-3, and was obtained by filtration of the precipitate. The individual isomers were obtained by prep SFC purification (Column: Chiralpak IG (250×30) mm, 5 μm; % CO₂: 65%; Co-solvent: 35% of 0.2% NH₃ in MeOH; Total Flow: 100.0 g/min; Back Pressure: 100 bar; Temperature: 35° C.; UV: 242 nm) to give Example 44 (Prep SFC RT=3.54 min, 100% ee), Intermediate 44-4 (Prep SFC RT=4.12 min, 99% ee), Intermediate 44-5 (Prep SFC RT=3.56 min, 100% ee), Intermediate 44-6 (Prep SFC RT=2.84 min, 100% ee). Analytical SFC conditions: (Column: Chiralpak IG (250×4.6) mm, 5 μm; % CO₂: 55%; Co-solvent: 45% of 0.2% NH3 in MeOH; Total Flow: 4.0 g/min; Back Pressure: 100 bar; Temperature: 35° C.; UV: 242 nm).

Example 44: (7.0 mg, 11 mol, 3% yield). LC-MS RT: 3.28 min; MS (ESI) m/z 603.3 (M+H)⁺; Method C.

Intermediate 44-4: (6.0 mg, 9.0 mol, 2% yield). LC-MS RT: 3.27 min; MS (ESI) m/z 603.3 (M+H)⁺; Method C.

Intermediate 44-5: (34 mg, 0.056 mmol, 13% yield). ¹H NMR (400 MHz, DMSO-d6) δ ppm 8.54 (br s, 1H), 7.98 (d, J=8.0 Hz, 1H), 7.74 (br d, J=8.5 Hz, 1H), 7.62 (br s, 1H), 7.36 (br s, 1H), 7.14 (br d, J=7.0 Hz, 1H), 4.49 (br s, 1H), 3.97 (s, 3H), 3.88 (br s, 2H), 2.66-2.58 (m, 2H), 2.05 (br d, J=13.1 Hz, 1H), 1.93-1.67 (m, 7H), 1.49-1.33 (m, 2H), 1.30-1.06 (m, 4H). LC-MS RT: 3.33 min; MS (ESI) m/z 603.2 (M+H)⁺; Method C.

Intermediate 44-6: (34 mg, 0.056 mmol, 13% yield). ¹H NMR (400 MHz, DMSO-d6) δ ppm 8.54 (br s, 1H), 7.98 (d, J=8.5 Hz, 1H), 7.74 (br d, J=8.0 Hz, 1H), 7.69-7.53 (m, 1H), 7.42-7.32 (m, 1H), 7.14 (br d, J=8.5 Hz, 1H), 4.49 (br s, 1H), 3.97 (s, 3H), 3.88 (br s, 2H), 2.65-2.57 (m, 2H), 2.56-2.52 (m, 2H), 2.46-2.34 (m, 1H), 2.05 (br d, J=13.1 Hz, 1H), 1.93-1.67 (m, 7H), 1.45-1.34 (m, 2H), 1.30-1.07 (m, 4H). LC-MS RT: 3.33 min; MS (ESI) m/z 603.2 (M+H)⁺; Method C.

### Example 45

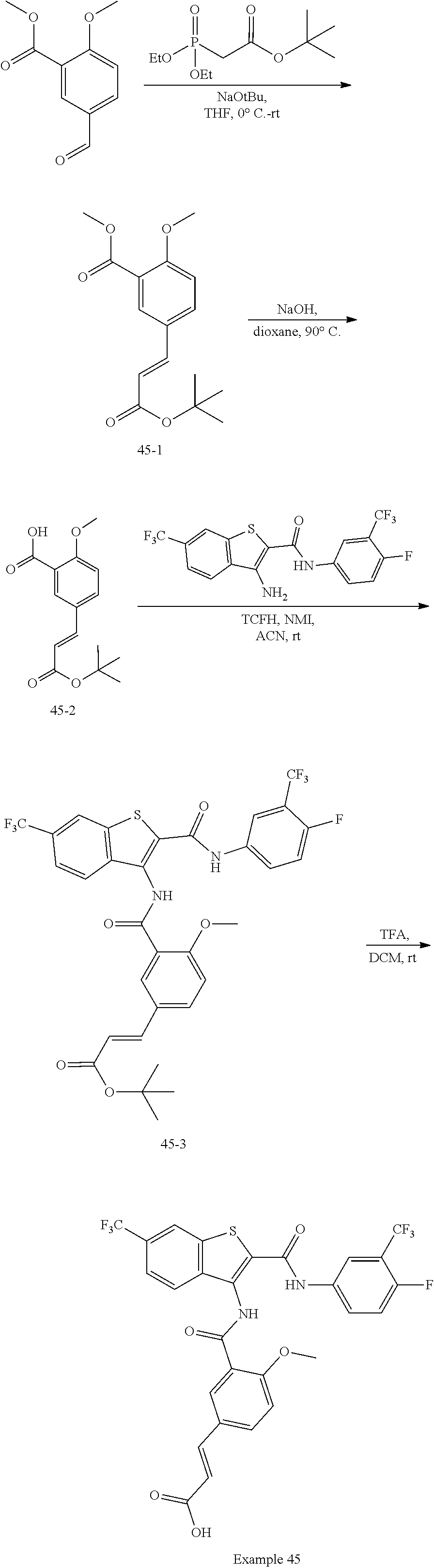

### Intermediate 45-1: Methyl (Z)-5-(3-(tert-butoxy)-3-oxoprop-1-en-1-yl)-2-methoxybenzoate

To a solution of tert-butyl diethylphosphonoacetate (2.04 mL, 8.65 mmol) in THF (10 mL) was added sodium tert-butoxide (831 mg, 8.65 mmol) at 0° C. and the reaction mixture was stirred at 0° C. for 30 min. Methyl 5-formyl-2-methoxybenzoate (1.40 g, 7.21 mmol) in THF (10 mL) was added to the reaction mixture and stirring continued at rt for 10 h. The reaction mass was diluted with EtOAc (25 mL), washed with water (2×20 mL) and brine (10 mL). The separated organic layer was dried over Na₂SO₄, filtered and concentrated under reduced pressure. The residue was purified by column chromatography to afford Intermediate 45-1 (1.40 g, 4.79 mmol, 70% yield). LC-MS RT: 2.96 min; MS (ESI) m/z 293.2 (M+H)⁺; Method C.

### Intermediate 45-2: methyl 5-(2-(tert-butoxycarbonyl) cyclopropyl)-2-methoxybenzoate

To a cooled (0° C.) solution of Intermediate 45-1 (500 mg, 1.71 mmol) in Dioxane (10 mL) was added NaOH (68.4 mg, 1.71 mmol), and the reaction was heated at 90° C. for 2 h. The reaction mass was concentrated and acidified with 1.5 N HCl to pH 4, and the aqueous layer was extracted with DCM (15 mL), then washed with water (2×10 mL) and brine (10 mL). The separated organic layer was dried over Na₂SO₄, filtered and concentrated under reduced pressure. The residue was purified by column chromatography to afford Intermediate 45-2 (150 mg, 0.539 mmol, 32% yield). ¹H NMR (400 MHz, DMSO-d) δ ppm δ ppm 7.97 (m, 2H), 7.65 (d, J=2.5 Hz, 1H), 7.26 (d, 8.40 Hz, 1H), 6.34 (d, J=16.1 Hz, 1H), 3.84 (s, 3H), 1.47 (s, 9H).

### Intermediate 45-3: tert-butyl (Z)-3-(3-((2-((4-fluoro-3-(trifluoromethyl) phenyl) carbamoyl)-6-(trifluoromethyl) benzo[b]thiophen-3-yl) carbamoyl)-4-methoxyphenyl) acrylate

Intermediate 45-3 (210 mg, 0.308 mmol, 52% yield) was prepared from Intermediate 1-2 (250 mg, 0.592 mmol) and Intermediate 45-2 (198 mg, 0.710 mmol) in a similar way as Intermediate 1-3. ¹H NMR (300 MHz, CDCl₃-d) δ ppm 10.81 (s, 1H), 9.41 (s, 1H), 8.47 (d, J=2.3 Hz, 1H), 8.01-7.91 (m, 2H), 7.85-7.65 (m, 3H), 7.56 (d, J=15.9 Hz, 1H), 7.41 (d, J=8.7 Hz, 2H), 7.23-7.07 (m, 1H), 6.36 (d, J=16.2 Hz, 1H), 4.13 (s, 3H), 1.66-1.40 (m, 9H).

### Example 45: (Z)-3-(3-((2-((4-fluoro-3-(trifluoromethyl) phenyl) carbamoyl)-6-(trifluoromethyl) benzo[b]thiophen-3-yl) carbamoyl)-4-methoxyphenyl) acrylic acid

Example 45 (2.6 mg, 4.0 mol, 13% yield) was prepared from Intermediate 45-3 (20 mg, 0.029 mmol) in a similar way as Intermediate 4-2, followed by purification by prep LCMS (Method 1). ¹H NMR (400 MHz, DMSO-d6) δ ppm 10.93-10.72 (m, 1H), 8.66 (s, 1H), 8.21-8.08 (m, 2H), 8.06 (br s, 1H), 8.04-7.96 (m, 1H), 7.88 (br d, J=8.3 Hz, 1H), 7.82 (d, J=8.6 Hz, 1H), 7.64-7.44 (m, 2H), 7.26 (br d, J=9.0 Hz, 1H), 6.44 (br d, J=15.9 Hz, 2H), 3.92 (s, 3H). LC-MS RT: 1.92 min; MS (ESI) m/z 625.1 (M−H)⁻; Method A.

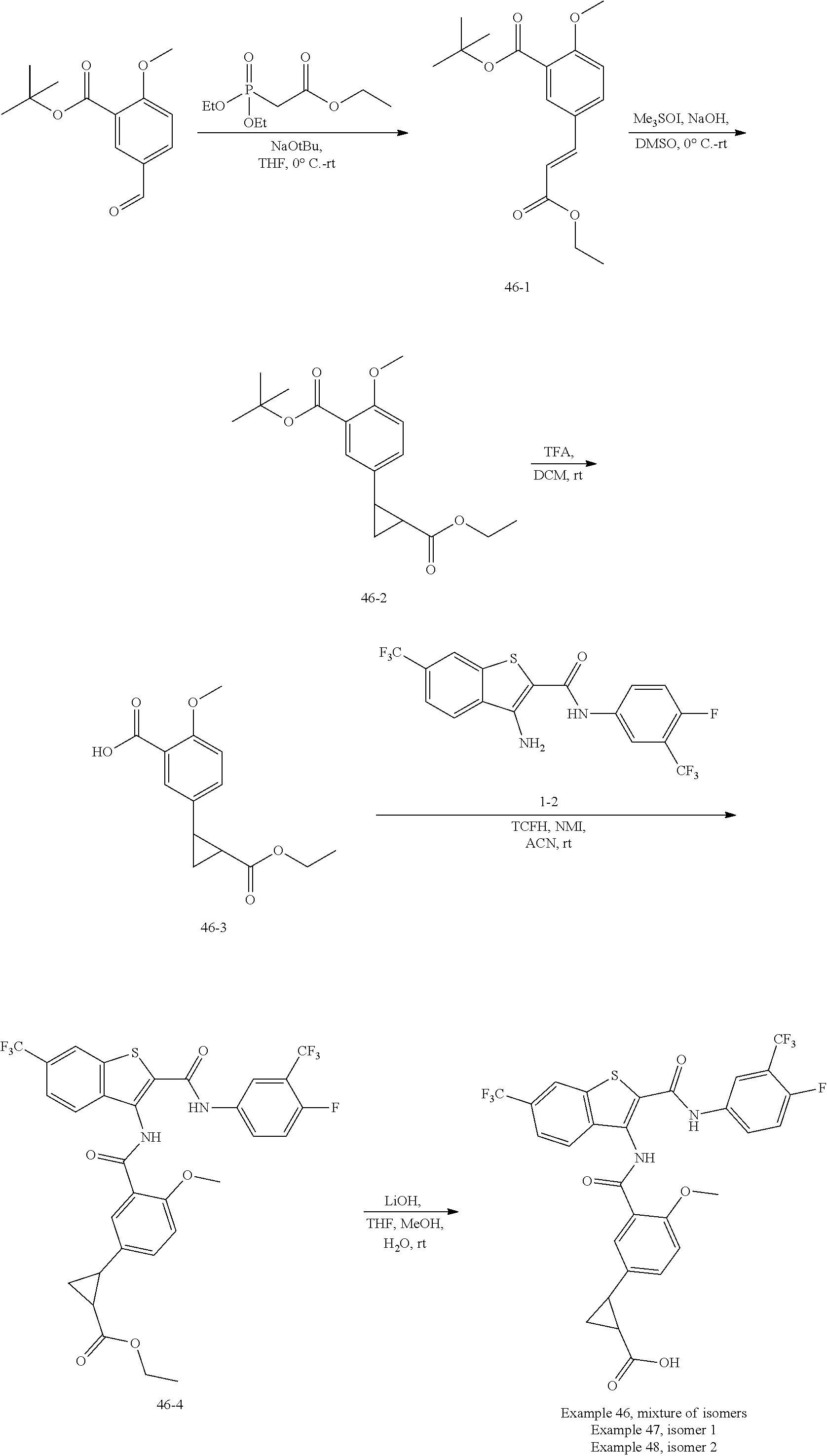

### Intermediate 46-1: Tert-butyl (Z)-5-(3-ethoxy-3-oxoprop-1-en-1-yl)-2-methoxybenzoate

Intermediate 46-1 (140 mg, 0.457 mmol, 54% yield) was prepared from triethyl phosphonoacetate (228 mg, 1.02 mmol) and tert-butyl 5-formyl-2-methoxybenzoate (200 mg, 0.846 mmol) in a similar way as Intermediate 45-1. ¹H NMR (400 MHz, CDCl₃-d) δ ppm 7.90 (d, J=2.01 Hz, 1H), 7.58-7.67 (m, 1H), 7.27 (s, 1H), 6.97 (d, J=8.53 Hz, 1H), 6.35 (d, J=15.56 Hz, 1H), 4.27 (q, J=7.36 Hz, 2H), 3.94 (s, 3H), 1.45-1.56 (m, 9H), 1.35 (t, J=7.03 Hz, 3H).

### Intermediate 46-2: Tert-butyl 5-(2-(ethoxycarbonyl) cyclopropyl)-2-methoxybenzoate

To a solution of Intermediate 46-1 (100 mg, 0.326 mmol) in DMSO (5 mL) was added trimethylsulfoxonium iodide (144 mg, 0.653 mmol) followed by NaOH (19.6 mg, 0.490 mmol) at 0° C. The resulting reaction mixture was allowed to stir at rt for 3 h. The reaction was diluted with EtOAc (50 mL), washed with water (2×25 mL) and then brine (25 mL). The separated organic layer was dried over Na₂SO₄, filtered and concentrated under reduced pressure. The residue was purified by column chromatography to give Intermediate 46-2 (75.0 mg, 0.234 mmol, 71% yield). ¹H NMR (400 MHz, CDCl₃-d) δ ppm 7.48 (d, J=2.4 Hz, 1H), 7.29 (s, 1H), 7.18 (dd, J=2.4, 8.6 Hz, 1H), 6.89 (d, J=8.6 Hz, 1H), 4.19 (q, J=7.1 Hz, 2H), 3.89 (s, 3H), 2.54-2.45 (m, 2H), 1.86 (ddd, J=4.3, 5.2, 8.4 Hz, 1H), 1.62-1.56 (m, 9H), 1.36-1.24 (m, 3H).

### Intermediate 46-3: 5-(2-(ethoxycarbonyl) cyclopropyl)-2-methoxybenzoic acid

Intermediate 46-3 (70.0 mg, 0.265 mmol, 85% yield) was prepared from Intermediate 46-2 (100 mg, 0.312 mmol) in a similar way as Intermediate 4-2. ¹H NMR (300 MHz, CDCl₃-d) d ppm 8.06 (br s, 1H), 7.80 (d, J=2.3 Hz, 1H), 7.31 (dd, J=2.5, 8.5 Hz, 1H), 6.93 (d, J=8.7 Hz, 1H), 4.11 (q, J=7.2 Hz, 2H), 4.00 (s, 3H), 2.50-2.41 (m, 1H), 1.85-1.77 (m, 1H), 1.59-1.43 (m, 1H), 1.30-1.14 (m, 5H).

### Intermediate 46-4: Ethyl 2-(3-((2-((4-fluoro-3-(trifluoromethyl) phenyl) carbamoyl)-6-(trifluoromethyl) benzo[b]thiophen-3-yl) carbamoyl)-4-methoxyphenyl) cyclopropane-1-carboxylate

Intermediate 46-4 (50 mg, 0.074 mmol, 63% yield) was prepared from Intermediate 1-2 (50 mg, 0.12 mmol) and Intermediate 46-3 (47 mg, 0.18 mmol) in a similar way as Intermediate 1-3. LC-MS RT: 1.64 min; MS (ESI) m/z 667.2 (M−H)⁻; Method E.

### Example 46, 47 and 48: 2-(3-((2-((4-fluoro-3-(trifluoromethyl) phenyl) carbamoyl)-6-(trifluoromethyl) benzo[b]thiophen-3-yl) carbamoyl)-4-methoxyphenyl) cyclopropane-1-carboxylic acid

Example 46 (3.0 mg, 4.7 mol, 6% yield) was prepared from Intermediate 46-4 (50 mg, 0.074 mmol) by the general procedure described in Example 2, followed by purification by prep LCMS conditions (Method 1). ¹H NMR (400 MHz, DMSO-d6) δ ppm 10.87-10.62 (m, 2H), 8.65 (s, 1H), 8.14 (dd, J=2.3, 6.5 Hz, 1H), 8.08 (d, J=8.6 Hz, 1H), 8.03-7.93 (m, 1H), 7.81 (dd, J=1.5, 8.6 Hz, 1H), 7.63-7.46 (m, 2H), 7.37 (dd, J=2.3, 8.4 Hz, 1H), 7.14 (d, J=8.6 Hz, 1H), 3.90 (s, 3H), 2.44-2.36 (m, 1H), 1.74 (td, J=4.8, 7.7 Hz, 1H), 1.40 (td, J=4.8, 9.0 Hz, 1H), 1.32-1.24 (m, 1H). LC-MS RT: 1.99 min; MS (ESI) m/z 641.1 (M+H)⁺; Method A.

Example 46 (287 mg, 0.449 mmol) was purified by prep SFC (Column: Chiralpak IG (250×30) mm, 5 μm; % CO₂: 50%; Co-solvent: 50% of 0.2% NH3 in MeOH; Flow Conditions: 140.0 g/min; Back Pressure: 100 Bar; Temperature: 40° C.; Detector Wavelength: 220 nm) to afford Example 47 (Peak 1, Prep SFC RT=4.8 min) and Example 48 (Peak 2, Prep SFC RT=8.3 min). Analytical SFC conditions: (Column: Chiralpak IG (250×4.6) mm, 5 m; % CO₂: 50%; Co-solvent: 50% of 0.2% NH3 in MeOH; Flow Conditions: 4.0 g/min; Back Pressure: 100 bar; Temperature: 40° C.; Detector Wavelength: 220 nm)

Example 47: (80.0 mg, 0.125 mmol, 28% yield)¹H NMR (400 MHz, DMSO-d6) δ 12.38-12.23 (m, 1H), 10.80 (br d, J=4.2 Hz, 1H), 10.73 (br d, J=1.2 Hz, 1H), 8.66 (s, 1H), 8.15 (dd, J=6.5, 2.3 Hz, 1H), 8.08 (d, J=8.3 Hz, 1H), 8.00 (dt, J=5.0, 3.6 Hz, 1H), 7.81 (d, J=8.1 Hz, 1H), 7.62-7.50 (m, 2H), 7.38 (dd, J=8.6, 2.0 Hz, 1H), 7.14 (d, J=8.8 Hz, 1H), 3.89 (s, 3H), 2.44-2.36 (m, 1H), 1.80-1.70 (m, 1H), 1.41 (dt, J=9.0, 4.7 Hz, 1H), 1.33-1.19 (m, 2H). LC-MS RT: 2.16 min; MS (ESI) m/z 639.1 (M−H)⁻; Method A.

Example 48: (80.0 mg, 0.125 mmol, 28% yield)¹H NMR (400 MHz, DMSO-d6) δ 10.80 (br d, J=10.0 Hz, 1H), 8.65 (s, 1H), 8.15 (dd, J=6.6, 2.4 Hz, 1H), 8.08 (d, J=8.6 Hz, 1H), 8.03-7.94 (m, 1H), 7.83-7.75 (m, 1H), 7.60-7.46 (m, 2H), 7.42-7.31 (m, 1H), 7.14 (d, J=8.8 Hz, 1H), 3.89 (s, 3H), 2.43-2.35 (m, 1H), 1.77-1.67 (m, 1H), 1.44-1.36 (m, 1H), 1.29-1.18 (m, 2H). LC-MS RT: 2.02 min; MS (ESI) m/z 639.1 (M−H)⁻; Method A.

### Example 49: N-(4-fluoro-3-(trifluoromethyl) phenyl)-3-(5-(2-(hydroxymethyl) cyclopropyl)-2-methoxybenzamido)-6-(trifluoromethyl) benzo[b]thiophene-2-carboxamide

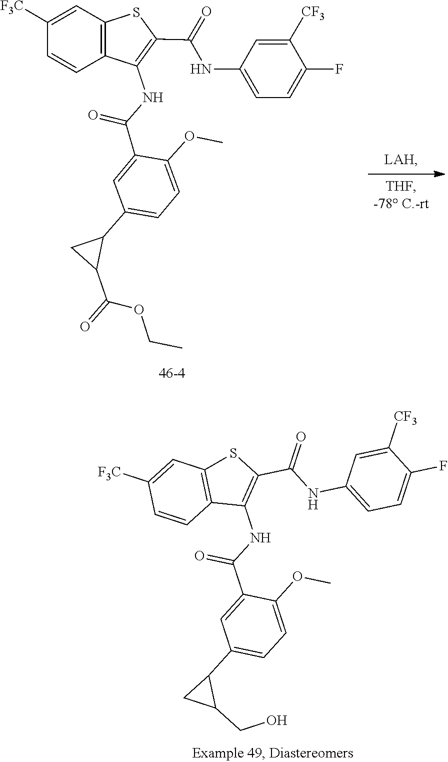 [SMILES: CCOC(=O)C1CC1c1ccc(OC)c(C(=O)Nc2c(C(=O)Nc3ccc(F)c(C(F)(F)F)c3)sc3cc(C(F)(F)F)ccc23)c1 ; COc1ccc(C2CC2CO)cc1C(=O)Nc1c(C(=O)Nc2ccc(F)c(C(F)(F)F)c2)sc2cc(C(F)(F)F)ccc12]

To a solution of Intermediate 46-4 (60 mg, 0.090 mmol) in THF (2 mL) was added LAH (0.045 mL, 0.090 mmol) at −78° C. The resulting reaction mixture was allowed to warm to rt and stirred for 30 min. The reaction solution was diluted with EtOAc (20 mL), washed with water (2×20 mL) and then brine (10 mL). The separated organic layer was dried over Na₂SO₄, filtered and concentrated under reduced pressure. The residue was purified by prep LCMS (Method 1) to give Example 49 (13 mg, 0.020 mmol, 23% yield). ¹H NMR (400 MHz, DMSO-d6) δ ppm 10.85-10.75 (m, 1H), 10.75-10.63 (m, 1H), 8.66 (s, 1H), 8.15 (dd, J=2.2, 6.4 Hz, 1H), 8.06 (d, J=8.6 Hz, 1H), 8.02-7.94 (m, 1H), 7.81 (dd, J=1.1, 8.7 Hz, 1H), 7.60-7.46 (m, 2H), 7.27 (dd, J=2.0, 8.3 Hz, 1H), 7.12 (d, J=8.3 Hz, 1H), 4.60 (t, J=5.6 Hz, 1H), 3.91 (s, 3H), 3.46 (td, J=5.8, 11.2 Hz, 1H), 1.86-1.77 (m, 2H), 1.24-1.17 (m, 1H), 0.88-0.81 (m, 1H), 0.81-0.72 (m, 1H). LC-MS RT: 2.39 min; MS (ESI) m/z 627.2 (M+H)⁺; Method A.

### Example 50

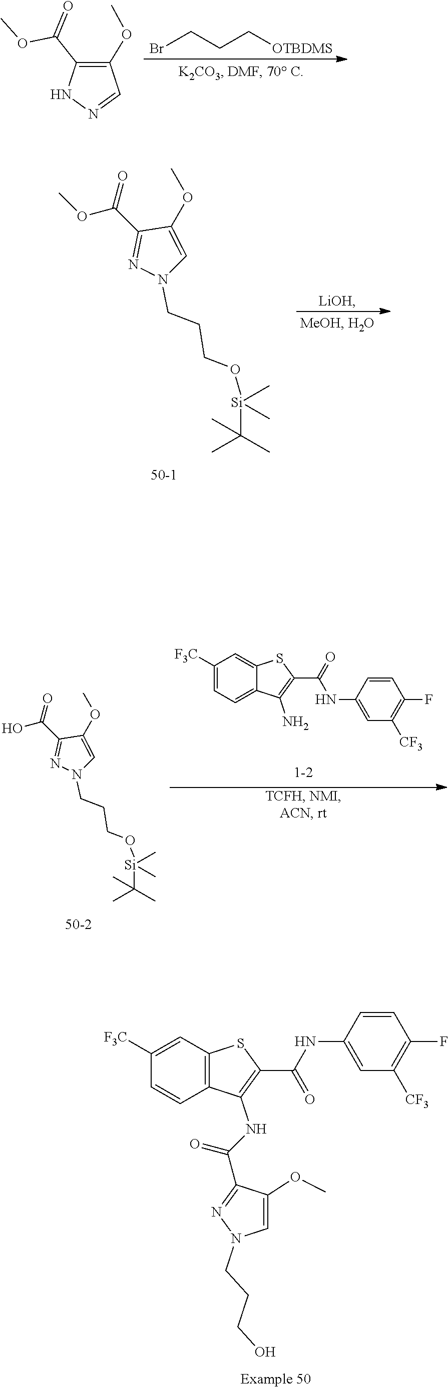

### Intermediate 50-1: methyl 1-(3-((tert-butyldimethylsilyl)oxy)propyl)-4-methoxy-1H-pyrazole-3-carboxylate

To a solution of methyl 4-methoxy-1H-pyrazole-5-carboxylate (320 mg, 2.05 mmol) in DMF (12 mL) was added K₂CO₃ (850 mg, 6.15 mmol) followed by (3-bromopropoxy)(tert-butyl) dimethylsilane (779 mg, 3.07 mmol). The resulting reaction mixture was stirred at 70° C. for 5 h. The reaction mass was filtered through celite, washed with EtOAc (50 mL). The filtrate was concentrated under reduced pressure, then the residue was purified by column chromatography to give Intermediate 50-1 (180 mg, 0.548 mmol, 27% yield). LC-MS RT: 2.07 min; MS (ESI) m/z 329.2 (M+H)⁺; Method E.

### Intermediate 50-2: 1-(3-((tert-butyldimethylsilyl)oxy)propyl)-4-methoxy-1H-pyrazole-5-carboxylic acid

Intermediate 50-2 (85 mg, 0.27 mmol, 49% yield) was prepared from Intermediate 50-1 (180 mg, 0.55 mmol) in a similar way as Example 2. LC-MS RT: 0.46 min; MS (ESI) m/z 315.1 (M+H)⁺; Method E.

### Example 50: N-(2-((4-fluoro-3-(trifluoromethyl) phenyl) carbamoyl)-6-(trifluoromethyl) benzo[b]thiophen-3-yl)-1-(3-hydroxypropyl)-4-methoxy-1H-pyrazole-3-carboxamide

Example 50 (10 mg, 0.017 mmol, 12% yield) was prepared from Intermediate 1-2 (30 mg, 0.071 mmol) and Intermediate 50-2 (45 mg, 0.14 mmol) in a similar way as Intermediate 1-3, followed by purification by prep LCMS (Method 1). ¹H NMR (400 MHz, DMSO-d6) δ ppm 10.76 (s, 1H), 10.11 (s, 1H), 8.68 (s, 1H), 8.14 (dd, J=2.4, 6.4 Hz, 1H), 8.03 (d, J=8.6 Hz, 1H), 8.01-7.92 (m, 1H), 7.81 (dd, J=1.3, 8.7 Hz, 1H), 7.59 (s, 1H), 7.54 (t, J=9.9 Hz, 1H), 4.50-4.41 (m, 3H), 3.95 (s, 3H), 3.31-3.28 (m, 2H), 1.84-1.72 (m, 2H). LC-MS RT: 2.21 min; MS (ESI) m/z 603.1 (M−H)⁻; Method A.

### Example 51

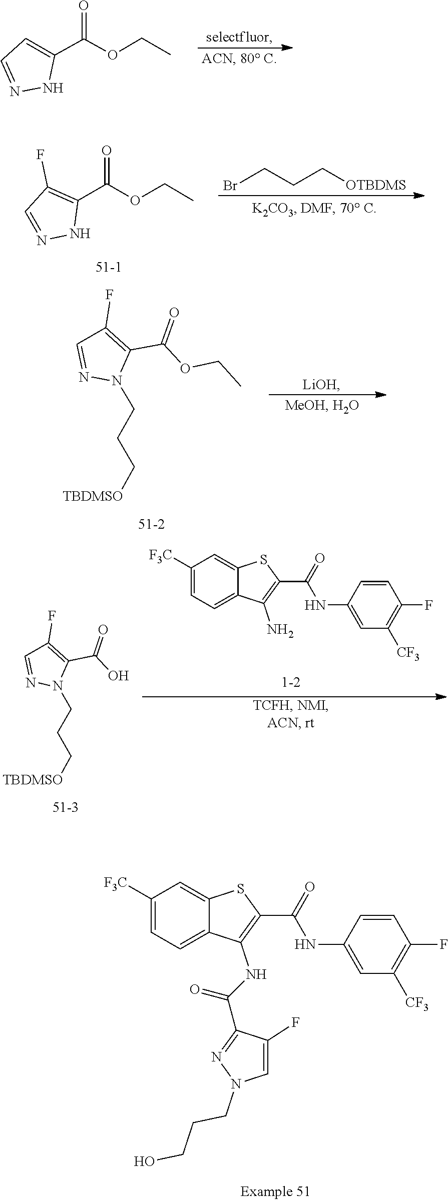

### Intermediate 51-1: ethyl 4-fluoro-1H-pyrazole-5-carboxylate

To a solution of ethyl 1H-pyrazole-5-carboxylate (1.00 g, 7.14 mmol) in ACN (10 mL) was added selectfluor (2.78 g, 7.85 mmol) and the reaction mixture was stirred at 80° C. for 48 h. The reaction mass was concentrated under reduced pressure and the residue was purified by column chromatography to afford Intermediate 51-1 (910 mg, 2.24 mmol, 32% yield). LC-MS RT: 0.84 min; MS (ESI) m/z 157.1 (M−H)⁻; Method E.

### Intermediate 51-2: ethyl 1-(3-((tert-butyldimethylsilyl)oxy)propyl)-4-fluoro-1H-pyrazole-5-carboxylate

Intermediate 51-2 (500 mg, 1.51 mmol, 44% yield) was prepared from Intermediate 51-1 (550 mg, 3.48 mmol) in a similar way as Intermediate 50-1. LC-MS RT: 1.54 min; MS (ESI) m/z 331.1 (M+H)⁺; Method E.

### Intermediate 51-3: 1-(3-((tert-butyldimethylsilyl)oxy)propyl)-4-fluoro-1H-pyrazole-5-carboxylic acid

Intermediate 51-3 (240 mg, 0.794 mmol, 53% yield) was prepared from Intermediate 51-2 (500 mg, 1.51 mmol) in a similar way as Example 2. LC-MS RT: 0.96 min; MS (ESI) m/z 303.2 (M+H)⁺; Method E.

### Example 51: 4-fluoro-N-(2-((4-fluoro-3-(trifluoromethyl)phenyl)carbamoyl)-6-(trifluoromethyl)benzo[b]thiophen-3-yl)-1-(3-hydroxypropyl)-1H-pyrazole-3-carboxamide

Example 51 (17 mg, 0.029 mmol, 24% yield) was prepared from Intermediate 51-3 (36 mg, 0.12 mmol) and Intermediate 1-2 (50 mg, 0.12 mmol) in a similar way as Intermediate 1-3, followed by purification by prep HPLC (Method 5). ¹H NMR (400 MHz, DMSO-d6) δ ppm 10.79 (br s, 1H), 10.66 (s, 1H), 8.71-8.66 (m, 1H), 8.18-8.08 (m, 1H), 8.18-8.08 (m, 1H), 8.02-7.94 (m, 1H), 7.86-7.80 (m, 1H), 7.73-7.68 (m, 1H), 7.59-7.51 (m, 1H), 4.59-4.49 (m, 1H), 4.41-4.32 (m, 2H), 4.37 (br t, J=7.0 Hz, 2H), 1.89-1.80 (m, 2H). LC-MS RT: 2.71 min; MS (ESI) m/z 593.2 (M+H)⁺; Method C.

### Example 52

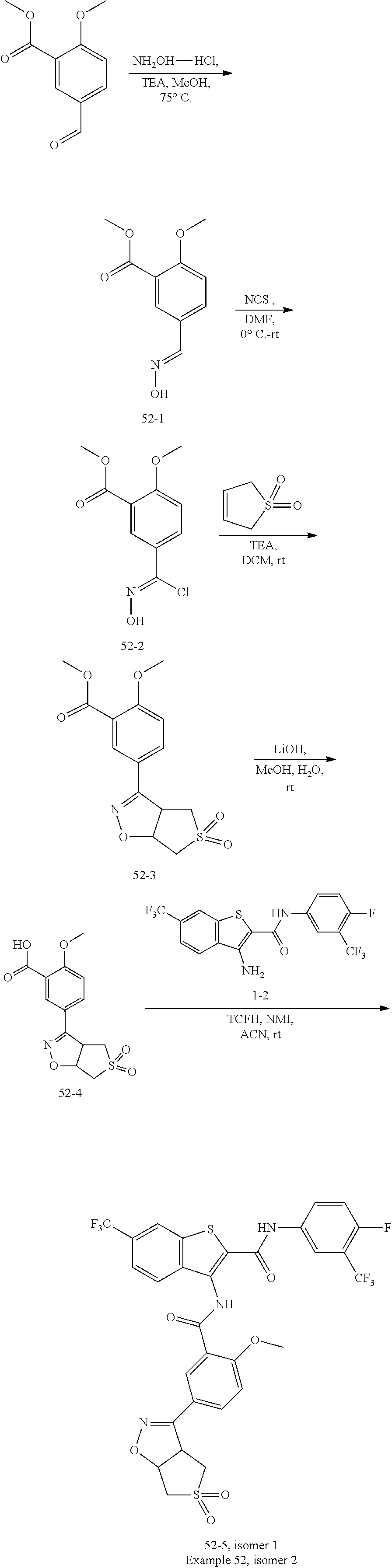

### Intermediate 52-1: Methyl (E)-5-((hydroxyimino) methyl)-2-methoxybenzoate

To a solution of methyl 5-formyl-2-methoxybenzoate (1.45 g, 7.47 mmol) in MeOH (30 mL) was added TEA (4.16 mL, 29.9 mmol) followed by hydroxylamine hydrochloride (0.778 g, 11.2 mmol). The reaction mixture was stirred at 75° C. for 2 h. The reaction mass was concentrated under vacuum. The residue was dissolved in EtOAc (50 mL) and washed with water (2×50 mL). The separated organic layer was dried over Na₂SO₄, filtered and concentrated under reduced pressure to give Intermediate 52-1 (1.41 g, 6.74 mmol, 90% yield). LC-MS RT: 1.11 min; MS (ESI) m/z 210.2 (M+H)⁺; Method E.

### Intermediate 52-2: Methyl (Z)-5-(chloro (hydroxyimino) methyl)-2-methoxybenzoate

To a solution of Intermediate 52-1 (170 mg, 0.813 mmol) in DMF (6 mL) at 0° C. was added NCS (163 mg, 1.22 mmol). The reaction solution was stirred for 1 h at rt and then 10 mL of ice cold water was added to the reaction mass and stirred for an additional 5 min. The solid precipitate was filtered and dried under vacuum to give Intermediate 52-2 (120 mg, 0.493 mmol, 61% yield). ¹H NMR (400 MHz, DMSO-d6) δ ppm 12.29-12.33 (m, 1H), 8.05-8.08 (m, 1H), 7.92-7.97 (m, 1H), 7.24-7.29 (m, 1H), 3.88 (s, 3H), 3.81 (s, 3H).

### Intermediate 52-3: Methyl 5-(5, 5-dioxido-3a, 4, 6, 6a-tetrahydrothieno [3,4-d]isoxazol-3-yl)-2-methoxybenzoate

To a solution of Intermediate 52-2 (60.0 mg, 0.246 mmol) and 2,5-dihydrothiophene 1,1-dioxide (145 mg, 1.23 mmol) in DCM (5 mL) was added TEA (0.172 mL, 1.23 mmol). The resulting reaction mixture was stirred at rt for 8 h. The reaction mixture was concentrated under reduced pressure and the residue was purified by column chromatography to afford Intermediate 52-3 (60.0 mg, 0.184 mmol, 75% yield). LC-MS RT: 1.15 min; MS (ESI) m/z 326.2 (M+H)⁺; Method E.

### Intermediate 52-4: 5-(5,5-dioxido-3a, 4,6,6a-tetrahydrothieno [3,4-d]isoxazol-3-yl)-2-methoxybenzoic acid

Intermediate 52-4 (25 mg, 0.080 mmol, 87% yield) was prepared from Intermediate 52-3 (30 mg, 0.092 mmol) by the procedure described in Example 2. LC-MS RT: 0.40 min; MS (ESI) m/z 310.2 (M−H)⁻; Method E.

### Example 52: 3-(5-(5,5-dioxido-3a, 4,6,6a-tetrahydrothieno[3,4-d]isoxazol-3-yl)-2-methoxybenzamido)-N-(4-fluoro-3-(trifluoromethyl)phenyl)-6-(trifluoromethyl)benzo[b]thiophene-2-carboxamide

Example 52 was prepared from Intermediate 1-2 (25 mg, 0.059 mmol) and Intermediate 52-4 (18 mg, 0.059 mmol) by a similar way as Intermediate 1-3, followed by purification by prep HPLC (Method 44), then prep SFC (Method 45) to give Example 52 (Prep SFC RT=6.0 min) and Intermediate 52-5 (Prep SFC RT=8.1 min).

### Example 52: (4.8 mg, 6.7 μmol, 11% yield). ¹H NMR (400 MHz, DMSO-d6) δ ppm 10.97

10.73 (m, 2H), 8.44 (d, J=0.7 Hz, 1H), 8.18 (d, J=8.6 Hz, 1H), 8.13-8.06 (m, 1H), 8.00 (d, J=2.2 Hz, 1H), 7.80-7.72 (m, 1H), 7.72-7.61 (m, 2H), 7.43 (t, J=9.8 Hz, 1H), 7.22 (d, J=8.6 Hz, 1H), 5.42 (ddd, J=10.9, 6.7, 2.2 Hz, 1H), 4.76 (td, J=10.2, 4.5 Hz, 1H), 3.85 (s, 3H), 3.74-3.66 (m, 2H), 3.52 (br s, 2H). LC-MS RT: 2.29 min; MS (ESI) m/z 714.0 (M−H)⁻; Method A.

Intermediate 52-5: (5.9 mg, 8.2 μmol, 14% yield). ¹H NMR (400 MHz, DMSO-d6) δ ppm 10.86-10.66 (m, 2H), 8.49 (s, 1H), 8.16 (d, J=8.3 Hz, 1H), 8.14-8.08 (m, 1H), 8.02 (d, J=2.2 Hz, 1H), 7.85-7.76 (in, 1H), 7.75-7.64 (m, 2H), 7.45 (t, J=9.7 Hz, 1H), 7.24 (d, J=8.6 Hz, 1H), 5.45-5.37 (m, 1H), 4.76 (td, J=10.1, 4.0 Hz, 1H), 3.87 (s, 3H), 3.74-3.66 (m, 2H), 3.52 (br s, 2H). LC-MS RT: 2.29 min; MS (ESI) m/z 714.1 (M−H)⁻; Method A.

Following intermediates (Table 2) were prepared according to the general method outlined for Intermediate 52-3 by substituting the appropriate alkenes or alkynes followed by hydrolysis.

| | | | LCMS | |
|---|---|---|---|---|
| Intermediate | | | RT, | |
| No | Structure | Reagent | method | MS data |
| 52-4A | 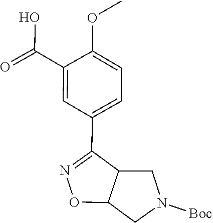 [SMILES: COc1ccc(C2=NOC3CN(C(=O)OC(C)(C)C)CC23)cc1C(=O)O] | 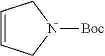 [SMILES: CC(C)(C)OC(=O)N1CC=CC1] | 0.73 E | 361.2 (M − H)⁻ |
| 52-4B | 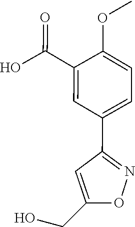 [SMILES: COc1ccc(-c2cc(CO)on2)cc1C(=O)O] | 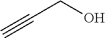 | 0.35 E | 250.2 (M + H)⁺ |
| 52-4C | 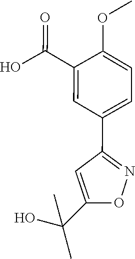 [SMILES: COc1ccc(-c2cc(C(C)(C)O)on2)cc1C(=O)O] | 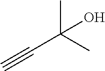 | 0.42 E | 278.2 (M + H)⁺ |
| 52-4D | 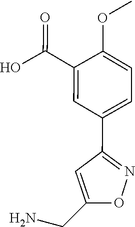 [SMILES: COc1ccc(-c2cc(CN)on2)cc1C(=O)O] | 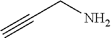 | 0.37 E | 249.2 (M + H)⁺ |
| 52-4E | 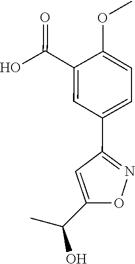 [SMILES: COc1ccc(-c2cc([C@H](C)O)on2)cc1C(=O)O] | 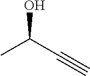 | 0.36 E | 263.1 (M + H)⁺ |
| 52-4F | 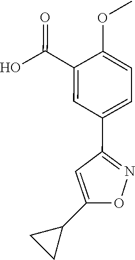 [SMILES: COc1ccc(-c2cc(C3CC3)on2)cc1C(=O)O] | 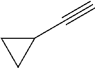 | 0.60 E | 260.2 (M + H)⁺ |
| 52-4G | 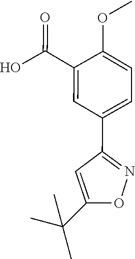 [SMILES: COc1ccc(-c2cc(C(C)(C)C)on2)cc1C(=O)O] | 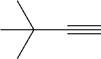 | 0.81 E | 276.2 (M + H)⁺ |
| 52-4H | 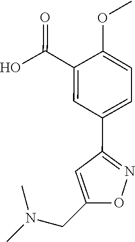 | 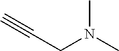 | 0.42 E | 277.2 (M + H)⁺ |
| 52-4I | 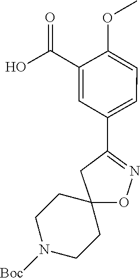 [SMILES: COc1ccc(C2=NOC3(CCN(C(=O)OC(C)(C)C)CC3)C2)cc1C(=O)O] | 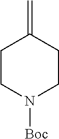 | 0.90 E | 391.2 (M + H)⁺ |
| 52-4J | 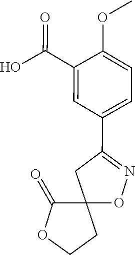 [SMILES: COc1ccc(C2=NOC3(CCOC3=O)C2)cc1C(=O)O] | 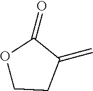 | 0.34 E | 292.3 (M + H)⁺ |
| 52-4K | 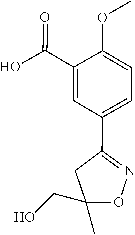 [SMILES: COc1ccc(C2=NOC(C)(CO)C2)cc1C(=O)O] | 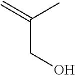 | 0.37 E | 266.2 (M + H)⁺ |
| 52-4L | 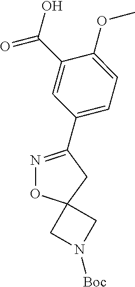 [SMILES: COc1ccc(C2=NOC3(C2)CN(C(=O)OC(C)(C)C)C3)cc1C(=O)O] | 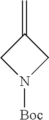 | 0.74 E | 363.2 (M + H)⁺ |
| 52-4M | 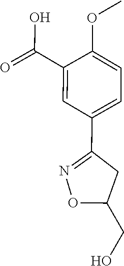 [SMILES: COc1ccc(C2=NOC(CO)C2)cc1C(=O)O] | 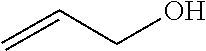 | 0.34 E | 252.2 (M + H)⁺ |
| 52-4N | 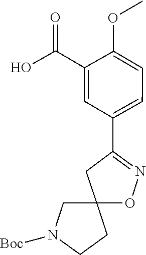 [SMILES: COc1ccc(C2=NOC3(CCN(C(=O)OC(C)(C)C)C3)C2)cc1C(=O)O] | 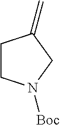 | 0.77 E | 375.2 (M − H)⁻ |
| 52-4O | 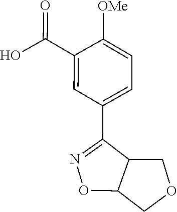 [SMILES: COc1ccc(C2=NOC3COCC23)cc1C(=O)O] | 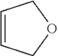 | 0.875 C | 264.2 (M + H)⁺ |
| 52-4P | 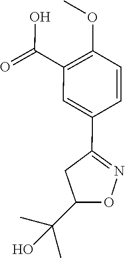 [SMILES: COc1ccc(C2=NOC(C(C)(C)O)C2)cc1C(=O)O] | 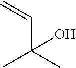 | 0.37 E | 280.3 (M + H)⁺ |
| 52-4Q | 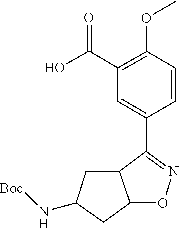 [SMILES: COc1ccc(C2=NOC3CC(NC(=O)OC(C)(C)C)CC23)cc1C(=O)O] | 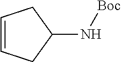 [SMILES: CC(C)(C)OC(=O)NC1CC=CC1] | 0.70 E | 321.1 [M + H − tBu]⁺ |
| 52-4R | 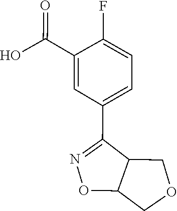 [SMILES: O=C(O)c1cc(C2=NOC3COCC23)ccc1F] | 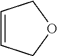 | 0.36 E | 250.2 (M − H)⁻ |
| 52-4S | 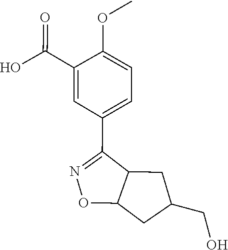 [SMILES: COc1ccc(C2=NOC3CC(CO)CC23)cc1C(=O)O] | 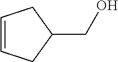 | 0.83 F | 292.3 (M + H)⁺ |

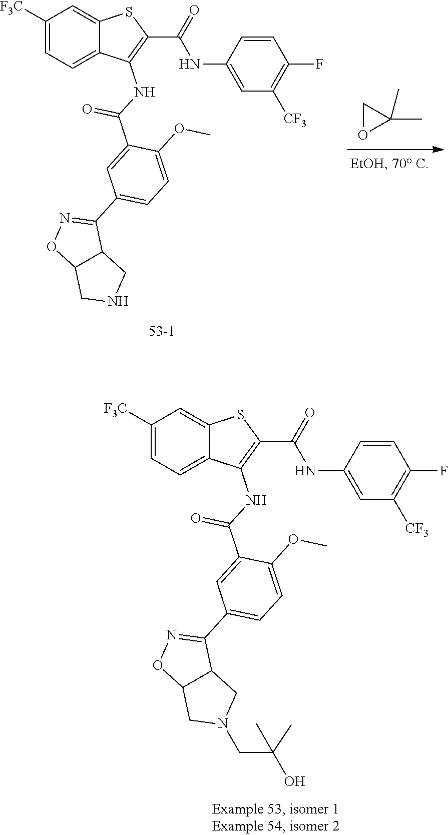

### Example 53 and 54: N-(4-fluoro-3-(trifluoromethyl)phenyl)-3-(5-(5-(2-hydroxy-2-methylpropyl)-3a, 5,6,6a-tetrahydro-4H-pyrrolo[3,4-d]isoxazol-3-yl)-2-methoxybenzamido)-6-(trifluoromethyl)benzo[b]thiophene-2-carboxamide

A solution of Intermediate 53-1 (100 mg, 0.15 mmol) and 2,2-dimethyloxirane (54 mg, 0.75 mmol) in EtOH (5 mL) was heated at 70° C. for 3 h. The reaction mass was concentrated under reduced pressure and purified by prep HPLC (Method 2) followed by prep SFC purification (Column: Chiralpak IC (250×21.2) mm, 5 μm; % CO₂: 65%; Co-solvent: 35% of 0.2% DEA in ACN:IPA; Total Flow: 80.0 g/min; Back Pressure: 100 bar; Temperature: 40° C.; UV: 265 nm) to afford Example 53 (Prep SFC RT=1.7 min) and Example 54 (Prep SFC RT=2.4 min, 99.3% ee). Analytical SFC conditions: (Column: Chiralpak IC (250×4.6) mm, 5 μm; % CO₂: 65%; Co-solvent: 35% of 0.2% DEA in ACN:IPA; Total Flow: 4.0 g/min; Back Pressure: 100 bar; Temperature: 40° C.; UV: 265 nm).

Example 53: (16 mg, 0.021 mmol, 14% yield)¹H NMR (400 MHz, DMSO-d6) δ ppm 8.61 (s, 1H), 8.39 (s, 2H), 8.17 (d, J=6.4 Hz, 1H), 8.11 (d, J=8.8 Hz, 1H), 8.04 (s, 1H), 7.93 (br s, 1H), 7.83-7.77 (m, 2H), 7.51 (t, J=9.8 Hz, 1H), 7.29 (d, J=8.5 Hz, 1H), 5.11 (dd, J=4.3, 9.3 Hz, 1H), 4.26 (br t, J=8.0 Hz, 1H), 3.94 (s, 3H), 3.07 (br d, J=9.5 Hz, 1H), 2.90-2.66 (m, 2H), 2.47-2.39 (m, 2H), 2.39-2.13 (m, 2H), 1.24 (s, 3H), 1.09 (t, J=7.3 Hz, 3H). LC-MS RT: 2.76 min; MS (ESI) m/z 739.2 (M+H)⁺; Method C.

Example 54: (22 mg, 0.029 mmol, 20% yield)¹H NMR (400 MHz, DMSO-d6) δ ppm 8.61 (s, 1H), 8.39 (s, 2H), 8.17 (d, J=6.4 Hz, 1H), 8.11 (d, J=8.8 Hz, 1H), 8.04 (s, 1H), 7.93 (br s, 1H), 7.83-7.77 (m, 2H), 7.51 (t, J=9.8 Hz, 1H), 7.29 (d, J=8.5 Hz, 1H), 5.11 (dd, J=4.3, 9.3 Hz, 1H), 4.26 (br t, J=8.0 Hz, 1H), 3.94 (s, 3H), 3.07 (br d, J=9.5 Hz, 1H), 2.90-2.66 (m, 2H), 2.47-2.39 (m, 2H), 2.39-2.13 (m, 2H), 1.24 (s, 3H), 1.09 (t, J=7.3 Hz, 3H). LC-MS RT: 2.76 min; MS (ESI) m/z 739.2 (M+H)⁺; Method C.

### Examples 55 and 56

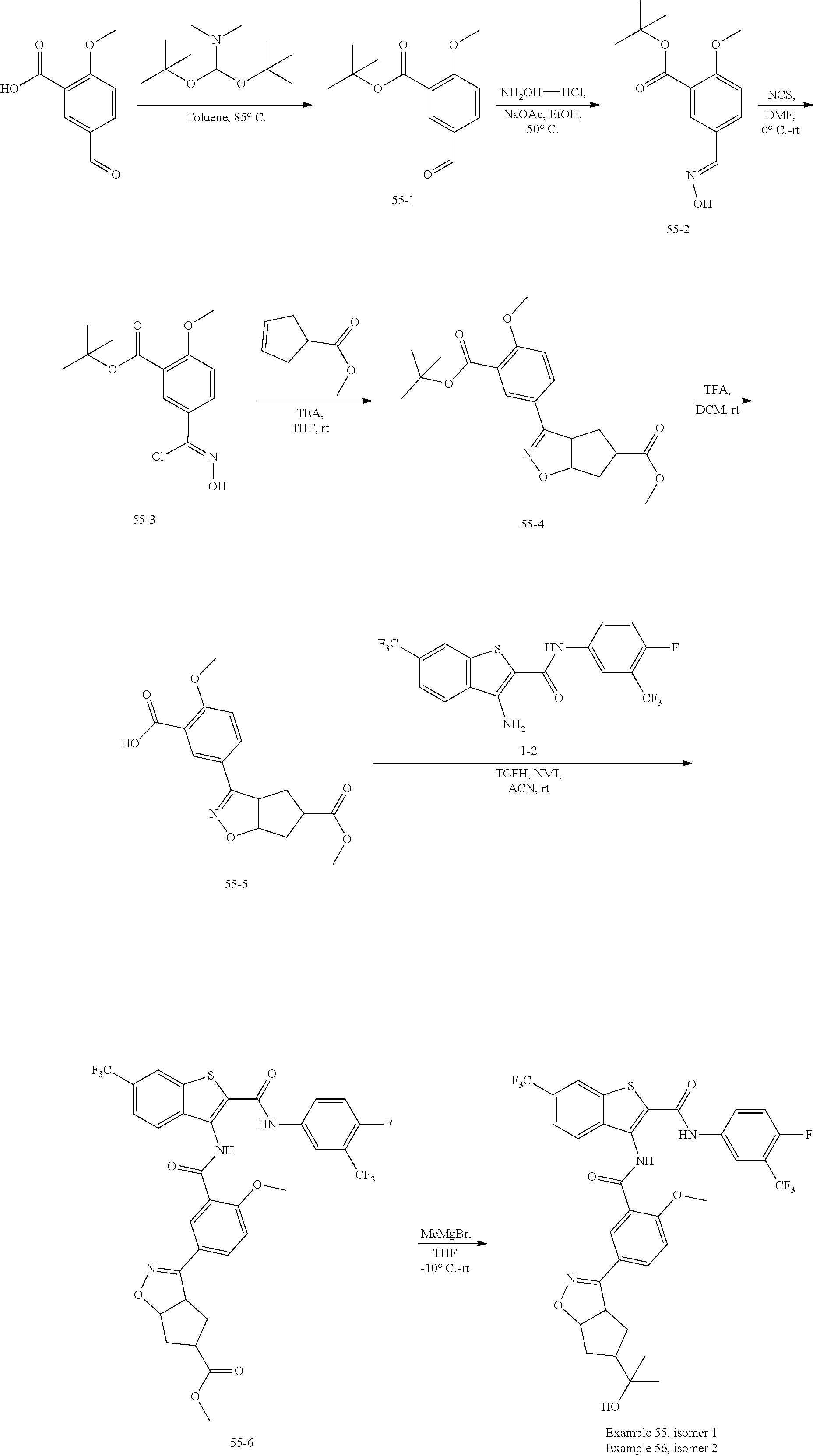

### Intermediate 55-1: tert-butyl 5-formyl-2-methoxybenzoate

Intermediate 55-1 (4.20 g, 17.8 mmol, 64% yield) was prepared from 5-formyl-2-methoxybenzoic acid (5.00 g, 27.8 mmol) in a similar way as Intermediate 27-2. LC-MS RT: 2.36 min; MS (ESI) m/z 181.1 (M-tBu+H)⁺; Method C.

### Intermediate 55-2: tert-butyl (E)-5-((hydroxyimino)methyl)-2-methoxybenzoate

To a solution of Intermediate 55-1 (5.00 g, 21.2 mmol), hydroxylamine hydrochloride (4.41 g, 63.5 mmol) in EtOH (70 mL) and H2O (70 mL) was added NaOAc (5.21 g, 63.5 mmol). The reaction solution was heated to 50° C. for 3 h. The cooled reaction mixture was concentrated under reduced pressure, and the resulting reaction mass was diluted with ice cold water (150 mL) and extracted with EtOAc (2×100 mL). The combined organic layers were washed with water, followed by brine, dried over Na₂SO₄, filtered, and concentrated under reduced pressure to give Intermediate 55-2 (5.20 g, 20.7 mmol, 98% yield). LC-MS RT: 2.10 min; MS (ESI) m/z 250.0 (M−H)⁻; Method C.

### Intermediate 55-3: tert-butyl (Z)-5-(chloro(hydroxyimino)methyl)-2-methoxybenzoate

Intermediate 55-3 (5.50 g, 19.2 mmol, 97% yield) was prepared from Intermediate 55-2 (5.00 g, 19.9 mmol) in a similar way as Intermediate 52-2. ¹H NMR (400 MHz, DMSO-d6) δ ppm 12.30 (s, 1H), 7.99-7.87 (m, 1H), 7.97-7.87 (m, 1H), 7.25-7.20 (m, 1H), 3.93-3.85 (m, 3H), 1.57-1.51 (m, 9H).

### Intermediate 55-4: methyl 3-(3-(tert-butoxycarbonyl)-4-methoxyphenyl)-3a, 5,6,6a-tetrahydro-4H-cyclopenta[d]isoxazole-5-carboxylate

Intermediate 55-4 (1.40 g, 3.73 mmol, 21% yield) was prepared from Intermediate 55-3 (5.00 g, 17.5 mmol) and methyl cyclopent-3-ene-1-carboxylate (11.0 g, 87.0 mmol) in a similar way as Intermediate 52-3. LC-MS RT: 2.67 min; MS (ESI) m/z 376.2 (M+H)⁺; Method C.

### Intermediate 55-5: 2-methoxy-5-(5-(methoxycarbonyl)-3a, 5,6,6a-tetrahydro-4H-cyclopenta[d]isoxazol-3-yl)benzoic acid

Intermediate 55-5 (280 mg, 0.877 mmol, 94% yield) was prepared from Intermediate 55-4 (350 mg, 0.932 mmol) in a similar way as Intermediate 4-2. LC-MS RT: 1.90 min; MS (ESI) m/z 320.0 (M+H)⁺; Method C.

### Intermediate 55-6: methyl 3-(3-((2-((4-fluoro-3-(trifluoromethyl)phenyl)carbamoyl)-6-(trifluoromethyl)benzo[b]thiophen-3-yl)carbamoyl)-4-methoxyphenyl)-3a, 5,6,6a-tetrahydro-4H-cyclopenta[d]isoxazole-5-carboxylate

Intermediate 55-6 (160 mg, 0.221 mmol, 42% yield) was prepared from Intermediate 55-4 (216 mg, 0.677 mmol) and Intermediate 1-2 (220 mg, 0.521 mmol) in a similar way as Intermediate 1-3. LC-MS RT: 1.24 min; MS (ESI) m/z 722.3 (M−H)⁻; Method E.

### Examples 55 and 56: N-(4-fluoro-3-(trifluoromethyl)phenyl)-3-(5-(5-(2-hydroxypropan-2-yl)-3a, 5,6,6a-tetrahydro-4H-cyclopenta[d]isoxazol-3-yl)-2-methoxybenzamido)-6-(trifluoromethyl)benzo[b]thiophene-2-carboxamide

Examples 55 and 56 were prepared from Intermediate 55-6 (100 mg, 0.138 mmol) in a similar way as Example 3. The residue was purified by prep LCMS (Method 2) followed by prep SFC purification (Column: Luxcellulose-4 (250×30) mm, 5 μm; % CO₂: 65%; % Co-solvent: 35% of 0.2% of 4M NH3 in MeOH; Total Flow: 80.0 g/min; Back Pressure: 100 bar; Temperature: 30° C.; UV: 265 nm) to give Example 55 (Prep SFC RT=4.2 min, 100% ee) and Example 56 (Prep SFC RT=5.7 min, 99.5% ee). Analytical SFC conditions: (Column Name: Luxcellulose-4 (250×4.6) mm, 5 μm; % CO₂: 65%; % Co-solvent: 35% of 0.2% of 4M NH3 in MeOH; Total Flow: 4.0 g/min; Back Pressure: 100 bar; Temperature: 30° C.; UV: 265 nm).

Example 55: (17.0 mg, 23.5 mol, 17% yield)¹H NMR (400 MHz, DMSO-d6) δ ppm 10.97-10.73 (m, 1H), 8.69-8.61 (m, 1H), 8.19-8.06 (m, 3H), 8.01-7.93 (m, 1H), 7.86-7.78 (m, 2H), 7.57-7.48 (m, 1H), 7.34-7.27 (m, 1H), 5.14-5.08 (m, 1H), 4.21-4.13 (m, 2H), 4.01-3.93 (m, 3H), 2.10-1.58 (m, 6H), 1.03 (d, J=13.6 Hz, 7H). LC-MS RT: 0.76 min; MS (ESI) m/z 724.2 (M+H)⁺; Method C.

Example 56: (16.0 mg, 22.1 mol, 16% yield)¹H NMR (400 MHz, DMSO-d6) δ ppm 10.97-10.75 (m, 1H), 8.69-8.62 (m, 1H), 8.19-8.06 (m, 3H), 8.01-7.93 (m, 1H), 7.86-7.76 (m, 2H), 7.58-7.49 (m, 1H), 7.35-7.28 (m, 1H), 5.14-5.07 (m, 1H), 4.22-4.13 (m, 2H), 4.01-3.92 (m, 3H), 1.97-1.62 (m, 6H), 1.03 (d, J=13.1 Hz, 7H). LC-MS RT: 0.73 min; MS (ESI) m/z 724.3 (M+H)⁺; Method C.

### Example 57

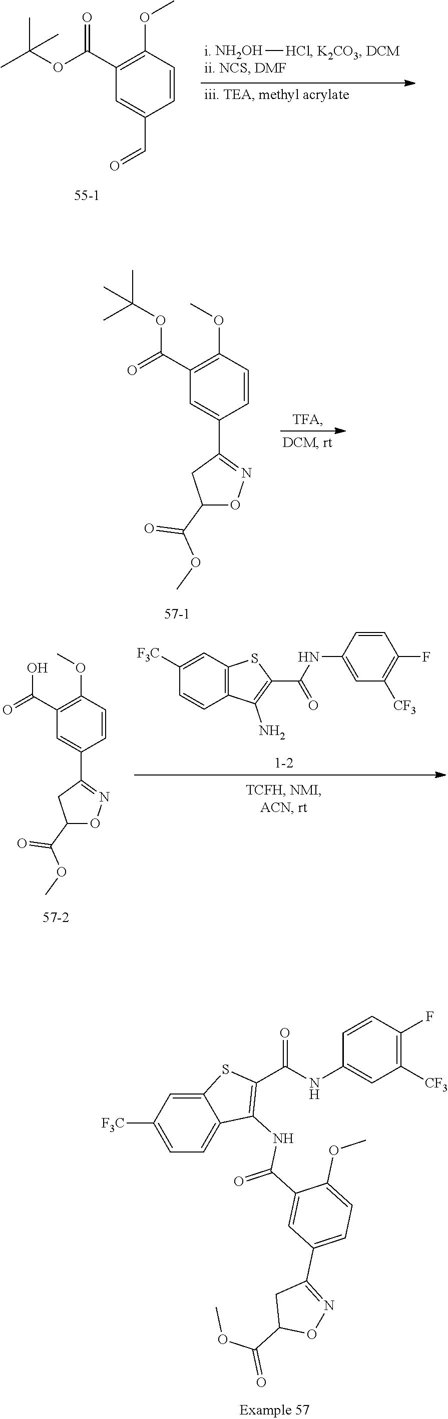

### Intermediate 57-1: methyl 3-(3-(tert-butoxycarbonyl)-4-methoxyphenyl)-4,5-dihydroisoxazole-5-carboxylate

To a stirred solution of Intermediate 55-1 (500 mg, 2.12 mmol) in DCM (10 mL) was added hydroxylamine hydrochloride (221 mg, 3.17 mmol) and K₂CO₃ (219 mg, 1.59 mmol). The reaction mixture was stirred at rt for 18 h, and then DMF (0.2 mL) and NCS (424 mg, 3.17 mmol) were added. The reaction mixture was stirred at rt for 3 h, and then Et3N (0.442 mL, 3.17 mmol) and methyl acrylate (182 mg, 2.12 mmol) were added. The reaction mixture was stirred at rt for 2 h, and then the reaction mass was quenched with aq NaHCO₃ solution. The reaction solution was extracted with DCM (2×), the combined organic layers were dried over Na₂SO₄ and then concentrated under reduced pressure. The resulting residue was purified by column chromatography to provide Intermediate 57-1 (400 mg, 1.19 mmol, 56% yield). LC-MS RT: 1.52 min; MS (ESI) m/z 336.2 (M+H)⁺; Method E.

### Intermediate 57-2: 2-methoxy-5-(5-(methoxycarbonyl)-4,5-dihydroisoxazol-3-yl)benzoic acid

Intermediate 57-2 (280 mg, 1.00 mmol, 96% yield) was prepared from Intermediate 57-1 (350 mg, 1.04 mmol) in a similar way as Intermediate 4-2. LC-MS RT: 0.45 min; MS (ESI) m/z 280.2 (M+H)⁺; Method E.

### Example 57: methyl 3-(3-((2-((4-fluoro-3-(trifluoromethyl)phenyl)carbamoyl)-6-(trifluoromethyl)benzo[b]thiophen-3-yl)carbamoyl)-4-methoxyphenyl)-4,5-dihydroisoxazole-5-carboxylate

Example 57 (1.0 mg, 1.5 mol, 0.4% yield) was prepared from Intermediate 57-2 (100 mg, 0.36 mmol) and Intermediate 1-2 (91 mg, 0.22 mmol) in a similar way as Intermediate 1-3, followed by purification by prep LCMS (Method 1). ¹H NMR (400 MHz, DMSO-d6) δ ppm 11.04-10.67 (m, 1H), 8.65 (br s, 1H), 8.16 (br d, J=4.0 Hz, 1H), 8.13-8.04 (m, 2H), 7.97 (br d, J=8.0 Hz, 1H), 7.86 (br d, J=8.0 Hz, 1H), 7.81 (br d, J=9.0 Hz, 1H), 7.53 (t, J=9.8 Hz, 1H), 7.32 (br d, J=8.5 Hz, 1H), 5.29 (dd, J=11.5, 6.5 Hz, 1H), 3.96 (s, 3H), 3.82-3.70 (m, 4H), 3.67-3.61 (m, 1H), 1.23 (s, 2H). LC-MS RT: 2.57 min; MS (ESI) m/z 682.1 (M−H)⁻; Method A.

### Example 58

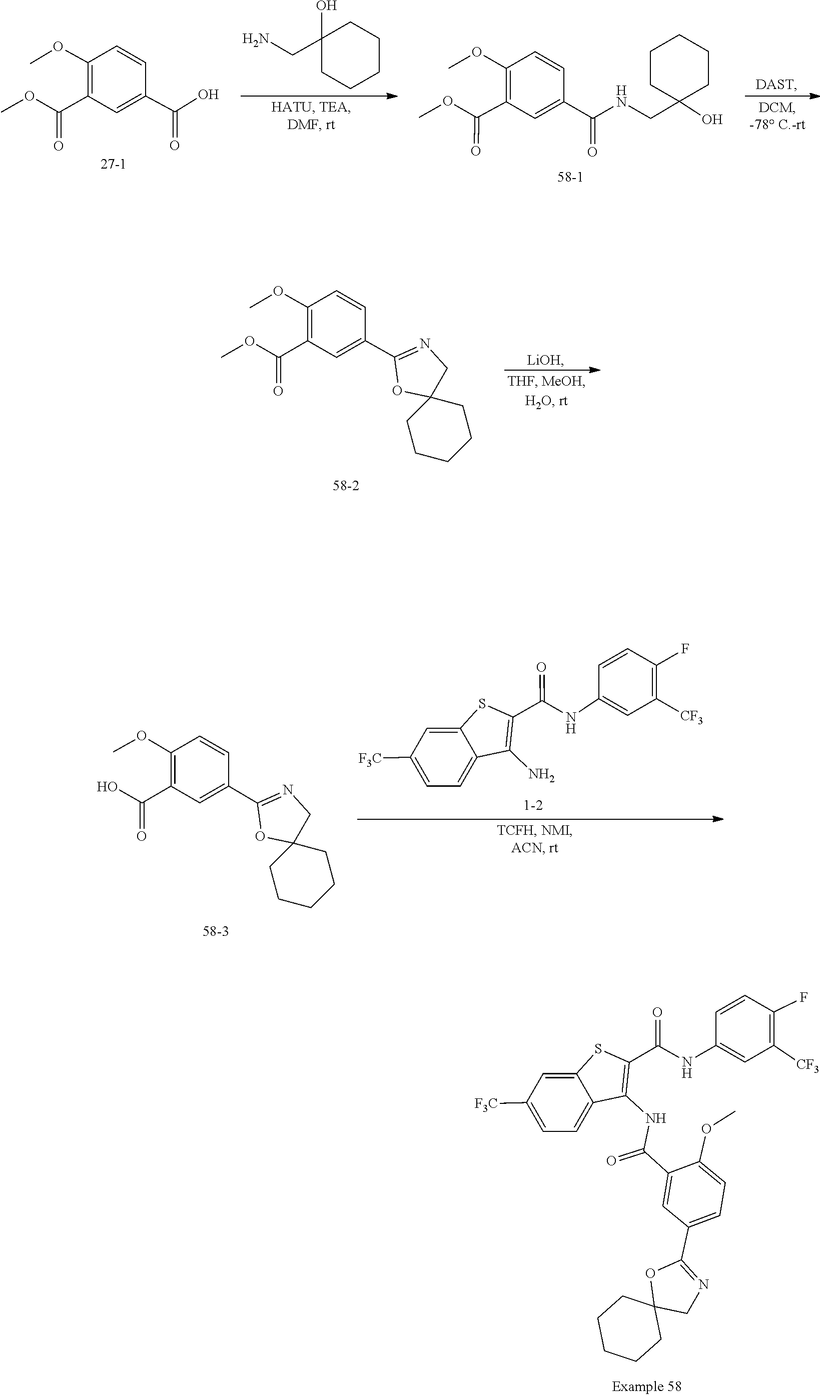

### Intermediate 58-1: methyl 5-(((1-hydroxycyclohexyl)methyl)carbamoyl)-2-methoxybenzoate

Intermediate 58-1 (220 mg, 0.685 mmol, 72% yield) was prepared from Intermediate 27-1 (200 mg, 0.952 mmol) and 1-(aminomethyl)cyclohexan-1-ol (148 mg, 1.14 mmol) by a similar way as Example 7 and 8. LC-MS RT: 1.00 min; MS (ESI) m/z 322.3 (M+H)⁺; Method E.

### Intermediate 58-2: methyl 2-methoxy-5-(1-oxa-3-azaspiro[4.5]dec-2-en-2-yl)benzoate

To a stirred solution of Intermediate 58-1 (300 mg, 0.933 mmol) in DCM (10 mL) cooled to −78° C., was added DAST (0.493 mL, 3.73 mmol) dropwise. The reaction solution was allowed to warm to rt gradually and was stirred at rt for 2 h. The reaction mass was diluted with water and extracted with DCM (2×). The combined organic layer was washed with saturated NaHCO₃ solution, dried over Na₂SO₄ and concentrated under reduced pressure. Purification by column chromatography provided Intermediate 58-2 (220 mg, 0.725 mmol, 78% yield). LC-MS RT: 1.62 min; MS (ESI) m/z 304.3 (M+H)⁺; Method E.

### Intermediate 58-3: 2-methoxy-5-(1-oxa-3-azaspiro[4.5]dec-2-en-2-yl)benzoic acid

Intermediate 58-3 (180 mg, 0.622 mmol, 70% yield) was prepared from Intermediate 58-2 (270 mg, 0.890 mmol) by the procedure described in Example 2. LC-MS RT: 0.74 min; MS (ESI) m/z 290.2 (M+H)⁺; Method E.

### Example 58: N-(4-fluoro-3-(trifluoromethyl)phenyl)-3-(2-methoxy-5-(1-oxa-3-azaspiro[4.5]dec-2-en-2-yl)benzamido)-6-(trifluoromethyl)benzo[b]thiophene-2-carboxamide

Example 58 (8.6 mg, 12 mol, 7% yield) was prepared from Intermediate 1-2 (44 mg, 0.10 mmol) and Intermediate 58-3 (50 mg, 0.17 mmol) in a similar way to Intermediate 1-3, followed by purification by prep LCMS (Method 1). ¹H NMR (400 MHz, DMSO-d6) δ ppm 10.86 (br dd, J=4.5, 2.8 Hz, 1H), 10.76 (br d, J=6.6 Hz, 1H), 8.73-8.66 (m, 1H), 8.66-8.54 (m, 1H), 8.39 (br d, J=3.9 Hz, 1H), 8.22-8.14 (m, 1H), 8.14-8.04 (m, 2H), 8.04-7.92 (m, 1H), 7.82 (d, J=9.3 Hz, 1H), 7.54 (t, J=9.7 Hz, 1H), 7.31 (d, J=8.8 Hz, 1H), 5.51 (br d, J=1.2 Hz, 1H), 3.99 (s, 3H), 3.81-3.73 (m, 1H), 3.53-3.41 (m, 1H), 2.01-1.88 (m, 2H), 1.76-1.63 (m, 1H), 1.61-1.40 (m, 6H), 1.31-1.19 (m, 1H). LC-MS RT: 2.73 mi; MS (ESI) m/z 694.2 (M+H)⁺; Method A.

### Example 59

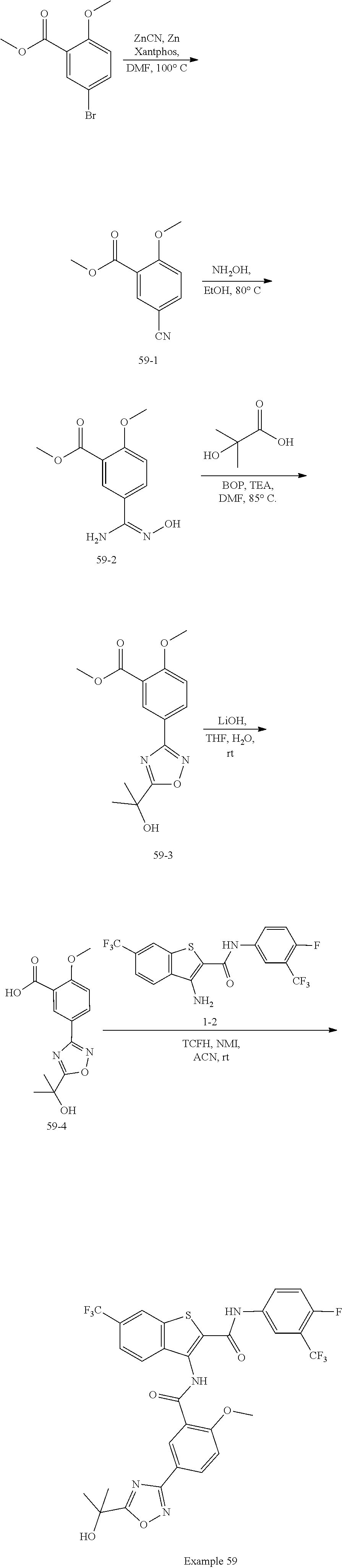

### Intermediate 59-1: Methyl 5-cyano-2-methoxybenzoate

To a solution of methyl 5-bromo-2-methoxybenzoate (1.00 g, 4.08 mmol) in DMF (3 mL) that was degassed with N₂ for 10 min was added zinc cyanide (963 mg, 8.16 mmol), dichloro (9,9-dimethyl-4,5-bis(diphenylphosphino)xanthene]palladium(II) (308 mg, 0.408 mmol), and zinc (400 mg, 6.12 mmol). The resulting solution was degassed for an additional 5 min and then was heated at 100° C. for 3 h. The reaction mass was filtered through a celite bed and the filtrate was diluted with EtOAc (100 mL), then washed with water (2×150 mL). The separated organic layer was dried over Na₂SO₄, filtered and concentrated under reduced pressure. The residue was purified by column chromatography to afford Intermediate 59-1 (650 mg, 3.40 mmol, 83% yield). LC-MS RT: 1.08 min; MS (ESI) m/z 192.2 (M+H)⁺; Method E.

### Intermediate 59-2: Methyl (E)-5-(N′-hydroxycarbamimidoyl)-2-methoxybenzoate

To a solution of Intermediate 59-1 (50 mg, 0.26 mmol) in EtOH (10 mL) was added hydroxylamine solution (43 mg, 0.65 mmol) and the reaction solution was heated to 80° C. for 1 h. The reaction mass was concentrated under reduced pressure to provide Intermediate 59-2 (40 mg, 0.18 mmol, 68% yield). LC-MS RT: 0.92 min; MS (ESI) m/z 225.2 (M+H)⁺; Method E.

### Intermediate 59-3: Methyl 5-(5-(2-hydroxypropan-2-yl)-1, 2, 4-oxadiazol-3-yl)-2-methoxybenzoate

To a solution of Intermediate 59-2 (50 mg, 0.22 mmol) in DMF (2 mL) was added 2-hydroxy-2-methylpropanoic acid (28 mg, 0.27 mmol), BOP (200 mg, 0.45 mmol) and Et₃N (0.093 mL, 0.67 mmol). The resulting mixture was heated at 85° C. for 24 h. The reaction mass was diluted with EtOAc (50 mL) and washed with water (2×50 mL). The separated organic layer was dried over Na₂SO₄, filtered and concentrated under reduced pressure to afford Intermediate 59-3 (40 mg, 0.14 mmol, 61% yield). LC-MS RT: 1.13 min; MS (ESI) m/z 293.2 (M+H)⁺; Method E.

### Intermediate 59-4: 5-(5-(2-hydroxypropan-2-yl)-1, 2, 4-oxadiazol-3-yl)-2-methoxybenzoic acid

Intermediate 59-4 (40 mg, 0.14 mmol, 84% yield) was prepared from Intermediate 59-3 (50 mg, 0.171 mmol) by the procedure described in Example 2. LC-MS RT: 0.42 min; MS (ESI) m/z 279.2 (M+H)⁺; Method E.

### Example 59: N-(4-fluoro-3-(trifluoromethyl) phenyl)-3-(5-(5-(2-hydroxypropan-2-yl)-1, 2, 4-oxadiazol-3-yl)-2-methoxybenzamido)-6-(trifluoromethyl) benzo[b]thiophene-2-carboxamide

Example 59 (25 mg, 0.036 mmol, 20% yield) was prepared from Intermediate 1-2 (49 mg, 0.11 mmol) and Intermediate 59-4 (50 mg, 0.18 mmol) in a similar way as Intermediate 1-3. ¹H NMR (400 MHz, DMSO-d6) δ ppm 10.90 (br s, 1H), 10.80-10.69 (m, 1H), 8.67 (s, 1H), 8.45 (d, J=2.4 Hz, 1H), 8.24-8.13 (m, 2H), 8.09 (d, J=8.3 Hz, 1H), 8.04-7.96 (m, 1H), 7.83 (dd, J=1.6, 8.9 Hz, 1H), 7.54 (t, J=10.0 Hz, 1H), 7.44 (d, J=9.0 Hz, 1H), 6.07 (s, 1H), 4.04 (s, 3H), 1.61 (s, 6H). LC-MS RT: 2.47 min; MS (ESI) m/z 683.2 (M+H)⁺; Method A.

TABLE 3

| | | | | | LC RT^{a} |
|---|---|---|---|---|---|
| | | | | | (min) and |
| | | | | | chiral |
| | | | | | conditions |
| Ex. | | | MS (ESI) | | when |
| No. | Structure | Name | (M + H)⁺ | ¹H NMR | separated |
| 60 | 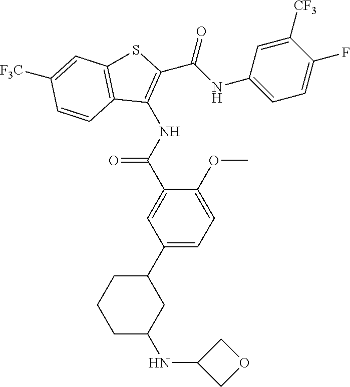 [SMILES: COc1ccc(C2CCCC(NC3COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(3- (oxetan-3- ylamino) cyclohexyl) benzamido)- 6- (trifluoromethyl) benzo[b] thiophene-2- carboxamide | 710.3 | ¹H NMR (400 MHz, DMSO-d6) δ ppm 10.68-10.86 (m, 2 H) 8.63-8.69 (m, 1 H) 8.10-8.19 (m, 1 H) 8.05-8.11 (m, 1 H) 7.92-8.01 (m, 1 H) 7.75-7.85 (m, 1 H) 7.58-7.65 (m, 1 H) 7.52-7.58 (m, 1 H) 7.35-7.44 (m, 1 H) 7.11-7.19 (m, 1 H) 4.54-4.67 (m, 1 H) 4.23-4.33 (m, 1 H) 3.90-3.96 (m, 3 H) 3.93 (s, 3 H) 3.37- 3.64 (m, 2 H) 1.64- 1.85 (m, 2 H) 1.30- 1.40 (m, 2 H) 1.18- 1.26 (m, 2 H) 1.09- 1.18 (m, 2 H) 0.93- 1.06 (m, 1 H) | 2.86 C Analytical SFC (Method 13) RT = 9.18 min. 100% ee HPLC (Method 11) provided two diastereomers (peak 1, RT = 14.562 min; peak 2, RT = 15.724 min). Each diastereomer was further purified by prep SFC (Method 12). |
| 61 | 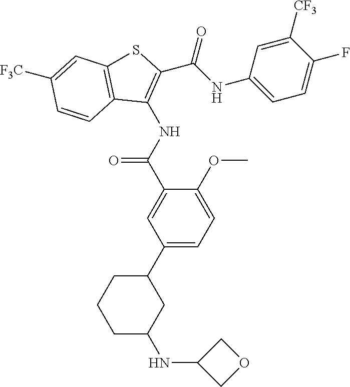 [SMILES: COc1ccc(C2CCCC(NC3COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(3- (oxetan-3- ylamino) cyclohexyl) benzamido)- 6- (trifluoromethyl) benzo[b] thiophene-2- carboxamide | 710.2 | ¹H NMR (400 MHz, DMSO-d6) δ ppm 10.52-11.00 (m, 2 H) 8.59-8.64 (m, 1 H) 8.06-8.13 (m, 1 H) 7.99-8.06 (m, 1 H) 7.84-7.96 (m, 1 H) 7.74-7.81 (m, 1 H) 7.57-7.61 (m, 1 H) 7.46-7.53 (m, 1 H) 7.33-7.39 (m, 1 H) 7.05-7.15 (m, 1 H) 4.54-4.63 (m, 1 H) 4.30-4.37 (m, 1 H) 3.86-3.91 (m, 3 H) 3.23-3.38 (m, 2 H) 2.80-2.86 (m, 2 H) 2.46 (dt, J = 3.76, 1.63 Hz, 2 H) 2.40-2.43 (m, 1 H) 2.38-2.42 (m, 1 H) 1.61-1.72 (m, 2 H) 1.55-1.61 (m, 1 H) 1.44-1.53 (m, 1 H) 1.29-1.45 (m, 1 H) 1.18-1.21 (m, 1 H) | 2.85 C Analytical SFC (Method 13) RT = 7.68 min. 98% ee HPLC (Method 11) provided two diastereomers (peak 1, RT = 14.562 min; peak 2, RT = 15.724 min). Each diastereomer was further purified by prep SFC (Method 12). |
| 62 | 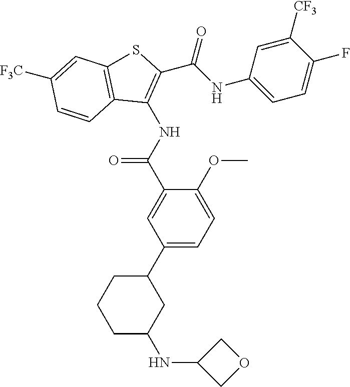 [SMILES: COc1ccc(C2CCCC(NC3COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(3- (oxetan-3- ylamino) cyclohexyl) benzamido)- 6- (trifluoromethyl) benzo[b] thiophene-2- carboxamide | 710.0 | ¹H NMR (400 MHz, DMSO-d6) δ ppm 10.44-11.09 (m, 2H) 8.43-8.71 (m, 1 H) 8.06-8.21 (m, 1 H) 7.85-8.01 (m, 1 H) 7.68-7.84 (m, 1 H) 7.56-7.68 (m, 1 H) 7.33-7.58 (m, 2 H) 7.35-7.22 (m, 1 H ) 7.01-7.18 (m, 1 H) 5.65 (s, 1H ) 4.61 (s, 2 H) 4.28 (d, J = 7.53 Hz, 2 H) 3.90 (br s, 3 H) 3.41 (br d, J = 17.57 Hz, 3H) 2.67-2.69 (m, 2 H) 2.53-2.60 (m,2 H) 1.59-1.72 (m, 2H) 1.18-1.32 (m, 1H) | 2.85 C Analytical SFC (Method 13) RT = 5.12 min. 100% ee HPLC (Method 11) provided two diastereomers (peak 1, RT = 14.562 min; peak 2, RT = 15.724 min). Each diastereomer was further purified by prep SFC (Method 12) |
| 63 | 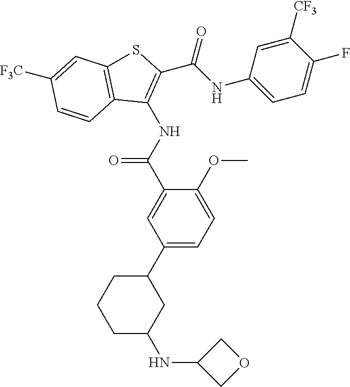 [SMILES: COc1ccc(C2CCCC(NC3COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(3- (oxetan-3- ylamino) cyclohexyl) benzamido)- 6- (trifluoromethyl) benzo[b] thiophene-2- carboxamide | 710.3 | 1H NMR (400 MHz, DMSO-d6) δ ppm 10.66-10.87 (m, 2 H) 8.60-8.72 (m, 1 H) 8.10-8.20 (m, 1 H) 8.05-8.11 (m, 1 H) 7.94-8.02 (m, 1 H) 7.77-7.88 (m, 1 H) 7.59-7.66 (m, 1 H) 7.51-7.59 (m, 1 H) 7.36-7.46 (m, 1 H) 7.12-7.19 (m, 1 H) 5.82 (s, 1H) 4.58- 4.68 (m, 2 H) 4.33- 4.44 (m, 2 H) 3.87- 3.98 (m, 3 H) 3.34- 3.42 (m, 2 H) 3.32 (s, 2 H) 2.51 (dt, J = 3.76, 1.63 Hz, 4 H) 1.65- 1.80 (m, 1 H) 1.27- 1.65 (m, 1 H) | 2.98 C Analytical SFC (Method 13) RT = 4.7 min. 98% ee HPLC (Method 11) provided two diastereomers (peak 1 RT = 14.562 min; peak 2, RT = 15.724 min). Each diastereomer was further purified by prep SFC (Method 12) |
| 64 | 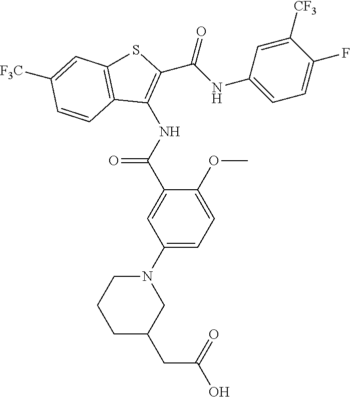 [SMILES: COc1ccc(N2CCCC(CC(=O)O)C2)cc1C(=O)Nc1c(C(=O)Nc2ccc(F)c(C(F)(F)F)c2)sc2cc(C(F)(F)F)ccc12] | 2-(1-(3-((2-((4- fluoro-3- (trifluoromethyl) phenyl) carbamoyl)-6- (trifluoromethyl) benzo[b] thiophen-3- yl)carbamoyl)- 4- methoxyphenyl) piperidin-3- yl)acetic acid | 698.2 | ¹H NMR (400 MHz, DMSO-d6) δ ppm 10.96-10.70 (m, 2H), 8.66 (d, J = 0.7 Hz, 1H), 8.15 (dd, J = 2.6, 6.7 Hz, 1H), 8.06 (d, J = 8.8 Hz, 2H), 7.99 (ddd, J = 2.8, 4.2, 8.9 Hz, 1H), 7.81 (dd, J = 1.8, 8.9 Hz, 1H), 7.54 (t, J = 9.8 Hz, 1H), 7.38 (br d, J = 2.2 Hz, 1H), 7.21-7.06 (m, 1H), 4.00-3.80 (m, 3H), 3.47-3.42 (m, 1H), 2.64-2.54 (m, 1H), 2.44-2.35 (m, 1H), 2.30-2.22 (m, 2H), 2.21-2.10 (m, 2H), 2.06-1.95 (m, 1H), 1.82-1.64 (m, 1H), 1.63-1.46 (m, 1H), 1.16-1.02 (m, 1H) | 2.11 A, From isomer 2 of methyl 2-(1-(3-((2- ((4-fluoro-3- (trifluoro- methyl)phenyl) carbamoyl)-6- (trifluoro- methyl) benzo[b] thiophen-3- yl)carbamoyl)- 4- methoxy- phenyl) piperidin- 3- yl)acetate. Isomers were separated by chiral prep HPLC (Method 14) to give Isomer 1 (Chiral prep HPLC RT = 5.649 min) and Isomer 2 (Chiral prep HPLC RT = 7.242 min). *BBRC: need %ee |
| 65 | 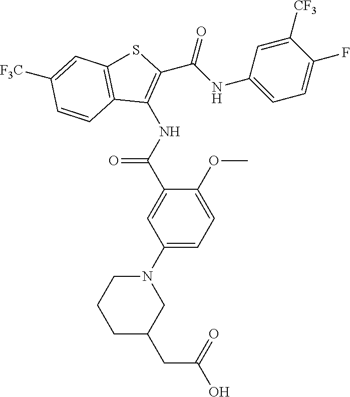 [SMILES: COc1ccc(N2CCCC(CC(=O)O)C2)cc1C(=O)Nc1c(C(=O)Nc2ccc(F)c(C(F)(F)F)c2)sc2cc(C(F)(F)F)ccc12] | 2-(1-(3-((2-((4- fluoro-3- (trifluoromethyl) phenyl) carbamoyl)-6- (trifluoromethyl) benzo[b] thiophen-3- yl)carbamoyl)- 4- methoxyphenyl) piperidin-3- yl)acetic acid | 698.2 | 1H NMR (400 MHz, DMSO-d6) δ ppm 10.91-10.75 (m, 2H), 8.66 (s, 1H), 8.17- 8.13 (m, 1H), 8.10- 8.03 (m, 1H), 8.01- 7.93 (m, 1H), 7.84- 7.78 (m, 1H), 7.62- 7.48 (m, 1H), 7.43- 7.36 (m, 2H), 7.20- 7.07 (m, 1H), 3.94- 3.86 (m, 3H), 3.48- 3.44 (m, 1H), 2.59 (br d, J = 1.0 Hz, 1H), 2.42-2.36 (m, 1H), 2.29-2.23 (m, 2H), 2.19-2.13 (m, 2H), 2.02-1.97 (m, 1H), 1.79-1.66 (m, 1H), 1.61-1.52 (m, 1H), 1.14-1.05 (m, 1H) | 2.11 A From isomer 1 of methyl 2-(1-(3-((2- ((4-fluoro-3- (trifluoro- methyl)phenyl) carbamoyl)-6- (trifluoro- methyl) benzo[b] thiophen-3- yl)carbamoyl)- 4- methoxyphenyl) piperidin- 3-yl)acetate. Isomers were separated by chiral prep HPLC (Method 14) to generate Isomer 1 (Chiral prep HPLC RT = 5.649 min) and Isomer 2 (Chiral prep HPLC RT = 7.242 min). *BBRC: need %ee |
| 66 | 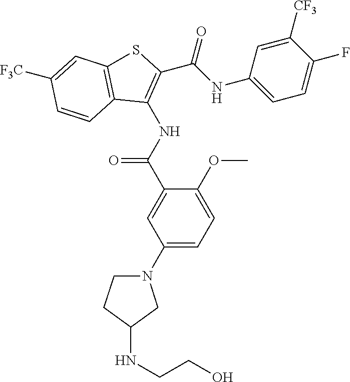 [SMILES: COc1ccc(N2CCC(NCCO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (3-((2- hydroxyethyl) amino) pyrrolidin- 1-yl)-2- methoxy- benzamido)-6- (trifluoromethyl) benzo[b] thiophene-2- carboxamide | 685.2 | 1H NMR (400 MHz, DMSO-d6) δ ppm 10.85 (br d, J = 2.2 Hz, 1H), 10.73 (br s, 1H), 8.66 (s, 1H), 8.16 (dd, J = 2.4, 6.6 Hz, 1H), 8.07-7.93 (m, 2H), 7.81 (dd, J = 1.3, 8.7 Hz, 2H), 7.54 (t, J = 9.9 Hz, 1H), 7.10 (d, J = 2.7 Hz, 1H), 7.04 (d, J = 8.8 Hz, 1H), 6.79 (dd, J = 3.1, 9.2 Hz, 1H), 5.66-5.54 (m, 1H), 4.57-4.35(m, 1H), 3.95-3.75 (m, 5H), 3.49 (br d, J = 4.9 Hz, 3H), 2.92-2.82 (m, 2H), 2.68 (br s, 1H), 2.18-2.09 (m, 1H), 1.60-1.48 (m, 1H) | 1.99 B Example prepared from Intermediate 9-5. |
| 67 | 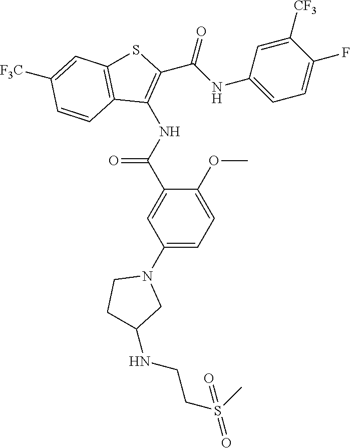 [SMILES: COc1ccc(N2CCC(NCCS(C)(=O)=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(3- ((2- (methylsulfonyl) ethyl)amino) pyrrolidin-1- yl)benzamido)- 6- (trifluoromethyl) benzo[b] thiophene-2- carboxamide | 747.2 | 1H NMR (400 MHz, DMSO-d6) δ ppm 10.89 (s, 1H), 10.77 (s, 1H), 8.68 (s, 1H), 8.19- 8.12 (m, 1H), 8.03- 7.95 (m, 2H), 7.81 (dd, J = 1.6, 8.9 Hz, 1H), 7.60-7.50 (m, 1H), 7.23-7.16 (m, 1H), 7.14-7.08 (m, 1H), 7.02-6.92 (m, 1H) 6.88-6.81 (m, 1H), 4.45-4.35(m, 1H), 3.87 (s, 3H), 3.81- 3.76 (m, 1H), 3.91 (s, 3H), 3.62 (br d, J = 4.2 Hz, 3H), 3.09 (s, 3H) 2.36-2.32 (m, 1H), 2.06-2.01 (m, 2H) | 2.33 A. Example prepared from Intermediate 9-4. |
| 68 | 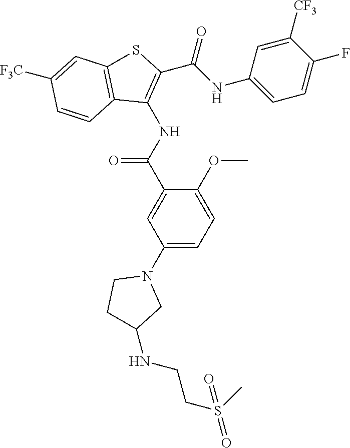 [SMILES: COc1ccc(N2CCC(NCCS(C)(=O)=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(3- ((2- (methylsulfonyl) ethyl)amino) pyrrolidin-1- yl)benzamido)- 6- (trifluoromethyl) benzo[b] thiophene-2- carboxamide | 747.2 | 1H NMR (400 MHz, DMSO-d6) δ ppm 10.89 (s, 1H), 10.77 (s, 1H), 8.68 (s, 1H), 8.22- 8.13 (m, 1H), 8.06- 7.92 (m, 2H), 7.81 (dd, J = 1.7, 8.8 Hz, 1H), 7.55 (t, J = 10.0 Hz, 1H), 7.23-7.16 (m, 1H), 7.15-7.09 (m, 1H), 7.05-6.92 (m, 1H) 6.84 (br dd, J = 2.9, 9.0 Hz, 1H), 4.20- 4.06 (m, 1H), 3.87 (s, 3H), 3.91 (s, 2H), 3.81-3.72 (3H), 3.65-3.57 (m, 3H), 3.09 (s, 2H), 2.39-2.34 (m, 1H), 2.08-2.04 (m, 2H) | 2.33 A. Example prepared from Intermediate 9-5. |
| 69 | 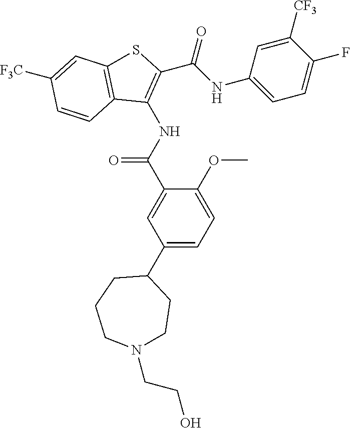 [SMILES: COc1ccc(C2CCCN(CCO)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(2- hydroxyethyl) azepan-4-yl)-2- methoxy- benzamido)-6- (trifluoromethyl) benzo[b] thiophene-2- carboxamide | 698.2 | 1H NMR (400 MHz, DMSO-d6) δ ppm 10.99-10.68 (m, 2H), 8.65 (s, 1H), 8.13 (dd, J = 2.8, 6.5 Hz, 1H), 8.07 (d, J = 8.8 Hz, 1H), 8.01-7.91 (m, 1H), 7.81 (dd, J = 1.6, 8.7 Hz, 1H), 7.61 (br d, J = 1.7 Hz, 1H), 7.53 (t, J = 9.8 Hz, 1H), 7.40 (br dd, J = 1.7, 8.6 Hz, 1H), 7.17- 7.08 (m, 1H), 4.39- 4.13 (m, 1H), 3.95- 3.88 (m, 3H), 3.47 (br t, J = 6.5 Hz, 3H), 2.83- 2.62 (m, 5H), 2.57 (t, J = 6.4 Hz, 2H), 1.81- 1.57 (m, 6H) | 2.17 A Example was prepared from Example 39 |
| 70 | 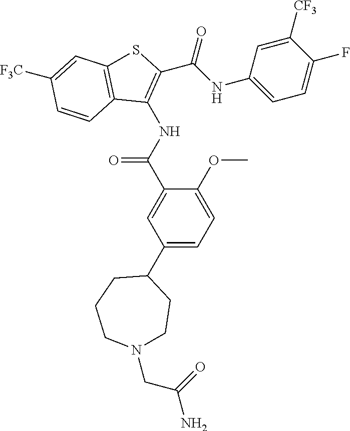 [SMILES: COc1ccc(C2CCCN(CC(N)=O)CC2)cc1C(=O)Nc1c(C(=O)Nc2ccc(F)c(C(F)(F)F)c2)sc2cc(C(F)(F)F)ccc12] | 3-(5-(1-(2- amino-2- oxoethyl) azepan-4-yl)-2- methoxy- benzamido)-N-(4- fluoro-3- (trifluoromethyl) phenyl)-6- (trifluoromethyl) benzo[b] thiophene-2- carboxamide | 711.2 | 1H NMR (400 MHz, DMSO-d6) δ ppm 10.92-10.58 (m, 2H), 8.67 (s, 1H), 8.15 (dd, J = 2.5, 6.5 Hz, 1H), 8.07 (d, J = 8.5 Hz, 1H), 8.03-7.94 (m, 1H), 7.82 (dd, J = 1.5, 8.5 Hz, 1H), 7.66 (d, J = 2.0 Hz, 1H), 7.55 (t, J = 9.8 Hz, 1H), 7.44 (dd, J = 2.3, 8.3 Hz, 1H), 7.25-7.13 (m, 2H), 7.11 (br s, 1H), 3.94 (s, 3H), 3.03 (d, J = 2.5 Hz, 2H), 2.89- 2.78 (m, 1H), 2.77- 2.68 (m, 4H), 1.85- 1.62 (m, 6H). | 2.40 A Example was prepared from Example 39. |
| 71 | 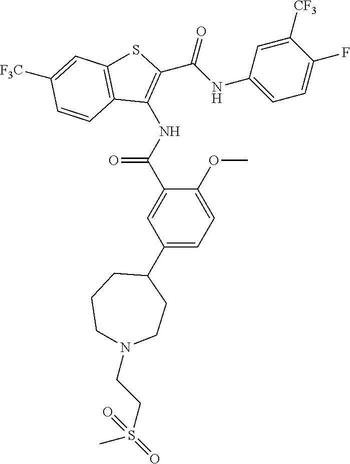 [SMILES: COc1ccc(C2CCCN(CCS(C)(=O)=O)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (2- (methylsulfonyl) ethyl)azepan- 4- yl)benzamido)- 6- (trifluoromethyl) benzo[b] thiophene-2- carboxamide | 760.2 | 1H NMR (400 MHz, DMSO-d6) δ ppm 10.84-10.66 (m, 2H), 8.66 (s, 1H), 8.14 (dd, J = 2.6, 6.5 Hz, 1H), 8.06 (d, J = 8.8 Hz, 1H), 8.02-7.92 (m, 1H), 7.82 (dd, J = 1.7, 8.8 Hz, 1H), 7.62 (d, J = 2.2 Hz, 1H), 7.54 (t, J = 9.8 Hz, 1H), 7.40 (dd, J = 2.3, 8.7 Hz, 1H), 7.15 (d, J = 8.8 Hz, 1H), 3.93 (s, 3H), 3.26 (dt, J = 2.6, 6.8 Hz, 2H), 3.03 (s, 3H), 2.89 (t, J = 6.8 Hz, 2H), 2.80-2.62 (m, 5H), 1.85-1.57 (m, 6H). | 2.50 A Example was prepared from Intermediate 39-7. |
| 72 | 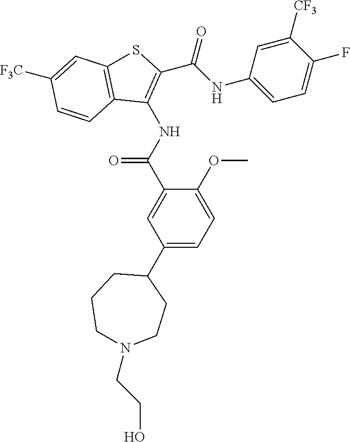 [SMILES: COc1ccc(C2CCCN(CCO)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(2- hydroxyethyl) azepan-4-yl)-2- methoxy- benzamido)-6- (trifluoromethyl) benzo[b] thiophene-2- carboxamide | 698.3 | 1H NMR (400 MHz, DMSO-d6) δ ppm 11.04-10.64 (m, 2H), 8.64 (s, 1H), 8.13 (dd, J = 2.6, 6.5 Hz, 1H), 8.07 (d, J = 8.6 Hz, 1H), 8.00-7.92 (m, 1H), 7.81 (dd, J = 1.6, 8.9 Hz, 1H), 7.61 (br s, 1H), 7.53 (t, J = 9.8 Hz, 1H), 7.39 (dd, J = 2.1, 8.4 Hz, 1H), 7.13 (d, J = 8.3 Hz, 1H), 5.89 (br, s ,1H) 3.91 (br s, 3H), 3.49-3.46 (m, 2H), 2.81-2.63 (m, 4H), 2.57 (t, J = 6.4 Hz, 2H), 1.89 (s, 1H), 1.80-1.52 (m, 6H) | 2.15 A Example was prepared from Intermediate 39-7. |
| 73 | 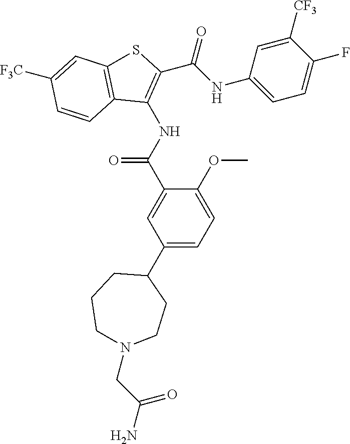 [SMILES: COc1ccc(C2CCCN(CC(N)=O)CC2)cc1C(=O)Nc1c(C(=O)Nc2ccc(F)c(C(F)(F)F)c2)sc2cc(C(F)(F)F)ccc12] | 3-(5-(1-(2- amino-2- oxoethyl)azepan- 4-yl)-2- methoxy- benzamido)-N-(4- fluoro-3- (trifluoromethyl) phenyl)-6- (trifluoromethyl) benzo[b] thiophene-2- carboxamide | 711.3 | 1H NMR (400 MHz, DMSO-d6) δ ppm 10.89-10.64 (m, 2H), 8.66 (s, 1H), 8.14 (dd, J = 2.7, 6.6 Hz, 1H), 8.06 (d, J = 8.6 Hz, 1H), 8.02-7.93 (m, 1H), 7.82 (dd,J = 1.5, 8.8 Hz, 1H), 7.65 (d, J = 2.0 Hz, 1H), 7.54 (t, J = 9.9 Hz, 1H), 7.43 (dd, J = 2.0, 8.6 Hz, 1H), 7.25-7.02 (m, 3H), 3.93 (s, 3H), 3.02 (d, J = 2.7 Hz, 2H), 2.81 (br dd, J = 1.3, 2.3 Hz, 1H), 2.75- 2.67 (m, 4H), 1.83- 1.57 (m, 6H) | 2.40 A Example was prepared from Intermediate 39-7. |
| 74 | 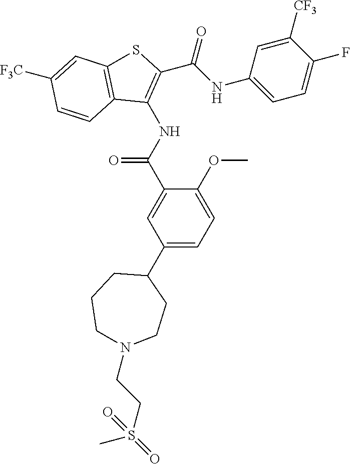 [SMILES: COc1ccc(C2CCCN(CCS(C)(=O)=O)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (2- (methylsulfonyl) ethyl)azepan- 4- yl)benzamido)- 6- (trifluoromethyl) benzo[b] thiophene-2- carboxamide | 760.2 | 1H NMR (400 MHz, DMSO-d6) δ ppm 10.82-10.63 (m, 2H), 8.66 (s, 1H), 8.17- 8.11 (m, 1H), 8.06 (d, J = 8.3 Hz, 1H), 8.02- 7.94 (m, 1H), 7.85- 7.76 (m, 1H), 7.62 (s, 1H), 7.54 (t, J = 9.8 Hz, 1H), 7.46-7.35 (m, 1H), 7.15 (d, J = 9.0 Hz, 1H), 3.93 (s, 3H), 3.26 (br d, J = 2.4 Hz, 2H), 3.03 (s, 3H), 2.90 (t, J = 7.0 Hz, 2H), 2.79-2.64 (m, 5H), 1.85-1.56 (m, 6H). | 2.50 A Example was prepared from Example 39. |
| 75 | 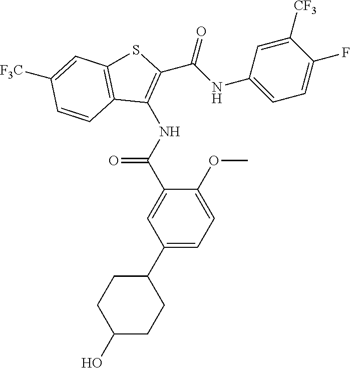 [SMILES: COc1ccc(C2CCC(O)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (4- hydroxycyclo- hexyl)-2- methoxy- benzamido)-6- (trifluoromethyl) benzo[b] thiophene-2- carboxamide | 653.2 | 1H NMR (400 MHz, DMSO-d6) δ ppm 10.57-11.09 (m, 2 H) 8.42-8.78 (m, 1 H) 7.99-8.23 (m, 2 H) 7.71-7.97 (m, 1 H) 7.55-7.68 (m, 1 H) 7.34-7.50 (m, 2 H) 7.01-7.26 (m, 1 H) 7.05-7.10 (m, 1 H) 4.27-4.58 (m, 2 H) 3.89 (br s, 3 H) 3.41 (br d, J = 19.58 Hz, 1 H) 2.52-2.61 (m, 1 H) 1.83-1.95 (m, 1 H) 1.69-1.82 (m, 2 H) 1.67-2.00 (m, 2H) 1.43-1.60 (m, 1 H) 1.09-1.16 (m, 1 H) | 3.93 C The diastereomers were separated by prep HPLC (Method 3) to give Example 75 (second eluting, RT = 18.94 min). 98% ee |
| 76 | 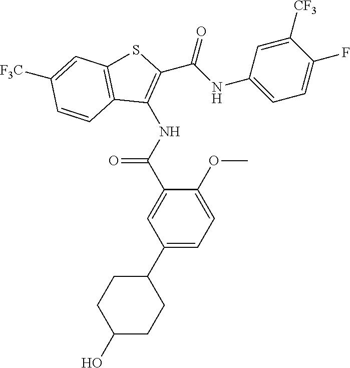 [SMILES: COc1ccc(C2CCC(O)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (4- hydroxycyclo- hexyl)-2- methoxy- benzamido)-6- (trifluoromethyl) benzo[b] thiophene-2- carboxamide | 653.2 | 1H NMR (400 MHz, DMSO-d6) δ ppm 10.51-11.04 (m, 2 H) 8.67 (s, 1 H) 8.13 (dd, J = 6.02, 2.51 Hz, 1 H) 8.08 (d, J = 8.53 Hz, 1 H) 7.83 (d, J = 8.84 Hz, 1 H) 7.52-7.62 (m, 2 H) 7.42 (br d, J = 8.78 Hz, 2 H) 7.15 (d, J = 8.53 Hz, 1 H) 4.57 (d, J = 4.02 Hz, 2 H) 3.93 (s, 3 H) 2.53- 2.53 (m, 1 H) 1.90 (br d, J = 9.04 Hz, 2 H) 1.73 (br d, J = 11.55 Hz, 2 H) 1.40 (br d, J = 13.05 Hz, 2 H) 1.28 (br d, J = 11.55 Hz, 2 H) | 3.74 C The diastereomers were separated by prep HPLC (Method 3) to give Example 76 (first eluting, RT = 16.68 min). 96% ee |
| 77 | 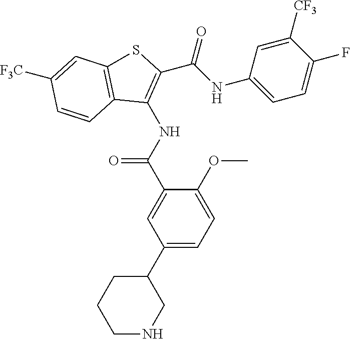 [SMILES: COc1ccc(C2CCCNC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5- (piperidin-3- yl)benzamido)- 6- (trifluoromethyl) benzo[b] thiophene-2- carboxamide | 640.2 | ¹H NMR (400 MHz, DMSO-d₆) δ ppm 8.68- 8.63 (m, 1H), 8.18- 8.12 (m, 1H), 8.10- 8.05 (m, 1H), 8.00- 7.94 (m, 1H), 7.83- 7.77 (m, 1H), 7.68- 7.62 (m, 1H), 7.58- 7.50 (m, 1H), 7.47- 7.40 (m, 1H), 7.22- 7.13 (m, 1H), 3.96- 3.87 (m, 4H), 3.10- 3.01 (m, 2H), 2.75- 2.55 (m, 3H), 1.86- 1.78 (m, 1H), 1.78- 1.67 (m, 1H), 1.62- 1.50 (m, 2H). | 2.11 A The isomers were separated by prep SFC (Method 42) to give isomer 1, Intermediate 77-1 (Prep SFC RT = 14.2 mins, 97.2% ee) and isomer 2, Example 77 (Prep SFC RT = 16.0 mins). 100% ee Analytical SFC (Method 43.). |
| 78 | 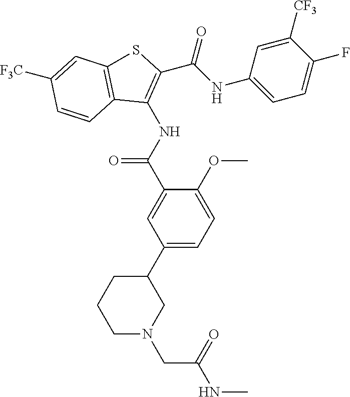 [SMILES: CNC(=O)CN1CCCC(c2ccc(OC)c(C(=O)Nc3c(C(=O)Nc4ccc(F)c(C(F)(F)F)c4)sc4cc(C(F)(F)F)ccc34)c2)C1] | N-(4-fluoro-3- (trifluoromethyl) phenyl)-3-(2- methoxy-5-(1- (2- (methylamino)- 2- oxoethyl) piperidin-3- yl)benzamido)- 6- (trifluoromethyl) benzo[b] thiophene-2- carboxamide | 711.3 | 1H NMR (400 MHz, DMSO-d6) δ ppm 10.87-10.68 (m, 2H), 8.66 (s, 1H), 8.15 (dd, J = 2.6, 6.7 Hz, 1H), 8.07 (d, J = 8.6 Hz, 1H), 8.01-7.92 (m, 1H), 7.82 (dd, J = 1.7, 8.8 Hz, 1H), 7.72- 7.61 (m, 2H), 7.54 (t, J = 9.7 Hz, 1H), 7.43 (dd, J = 2.2, 8.6 Hz, 1H), 7.17 (d, J = 8.8 Hz, 1H), 3.93 (s, 3H), 2.90 (s, 2H), 2.86- 2.73 (m, 3H), 2.61 (d, J = 4.6 Hz, 3H), 2.14- 1.96 (m, 2H), 1.82- 1.73 (m, 1H), 1.71- 1.54 (m, 2H), 1.43- 1.26 (m, 1H) | 2.45 A Example was prepared from Example 77. |
| 79 | 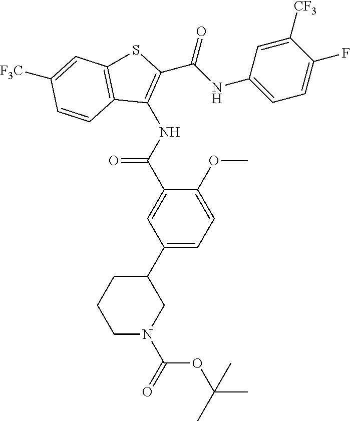 [SMILES: COc1ccc(C2CCCN(C(=O)OC(C)(C)C)C2)cc1C(=O)Nc1c(C(=O)Nc2ccc(F)c(C(F)(F)F)c2)sc2cc(C(F)(F)F)ccc12] | tert-butyl 3-(3- ((2-((4-fluoro- 3- (trifluoromethyl) phenyl) carbamoyl)-6- (trifluoromethyl) benzo[b] thiophen-3- yl)carbamoyl)- 4- methoxyphenyl) piperidine-1- carboxylate | 738.2 | 1H NMR (400 MHz, DMSO-d6) δ ppm 10.85-10.65 (m, 2H), 8.66 (s, 1H), 8.15 (br d, J = 3.9 Hz, 1H), 8.08 (d, J = 8.8 Hz, 1H), 8.03-7.96 (m, 1H), 7.82 (dd, J = 1.6, 8.7 Hz, 1H), 7.66 (d, J = 2.2 Hz, 1H), 7.54 (t, J = 9.9 Hz, 1H), 7.46 (dd, J = 2.2, 8.8 Hz, 1H), 7.19 (d, J = 8.8 Hz, 1H), 3.93 (s, 5H), 2.89-2.69 (m, 2H), 2.64-2.54 (m, 2H), 1.83 (br s, 1H), 1.75-1.65 (m, 1H), 1.63-1.52 (m, 1H), 1.49-1.25 (m, 9H) | 2.88 A Example was prepared from Example 77. |
| 80 | 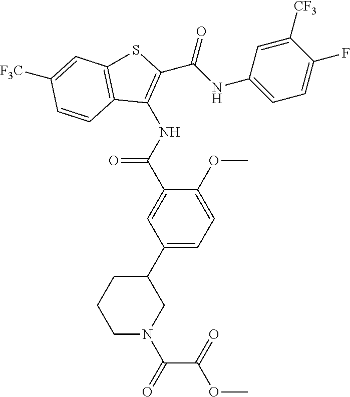 [SMILES: COC(=O)C(=O)N1CCCC(c2ccc(OC)c(C(=O)Nc3c(C(=O)Nc4ccc(F)c(C(F)(F)F)c4)sc4cc(C(F)(F)F)ccc34)c2)C1] | methyl 2-(3-(3- ((2-((4-fluoro- 3- (trifluoromethyl) phenyl) carbamoyl)-6- (trifluoromethyl) benzo[b] thiophen-3- yl)carbamoyl)- 4- methoxyphenyl) piperidin-1- yl)-2- oxoacetate | 724.1 | 1H NMR (400 MHz, DMSO-d6) δ ppm 10.82 (d, J = 7.1 Hz, 1H), 10.74 (s, 1H), 8.67 (s, 1H), 8.15 (ddd, J = 2.6, 4.3, 6.8 Hz, 1H), 8.07 (dd, J = 5.5, 8.7 Hz, 1H), 8.02- 7.94 (m, 1H), 7.82 (dd, J = 1.5, 8.6 Hz, 1H), 7.71 (dd, J = 2.4, 13.4 Hz, 1H), 7.60-7.41 (m, 2H), 7.21 (d, J = 8.6 Hz, 1H), 4.34- 4.20 (m, 1H), 3.93 (s, 3H), 3.86-3.72 (m, 3H), 3.58-3.42 (m, 1H), 3.31-3.14 (m, 2H), 2.87-2.65 (m, 2H), 1.96-1.67 (m, 2H), 1.56-1.43 (m, 1H) | 2.51 A Example was prepared from Example 77. |
| 81 | 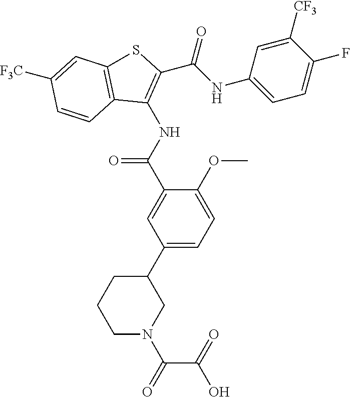 [SMILES: COc1ccc(C2CCCN(C(=O)C(=O)O)C2)cc1C(=O)Nc1c(C(=O)Nc2ccc(F)c(C(F)(F)F)c2)sc2cc(C(F)(F)F)ccc12] | 2-(3-(3-((2-((4- fluoro-3- (trifluoromethyl) phenyl) carbamoyl)-6- (trifluoromethyl) benzo[b] thiophen-3- yl)carbamoyl)- 4- methoxyphenyl) piperidin-1- yl)-2-oxoacetic acid | 711.2 | 1H NMR (400 MHz, DMSO-d6) δ ppm 10.89-10.80 (m, 1H), 10.75 (s, 1H), 8.66 (s, 1H), 8.17 (dd, J = 2.7, 6.6 Hz, 1H), 8.11- 8.04 (m, 1H), 8.02- 7.94 (m, 1H), 7.86- 7.77 (m, 1H), 7.71 (dd, J = 2.3, 12.6 Hz, 1H), 7.59-7.37 (m, 2H), 7.28-7.15 (m, 1H), 4.31-4.23 (m, 1H), 3.92 (d, J = 3.7 Hz, 3H), 3.57 (br s, 1H), 3.21-3.16 (m, 2H), 2.77-2.63 (m, 2H), 1.95-1.84 (m, 1H), 1.83-1.63 (m, 2H), 1.59-1.40 (m, 1H). | 1.93 A Example was prepared from Example 80. |
| 82 | 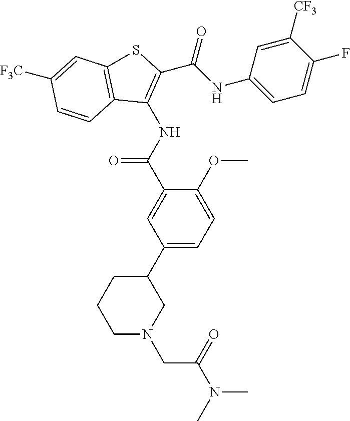 [SMILES: COc1ccc(C2CCCN(CC(=O)N(C)C)C2)cc1C(=O)Nc1c(C(=O)Nc2ccc(F)c(C(F)(F)F)c2)sc2cc(C(F)(F)F)ccc12] | 3-(5-(1-(2- (dimethylamino)- 2- oxoethyl) piperidin-3-yl)-2- methoxy- benzamido)-N-(4- fluoro-3- (trifluoromethyl) phenyl)-6- (trifluoromethyl) benzo[b] thiophene-2- carboxamide | 725.4 | 1H NMR (400 MHz, DMSO-d6) δ ppm 10.89-10.68 (m, 2H), 8.66 (s, 1H), 8.15 (dd, J = 2.7, 6.4 Hz, 1H), 8.07 (d, J = 8.8 Hz, 1H), 8.02-7.94 (m, 1H), 7.82 (d, J = 8.8 Hz, 1H), 7.65 (d, J = 2.0 Hz, 1H), 7.55 (t, J = 9.8 Hz, 1H), 7.45 (dd, J = 1.6, 8.7 Hz, 1H), 7.16 (d, J = 8.8 Hz, 1H), 3.99-3.89 (m, 5H), 3.23-3.11 (m, 2H), 3.02 (s, 3H), 2.89-2.68 (m, 5H), 2.16-2.02 (m, 2H), 1.81-1.72 (m, 1H), 1.72-1.63 (m, 1H), 1.63-1.49 (m, 1H) | 2.42 A Example was prepared from Example 77. |
| 83 | 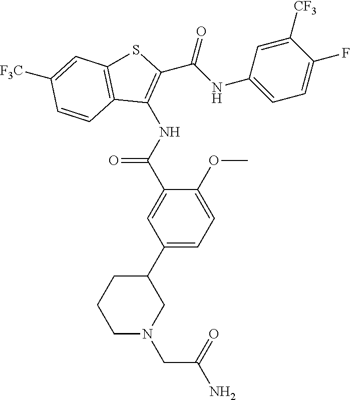 [SMILES: COc1ccc(C2CCCN(CC(N)=O)C2)cc1C(=O)Nc1c(C(=O)Nc2ccc(F)c(C(F)(F)F)c2)sc2cc(C(F)(F)F)ccc12] | 3-(5-(1-(2- amino-2- oxoethyl) piperidin-3-yl)-2- methoxy- benzamido)-N-(4- fluoro-3- (trifluoromethyl) phenyl)-6- (trifluoromethyl) benzo[b] thiophene-2- carboxamide | 697.2 | 1H NMR (400 MHz, DMSO-d6) δ ppm 10.80 (br s, 2H), 8.66 (s, 1H), 8.15 (dd, J = 2.4, 6.4 Hz, 1H), 8.07 (d, J = 8.8 Hz, 1H), 8.02-7.93 (m, 1H), 7.88-7.78 (m, 1H), 7.65 (d, J = 2.2 Hz, 1H), 7.55 (t, J = 9.8 Hz, 1H), 7.44 (dd, J = 2.1, 8.9 Hz, 1H), 7.24- 7.14 (m, 2H), 7.12 (br s, 1H), 3.93 (s, 3H), 2.94-2.75 (m, 5H), 2.14-1.99 (m, 2H), 1.87-1.53 (m, 3H), 1.40-1.25 (m, 1H) | 2.37 A Example was prepared from Example 77. |
| 84 | 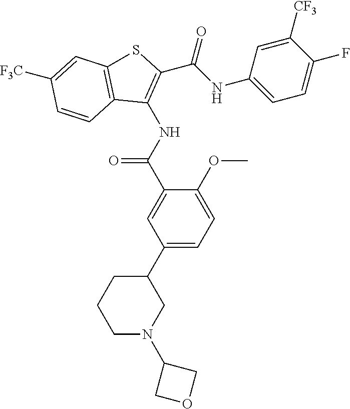 [SMILES: COc1ccc(C2CCCN(C3COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (oxetan-3- yl)piperidin-3- yl)benzamido)- 6- (trifluoromethyl) benzo[b] thiophene-2- carboxamide | 696.2 | 1H NMR (400 MHz, DMSO-d6) δ ppm 10.84-10.64 (m, 2H), 8.67 (s, 1H), 8.14 (dd, J = 2.6, 6.5 Hz, 1H), 8.07 (d, J = 8.6 Hz, 1H), 8.03-7.95 (m, 1H), 7.83 (dd, J = 1.6, 8.7 Hz, 1H), 7.65 (d, J = 2.2 Hz, 1H), 7.55 (t, J = 9.8 Hz, 1H), 7.47 (dd, J = 2.2, 8.6 Hz, 1H), 7.17 (d, J = 8.6 Hz, 1H), 4.57-4.38 (m, 5H), 3.94 (s, 3H), 2.80-2.66 (m, 4H), 1.86-1.67 (m, 4H), 1.65-1.52 (m, 1H). | 2.49 A Example was prepared from Example 77. |
| 85 | 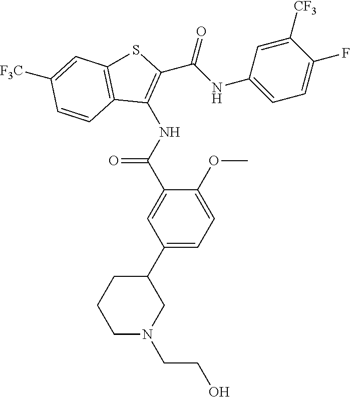 [SMILES: COc1ccc(C2CCCN(CCO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(2- hydroxyethyl) piperidin-3-yl)- 2- methoxy- benzamido)-6- (trifluoromethyl) benzo[b] thiophene-2- carboxamide | 684.2 | 1H NMR (400 MHz, DMSO-d6) δ ppm 10.86-10.67 (m, 2H), 8.66 (s, 1H), 8.15 (dd, J = 2.7, 6.6 Hz, 1H), 8.10-8.04 (m, 1H), 8.02-7.93 (m, 1H), 7.82 (dd, J = 1.7, 8.8 Hz, 1H), 7.65 (d, J = 2.0 Hz, 1H), 7.54 (t, J = 9.8 Hz, 1H), 7.48- 7.35 (m, 1H), 7.16 (d, J = 8.6 Hz, 1H), 4.46- 4.33 (m, 1H), 3.93 (s, 3H), 3.50 (br t, J = 6.1 Hz, 2H), 2.95-2.83 (m, 2H), 2.79-2.68 (m, 1H), 2.42 (br t, J = 5.6 Hz, 2H), 2.05- 1.93 (m, 2H), 1.81- 1.71 (m, 1H), 1.71- 1.63 (m, 1H), 1.63- 1.48(m, 1H), 1.40- 1.26 (m, 1H) | 2.26 A Example was prepared from Example 77. |
| 86 | 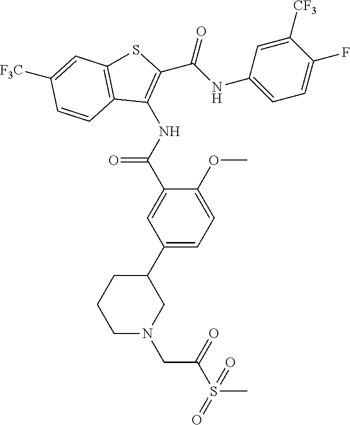 [SMILES: COc1ccc(C2CCCN(CC(=O)S(C)(=O)=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (2- (methylsulfonyl) ethyl)piperidin- 3- yl)benzamido)- 6- (trifluoromethyl) benzo[b] thiophene-2- carboxamide | 746.2 | 1H NMR (400 MHz, DMSO-d6) δ ppm 10.85-10.66 (m, 2H), 8.67 (s, 1H), 8.15 (dd, J = 2.7, 6.6 Hz, 1H), 8.07 (d, J = 8.8 Hz, 1H), 8.02-7.93 (m, 1H), 7.82 (dd, J = 1.6, 8.7 Hz, 1H), 7.66 (d, J = 2.2 Hz, 1H), 7.55 (t, J = 9.8 Hz, 1H), 7.47 (dd, J = 2.2, 8.6 Hz, 1H), 7.17 (d, J = 8.8 Hz, 1H), 3.94 (s, 3H), 3.31-3.24 (m,2H), 3.02 (s, 3H), 2.92 (br d, J = 11.0 Hz, 2H), 2.72 (br t, J = 6.7 Hz, 3H), 2.03-1.90 (m, 2H), 1.83-1.64 (m, 2H), 1.62-1.47 (m, 1H), 1.40-1.25 (m, 1H) | 2.46 A Example was prepared from Example 77. |
| 87 | 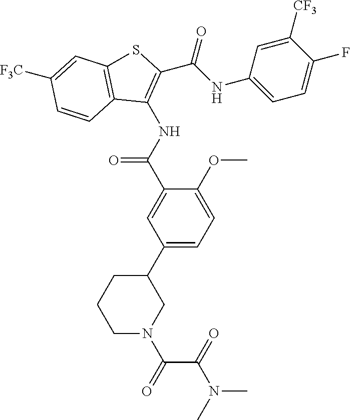 [SMILES: COc1ccc(C2CCCN(C(=O)C(=O)N(C)C)C2)cc1C(=O)Nc1c(C(=O)Nc2ccc(F)c(C(F)(F)F)c2)sc2cc(C(F)(F)F)ccc12] | 3-(5-(1- (dimethylglycyl) piperidin-3- yl)-2- methoxy- benzamido)-N-(4- fluoro-3- (trifluoromethyl) phenyl)-6- (trifluoromethyl) benzo[b] thiophene-2- carboxamide | 725.2 | 1H NMR (400 MHz, DMSO-d6) δ ppm 10.88-10.66 (m, 2H), 8.66 (s, 1H), 8.22-8.14 (m, 1H), 8.11-8.03 (m, 1H), 8.02-7.95 (m, 1H), 7.82 (d, J = 8.8 Hz, 1H), 7.75-7.66 (m, 1H), 7.55 (t, J = 9.7 Hz, 1H), 7.50-7.40 (m, 1H), 7.20 (br t, J = 8.2 Hz, 1H), 4.48-4.33 (m, 1H), 3.94 (br d, J = 8.1 Hz, 4H), 3.25 (br d, J = 13.2 Hz, 1H), 3.14-2.89 (m, 2H), 2.71 (br d, J = 1.2 Hz, 1H), 2.63-2.54 (m, 2H), 2.20 (br d, J = 17.1 Hz, 6H), 1.95-1.81 (m, 1H), 1.78-1.59 (m, 2H) | 2.30 A Example was prepared from Example 77. |
| 88 | 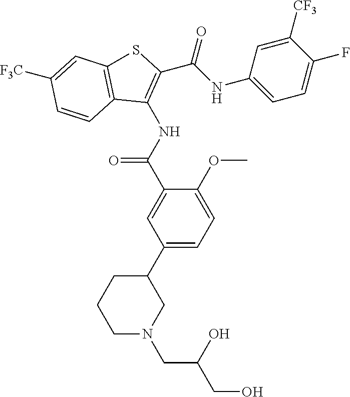 [SMILES: COc1ccc(C2CCCN(CC(O)CO)C2)cc1C(=O)Nc1c(C(=O)Nc2ccc(F)c(C(F)(F)F)c2)sc2cc(C(F)(F)F)ccc12] | 3-(5-(1-(2,3- dihydroxypropyl) piperidin-3- yl)-2- methoxy- benzamido)-N-(4- fluoro-3- (trifluoromethyl) phenyl)-6- (trifluoromethyl) benzo[b] thiophene-2- carboxamide | 714.2 | 1H NMR (400 MHz, DMSO-d6) δ ppm 10.81 (br d, J = 1.2 Hz, 1H), 10.74 (br s, 1H), 8.67 (s, 1H), 8.19-8.12 (m, 1H), 8.07 (d, J = 8.1 Hz, 1H), 8.01-7.94 (m, 1H), 7.82 (dd, J = 1.7, 8.6 Hz, 1H), 7.69- 7.64 (m, 1H), 7.55 (t, J = 9.7 Hz, 1H), 7.46 (dd, J = 1.7, 8.8 Hz, 1H), 7.18 (d, J = 8.1 Hz, 1H), 5.67-4.37 (m, 2H) 3.93 (s, 3H), 3.68-3.59 (m, 2H), 3.10-2.72 (m, 3H), 2.13-1.90 (m, 2H), 1.81-1.57 (m, 4H), 1.43-1.33 (m, 2H), 1.23 (s, 1H) | 2.18 A Example was prepared from Example 77. |
| 89 | 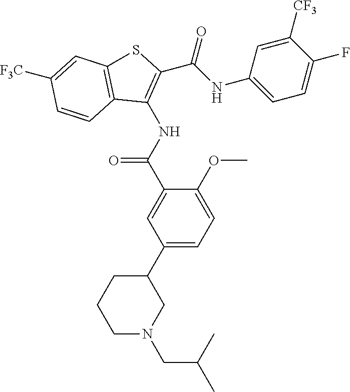 [SMILES: COc1ccc(C2CCCN(CC(C)C)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1- isobutylpiperidin- 3-yl)-2- methoxy- benzamido)-6- (trifluoromethyl) benzo[b] thiophene-2- carboxamide | 696.3 | 1H NMR (400 MHz, DMSO-d6) δ ppm 10.79 (br s, 1H), 10.73 (br s, 1H), 8.67 (s, 1H), 8.14 (dd, J = 2.6, 6.5 Hz, 1H), 8.07 (d, J = 8.6 Hz, 1H), 8.01-7.91 (m, 1H), 7.82 (dd, J = 1.7, 8.6 Hz, 1H), 7.65 (br d, J = 0.7 Hz, 1H), 7.54 (t, J = 9.8 Hz, 1H), 7.46 (dd, J = 2.1, 8.4 Hz, 1H), 7.17 (br d, J = 8.3 Hz, 1H), 3.94 (s, 3H), 3.01-2.69 (m, 4H), 2.18-1.51 (m, 7H), 1.44-1.25 (m, 1H), 0.85 (br d, J = 5.9 Hz, 6H) | 2.77 A Example was prepared from Example 77. |
| 90 | 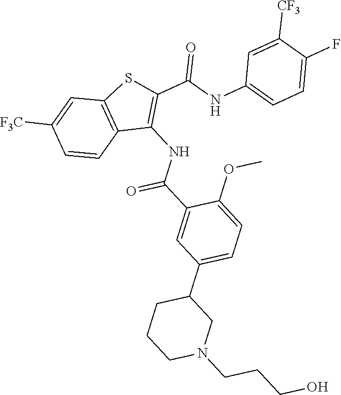 [SMILES: COc1ccc(C2CCCN(CCCO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(3- hydroxypropyl) piperidin-3-yl)- 2- methoxy- benzamido)-6- (trifluoromethyl) benzo[b] thiophene-2- carboxamide | 698.2 | 1H NMR (400 MHz, DMSO-d6) δ ppm 10.88-10.63 (m, 2H), 8.66 (s, 1H), 8.14 (dd, J = 2.6, 6.5 Hz, 1H), 8.07 (d, J = 8.6 Hz, 1H), 8.02-7.93 (m, 1H), 7.82 (dd, J = 1.6, 8.9 Hz, 1H), 7.65 (d, J = 2.0 Hz, 1H), 7.54 (t, J = 9.8 Hz, 1H), 7.46 (dd, J = 2.1, 8.7 Hz, 1H), 7.17 (d, J = 8.6 Hz, 1H), 3.93 (s, 3H), 3.58-3.56 (m, 1H), 3.45-3.43 (m, 2H), 3.02-2.86 (m, 2H), 2.78-2.67 (m, 1H), 2.46-2.35 (m, 2H), 2.07-1.90 (m, 2H), 1.85-1.66 (m, 2H), 1.65-1.51 (m, 3H), 1.45-1.26 (m, 1H) | 2.20 A Example was prepared from Example 77. |
| 91 | 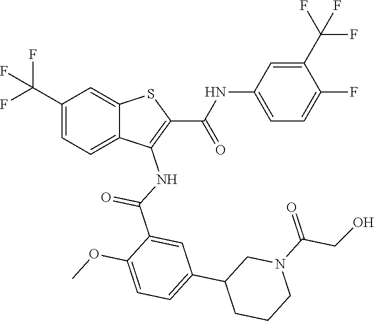 [SMILES: COc1ccc(C2CCCN(C(=O)CO)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [1-(2- hydroxyacetyl) piperidin-3-yl]- 2- methoxy- benzamido}-6- (trifluoromethyl)- 1- benzothiophene- 2- carboxamide | 698.2 | 1H NMR (400 MHz, DMSO-d6) δ ppm 10.83 (s, 1H), 10.74 (s, 1H), 8.67 (s, 1H), 8.22- 8.12 (m, 1H), 8.12- 8.03 (m, 1H), 8.03- 7.92 (m, 1H), 7.86- 7.78 (m, 1H), 7.77- 7.68 (m, 1H), 7.61- 7.44 (m, 2H), 7.21 (d, J = 8.6 Hz, 1H), 4.56- 4.46 (m, 1H), 4.46- 4.33 (m, 1H), 4.18- 4.05 (m, 2H), 3.94 (s, 3H), 3.76-3.63 (m, 1H), 3.05-2.93 (m, 1H), 2.77-2.57 (m, 2H), 1.92-1.82 (m, 1H), 1.77-1.59 (m, 2H), 1.55-1.33 (m, 1H). | 2.52 A Example was prepared from Example 77. |
| 92 | 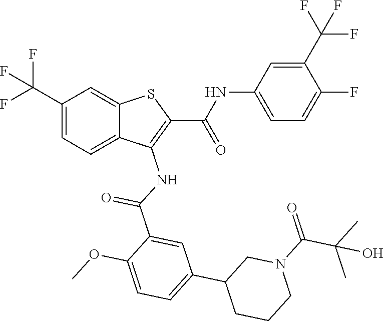 [SMILES: COc1ccc(C2CCCN(C(=O)C(C)(C)O)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [1-(2-hydroxy- 2- methylpropanoyl) piperidin-3- yl]-2- methoxy- benzamido}-6- (trifluoromethyl)- 1- benzothiophene- 2- carboxamide | 726.2 | 1H NMR (400 MHz, DMSO-d6) δ ppm 10.91-10.62 (m, 2H), 8.67 (s, 1H), 8.16 (dd, J = 6.2, 2.3 Hz, 1H), 8.08 (d, J = 8.8 Hz, 1H), 8.03-7.92 (m, 1H), 7.82 (dd, J = 8.9, 1.3 Hz, 1H), 7.68 (d, J = 2.2 Hz, 1H), 7.55 (t, J = 9.8 Hz, 1H), 7.49-7.39 (m, 1H), 7.20 (d, J = 8.6 Hz, 1H), 5.40 (s, 1H), 4.95- 4.78 (m, 1H), 4.57- 4.40 (m, 1H), 3.94 (s, 3H), 3.04-2.83 (m, 1H), 2.67 (br dd, J = 3.5, 1.8 Hz, 1H), 1.95-1.84 (m, 1H), 1.78-1.59 (m, 2H), 1.55-1.40 (m, 1H), 1.37-1.17 (m, 7H). | 2.63 A Example was prepared from Example 77. |
| 93 | 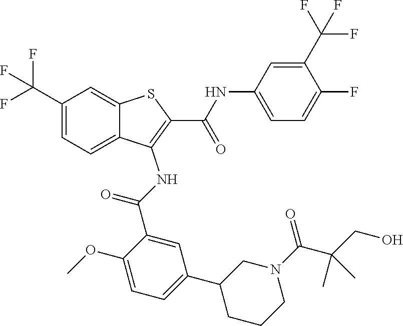 [SMILES: COc1ccc(C2CCCN(C(=O)C(C)(C)CO)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [1-(3-hydroxy- 2,2- dimethyl- propanoyl) piperidin- 3-yl]-2- methoxy- benzamido}-6- (trifluoromethyl)- 1- benzothiophene- 2- carboxamide | 740.2 | 1H NMR (400 MHz, DMSO-d6) δ ppm 10.80 (s, 1H), 10.72 (br d, J = 0.7 Hz, 1H), 8.66 (s, 1H), 8.20-8.13 (m, 1H), 8.08 (d, J = 8.3 Hz, 1H), 8.03-7.94 (m, 1H), 7.82 (d, J = 9.0 Hz, 1H), 7.68 (s, 1H), 7.54 (t, J = 10.0 Hz, 1H), 7.49- 7.40 (m, 1H), 7.21 (d, J = 8.6 Hz, 1H), 4.56- 4.48 (m, 1H), 4.37- 4.21 (m, 2H), 3.94 (s, 3H), 3.41 (br d, J = 5.6 Hz, 2H), 2.87-2.70 (m, 2H), 2.65-2.57 (m, 1H), 1.93-1.82 (m, 1H), 1.79-1.63 (m, 2H), 1.53-1.40 (m, 1H), 1.16 (d, J = 2.2 Hz, 6H). | 2.66 A Example was prepared from Example 77. |
| 94 | 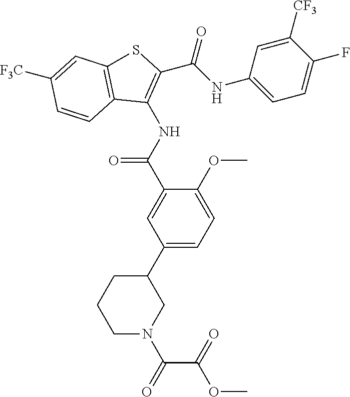 [SMILES: COC(=O)C(=O)N1CCCC(c2ccc(OC)c(C(=O)Nc3c(C(=O)Nc4ccc(F)c(C(F)(F)F)c4)sc4cc(C(F)(F)F)ccc34)c2)C1] | methyl 2-(3-(3- ((2-((4-fluoro- 3- (trifluoromethyl) phenyl) carbamoyl)-6- (trifluoromethyl) benzo[b] thiophen-3- yl)carbamoyl)- 4- methoxyphenyl) piperidin-1- yl)-2- oxoacetate | 724.3 | 1H NMR (400 MHz, DMSO-d6) δ ppm 10.80 (br s, 1H), 8.66 (s, 1H), 8.57-8.50 (m, 1H), 8.18-8.03 (m, 2H), 7.89-7.82 (m, 1H), 7.77-7.70 (m, 1H), 7.65 (br dd, J = 1.1, 13.8 Hz, 1H), 7.51- 7.36 (m, 2H), 7.16 (dd, J = 2.6, 8.4 Hz, 1H), 4.32-4.22 (m, 1H), 3.88-3.80 (m, 3H), 3.76 (s, 2H), 3.52 (br s, 1H), 3.22-3.19 (m, 1H), 2.87-2.70 (m, 2H), 1.95-1.88 (m, 1H), 1.86 (s, 1H), 1.83- 1.69 (m, 2H), 1.57- 1.45 (m, 1H) | 2.51 A Example was prepared from Intermediate 77-1. |
| 95 | 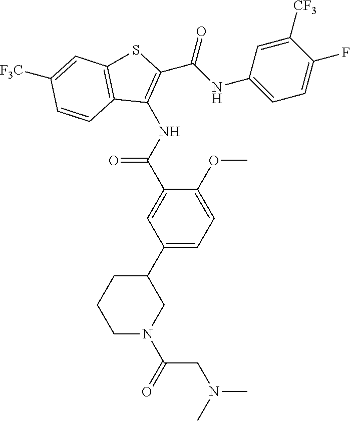 [SMILES: COc1ccc(C2CCCN(C(=O)CN(C)C)C2)cc1C(=O)Nc1c(C(=O)Nc2ccc(F)c(C(F)(F)F)c2)sc2cc(C(F)(F)F)ccc12] | 3-(5-(1- (dimethylglycyl) piperidin-3- yl)-2- methoxy- benzamido)-N-(4- fluoro-3- (trifluoromethyl) phenyl)-6- (trifluoromethyl) benzo[b] thiophene-2- carboxamide | 725.3 | 1H NMR (400 MHz, DMSO-d6) δ ppm 10.95-10.57 (m, 2H), 8.67 (s, 1H), 8.22-8.12 (m, 1H), 8.12-8.03 (m, 1H), 8.03-7.92 (m, 1H), 7.82 (dd, J = 1.5, 8.5 Hz, 1H), 7.76-7.66 (m, 1H), 7.55 (t, J = 9.8 Hz, 1H), 7.48 (br d, J = 8.0 Hz, 1H), 7.21 (br t, J = 7.8 Hz, 1H), 4.48- 4.34 (m, 1H), 4.15- 4.02 (m, 1H), 3.95(br d, J = 7.0 Hz, 3H), 3.22- 3.15 (m, 2H), 3.08- 2.82 (m, 3H), 2.72 (br s, 2H), 2.63-2.54 (m, 2H), 2.18 (d, J = 17.1 Hz, 6H) | 2.31 A Example was prepared from Intermediate 77-1. |
| 96 | 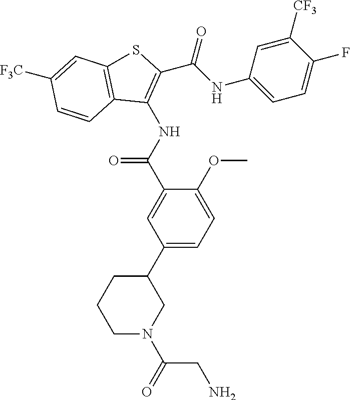 [SMILES: COc1ccc(C2CCCN(C(=O)CN)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1- glycylpiperidin- 3-yl)-2- methoxy- benzamido)-6- (trifluoromethyl) benzo[b] thiophene-2- carboxamide | 697.2 | 1H NMR (400 MHz, DMSO-d6) δ ppm 10.90-10.63 (m, 2H), 8.66 (s, 1H), 8.16 (dd, J = 2.6, 6.5 Hz, 1H), 8.07 (d, J = 8.6 Hz, 1H), 7.98 (td, J = 4.3, 7.5 Hz, 1H), 7.82 (dd, J = 1.8, 8.9 Hz, 1H), 7.66 (d, J = 2.2 Hz, 1H), 7.55 (t, J = 9.7 Hz, 1H), 7.44 (dd, J = 2.3, 8.7 Hz, 1H), 7.28- 7.04 (m, 3H), 3.93 (s, 3H), 2.93-2.72 (m, 5H), 2.15-1.98 (m, 2H), 1.83-1.73 (m, 1H), 1.73-1.57 (m, 2H), 1.41-1.25 (m, 1H) | 2.37 A Example was prepared from Intermediate 77-1. |
| 97 | 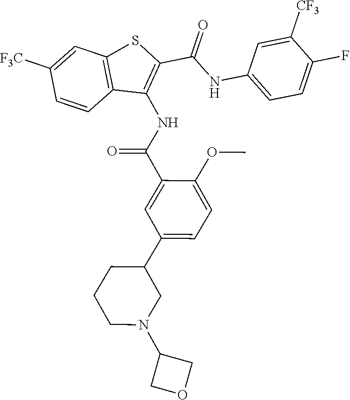 [SMILES: COc1ccc(C2CCCN(C3COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (oxetan-3- yl)piperidin-3- yl)benzamido)- 6- (trifluoromethyl) benzo[b] thiophene-2- carboxamide | 696.2 | 1H NMR (400 MHz, DMSO-d6) δ ppm 10.86-10.64 (m, 2H), 8.66 (s, 1H), 8.14 (dd, J = 2.6, 6.5 Hz, 1H), 8.07 (d, J = 8.8 Hz, 1H), 8.02-7.92 (m, 1H), 7.82 (dd, J = 1.6, 8.9 Hz, 1H), 7.64 (d, J = 2.4 Hz, 1H), 7.55 (t, J = 9.8 Hz, 1H), 7.46 (dd, J = 2.2, 8.6 Hz, 1H), 7.16 (d, J = 8.6 Hz, 1H), 4.58-4.49 (m, 1H), 4.49- 4.35 (m, 3H), 3.93 (s, 3H), 3.44-3.37 (m, 1H), 2.81-2.61 (m, 3H), 1.87-1.67 (m, 4H), 1.65-1.53 (m, 1H), 1.42-1.27 (m, 1H) | 2.51 A Example was prepared from Intermediate 77-1. |
| 98 | 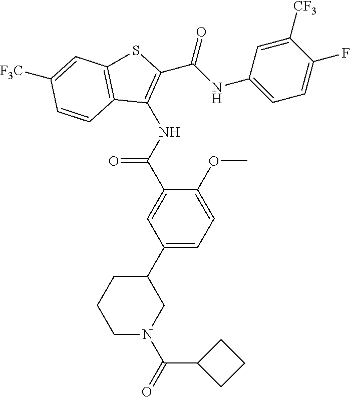 [SMILES: COc1ccc(C2CCCN(C(=O)C3CCC3)C2)cc1C(=O)Nc1c(C(=O)Nc2ccc(F)c(C(F)(F)F)c2)sc2cc(C(F)(F)F)ccc12] | 3-(5-(1- (cyclobutane- carbonyl) piperidin-3-yl)-2- methoxy- benzamido)-N-(4- fluoro-3- (trifluoromethyl) phenyl)-6- (trifluoromethyl) benzo[b] thiophene-2- carboxamide | 722.2 | 1H NMR (400 MHz, DMSO-d6) δ ppm 10.74 (br d, J = 0.7 Hz, 2H), 8.67 (s, 1H), 8.15 (dt, J = 2.8, 6.4 Hz, 1H), 8.07 (dd, J = 2.7, 8.6 Hz, 1H), 8.02- 7.93 (m,1H), 7.88- 7.79 (m, 1H), 7.67 (t, J = 2.4 Hz, 1H), 7.55 (t, J = 9.8 Hz, 1H), 7.47 (ddd, J = 2.6, 8.7, 11.0 Hz, 1H), 7.20 (dd, J = 5.0, 8.7 Hz, 1H), 4.46- 4.35 (m,1H), 3.94 (d, J = 6.4 Hz, 3H), 3.75- 3.56 (m, 1H), 2.95 (br t, J = 12.6 Hz, 1H), 2.63-2.53 (m, 2H), 2.25-2.00 (m, 4H), 1.98-1.80 (m, 3H), 1.78-1.53 (m, 3H), 1.47-1.31 (m, 1H) | 2.66 A Example was prepared from Intermediate 77-1. |
| 99 | 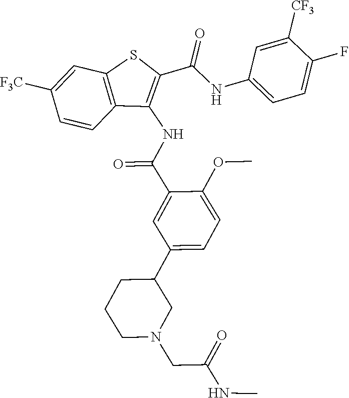 [SMILES: CNC(=O)CN1CCCC(c2ccc(OC)c(C(=O)Nc3c(C(=O)Nc4ccc(F)c(C(F)(F)F)c4)sc4cc(C(F)(F)F)ccc34)c2)C1] | N-(4-fluoro-3- (trifluoromethyl) phenyl)-3-(2- methoxy-5-(1- (2- (methylamino)- 2- oxoethyl) piperidin-3- yl)benzamido)- 6- (trifluoromethyl) benzo[b] thiophene-2- carboxamide | 711.2 | 1H NMR (400 MHz, DMSO-d6) δ ppm 10.81 (br d, J = 1.0 Hz, 1H), 10.74 (br s, 1H), 8.66 (s, 1H), 8.15 (dd, J = 2.7, 6.6 Hz, 1H), 8.07 (d, J = 8.8 Hz, 1H), 8.02-7.93 (m, 1H), 7.82 (dd, J = 1.5, 8.8 Hz, 1H), 7.73- 7.62 (m, 2H), 7.55 (t, J = 9.9 Hz, 1H), 7.44 (dd, J = 2.3, 8.4 Hz, 1H), 7.17 (d, J = 8.8 Hz, 1H), 3.93 (s, 3H), 2.91 (s, 2H), 2.87- 2.70 (m, 3H), 2.61 (d, J = 4.9 Hz, 3H), 2.14- 1.99 (m, 2H), 1.82- 1.74 (m, 1H), 1.72- 1.58 (m, 2H), 1.40- 1.26 (m, 1H) | 2.45 A Example was prepared from Intermediate 77-1. |
| 100 | 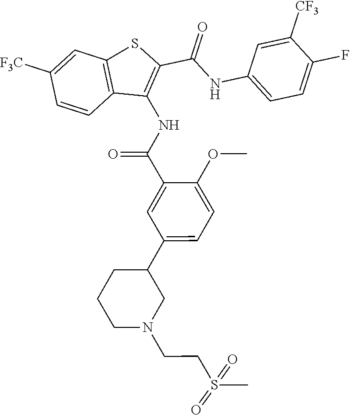 [SMILES: COc1ccc(C2CCCN(CCS(C)(=O)=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (2- (methylsulfonyl) ethyl) piperidin-3- yl)benzamido)- 6- (trifluoromethyl) benzo[b] thiophene-2- carboxamide | 746.2 | 1H NMR (400 MHz, DMSO-d6) δ ppm 10.90-10.57 (m, 2H), 8.66 (s, 1H), 8.15 (dd, J = 2.4, 6.4 Hz, 1H), 8.07 (d, J = 8.6 Hz, 1H), 7.99 (td, J = 3.8, 8.8 Hz, 1H), 7.82 (dd, J = 1.6, 8.7 Hz, 1H), 7.66 (d, J = 2.0 Hz, 1H), 7.54 (t, J = 9.8 Hz, 1H), 7.47 (dd, J = 2.2, 8.6 Hz, 1H), 7.17 (d, J = 8.8 Hz, 1H), 3.94 (s, 3H), 3.29 (t, J = 6.8 Hz, 2H), 3.02 (s, 3H), 2.91 (br d, J = 11.0 Hz, 2H), 2.72 (br t, J = 6.7 Hz, 3H), 1.97 (br t, J = 11.1 Hz, 2H), 1.80-1.64 (m, 2H), 1.62-1.45 (m, 1H), 1.41-1.25 (m, 1H) | 2.46 A Example was prepared from Intermediate 77-1. |
| 101 | 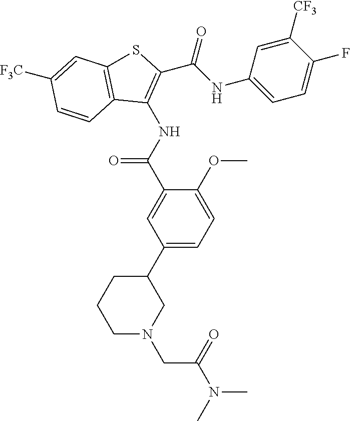 [SMILES: COc1ccc(C2CCCN(CC(=O)N(C)C)C2)cc1C(=O)Nc1c(C(=O)Nc2ccc(F)c(C(F)(F)F)c2)sc2cc(C(F)(F)F)ccc12] | 3-(5-(1-(2- (dimethylamino)- 2- oxoethyl) piperidin-3-yl)-2- methoxy- benzamido)-N-(4- fluoro-3- (trifluoromethyl) phenyl)-6- (trifluoromethyl) benzo[b] thiophene-2- carboxamide | 725.2 | 1H NMR (400 MHz, DMSO-d6) δ ppm 10.87-10.66 (m, 2H), 8.65 (s, 1H), 8.15 (dd, J = 2.4, 6.6 Hz, 1H), 8.08 (d, J = 8.3 Hz, 1H), 8.01-7.94 (m, 1H), 7.82 (dd, J = 1.7, 8.6 Hz, 1H), 7.64 (d, J = 2.2 Hz, 1H), 7.55 (t, J = 9.8 Hz, 1H), 7.49- 7.37 (m, 1H), 7.16 (d, J = 8.8 Hz, 1H), 3.92 (s, 3H), 3.17-3.08 (m, 2H), 3.02 (s,3H), 2.89- 2.69 (m, 6H), 2.13- 2.01 (m, 2H), 1.82- 1.73 (m, 1H), 1.72- 1.64 (m, 1H), 1.61- 1.49 (m, 1H), 1.40- 1.28 (m, 1H) | 2.43 A Example was prepared from Intermediate 77-1. |
| 102 | 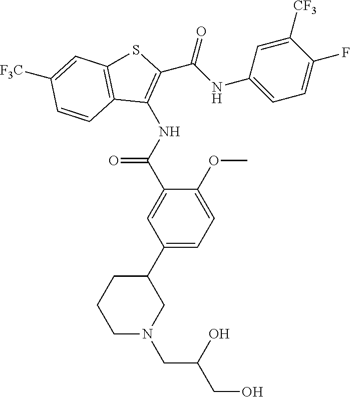 [SMILES: COc1ccc(C2CCCN(CC(O)CO)C2)cc1C(=O)Nc1c(C(=O)Nc2ccc(F)c(C(F)(F)F)c2)sc2cc(C(F)(F)F)ccc12] | 3-(5-(1-(2,3- dihydroxypropyl) piperidin-3- yl)-2- methoxy- benzamido)-N-(4- fluoro-3- (trifluoromethyl) phenyl)-6- (trifluoromethyl) benzo[b] thiophene-2- carboxamide | 714.2 | 1H NMR (400 MHz, DMSO-d6) δ ppm 10.87-10.70 (m, 2H), 8.66 (s, 1H), 8.20- 8.12 (m, 1H), 8.07 (d, J = 8.6 Hz, 1H), 8.02- 7.95 (m, 1H), 7.82 (dd, J = 1.5, 8.8 Hz, 1H), 7.66 (d, J = 2.0 Hz, 1H), 7.55 (t, J = 9.8 Hz, 1H), 7.45 (dd, J = 1.8, 8.4 Hz, 1H), 7.17 (d, J = 8.6 Hz, 1H), 5.65-5.36 (m, 1H) 4.58-4.29 (m, 1H), 3.93 (s, 3H), 3.67- 3.60 (m, 1H), 3.32- 3.32 (m, 1H), 2.99- 2.88 (m, 2H), 2.79- 2.70 (m, 2H), 2.47- 2.39 (m, 1H), 2.36- 2.26 (m, 1H), 2.14- 1.95 (m, 2H), 1.81- 1.52 (m, 3H), 1.41- 1.29 (m, 1H) | 2.14 A Example was prepared from Intermediate 77-1. |
| 103 | 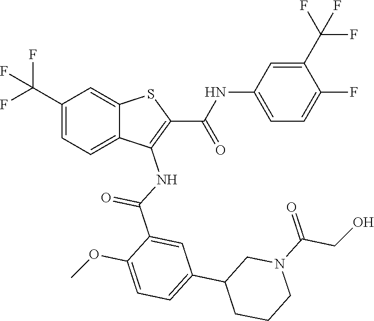 [SMILES: COc1ccc(C2CCCN(C(=O)CO)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [1-(2- hydroxyacetyl) piperidin-3-yl]- 2- methoxy- benzamido}-6- (trifluoromethyl)- 1- benzothiophene- 2- carboxamide | 698.2 | 1H NMR (400 MHz, DMSO-d6) δ ppm 8.67 (s, 1H), 8.15 (dd, J = 5.9, 2.0 Hz, 1H), 8.11-8.03 (m, 1H), 8.03-7.92 (m, 1H), 7.86-7.76 (m, 1H), 7.74-7.67 (m, 1H), 7.60-7.41 (m, 2H), 7.20 (d, J = 8.3 Hz, 1H), 4.58-4.49 (m, 1H), 4.45-4.32 (m, 1H), 4.17-4.04 (m, 2H), 3.97-3.90 (m, 3H), 3.75-3.64 (m, 1H), 3.04-2.92 (m, 1H), 2.77-2.69 (m, 1H), 2.64-2.58 (m, 1H), 1.92-1.81 (m, 1H), 1.78-1.63 (m, 2H), 1.56-1.39 (m, 1H). | 2.53 A Example was prepared from Intermediate 77-1. |
| 104 | 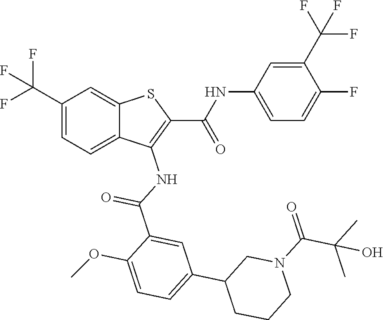 [SMILES: COc1ccc(C2CCCN(C(=O)C(C)(C)O)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [1-(2-hydroxy- 2- methylpropanoyl) piperidin-3- yl]-2- methoxy- benzamido}-6- (trifluoromethyl)- 1- benzothiophene- 2- carboxamide | 726.3 | 1H NMR (400 MHz, DMSO-d6) δ ppm 10.82 (br s, 1H), 10.72 (br s, 1H), 8.66 (s, 1H), 8.20-8.13 (m, 1H), 8.08 (d, J = 8.3 Hz, 1H), 8.03-7.93 (m, 1H), 7.82 (d, J = 8.3 Hz, 1H), 7.68 (s, 1H), 7.54 (t, J = 9.8 Hz, 1H), 7.50- 7.38 (m, 1H), 7.21 (d, J = 8.8 Hz, 1H), 5.39 (s, 1H), 4.95-4.78 (m, 1H), 4.58-4.40 (m, 1H), 3.94 (s, 3H), 3.07- 2.64 (m, 2H), 1.94- 1.83 (m, 1H), 1.78- 1.60 (m, 2H), 1.56- 1.40 (m, 1H), 1.36- 1.25 (m, 7H). | 2.26 A Example was prepared from Intermediate 77-1. |
| 105 | 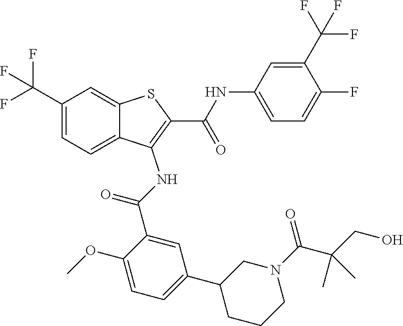 [SMILES: COc1ccc(C2CCCN(C(=O)C(C)(C)CO)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [1-(3-hydroxy- 2,2- dimethyl- propanoyl) piperidin- 3-yl]-2- methoxy- benzamido}-6- (trifluoromethyl)- 1- benzothiophene- 2- carboxamide | 740.3 | 1H NMR (400 MHz, DMSO-d6) δ ppm 10.89-10.65 (m, 2H), 8.67 (s, 1H), 8.16 (dd, J = 6.4, 2.4 Hz, 1H), 8.08 (d, J = 8.8 Hz, 1H), 8.03-7.94 (m, 1H), 7.82 (dd, J = 8.4, 1.3 Hz, 1H), 7.67 (d, J = 2.0 Hz, 1H), 7.55 (t, J = 9.9 Hz, 1H), 7.47 (dd, J = 8.7, 1.8 Hz, 1H), 7.20 (d, J = 8.8 Hz, 1H), 4.60-4.50 (m, 1H), 4.40-4.23 (m, 2H), 3.93 (s, 3H), 3.41 (br d, J = 3.2 Hz, 2H), 2.88- 2.72 (m, 2H), 2.68- 2.58 (m, 1H), 1.94- 1.84 (m, 1H), 1.79- 1.62 (m, 2H), 1.52- 1.39 (m, 1H), 1.15 (d, J = 2.7 Hz, 6H). | 2.26 A Example was prepared from Intermediate 77-1. |
| 106 | 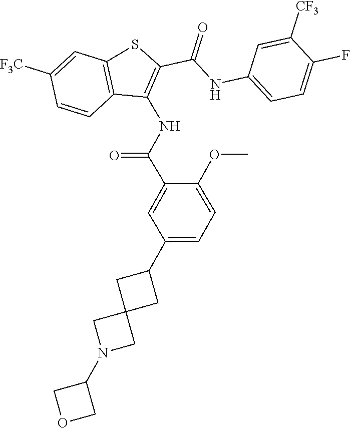 [SMILES: COc1ccc(C2CC3(C2)CN(C2COC2)C3)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(2- (oxetan-3-yl)- 2- azaspiro[3.3] heptan-6- yl)benzamido)- 6- (trifluoromethyl) benzo[b] thiophene-2- carboxamide | 708.3 | 1H NMR (400 MHz, DMSO-d6) δ ppm 10.81 (s, 1H), 10.74 (s, 1H), 8.67 (s, 1H), 8.14 (dd, J = 2.7, 6.6 Hz, 1H), 8.06 (d, J = 8.3 Hz, 1H), 8.02-7.95 (m, 1H), 7.82 (dd, J = 1.5, 8.8 Hz, 1H), 7.63 (d, J = 2.4 Hz, 1H), 7.55 (t, J = 9.8 Hz, 1H), 7.41 (dd, J = 2.3, 8.7 Hz, 1H), 7.17 (d, J = 8.6 Hz, 1H), 4.57 (t, J = 6.7 Hz, 2H), 4.33 (dd, J = 5.1, 6.8 Hz, 2H), 3.93 (s, 3H), 3.85- 3.75 (m, 2H), 3.48 (td, J = 1.7, 3.5 Hz, 3H), 3.28-3.23 (m, 2H), 2.48-2.43 (m, 1H), 2.20-2.08 (m, 2H) | 2.08 B |
| 107 | 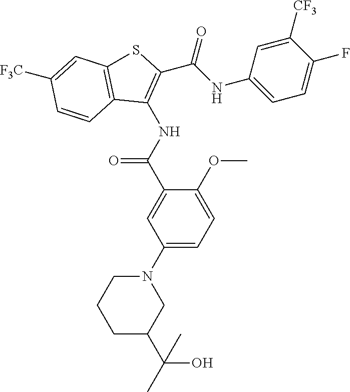 [SMILES: COc1ccc(N2CCCC(C(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (3-(2- hydroxypropan- 2-yl)piperidin- 1-yl)-2- methoxy- benzamido)-6- (trifluoromethyl) benzo[b] thiophene-2- carboxamide | 698.2 | 1H NMR (400 MHz, DMSO-d6) δ ppm 10.90-10.70 (m, 2H), 8.66 (s, 1H), 8.15 (dd, J = 2.7, 6.6 Hz, 1H), 8.06 (d, J = 8.6 Hz, 1H), 8.02-7.94 (m, 1H), 7.81 (dd, J = 1.8, 8.9 Hz, 1H), 7.54 (t, J = 9.7 Hz, 1H), 7.39 (d, J = 2.2 Hz, 1H), 7.21- 7.06 (m, 2H), 4.26 (s, 1H), 3.90 (s, 3H), 3.76- 3.63 (m, 1H), 3.54- 3.46 (m, 2H), 2.47- 2.40 (m, 1H), 2.39- 2.30 (m, 1H), 1.86- 1.77 (m, 1H), 1.77- 1.65 (m, 1H), 1.59- 1.46 (m, 2H), 1.09 (s, 3H), 1.05 (s, 3H). | 2.61 A The isomers were separated by chiral prep HPLC (Method 15) Example 107 (Chiral Prep HPLC RT = 6.548 min) 98% ee |
| 108 | 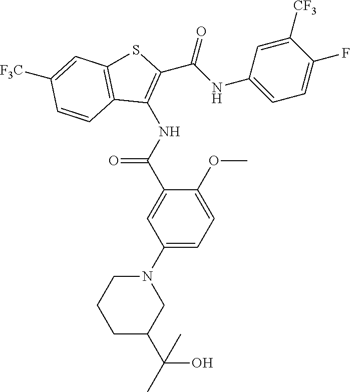 [SMILES: COc1ccc(N2CCCC(C(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (3-(2- hydroxypropan- 2-yl)piperidin- 1-yl)-2- methoxy- benzamido)-6- (trifluoromethyl) benzo[b] thiophene-2- carboxamide | 698.3 | 1H NMR (400 MHz, DMSO-d6) δ ppm 10.91-10.68 (m, 2H), 8.66 (s, 1H), 8.15 (dd, J = 2.6, 6.5 Hz, 1H), 8.06 (d, J = 8.6 Hz, 1H), 8.02-7.94 (m, 1H), 7.81 (dd, J = 1.6, 8.7 Hz, 1H), 7.54 (t, J = 9.7 Hz, 1H), 7.38 (d, J = 2.2 Hz, 1H), 7.20- 7.05 (m, 2H), 4.26 (s, 2H), 3.90 (s, 3H), 3.69 (br d, J = 11.0 Hz, 1H), 3.50 (br d, J = 7.8 Hz, 1H), 2.45 (br d, J = 2.2 Hz, 1H), 2.34 (s, 1H), 1.86-1.77 (m, 1H), 1.76-1.67 (m, 1H), 1.60-1.48 (m, 2H), 1.09 (s, 3H), 1.05 (s, 3H) | 2.61 A The isomers were separated by chiral prep HPLC (Method 15) Example 108 (Chiral Prep HPLC RT = 11.057 min). 98% ee |
| 109 | 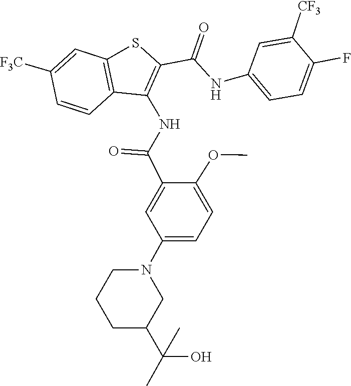 [SMILES: COc1ccc(N2CCCC(C(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (2-(3-hydroxy- 3- methylbutanoyl)- 2- azaspiro[3.3] heptan-6-yl)-2- methoxy- benzamido)-6- (trifluoromethyl) benzo[b] thiophene-2- carboxamide | 752.3 | 1H NMR (400 MHz, DMSO-d6) δ ppm 10.82 (s, 1H), 10.74 (s, 1H), 8.66 (s, 1H), 8.19- 8.12 (m, 1H), 8.06 (d, J = 8.6 Hz, 1H), 8.02- 7.95 (m, 1H), 7.82 (dd, J = 1.6, 8.7 Hz, 1H), 7.64 (d, J = 2.4 Hz, 1H), 7.55 (t, J = 9.8 Hz, 1H), 7.41 (td, J = 2.8, 8.5 Hz, 1H), 7.17 (d, J = 8.6 Hz, 1H), 4.75 (d, J = 4.2 Hz, 1H), 4.27 (s, 1H), 4.04 (s, 2H), 3.99 (s, 2H), 3.93 (s, 3H), 3.76 (s, 1H), 2.57-2.53 (m, 1H), 2.25-2.07 (m, 4H), 1.16 (d, J = 5.9 Hz, 6H) | 2.46 B |
| 110 | 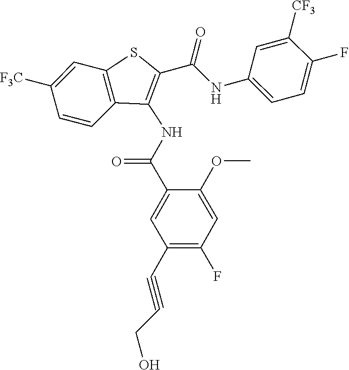 [SMILES: COc1cc(F)c(C#CCO)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(4- fluoro-5-(3- hydroxyprop- 1-yn-1-yl)-2- methoxy- benzamido)-6- (trifluoromethyl) benzo[b] thiophene-2- carboxamide | 629.3 | 1H NMR (400 MHz, DMSO-d6) δ ppm 10.75 (br d, J = 1.5 Hz, 2H), 8.66 (s, 1H), 8.15 (dd, J = 2.4, 6.1 Hz, 1H), 8.07 (d, J = 8.6 Hz, 1H), 8.02-7.95 (m, 1H), 7.89 (d, J = 8.6 Hz, 1H), 7.84- 7.78 (m, 1H), 7.55 (t, J = 9.8 Hz, 1H), 7.28 (d, J = 11.7 Hz, 1H), 5.39 (br s, 1H), 4.32 (br d, J = 0.7 Hz, 2H), 3.94 (s, 3H) | 2.39 A |
| 111 | 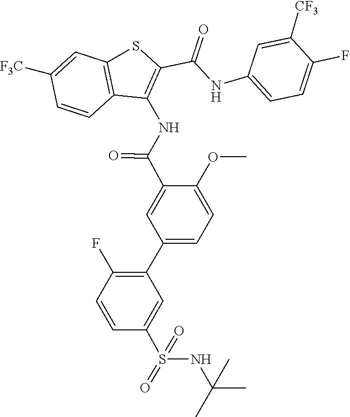 [SMILES: COc1ccc(-c2cc(S(=O)(=O)NC(C)(C)C)ccc2F)cc1C(=O)Nc1c(C(=O)Nc2ccc(F)c(C(F)(F)F)c2)sc2cc(C(F)(F)F)ccc12] | 3-(5′-(N-(tert- butyl)sulfamoyl)- 2′-fluoro-4- methoxy-[1,1′- biphenyl]-3- carboxamido)- N-(4-fluoro-3- (trifluoromethyl) phenyl)-6- (trifluoromethyl) benzo[b] thiophene-2- carboxamide | 786.2 | 1H NMR (400 MHz, DMSO-d6) δ ppm 10.88 (br s, 1H), 10.76 (br s, 1H), 8.67 (s, 1H), 8.19-8.07 (m, 2H), 8.04-7.94 (m, 3H), 7.91-7.76 (m, 3H), 7.64 (s, 1H), 7.52 (ddd, J = 2.3, 8.7, 10.5 Hz, 2H), 7.41 (d, J = 8.8 Hz, 1H), 4.01 (s, 3H), 1.10 (s, 9H) | 2.71 B |
| 112 | 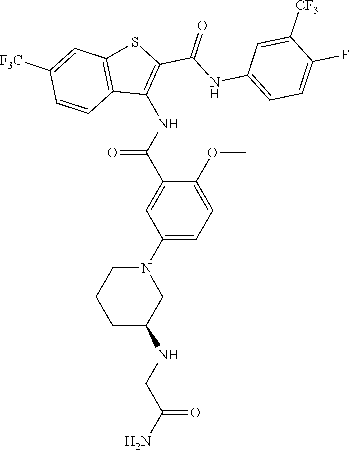 [SMILES: COc1ccc(N2CCC[C@H](NCC(N)=O)C2)cc1C(=O)Nc1c(C(=O)Nc2ccc(F)c(C(F)(F)F)c2)sc2cc(C(F)(F)F)ccc12] | (R)-3-(5-(3- ((2-amino-2- oxoethyl) amino)piperidin-1- yl)-2- methoxy- benzamido)-N-(4- fluoro-3- (trifluoromethyl) phenyl)-6- (trifluoromethyl) benzo[b] thiophene-2- carboxamide | 712.2 | 1H NMR (400 MHz, DMSO-d6) δ ppm 10.48 (s, 1H), 9.76 (d, J = 7.1 Hz, 1H), 8.22 (dd, J = 2.7, 6.6 Hz, 1H), 7.82-7.69 (m, 1H), 7.60 (s, 1H), 7.48 (t, J = 9.5 Hz, 1H), 6.88 (s, 1H), 4.67 (d, J = 9.5 Hz, 1H), 4.43 (br s, 1H), 3.94 (s, 3H), 3.57- 3.45 (m, 4H), 3.25 (s, 3H), 3.14 (dt, J = 4.9, 10.0 Hz, 1H), 3.06 (br s, 1H), 2.85-2.77 (m, 2H), 2.74-2.66 (m, 3H), 2.62 (t, J = 5.9 Hz, 2H), 1.91-1.81 (m, 1H), 1.81-1.72 (m, 1H), 1.54-1.45 (m, 1H), 1.45-1.29 (m, 2H), 0.79-0.67 (m, 2H), 0.34 (dd, J = 2.1, 4.8 Hz, 2H) | 2.26 A |
| 114 | 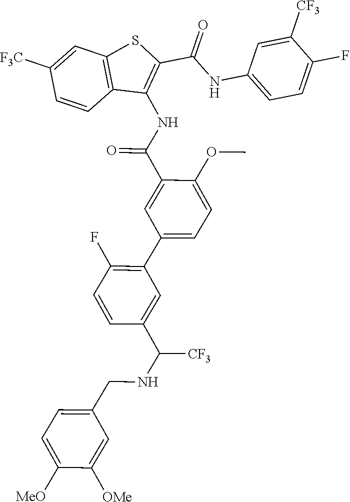 [SMILES: COc1ccc(CNC(c2ccc(F)c(-c3ccc(OC)c(C(=O)Nc4c(C(=O)Nc5ccc(F)c(C(F)(F)F)c5)sc5cc(C(F)(F)F)ccc45)c3)c2)C(F)(F)F)cc1OC] | 3-(5′-(1-((3,4- dimethoxybenzyl) amino)- 2,2,2- trifluoroethyl)- 2′-fluoro-4- methoxy-[1,1′- biphenyl]-3- carboxamido)- N-(4-fluoro-3- (trifluoromethyl) phenyl)-6- (trifluoromethyl) benzo[b] thiophene-2- carboxamide | 898.3 | 1H NMR (400 MHz, DMSO-d6) δ ppm 10.59-11.17 (m, 2 H) 8.50-8.73 (m, 1 H) 8.15 (br d, J = 8.53 Hz, 2 H) 8.09 (br s, 2 H) 7.94 (br s, 2 H) 7.62- 7.85 (m, 2 H) 7.50 (br s, 2 H) 7.26-7.44 (m, 2 H) 6.82-6.90 (m, 2 H) 6.76 (d, J = 7.77 Hz, 1 H) 5.80 (s, 1H) 4.33 (br d, J = 8.53 Hz, 2 H) 3.96 (s, 3 H) 3.87 (s, 6 H) | 3.53 D The isomers were separated by prep SFC (Method 16) to give Example 114. Analytical SFC (Method 17) RT = 6.19 min. 100% ee |
| 115 | 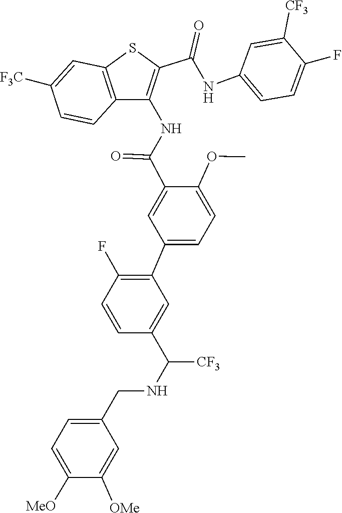 [SMILES: COc1ccc(CNC(c2ccc(F)c(-c3ccc(OC)c(C(=O)Nc4c(C(=O)Nc5ccc(F)c(C(F)(F)F)c5)sc5cc(C(F)(F)F)ccc45)c3)c2)C(F)(F)F)cc1OC] | 3-(5′-(1-((3,4- dimethoxybenzyl) amino)- 2,2,2- trifluoroethyl)- 2′-fluoro-4- methoxy-[1,1′- biphenyl]-3- carboxamido)- N-(4-fluoro-3- (trifluoromethyl) phenyl)-6- (trifluoromethyl) benzo[b] thiophene-2- carboxamide | 898.1 | 1H NMR (400 MHz, DMSO-d6) δ ppm 10.88 (s, 1 H) 10.75 (s, 1 H) 8.68 (s, 1 H) 8.08- 8.17 (m, 2 H) 7.96- 8.03 (m, 2 H) 7.74- 7.85 (m, 1 H) 7.65 (br d, J = 6.53 Hz, 1 H) 7.51 (brt, J = 9.54 Hz, 2 H) 7.30-7.41 (m, 2 H) 6.89 (s, 2 H) 6.85 (d, J = 8.31 Hz, 2 H) 6.77 (d, J = 8.37 Hz, 1 H) 5.69 (s,1H) 4.34 (br d, J = 9.04 Hz, 2 H) 4.01 (s, 3 H) 3.68 (d, J = 5.02 Hz, 6 H) | 2.24 D The isomers were separated by prep SFC (Method 16) to give Example 115. Analytical SFC (Method 17) RT = 3.04 min) 100% ee |
| 116 | 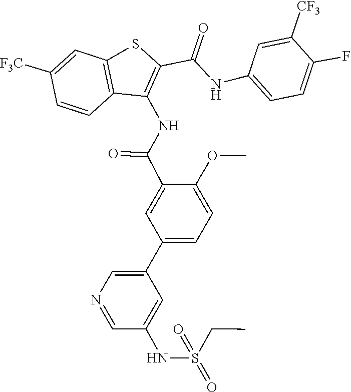 [SMILES: CCS(=O)(=O)Nc1cncc(-c2ccc(OC)c(C(=O)Nc3c(C(=O)Nc4ccc(F)c(C(F)(F)F)c4)sc4cc(C(F)(F)F)ccc34)c2)c1] | 3-(5-(5- (ethylsulfonamido) pyridin-3- yl)-2- methoxy- benzamido)-N-(4- fluoro-3- (trifluoromethyl) phenyl)-6- (trifluoromethyl) benzo[b] thiophene-2- carboxamide | 741.2 | 1H NMR (400 MHz, DMSO-d6) δ ppm 10.91 (s, 1H), 10.78 (s, 1H), 10.17 (s, 1H), 8.68 (s, 1H), 8.62 (s, 1H), 8.44 (br s, 1H), 8.20-8.07 (m, 3H), 8.05-7.97 (m, 1H), 7.91 (dd, J = 2.3, 8.7 Hz, 1H), 7.86-7.76 (m, 2H), 7.53 (t, J = 9.7 Hz, 1H), 7.39 (d, J = 8.8 Hz, 1H), 3.99 (s, 3H), 3.22 (q, J = 7.3 Hz, 2H), 1.23 (t, J = 7.3 Hz, 3H) | 2.33 A |
| 117 | 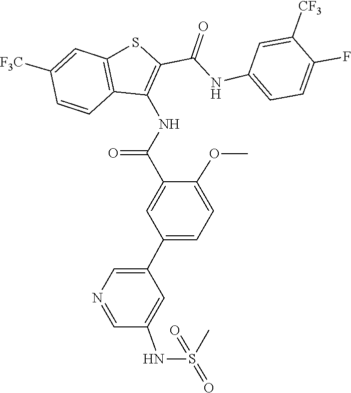 [SMILES: COc1ccc(-c2cncc(NS(C)(=O)=O)c2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(5- (methyl- sulfonamido) pyridin-3- yl)benzamido)- 6- (trifluoromethyl) benzo[b] thiophene-2- carboxamide | 727.1 | 1H NMR (400 MHz, DMSO-d6) δ ppm 10.91 (s, 1H), 10.78 (s, 1H), 10.11 (s, 1H), 8.68 (s, 1H), 8.64 (d, J = 2.0 Hz, 1H), 8.43 (d, J = 2.0 Hz, 1H), 8.19- 8.10 (m, 3H), 8.05- 7.98 (m, 1H), 7.93 (dd, J = 2.3, 8.4 Hz, 1H), 7.86-7.77 (m, 2H), 7.57-7.49 (m, 1H), 7.40 (d, J = 8.6 Hz, 1H), 3.98 (s, 3H), 3.11 (s, 3H) | 2.29 A |
| 118 | 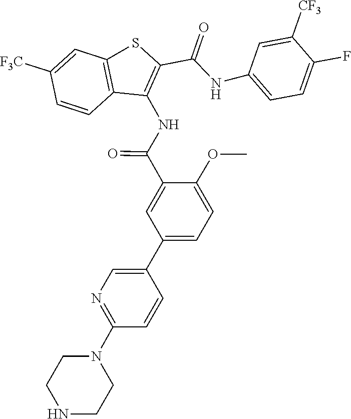 [SMILES: COc1ccc(-c2ccc(N3CCNCC3)nc2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(6- (piperazin-1- yl)pyridin-3- yl)benzamido)- 6- (trifluoromethyl) benzo[b] thiophene-2- carboxamide | 718.2 | 1H NMR (400 MHz, DMSO-d6) δ ppm 10.74-10.94 (m, 2 H) 8.63-8.73 (m, 1 H) 8.36-8.46 (m, 2 H) 8.08-8.20 (m, 1 H) 7.96-8.05 (m, 2 H) 7.75-7.87 (m, 1 H) 7.49-7.59 (m, 1 H) 7.23-7.33 (m, 2 H) 6.86-6.96 (m, 2 H) 3.92 (s, 3 H) 3.48- 3.58 (m, 2 H) 3.31 (s, 2 H) 2.86-2.95 (m, 2 H) 2.50 (dt, J = 3.64, 1.95 Hz, 2 H) | 3.50 A |
| 119 | 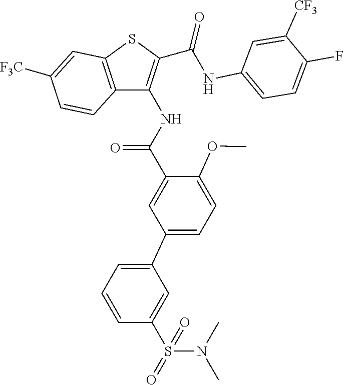 [SMILES: COc1ccc(-c2cccc(S(=O)(=O)N(C)C)c2)cc1C(=O)Nc1c(C(=O)Nc2ccc(F)c(C(F)(F)F)c2)sc2cc(C(F)(F)F)ccc12] | 3-(3′-(N,N- dimethyl- sulfamoyl)-4- methoxy-[1,1′- biphenyl]-3- carboxamido)- N-(4-fluoro-3- (trifluoromethyl) phenyl)-6- (trifluoromethyl) benzo[b] thiophene-2- carboxamide | 740.2 | 1H NMR (400 MHz, DMSO-d6) δ ppm 10.91 (s, 1H), 10.79 (s, 1H), 8.68 (s, 1H), 8.10-8.22 (m, 3H), 7.94-8.05 (m, 3H), 7.91 (s, 1H), 7.83 (dd, J = 8.8, 1.5 Hz, 1H), 7.68-7.78 (m, 2H), 7.52 (t, J = 9.8 Hz, 1H), 7.38 (d, J = 9.0 Hz, 1H), 3.99 (s, 3H), 2.65 ppm (s, 6H) | 2.62 B |
| 120 | 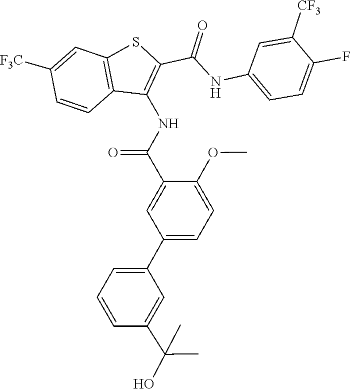 [SMILES: COc1ccc(-c2cccc(C(C)(C)O)c2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(3′- (2- hydroxypropan- 2-yl)-4- methoxy-[1,1′- biphenyl]-3- carboxamido)- 6- (trifluoromethyl) benzo[b] thiophene-2- carboxamide | 689.1 | 1H NMR (400 MHz, DMSO-d6) δ ppm 10.85-10.94 (m, 1H), 10.67-10.81 (m, 1H), 8.67 (s, 1H), 8.18 (br dd, J = 3.5, 2.8 Hz, 1H), 8.10-8.15 (m, 1H), 8.06-8.10 (m, 2H), 7.96-8.03 (m, 1H), 7.80-7.90 (m, 2H), 7.74 (s, 2H), 7.49-7.57 (m, 1H), 7.27-7.46 (m, 2H), 6.35 (m, 1H) 3.94-4.02 (m, 3H), 1.47 ppm (s, 6H). | 2.63 A |
| 121 | 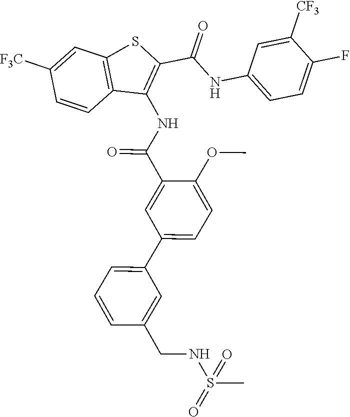 [SMILES: COc1ccc(-c2cccc(CNS(C)(=O)=O)c2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(4- methoxy-3′- (methylsulfon- amidomethyl)- [1,1′-biphenyl]- 3- carboxamido)- 6- (trifluoromethyl) benzo[b] thiophene-2- carboxamide | 738.1 (M − H)⁻ | 1H NMR (400 MHz, DMSO-d6) δ ppm 10.96-10.88 (m, 1H), 10.81-10.68 (m, 1H), 8.68 (s, 1H), 8.18 (br dd, J = 2.1, 4.3 Hz, 1H), 8.15-8.07 (m, 2H), 8.04-7.97 (m, 1H), 7.88 (dd, J = 2.6, 8.7 Hz, 1H), 7.85- 7.78 (m, 1H), 7.68- 7.61 (m, 2H), 7.59- 7.50 (m, 2H), 7.45 (t, J = 7.8 Hz, 1H), 7.39- 7.29 (m, 1H), 4.89 (m, 1H) 4.24 (d, J = 6.4 Hz, 2H), 3.99 (s, 3H), 2.88 (s, 3H | 2.49 A |
| 122 | 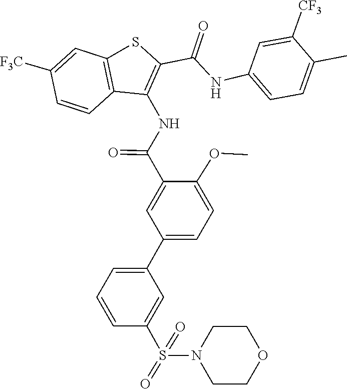 [SMILES: COc1ccc(-c2cccc(S(=O)(=O)N3CCOCC3)c2)cc1C(=O)Nc1c(C(=O)Nc2ccc(C)c(C(F)(F)F)c2)sc2cc(C(F)(F)F)ccc12] | N-(4-fluoro-3- (trifluoromethyl) phenyl)-3-(4- methoxy-3′- (morpholino- sulfonyl)-[1,1′- biphenyl]-3- carboxamido)- 6- (trifluoromethyl) benzo[b] thiophene-2- carboxamide | 780.1 | 1H NMR (400 MHz, DMSO-d6) δ ppm 10.91 (s, 1H), 10.78 (s, 1H), 8.68 (s, 1H), 8.11-8.20 (m, 3H), 7.93-8.08 (m, 3H), 7.90 (s, 1H), 7.83 (dd, J = 8.6, 1.5 Hz, 1H), 7.67-7.80 (m, 2H), 7.52 (t, J = 9.9 Hz, 1H), 7.38 (d, J = 8.6 Hz, 1H), 3.99 (s, 3H), 3.60-3.70 (m, 4H), 2.80-2.98 ppm (m, 4H) | 2.60 A |
| 123 | 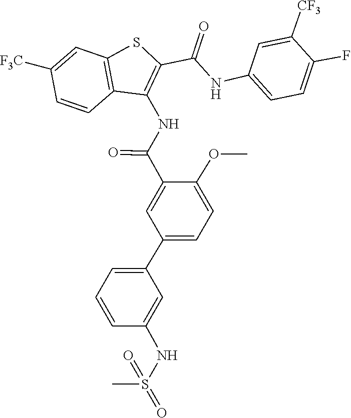 [SMILES: COc1ccc(-c2cccc(NS(C)(=O)=O)c2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(4- methoxy-3′- (methylsulfon- amido)-[1,1′- biphenyl]-3- carboxamido)- 6- (trifluoromethyl) benzo[b] thiophene-2- carboxamide | 724.3 | 1H NMR (400 MHz, DMSO-d6) δ ppm 10.91 (s, 1H), 10.79 (s, 1H), 9.84 (s, 1H), 8.68 (s, 1H), 8.18 (dd, J = 6.7, 2.6 Hz, 1H), 8.11 (d, J = 8.1 Hz, 1H), 8.07 (d, J = 2.4 Hz, 1H), 7.95-8.05 (m,1H), 7.75-7.87 (m, 2H), 7.54 (br t, J = 9.8 Hz, 1H), 7.48 (s, 1H), 7.39-7.46 (m, 1H), 7.36 (br d, J = 8.6 Hz,2H), 7.16- 7.26 (m, 1H), 3.99 (s, 3H), 3.02 ppm (s, 3H) | 2.49 A |
| 124 | 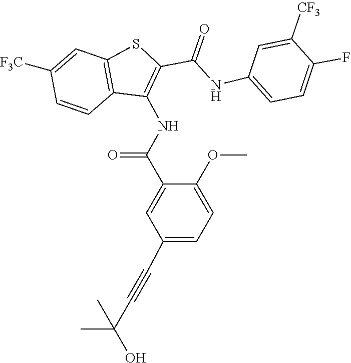 | N-(4-fluoro-3- (trifluoromethyl) phenyl)-3-(5- (3-hydroxy-3- methylbut-1- yn-1-yl)-2- methoxybenz- amido)-6- (trifluoromethyl) benzo[b] thiophene-2- carboxamide | 639.0 | 1H NMR (400 MHz, DMSO-d6) δ ppm 10.83 (s, 1 H) 10.75 (s, 1 H) 8.67 (s, 1 H) 8.16 (dd, J = 6.02, 2.51 Hz, 1 H) 7.98-8.09 (m, 2 H) 7.82 (d, J = 8.28 Hz, 1 H) 7.77 (s, 1 H) 7.53- 7.59 (m, 2 H) 7.23 (d, J = 8.53 Hz, 1 H) 5.45 (s, 1 H) 3.94 (s, 3 H) 1.46 (s, 6 H) | 3.80 C |
| 127 | 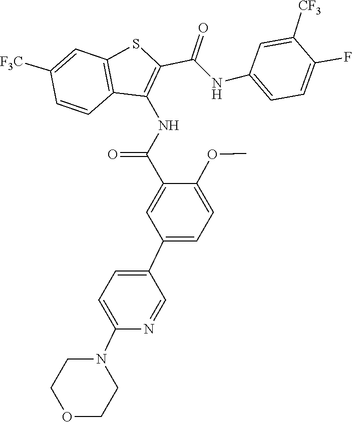 [SMILES: COc1ccc(-c2ccc(N3CCOCC3)nc2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(6- morpholino- pyridin-3- yl)benzamido)- 6- (trifluoromethyl) benzo[b] thiophene-2- carboxamide | 719.2 | 1H NMR (400 MHz, DMSO-d6) δ ppm 10.87 (s, 2 H) 10.76 (s, 1 H) 8.68 (s, 1 H) 8.43 (d, J = 2.01 Hz, 1 H) 8.10-8.18 (m, 2 H) 8.01 (br d, J = 2.51 Hz, 2 H) 7.75-7.92 (m, 2 H) 7.54 (br t, J = 9.54 Hz, 1 H) 7.31 (d, J = 8.53 Hz, 1 H) 6.96 (br d, J = 8.53 Hz, 1 H) 3.95 (s, 3 H) 3.62- 3.78 (m, 4 H) 3.49- 3.60 (m, 2 H) 2.53- 2.61 (m, 2 H) | 3.14 C |
| 128 | 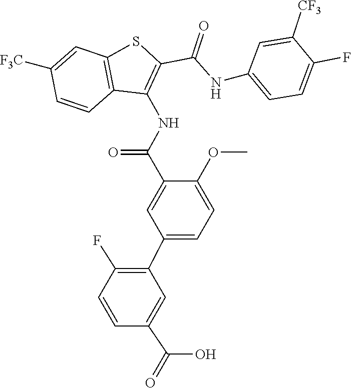 [SMILES: COc1ccc(-c2cc(C(=O)O)ccc2F)cc1C(=O)Nc1c(C(=O)Nc2ccc(F)c(C(F)(F)F)c2)sc2cc(C(F)(F)F)ccc12] | 6-fluoro-3′-((2- ((4-fluoro-3- (trifluoromethyl) phenyl) carbamoyl)-6- (trifluoromethyl) benzo[b] thiophen-3- yl)carbamoyl)- 4′-methoxy- [1,1′-biphenyl]- 3-carboxylic acid | 695.1 | 1H NMR (400 MHz, DMSO-d6) δ ppm 13.16 (br d, J = 3.91 Hz, 1 H) 10.67-11.03 (m, 2 H) 8.54-8.82 (m, 4 H) 7.92-8.29 (m, 4 H) 7.73-7.92 (m, 2 H) 7.28-7.66 (m, 2 H) 3.99 (s, 3 H) | 3.06 C |
| 129 | 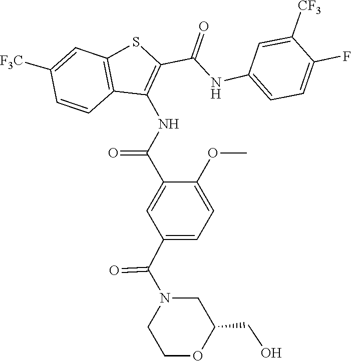 [SMILES: COc1ccc(C(=O)N2CCO[C@@H](CO)C2)cc1C(=O)Nc1c(C(=O)Nc2ccc(F)c(C(F)(F)F)c2)sc2cc(C(F)(F)F)ccc12] | (R)-N-(4- fluoro-3- (trifluoromethyl) phenyl)-3-(5- (2- (hydroxymethyl) morpholine- 4-carbonyl)-2- methoxy- benzamido)-6- (trifluoromethyl) benzo[b] thiophene-2- carboxamide | 700.0 | 1H NMR (400 MHz, DMSO-d6) δ ppm 10.85 (s, 1H), 10.77- 10.68 (m, 1H), 8.67 (s, 1H), 8.21-8.14 (m, 1H), 8.08 (d, J = 8.8 Hz, 1H), 8.03-7.96 (m, 1H), 7.89-7.75 (m, 2H), 7.64 (dd, J = 2.2, 8.6 Hz, 1H), 7.55 (t, J = 9.8 Hz, 1H), 7.29 (d, J = 8.8 Hz, 1H), 5.21 (m 1H) 4.83- 4.74 (m, 2H), 3.97 (s, 3H), 3.89-3.73 (m, 2H), 3.50-3.36 (m, 4H). | 2.10 C |
| 130 | 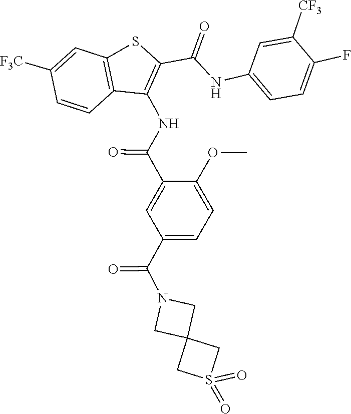 [SMILES: COc1ccc(C(=O)N2CC3(C2)CS(=O)(=O)C3)cc1C(=O)Nc1c(C(=O)Nc2ccc(F)c(C(F)(F)F)c2)sc2cc(C(F)(F)F)ccc12] | 3-(5-(2,2- dioxido-2-thia- 6- azaspiro[3.3] heptane-6- carbonyl)-2- methoxy- benzamido)-N-(4- fluoro-3- (trifluoromethyl) phenyl)-6- (trifluoromethyl) benzo[b] thiophene-2- carboxamide | 728.1 | 1H NMR (400 MHz, DMSO-d6) δ ppm 10.85 (br s, 1H), 10.75 (br s, 1H), 8.67 (s, 1H), 8.16 (dd, J = 2.6, 6.5 Hz, 1H), 8.12- 8.03 (m, 2H), 8.02- 7.94 (m, 1H), 7.83 (ddd, J = 2.0, 6.9, 8.7 Hz, 2H), 7.55 (t, J = 9.9 Hz, 1H), 7.30 (d, J = 8.8 Hz, 1H), 4.63- 4.54 (m, 2H), 4.47 (br d, J = 10.3 Hz, 4H), 4.28 (br s, 2H), 3.98 (s, 3H). | 2.18 C |
| 131 | 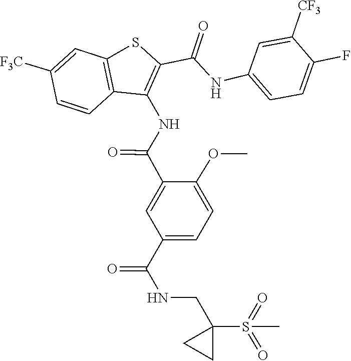 [SMILES: COc1ccc(C(=O)NCC2(S(C)(=O)=O)CC2)cc1C(=O)Nc1c(C(=O)Nc2ccc(F)c(C(F)(F)F)c2)sc2cc(C(F)(F)F)ccc12] | N3-(2-((4- fluoro-3- (trifluoromethyl) phenyl) carbamoyl)-6- (trifluoromethyl) benzo[b] thiophen-3-yl)-4- methoxy-N1- ((1- (methylsulfonyl) cyclopropyl) methyl) isophthalamide | 730.2 | 1H NMR (400 MHz, DMSO-d6) δ ppm 10.86 (br s, 1H), 10.75 (br s, 1H), 8.73 (br t, J = 6.1 Hz, 1H), 8.68 (s, 1H), 8.37 (d, J = 2.2 Hz, 1H), 8.16 (dd, J = 2.4, 6.4 Hz, 1H), 8.12- 8.04 (m, 2H), 7.99 (td, J = 4.1, 8.1 Hz, 1H), 7.82 (dd, J = 1.6, 8.7 Hz, 1H), 7.54 (t, J = 9.7 Hz, 1H), 7.34 (d, J = 8.8 Hz, 1H), 3.99 (s, 3H), 3.83 (d, J = 6.1 Hz, 2H), 3.06 (s, 3H), 1.29-1.18 (m, 2H), 1.17-1.08 (m, 2H). | 2.24 C |
| 132 | 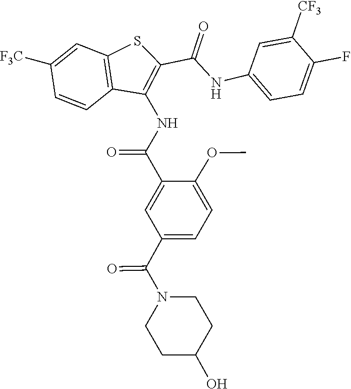 [SMILES: COc1ccc(C(=O)N2CCC(O)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (4- hydroxy- piperidine-1- carbonyl)-2- methoxy- benzamido)-6- (trifluoromethyl) benzo[b] thiophene-2- carboxamide | 684.2 | 1H NMR (400 MHz, DMSO-d6) δ ppm 10.94-10.65 (m, 2H), 8.67 (s, 1H), 8.17 (dd, J = 2.7, 6.6 Hz, 1H), 8.08 (d, J = 8.6 Hz, 1H), 7.98 (ddd, J = 2.8, 4.3, 9.0 Hz, 1H), 7.81 (dt, J = 1.8, 4.3 Hz, 2H), 7.61 (dd, J = 2.1, 8.4 Hz, 1H), 7.54 (t, J = 9.9 Hz, 1H), 7.28 (d, J = 8.8 Hz, 1H), 4.79 (m, 2H), 3.97 (s,3H), 3.77-3.69 (m, 1H), 3.51 (s, 1H), 3.18 (br d, J = 1.0 Hz, 2H), 1.84-1.57 (m, 2H), 1.44-1.23 (m, 2H) | 2.13 C |
| 133 | 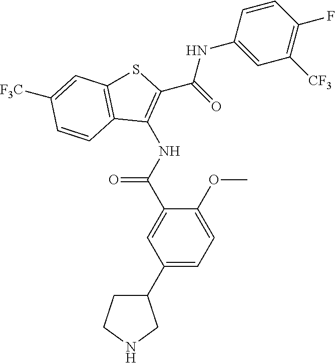 [SMILES: COc1ccc(C2CCNC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5- (pyrrolidin-3- yl)benzamido)- 6- (trifluoromethyl) benzo[b] thiophene-2- carboxamide | 626.2 | 1H NMR (400 MHz, DMSO-d6) δ ppm 10.81 (s, 1H), 10.73 (s, 1H), 8.65 (s, 1H), 8.18- 8.12 (m, 1H), 8.06 (d, J = 8.3 Hz, 1H), 7.98 (br dd, J = 3.9, 8.1 Hz, 1H), 7.83-7.77 (m, 1H), 7.75 (d, J = 2.0 Hz, 1H), 7.59-7.43 (m, 2H), 7.20 (d, J = 8.8 Hz, 1H), 3.93 (s, 3H), 3.23- 3.21 (m, 1H), 3.12- 3.07 (m, 1H), 2.86 (br t, J = 10.0 Hz, 2H), 2.29- 2.16 (m, 1H), 1.89 (s, 1H), 1.79 (br dd, J = 9.5, 12.2 Hz, 1H) | 2.08 A The isomers were separated by prep SFC (Method 20) to give isomer 1, Intermediate 133-1 (Prep SFC RT = 3.6 min, 100% ee) and isomer 2, Example 133 (Prep SFC RT = 5.8 min, 98% ee). Analytical SFC (Method 21) |
| 134 | 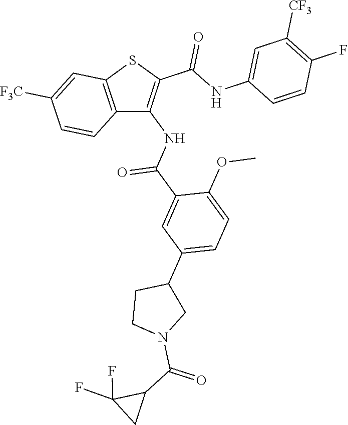 | 3-(5-(1-(2,2- difluorocyclo- propane-1- carbonyl) pyrrolidin-3-yl)-2- methoxy- benzamido)-N-(4- fluoro-3- (trifluoromethyl) phenyl)-6- (trifluoromethyl) benzo[b] thiophene-2- carboxamide | 474.2 (M + H + NH₃)⁺ | 1H NMR (400 MHz, DMSO-d6) δ ppm 10.69-10.85 (m, 2 H) 8.61-8.67 (m, 1 H) 8.12-8.17 (m, 1 H) 7.93-8.00 (m, 1 H) 7.78-7.84 (m, 1 H) 7.70-7.75 (m, 1 H) 7.58-7.58 (m, 1 H) 7.53 (br d, J = 9.29 Hz, 1 H) 7.45-7.57 (m, 1 H) 7.17-7.24 (m, 1 H) 4.02 (br d, J = 8.80 Hz, 2 H) 3.93 (br s, 3 H) 3.60 (br s, 2 H) 3.52 (br s, 2 H) 3.16-3.34 (m, 4 H) | 2.50 A Example was prepared from Example 133. |
| 135 | 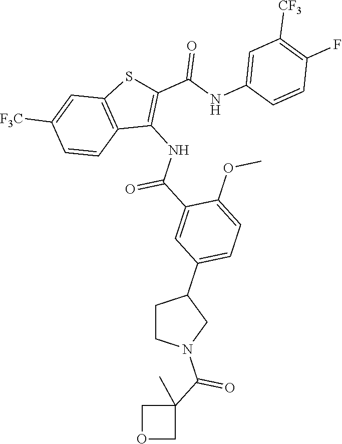 | N-(4-fluoro-3- (trifluoromethyl) phenyl)-3-(2- methoxy-5-(1- (3- methyloxetane- 3- carbonyl) pyrrolidin-3- yl)benzamido)- 6- (trifluoromethyl) benzo[b] thiophene-2- carboxamide | (M + H + NH₃)⁺ | 1H NMR (400 MHz, DMSO-d6) δ ppm 10.69-10.85 (m, 2 H) 8.61-8.67 (m, 1 H) 8.12-8.17 (m, 1 H) 7.93-8.00 (m, 1 H) 7.78-7.84 (m, 1 H) 7.70-7.75 (m, 1 H) 7.58-7.58 (m, 1 H) 7.53 (br d, J = 9.29 Hz, 1 H) 7.45-7.57 (m, 1 H) 7.17-7.24 (m, 1 H) 4.02 (br d, J = 8.80 Hz, 2 H) 3.93 (br s, 4 H) 3.60 (br s, 2 H) 3.52 (br s, 2 H) 3.16-3.34 (m, 4 H) | 2.36 A Example was prepared from Example 133. |
| 136 | 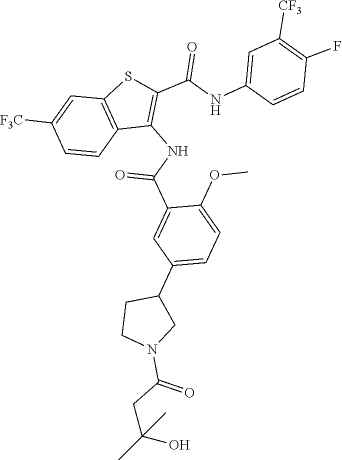 [SMILES: COc1ccc(C2CCN(C(=O)CC(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(3-hydroxy- 3- methylbutanoyl) pyrrolidin-3- yl)-2- methoxy- benzamido)-6- (trifluoromethyl) benzo[b] thiophene-2- carboxamide | 726.1 | 1H NMR (400 MHz, DMSO-d6) δ ppm 10.69-10.85 (m, 2 H) 8.61-8.67 (m, 1 H) 8.12-8.17 (m, 1 H) 7.93-8.00 (m, 1 H) 7.78-7.84 (m, 1 H) 7.70-7.75 (m, 1 H) 7.58-7.58 (m, 1 H) 7.53 (br d, J = 9.29 Hz, 1 H) 7.45-7.57 (m, 1 H) 7.17-7.24 (m, 1 H) 4.02 (br d, J = 8.80 Hz, 1 H) 3.93 (br s, 4 H) 3.60 (br s, 2 H) 3.52 (br s, 2 H) 3.16-3.34 (m, 4 H), 2.12-2.09 (m, 6H) | 2.40 A Example was prepared from Example 133. |
| 137 | 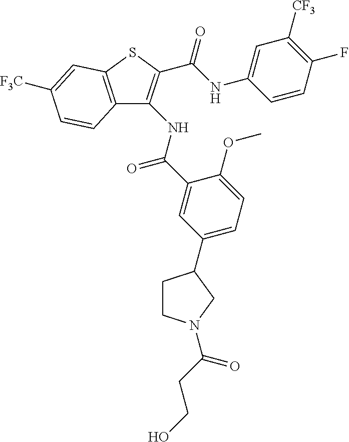 [SMILES: COc1ccc(C2CCN(C(=O)CCO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(3- hydroxypropanoyl) pyrrolidin- 3-yl)-2- methoxy- benzamido)-6- (trifluoromethyl) benzo[b] thiophene-2- carboxamide | 698.2 | 1H NMR (400 MHz, DMSO-d6) δ ppm 10.51-11.19 (m, 2 H) 8.64 (s, 1 H) 8.14 (br s, 1 H) 8.08 (br d, J = 9.05 Hz, 2 H) 7.81 (br d, J = 8.07 Hz, 2 H) 7.52- 7.57 (m, 1 H) 7.47- 7.52 (m, 1 H) 7.17- 7.24 (m, 1 H) 3.92 (br s, 3 H) 3.57-3.79 (m, 4 H) 3.51 (br s, 2 H) 3.17 (br s, 2 H) 2.35- 2.47 (m, 2 H) 2.07 (s, 2 H) | 2.24 A Example was prepared from Example 133. |
| 138 | 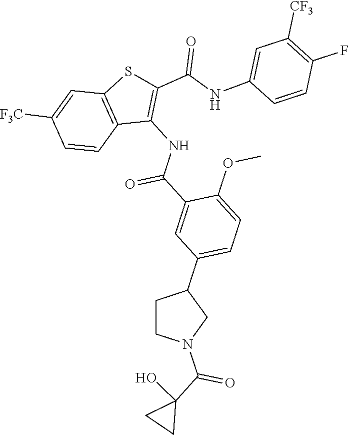 [SMILES: COc1ccc(C2CCN(C(=O)C3(O)CC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(1- hydroxycyclo- propane-1- carbonyl) pyrrolidin-3-yl)-2- methoxy- benzamido)-6- (trifluoromethyl) benzo[b] thiophene-2- carboxamide | 710.2 | 1H NMR (400 MHz, DMSO-d6) δ ppm 10.73-10.85 (m, 2 H) 8.62-8.68 (m, 1 H) 8.12-8.18 (m, 1 H) 8.03-8.10 (m, 1 H) 7.99-8.01 (m, 1 H) 7.94-7.96 (m, 1 H) 7.78-7.85 (m, 1 H) 7.72-7.78 (m, 1 H) 7.47-7.59 (m, 1 H) 7.17-7.26 (m, 1 H) 6.07-6.18 (m, 1 H) 4.23-4.31 (m, 1 H) 3.99 (s, 3 H) 3.90- 3.97 (m, 2 H) 3.72- 3.83 (m, 2 H) 3.36- 3.41 (m, 2 H) 1.82- 1.99 (m, 4 H) | 2.34 A Example was prepared from Example 133. |
| 139 | 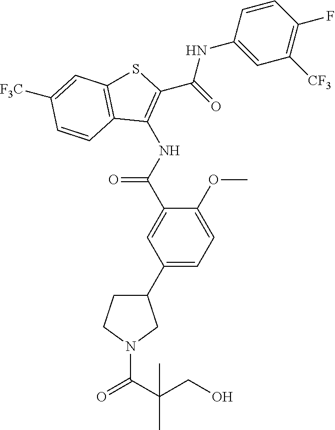 [SMILES: COc1ccc(C2CCN(C(=O)C(C)(C)CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(3-hydroxy- 2,2- dimethyl- propanoyl) pyrrolidin- 3-yl)-2- methoxy- benzamido)-6- (trifluoromethyl) benzo[b] thiophene-2- carboxamide | 726.3 | 1H NMR (400 MHz, DMSO-d6) δ ppm 10.91-10.70 (m, 2H), 8.66 (s, 1H), 8.16 (dd, J = 2.6, 6.7 Hz, 1H), 8.07 (d, J = 8.8 Hz, 1H), 7.98 (td, J = 3.5, 8.7 Hz, 1H),7.85-7.78 (m, 1H), 7.74 (s, 1H), 7.60- 7.41 (m, 2H), 7.26- 7.15 (m, 1H), 4.73- 4.59 (m, 1H), 3.93 (s, 4H), 3.51-3.38 (m, 4H), 2.26-2.12 (m, 2H), 1.93-1.76 (m, 2H), 1.13 (d, J = 6.8 Hz, 6H) | 2.38 A Example was prepared from Example 133. |
| 140 | 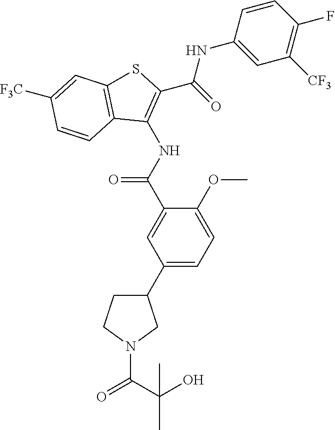 [SMILES: COc1ccc(C2CCN(C(=O)C(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(2-hydroxy- 2- methylpropanoyl) pyrrolidin-3- yl)-2- methoxy- benzamido)-6- (trifluoromethyl) benzo[b] thiophene-2- carboxamide | 712.2 | 1H NMR (400 MHz, DMSO-d6) δ ppm 10.94-10.61 (m, 2H), 8.65 (s, 1H), 8.15 (dd, J = 2.1, 6.5 Hz, 1H), 8.07 (br d, J = 8.1 Hz, 1H), 8.02-7.93 (m, 1H), 7.81 (dd, J = 1.1, 8.9 Hz, 1H), 7.74 (br d, J = 5.1 Hz, 1H), 7.59- 7.40 (m, 2H), 7.25- 7.16 (m, 1H), 5.29- 5.14 (m, 1H), 4.35- 4.27 (m, 1H), 4.13- 4.05 (m, 1H), 3.93 (brs, 3H), 3.85-3.63 (m, 1H), 3.61-3.47 (m, 1H), 3.28-3.20 (m, 1H), 2.27-2.08 (m, 1H), 1.97-1.72 (m, 1H), 1.35-1.23 (m, 6H) | 2.35 A Example was prepared from Example 133. |
| 141 | 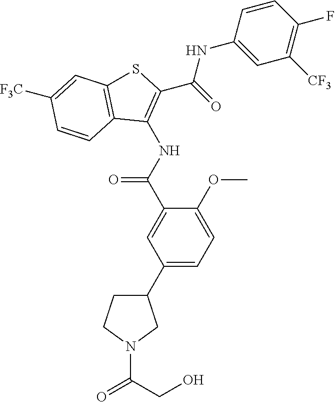 [SMILES: COc1ccc(C2CCN(C(=O)CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(2- hydroxyacetyl) pyrrolidin-3- yl)-2- methoxy- benzamido)-6- (trifluoromethyl) benzo[b] thiophene-2- carboxamide | 684.2 | 1H NMR (400 MHz, DMSO-d6) δ ppm 10.90-10.68 (m, 2H), 8.67 (s, 1H), 8.20- 8.12 (m, 1H), 8.07 (dd, J = 4.4, 8.6 Hz, 1H), 8.02-7.93 (m, 1H), 7.85-7.78 (m,1H), 7.77-7.68 (m, 1H), 7.59-7.41 (m, 2H), 7.21 (dd, J = 5.5, 8.7 Hz, 1H), 4.59-4.49 (m, 1H), 4.13-3.98 (m, 2H), 3.93 (d, J = 3.9 Hz, 3H), 3.91-3.73 (m, 1H), 3.68-3.51 (m, 1H), 3.46-3.38 (m, 1H), 3.27-3.18 (m, 1H), 2.30-2.14 (m, 1H), 2.01-1.79 (m, 1H) | 2.24 A Example was prepared from Example 133. |
| 142 | 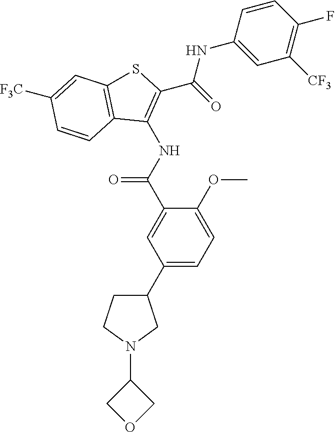 [SMILES: COc1ccc(C2CCN(C3COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (oxetan-3- yl)pyrrolidin-3- yl)benzamido)- 6- (trifluoromethyl) benzo[b] thiophene-2- carboxamide | 682.2 | 1H NMR (400 MHz, DMSO-d6) δ ppm 10.81 (s, 1H), 10.74 (s, 1H), 8.67 (s, 1H), 8.14 (dd, J = 2.4, 6.6 Hz, 1H), 8.06 (d, J = 8.8 Hz, 1H), 8.02-7.96 (m, 1H), 7.82 (dd, J = 1.3, 8.7 Hz, 1H), 7.72 (d, J = 2.2 Hz, 1H), 7.60- 7.41 (m, 2H), 7.18 (d, J = 8.6 Hz, 1H), 4.61- 4.53 (m, 2H), 4.47 (q, J = 6.2 Hz, 2H), 3.94 (s, 3H), 3.67-3.59 (m, 1H), 2.95-2.86 (m, 1H), 2.70-2.58 (m, 2H), 2.36 (br s, 2H), 2.29-2.16 (m, 1H), 1.79-1.65 (m, 1H) | 2.40 A Example was prepared from Example 133. |
| 143 | 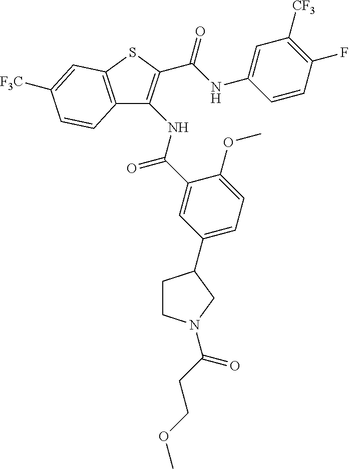 [SMILES: COCCC(=O)N1CCC(c2ccc(OC)c(C(=O)Nc3c(C(=O)Nc4ccc(F)c(C(F)(F)F)c4)sc4cc(C(F)(F)F)ccc34)c2)C1] | N-(4-fluoro-3- (trifluoromethyl) phenyl)-3-(2- methoxy-5-(1- (3- methoxypropanoyl) pyrrolidin- 3- yl)benzamido)- 6- (trifluoromethyl) benzo[b] thiophene-2- carboxamide | 712.1 | 1H NMR (400 MHz, DMSO-d6) δ ppm 10.69-10.85 (m, 2 H) 8.61-8.67 (m, 1 H) 8.12-8.17 (m, 1 H) 7.93-8.00 (m, 1 H) 7.78-7.84 (m, 1 H) 7.70-7.75 (m, 1 H) 7.58-7.58 (m, 1 H) 7.53 (br d, J = 9.29 Hz, 1 H) 7.45-7.57 (m, 1 H) 7.17-7.24 (m, 1 H) 4.02 (br d, J = 8.80 Hz, 2 H) 3.93 (br s, 3 H) 3.60 (br s, 2 H) 3.52 (br s, 2 H) 3.16-3.34 (m, 8 H) | 2.38 A Example was prepared from Example 133. |
| 144 | 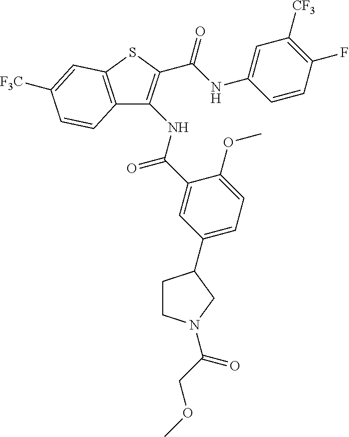 [SMILES: COCC(=O)N1CCC(c2ccc(OC)c(C(=O)Nc3c(C(=O)Nc4ccc(F)c(C(F)(F)F)c4)sc4cc(C(F)(F)F)ccc34)c2)C1] | N-(4-fluoro-3- (trifluoromethyl) phenyl)-3-(2- methoxy-5-(1- (2- methoxyacetyl) pyrrolidin-3- yl)benzamido)- 6- (trifluoromethyl) benzo[b] thiophene-2- carboxamide | 698.2 | 1H NMR (400 MHz, DMSO-d6) δ ppm 10.69-10.85 (m, 2 H) 8.61-8.67 (m, 1 H) 8.12-8.17 (m, 1 H) 7.93-8.00 (m, 1 H) 7.78-7.84 (m, 1 H) 7.70-7.75 (m, 1 H) 7.58-7.58 (m, 1 H) 7.53 (br d, J = 9.29 Hz, 1 H) 7.45-7.57 (m, 1 H) 7.17-7.24 (m, 1 H) 4.02 (br d, J = 8.80 Hz, 2 H) 3.93 (br s, 3 H) 3.60 (br s, 2 H) 3.52 (br s, 1 H) 3.16-3.34 (m, 8 H) | 2.34 A Example was prepared from Example 133. |
| 145 | 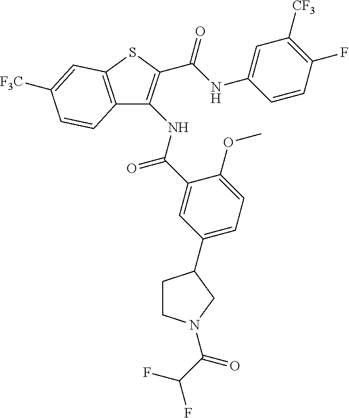 [SMILES: COc1ccc(C2CCN(C(=O)C(F)F)C2)cc1C(=O)Nc1c(C(=O)Nc2ccc(F)c(C(F)(F)F)c2)sc2cc(C(F)(F)F)ccc12] | 3-(5-(1-(2,2- difluoroacetyl) pyrrolidin-3- yl)-2- methoxy- benzamido)-N-(4- fluoro-3- (trifluoromethyl) phenyl)-6- (trifluoromethyl) benzo[b] thiophene-2- carboxamide | 721.1 (M + H + NH₃)⁺ | 1H NMR (400 MHz, DMSO-d6) δ ppm 10.53-10.79 (m, 2 H) 8.66 (s, 1 H) 8.16 (br s, 1 H) 8.08 (d, J = 8.57 Hz, 1 H) 7.96-7.99 (m, 1 H) 7.82 (br d, J = 7.09 Hz, 2 H) 7.50- 7.64 (m, 2 H) 7.28 (d, J = 8.80 Hz, 1 H) 4.81 (s, 1H), 3.97 (s, 3 H) 3.24 (br s, 2 H) 3.17 (br s, 4 H) 1.23 (s, 1 H) | 2.47 A Example was prepared from Example 133. |
| 146 | 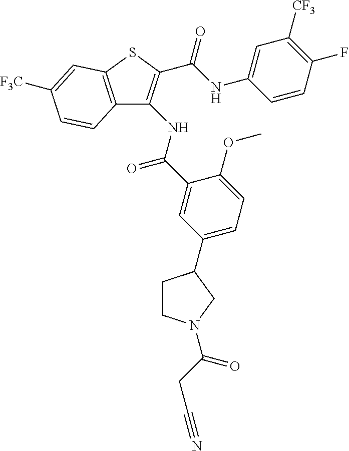 [SMILES: COc1ccc(C2CCN(C(=O)CC#N)C2)cc1C(=O)Nc1c(C(=O)Nc2ccc(F)c(C(F)(F)F)c2)sc2cc(C(F)(F)F)ccc12] | 3-(5-(1-(2- cyanoacetyl) pyrrolidin-3-yl)- 2- methoxy- benzamido)-N-(4- fluoro-3- (trifluoromethyl) phenyl)-6- (trifluoromethyl) benzo[b] thiophene-2- carboxamide | 693.2 | 1H NMR (400 MHz, DMSO-d6) δ ppm 10.53-10.79 (m, 2 H) 8.66 (s, 1 H) 8.16 (br s, 1 H) 8.08 (d, J = 8.57 Hz, 1 H) 7.96-7.99 (m, 1 H) 7.82 (br d, J = 7.09 Hz, 2 H) 7.50- 7.64 (m, 2 H) 7.28 (d, J = 8.80 Hz, 1 H) 4.81 (s, 1H), 3.97 (s, 3 H) 3.24 (br s, 2 H) 3.17 (br s, 4 H) 1.23 (s, 2 H) | 2.37 B Example was prepared from Example 133. |
| 147 | 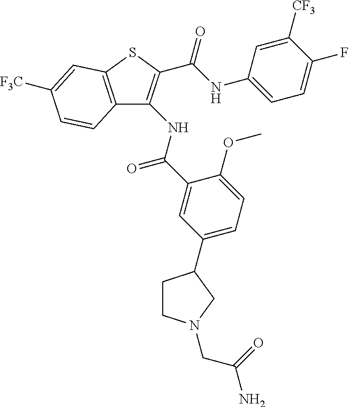 [SMILES: COc1ccc(C2CCN(CC(N)=O)C2)cc1C(=O)Nc1c(C(=O)Nc2ccc(F)c(C(F)(F)F)c2)sc2cc(C(F)(F)F)ccc12] | 3-(5-(1-(2- amino-2- oxoethyl) pyrrolidin-3-yl)-2- methoxy- benzamido)-N-(4- fluoro-3- (trifluoromethyl) phenyl)-6- (trifluoromethyl) benzo[b] thiophene-2- carboxamide | 683.2 | 1H NMR (400 MHz, DMSO-d6) δ ppm 10.93-10.67 (m, 2H), 8.66 (s, 1H), 8.14 (dd, J = 2.2, 6.6 Hz, 1H), 8.07 (d, J = 8.8 Hz, 1H), 8.03-7.95 (m, 1H), 7.81 (dd, J = 1.5, 8.6 Hz, 1H), 7.72 (s, 1H), 7.60- 7.42 (m, 2H), 7.24- 7.00 (m, 3H), 3.93 (s, 3H), 3.11-2.95 (m, 3H), 2.81-2.68 (m, 2H), 2.29-2.16 (m, 2H), 1.79-1.67 (m, 2H) | 2.27 A Example was prepared from Example 133. |
| 148 | 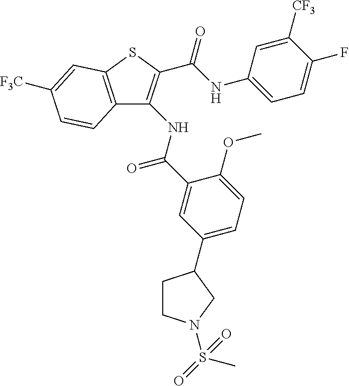 [SMILES: COc1ccc(C2CCN(S(C)(=O)=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (methylsulfonyl) pyrrolidin-3- yl)benzamido)- 6- (trifluoromethyl) benzo[b] thiophene-2- carboxamide | 702.1 (M − H)⁻ | 1H NMR (400 MHz, DMSO-d6) δ ppm 10.92-10.60 (m, 2H), 8.66 (s, 1H), 8.17 (dd, J = 2.4, 6.6 Hz, 1H), 8.07 (d, J = 8.6 Hz, 1H), 8.02-7.92 (m, 1H), 7.86-7.67 (m, 2H), 7.61-7.46 (m, 2H), 7.22 (d, J = 8.6 Hz, 1H), 3.94 (s, 3H), 3.70 (dd, J = 7.9, 9.2 Hz, 1H), 3.53-3.39 (m, 1H), 3.33 (br s, 1H), 3.20 (s, 1H), 3.12 (t, J = 9.8 Hz, 1H), 2.96 (s, 3H), 2.31- 2.16 (m, 1H), 1.96 (quin, J = 10.3 Hz, 1H) | 2.40 A Example was prepared from Example 133. |
| 149 | 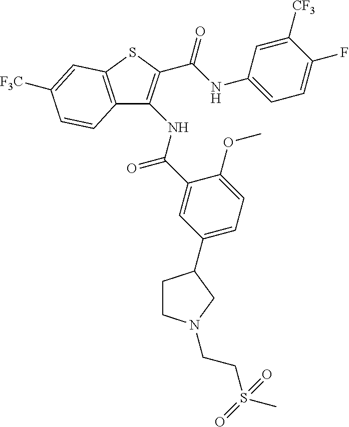 [SMILES: COc1ccc(C2CCN(CCS(C)(=O)=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (2- (methylsulfonyl) ethyl)pyrrolidin- 3- yl)benzamido)- 6- (trifluoromethyl) benzo[b] thiophene-2- carboxamide | 732.0 | 1H NMR (400 MHz, DMSO-d6) δ ppm 10.82 (br s, 1H), 10.74 (br d, J = 1.2 Hz, 1H), 8.67 (s, 1H), 8.15 (dd, J = 2.4, 6.4 Hz, 1H), 8.06 (d, J = 8.6 Hz, 1H), 7.99 (td, J = 3.9, 7.9 Hz, 1H), 7.85-7.79 (m, 1H), 7.72 (d, J = 2.2 Hz, 1H), 7.55 (t, J = 9.8 Hz, 1H), 7.48 (dd, J = 2.3, 8.4 Hz, 1H), 7.17 (d, J = 8.6 Hz, 1H), 3.94 (s, 3H), 3.32-3.27 (m, 3H), 3.02 (s, 3H), 2.95 (br t, J = 8.3 Hz, 1H), 2.89-2.79 (m, 2H), 2.48-2.42 (m, 2H), 2.28-2.17 (m, 1H), 1.77-1.62 (m, 2H) | 2.38 A Example was prepared from Example 133. |
| 150 | 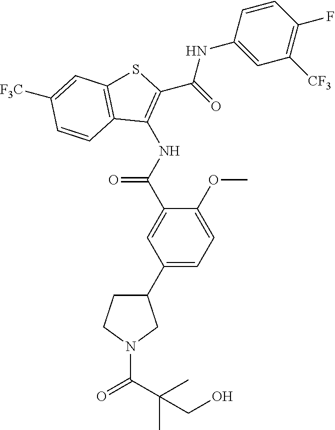 [SMILES: COc1ccc(C2CCN(C(=O)C(C)(C)CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(3-hydroxy- 2,2- dimethyl- propanoyl) pyrrolidin- 3-yl)-2- methoxy- benzamido)-6- (trifluoromethyl) benzo[b] thiophene-2- carboxamide | 726.3 | 1H NMR (400 MHz, DMSO-d6) δ ppm 10.83 (s, 1H), 10.74 (s, 1H), 8.67 (s, 1H), 8.16 (dd, J = 2.4, 6.4 Hz, 1H), 8.07 (d, J = 8.6 Hz, 1H), 7.99 (td, J = 3.6, 8.7 Hz, 1H), 7.81 (dd, J = 1.5, 8.8 Hz, 1H), 7.75 (d, J = 2.2 Hz, 1H), 7.60-7.46 (m, 2H), 7.21 (d, J = 8.8 Hz, 1H), 4.65 (t, J = 5.6 Hz, 3H), 3.94 (s, 3H), 3.50- 3.39 (m,3H), 2.25- 2.11 (m, 2H), 1.96- 1.79 (m, 2H), 1.13 (d, J = 6.8 Hz, 6H) | 2.39 A Example was prepared from Intermediate 133-1. |
| 151 | 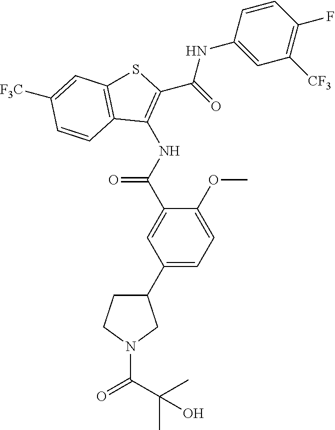 [SMILES: COc1ccc(C2CCN(C(=O)C(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(2-hydroxy- 2- methylpropanoyl) pyrrolidin-3- yl)-2- methoxy- benzamido)-6- (trifluoromethyl) benzo[b] thiophene-2- carboxamide | 712.3 | 1H NMR (400 MHz, DMSO-d6) δ ppm 10.84 (br d, J = 2.0 Hz, 1H), 10.74 (s, 1H), 8.67 (s, 1H), 8.16 (dd, J = 2.4, 6.6 Hz, 1H), 8.10-8.04 (m, 1H), 8.02-7.93 (m,1H), 7.81 (dd, J = 1.3, 8.7 Hz, 1H), 7.75 (br d, J = 4.4 Hz, 1H), 7.61- 7.40 (m, 2H), 7.26- 7.11 (m, 1H), 5.18 (d, J = 4.6 Hz, 1H), 4.35- 4.03 (m, 1H), 3.94 (d, J = 2.7 Hz, 3H), 3.83- 3.66 (m, 1H), 3.61- 3.45 (m, 1H), 3.37 (br s, 1H), 3.30-3.20 (m, 1H), 2.27-2.10 (m, 1H), 1.94-1.78 (m, 1H), 1.35-1.24 (m, 6H) | 2.36 A Example was prepared from Intermediate 133-1. |
| 152 | 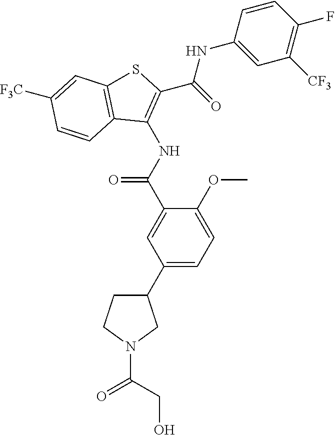 [SMILES: COc1ccc(C2CCN(C(=O)CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1-(2- hydroxyacetyl) pyrrolidin-3- yl)-2- methoxy- benzamido)-6- (trifluoromethyl) benzo[b] thiophene-2- carboxamide | 684.2 | 1H NMR (400 MHz, DMSO-d6) δ ppm 10.82 (s, 1H), 10.72 (s, 1H), 8.66 (s, 1H), 8.19- 8.11 (m, 1H), 8.09- 8.03 (m, 1H), 8.02- 7.92 (m, 1H), 7.85- 7.78 (m, 1H), 7.75 (dd, J = 2.3, 3.3 Hz, 1H), 7.61-7.45 (m, 2H), 7.28-7.13 (m, 1H), 4.56-4.48 (m, 1H), 4.08-3.98 (m, 2H), 3.96-3.76 (m, 4H), 3.66-3.54 (m, 1H), 3.47-3.40 (m, 1H), 3.27-3.20 (m, 1H), 2.30-2.15 (m, 2H), 1.99-1.77 (m, 1H) | 2.24 A Example was prepared from Intermediate 133-1. |
| 153 | 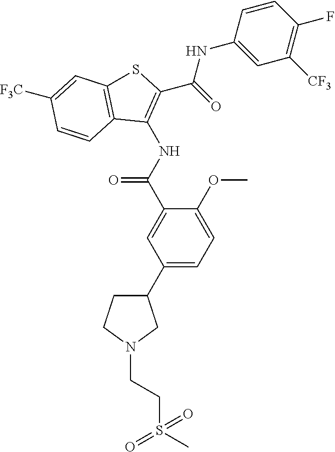 [SMILES: COc1ccc(C2CCN(CCS(C)(=O)=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (2- (methylsulfonyl) ethyl) pyrrolidin-3- yl)benzamido)- 6- (trifluoromethyl) benzo[b] thiophene-2- carboxamide | 732.2 | 1H NMR (400 MHz, DMSO-d6) δ ppm 10.83 (s, 1H), 10.74 (s, 1H), 8.67 (s, 1H), 8.15 (dd, J = 2.7, 6.6 Hz, 1H), 8.06 (d, J = 8.6 Hz, 1H), 8.02-7.95 (m, 1H), 7.82 (dd, J = 1.5, 8.8 Hz, 1H), 7.72 (d, J = 2.0 Hz, 1H), 7.55 (t, J = 9.8 Hz, 1H), 7.50- 7.39 (m, 1H), 7.17 (d, J = 8.6 Hz, 1H), 3.94 (s, 3H), 3.32-3.26 (m, 3H), 3.02 (s, 3H), 2.99- 2.92 (m, 1H), 2.90- 2.79 (m, 2H), 2.74- 2.66 (m, 2H), 2.48- 2.42 (m, 1H), 2.28- 2.17 (m, 1H), 1.75- 1.64 (m, 1H) | 2.38 A Example was prepared from Intermediate 133-1. |
| 154 | 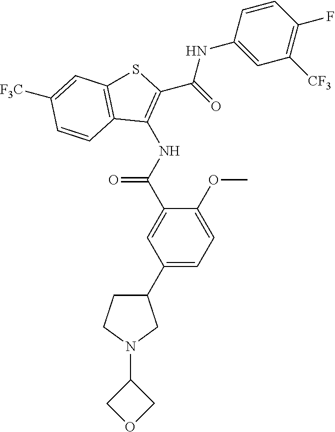 [SMILES: COc1ccc(C2CCN(C3COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- (oxetan-3- yl)pyrrolidin-3- yl)benzamido)- 6- (trifluoromethyl) benzo[b] thiophene-2- carboxamide | 682.3 | 1H NMR (400 MHz, DMSO-d6) δ ppm 10.81 (s, 1H), 10.73 (s, 1H), 8.67 (s, 1H), 8.14 (dd, J = 2.6, 6.5 Hz, 1H), 8.06 (d, J = 9.0 Hz, 1H), 8.02-7.94 (m, 1H), 7.85-7.78 (m, 1H), 7.71 (d, J = 2.4 Hz, 1H), 7.57-7.45 (m, 2H), 7.18 (d, J = 8.6 Hz, 1H), 4.57 (dt, J = 1.8, 6.4 Hz, 2H), 4.47 (q, J = 6.0 Hz, 2H), 3.94 (s, 3H), 3.62 (brd, J = 2.2 Hz, 1H), 2.93-2.88 (m, 1H), 2.65-2.55 (m, 2H), 2.29-2.20 (m, 2H), 1.75-1.65 (m, 1H), 1.23 (s, 1H) | 1.97 B Example was prepared from Intermediate 133-1. |
| 155 | 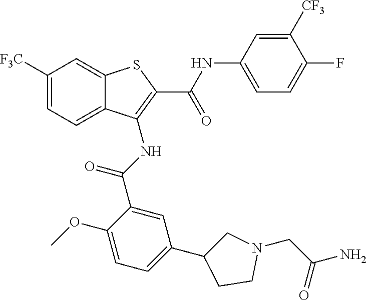 [SMILES: COc1ccc(C2CCN(CC(N)=O)C2)cc1C(=O)Nc1c(C(=O)Nc2ccc(F)c(C(F)(F)F)c2)sc2cc(C(F)(F)F)ccc12] | 3-{5-[1- (carbamoyl- methyl)pyrrolidin- 3-yl]-2- methoxy- benzamido}-N-[4- fluoro-3- (trifluoromethyl) phenyl]-6- (trifluoromethyl)- 1- benzothiophene- 2- carboxamide | 683.2 | 1H NMR (400 MHz, DMSO-d6) δ ppm 10.83 (s, 1H), 10.75 (s, 1H), 8.67 (s, 1H), 8.15 (dd, J = 2.6, 6.5 Hz, 1H), 8.06 (d, J = 8.6 Hz, 1H), 8.02-7.96 (m, 1H), 7.85-7.79 (m, 1H), 7.74 (d, J = 2.0 Hz, 1H), 7.60-7.41 (m, 2H), 7.25 (br dd, J = 2.4, 3.9 Hz, 1H), 7.22-7.08 (m, 2H), 3.94 (s, 3H), 3.90 (s, 1H), 3.21-3.02 (m, 3H), 2.90-2.75 (m, 2H), 2.65-2.55 (m, 1H), 2.30-2.18 (m, 1H), 1.84-1.69 (m, 1H). | 2.27 A Example was prepared from Intermediate 133-1. |
| 156 | 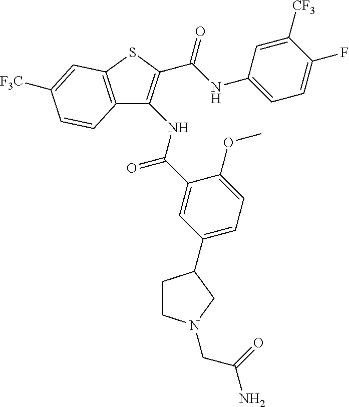 [SMILES: COc1ccc(C2CCN(CC(N)=O)C2)cc1C(=O)Nc1c(C(=O)Nc2ccc(F)c(C(F)(F)F)c2)sc2cc(C(F)(F)F)ccc12] | 3-(5-(1-(2- amino-2- oxoethyl) pyrrolidin-3-yl)-2- methoxy- benzamido)-N-(4- fluoro-3- (trifluoromethyl) phenyl)-6- (trifluoromethyl) benzo[b] thiophene-2- carboxamide | 683.2 | 1H NMR (400 MHz, DMSO-d6) δ ppm 10.83 (s, 1H), 10.75 (s, 1H), 8.67 (s, 1H), 8.15 (dd, J = 2.6, 6.2 Hz, 1H), 8.06 (d, J = 8.8 Hz, 1H), 8.02-7.94 (m, 2H), 7.82 (dd, J = 1.6, 8.7 Hz, 1H), 7.75 (d, J = 2.0 Hz, 1H), 7.59- 7.43 (m, 2H), 7.32- 7.09 (m, 2H), 3.94 (s, 3H), 3.90 (s, 1H), 3.25- 3.05 (m, 3H), 2.93- 2.76 (m, 2H), 2.66- 2.58 (m, 1H), 2.31- 2.16 (m, 1H), 1.85- 1.70 (m, 1H) | 2.27 A (Example was prepared from Intermediate 133-1. |
| 157 | 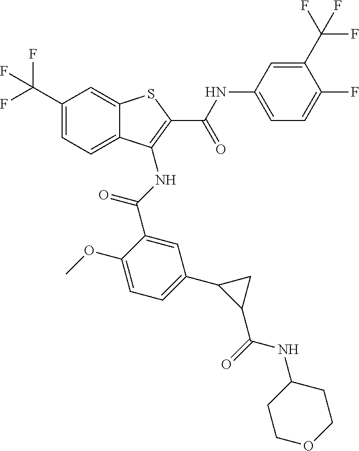 [SMILES: COc1ccc(C2CC2C(=O)NC2CCOCC2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-(2- methoxy-5-{2- [(oxan-4- yl)carbamoyl] cyclopropyl} benzamido)-6- (trifluoromethyl)- 1- benzothiophene- 2- carboxamide | 724.1 | 1H NMR (400 MHz, DMSO-d6) δ ppm 10.92-10.64 (m, 2H), 8.66 (s, 1H), 8.14 (dd, J = 6.2, 2.3 Hz, 1H), 8.07 (d, J = 8.6 Hz, 1H), 8.00 (br dd, J = 8.4, 3.8 Hz, 1H), 7.85-7.74 (m, 1H), 7.66-7.47 (m, 2H), 7.38 (br d, J = 8.1 Hz, 1H), 7.15 (br d, J = 8.8 Hz, 1H), 4.75 (br s, 1H), 3.99-3.81 (m, 5H), 3.74-3.60 (m, 1H), 3.30-3.19 (m, 1H), 3.11-2.95 (m, 1H), 2.31-2.23 (m, 1H), 2.23-2.09 (m, 1H), 1.83-1.61 (m, 2H), 1.44-1.33 (m, 1H), 1.33-1.18 (m, 2H), 1.14 (br t, J = 8.8 Hz, 1H). | 2.30 B Example prepared from Example 47. |
| 158 | 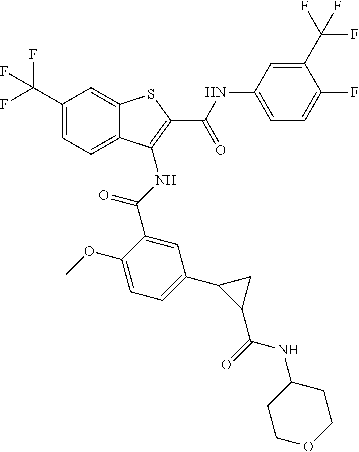 [SMILES: COc1ccc(C2CC2C(=O)NC2CCOCC2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-(2- methoxy-5-{2- [(oxan-4- yl)carbamoyl] cyclopropyl} benzamido)-6- (trifluoromethyl)- 1- benzothiophene- 2- carboxamide | 724.2 | 1H NMR (400 MHz, DMSO-d6) δ ppm 10.94-10.62 (m, 2H), 8.65 (s, 1H), 8.14 (dd, J = 6.4, 2.4 Hz, 1H), 8.08 (d, J = 8.8 Hz, 1H), 8.03-7.91 (m, 1H), 7.80 (dd, J = 8.7, 1.3 Hz, 1H), 7.59 (br s, 1H), 7.53 (t, J = 9.8 Hz, 1H), 7.38 (br d, J = 9.0 Hz, 1H), 7.14 (br d, J = 8.6 Hz, 1H), 4.81- 4.65 (m, 1H), 3.98- 3.80 (m, 5H), 3.74- 3.62 (m, 1H), 3.28- 3.19 (m, 1H), 3.08- 2.95 (m, 1H), 2.32- 2.24 (m, 1H), 2.24- 2.11 (m, 1H), 1.79- 1.61 (m, 2H), 1.43- 1.34 (m, 1H), 1.34- 1.19 (m, 2H), 1.19- 1.06 (m, 1H). | 2.34 A Example prepared from Example 48. |
| 159 | 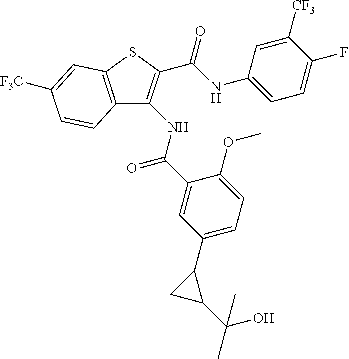 [SMILES: COc1ccc(C2CC2C(C)(C)O)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (2-(2- hydroxypropan- 2- yl)cyclopropyl)- 2- methoxy- benzamido)-6- (trifluoromethyl) benzo[b] thiophene-2- carboxamide | 653.2 (M − H)⁻ | 1H NMR (400 MHz, DMSO-d6) δ ppm 10.87-10.65 (m, 2H), 8.65 (s, 1H), 8.14 (dd, J = 6.6, 2.2 Hz, 1H), 8.07 (d, J = 8.8 Hz, 1H), 7.98 (dt, J = 7.4, 4.4 Hz, 1H), 7.81 (dd, J = 8.4, 1.3 Hz, 1H), 7.53 (t, J = 9.9 Hz, 1H), 7.47 (s, 1H), 7.28 (dd, J = 8.2, 2.1 Hz, 1H), 7.11 (d, J = 8.6 Hz, 1H), 4.14 (s, 1H), 3.95- 3.85 (m, 3H), 1.98- 1.88 (m, 1H), 1.29- 1.21 (m, 2H), 1.14 (s, 6H), 1.07-0.92 (m, 1H) | 2.55 A Prepared from Intermediate 46-4. |
| 160 | 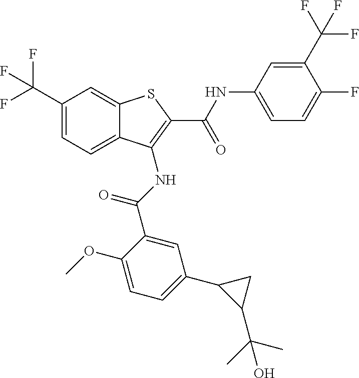 [SMILES: COc1ccc(C2CC2C(C)(C)O)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [2-(2- hydroxypropan- 2- yl)cyclopropyl]- 2- methoxy- benzamido}-6- (trifluoromethyl)- 1- benzothiophene- 2- carboxamide | 653.1 (M − H)⁻ | 1H NMR (400 MHz, DMSO-d6) δ ppm 8.33 (s, 1H), 8.20 (d, J = 8.1 Hz, 1H), 8.01 (dd, J = 2.3, 7.2 Hz, 1H), 7.69-7.62 (m, 2H), 7.62-7.55 (m, 1H), 7.35 (dd, J = 1.8, 8.4 Hz, 1H), 7.30-7.23 (m, 1H), 7.06 (d, J = 8.6 Hz, 1H), 5.15-5.08 (m, 1H), 4.51 (t, J = 6.5 Hz, 1H), 3.78 (s, 3H), 2.31-2.23 (m, 1H), 1.67 (s, 4H), 1.60 (s, 3H), 1.47 (s, 3H). | 2.69 A Example 159 was purified by prep SFC (Method 22) to give Example 160 (Peak 1, Prep SFC RT = 10.81 min) 98% ee Analytical SFC (Method 23) |
| 161 | 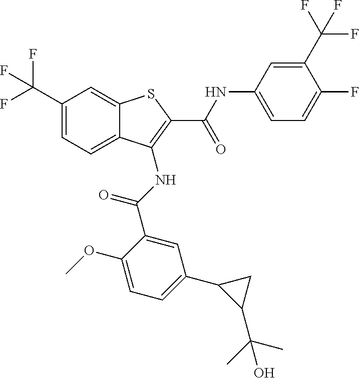 [SMILES: COc1ccc(C2CC2C(C)(C)O)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [2-(2- hydroxypropan- 2- yl)cyclopropyl]- 2- methoxy- benzamido}-6- (trifluoromethyl)- 1- benzothiophene- 2- carboxamide | 653.1 (M − H)⁻ | 1H NMR (400 MHz, DMSO-d6) δ ppm 8.33 (s, 1H), 8.20 (d, J = 8.1 Hz, 1H), 8.01 (dd, J = 2.3, 7.2 Hz, 1H), 7.69-7.62 (m, 2H), 7.62-7.55 (m, 1H), 7.35 (dd, J = 1.8, 8.4 Hz, 1H), 7.30-7.23 (m, 1H), 7.06 (d, J = 8.6 Hz, 1H), 5.15-5.08 (m, 1H), 4.51 (t, J = 6.5 Hz, 1H), 3.78 (s, 3H), 2.31-2.23 (m, 1H), 1.67 (s, 4H), 1.60 (s, 3H), 1.47 (s, 3H). | 2.69 A Example 159 was purified by prep SFC (Method 22) to give Example 161 (Peak 2, Prep SFC RT = 13.95 min). 98% ee Analytical SFC (Method 23) |
| 162 | 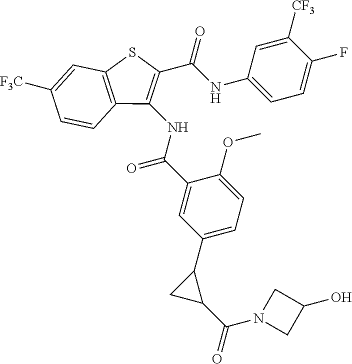 [SMILES: COc1ccc(C2CC2C(=O)N2CC(O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (2-(3- hydroxyazetidine- 1- carbonyl) cyclopropyl)-2- methoxy- benzamido)-6- (trifluoromethyl) benzo[b] thiophene-2- carboxamide | 696.3 | 1H NMR (400 MHz, DMSO-d6) δ ppm 10.81 (br s, 2H), 8.64 (s, 1H), 8.19-8.10 (m, 1H), 8.08 (d, J = 8.6 Hz, 1H), 8.01-7.87 (m, 1H), 7.80 (dd, J = 1.5, 8.6 Hz, 1H), 7.62-7.48 (m, 2H), 7.41-7.28 (m, 1H), 7.20-7.07 (m, 1H), 5.72 (br d, J = 2.9 Hz, 1H), 4.50- 4.22 (m, 2H), 4.10- 4.00 (m, 1H), 3.97- 3.91 (m, 3H), 3.59 (br dd, J = 3.5, 10.1 Hz, 1H), 2.30-2.21 (m, 1H), 1.77-1.65 (m, 2H), 1.37-1.28 (m, 1H), 1.22-1.10 (m, 1H) | 2.20 B Example prepared from Example 46. |
| 163 | 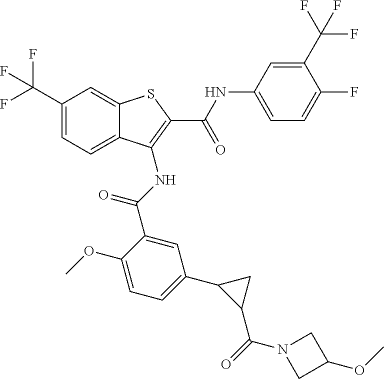 [SMILES: COc1ccc(C2CC2C(=O)N2CC(OC)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{2- methoxy-5-[2- (3- methoxyazetidine- 1- carbonyl) cyclopropyl] benzamido}-6- (trifluoromethyl)- 1- benzothiophene- 2- carboxamide | 710.2 | 1H NMR (400 MHz, DMSO-d6) δ ppm 10.81 (s, 1H), 10.73 (s, 1H), 8.67 (s, 1H), 8.14 (dd, J = 6.5, 2.6 Hz, 1H), 8.07 (d, J = 8.3 Hz, 1H), 8.03-7.94 (m, 1H), 7.81 (dd, J = 8.8, 1.5 Hz, 1H), 7.62-7.44 (m, 2H), 7.43-7.30 (m, 1H), 7.15 (d, J = 8.6 Hz, 1H), 4.43-4.27 (m, 1H), 4.24-4.15 (m, 1H), 4.07-4.00 (m, 2H), 3.96-3.81 (m, 4H), 3.68-3.58 (m, 1H), 3.19 (d, J = 5.6 Hz, 3H), 2.32-2.24 (m, 1H), 1.78-1.70 (m, 1H), 1.39-1.14 (m, 3H). | 2.42 A Example was prepared from Example 47. |
| 164 | 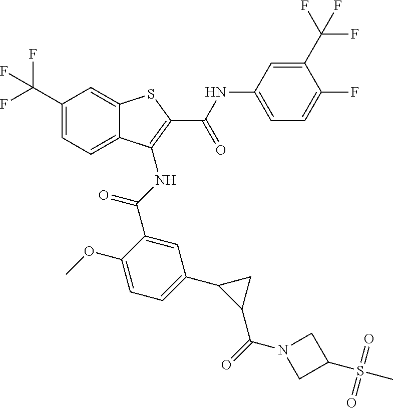 [SMILES: COc1ccc(C2CC2C(=O)N2CC(S(C)(=O)=O)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [2-(3- methane- sulfonylazetidine-1- carbonyl) cyclopropyl]-2- methoxy- benzamido}-6- (trifluoromethyl)- 1- benzothiophene- 2- carboxamide | 758.2 | 1H NMR (400 MHz, DMSO-d6) δ ppm 10.82 (s, 1H), 10.73 (s, 1H), 8.67 (s, 1H), 8.15 (dt, J = 3.9, 2.2 Hz, 1H), 8.12-8.02 (m, 1H), 8.02-7.91 (m, 1H), 7.81 (d, J = 7.8 Hz, 1H), 7.66-7.48 (m, 2H), 7.45-7.31 (m, 1H), 7.16 (d, J = 8.8 Hz, 1H), 4.60-4.24 (m, 3H), 4.20-4.12 (m, 1H), 4.08-4.00 (m, 1H), 3.91 (d, J = 5.1 Hz, 4H), 3.02 (d, J = 3.7 Hz, 3H), 2.38-2.30 (m, 1H), 1.85-1.77 (m, 1H), 1.40-1.31 (m, 1H), 1.23 (br s, 2H). | 2.28 B Example was prepared from Example 47. |
| 165 | 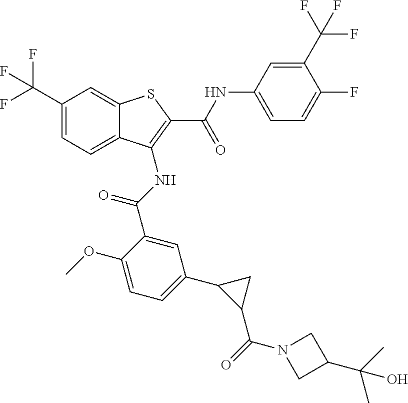 [SMILES: COc1ccc(C2CC2C(=O)N2CC(C(C)(C)O)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-(5- {2-[3-(2- hydroxypropan- 2-yl)azetidine- 1- carbonyl] cyclopropyl}-2- methoxy- benzamido)-6- (trifluoromethyl)- 1- benzothiophene- 2- carboxamide | 738.3 | 1H NMR (400 MHz, DMSO-d6) δ ppm 10.81 (s, 1H), 10.73 (s, 1H), 8.66 (s, 1H), 8.21- 8.11 (m, 1H), 8.07 (d, J = 8.8 Hz, 1H), 8.03- 7.90 (m, 1H), 7.85- 7.76 (m, 1H), 7.61- 7.48 (m, 2H), 7.42- 7.28 (m, 1H), 7.15 (d, J = 8.6 Hz, 1H), 4.53 (d, J = 15.9 Hz, 1H), 4.15- 4.07 (m, 1H), 4.05- 3.96 (m, 1H), 3.93- 3.87 (m, 3H), 3.80- 3.70 (m, 2H), 2.60- 2.52 (m, 1H), 2.30- 2.22 (m, 1H), 1.79- 1.70 (m, 1H), 1.38- 1.27 (m, 1H), 1.17 (br dd, J = 5.6, 2.4 Hz, 1H), 1.05-0.88 (m, 6H). | 2.37 A Example was prepared from Example 47. |
| 166 | 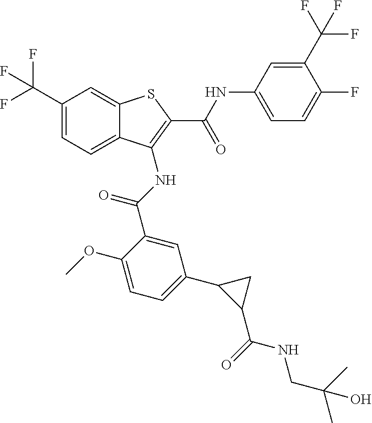 [SMILES: COc1ccc(C2CC2C(=O)NCC(C)(C)O)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-(5- {2-[(2- hydroxy-2- methylpropyl) carbamoyl] cyclopropyl}-2- methoxy- benzamido)-6- (trifluoromethyl)- 1- benzothiophene- 2- carboxamide | 712.2 | 1H NMR (400 MHz, DMSO-d6) δ ppm 10.90-10.62 (m, 2H), 8.67 (s, 1H), 8.15 (dd, J = 6.4, 2.2 Hz, 1H), 8.06 (d, J = 8.6 Hz, 1H), 8.03-7.91 (m, 2H), 7.81 (dd, J = 8.6, 1.2 Hz, 1H), 7.60-7.45 (m, 2H), 7.35 (dd, J = 8.4, 2.3 Hz, 1H), 7.16 (d, J = 8.6 Hz, 1H), 4.44 (s, 1H), 3.92 (s, 3H), 3.11- 2.99 (m, 2H), 2.29- 2.20 (m, 1H), 2.07- 1.99 (m, 1H), 1.32 (dt, J = 9.0, 4.5 Hz, 1H), 1.23 (s, 1H), 1.04 (d, J = 3.7 Hz, 6H). | 2.38 A Example was prepared from Example 47. |
| 167 | 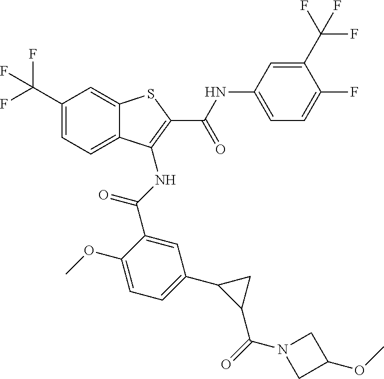 [SMILES: COc1ccc(C2CC2C(=O)N2CC(OC)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{2- methoxy-5-[2- (3- methoxyazetidine- 1- carbonyl) cyclopropyl] benzamido}-6- (trifluoromethyl)- 1- benzothiophene- 2- carboxamide | 710.2 | 1H NMR (400 MHz, DMSO-d6) δ ppm 10.91-10.64 (m, 2H), 8.66 (s, 1H), 8.14 (dd, J = 6.2, 2.6 Hz, 1H), 8.11-8.03 (m, 1H), 8.02-7.94 (m, 1H), 7.85-7.75 (m, 1H), 7.63-7.48 (m, 2H), 7.42-7.32 (m, 1H), 7.15 (d, J = 8.8 Hz, 1H), 4.44-4.26 (m, 1H), 4.24-4.13 (m, 1H), 4.09-3.98 (m, 2H), 3.96-3.86 (m, 4H), 3.65 (br dd, J = 10.5, 3.4 Hz, 1H), 3.22-3.16 (m, 3H), 2.33-2.25 (m, 1H), 1.79-1.70 (m, 1H), 1.36-1.27 (m, 1H), 1.26-1.13 (m, 2H). | 2.46 A Example was prepared from Example 48. |
| 168 | 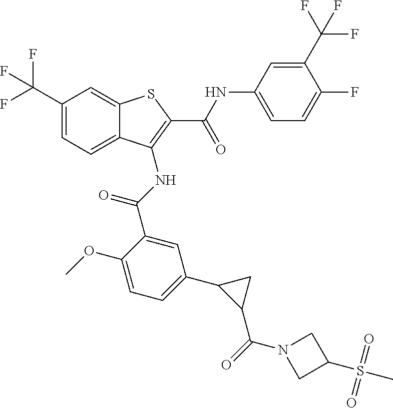 [SMILES: COc1ccc(C2CC2C(=O)N2CC(S(C)(=O)=O)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [2-(3- methane- sulfonylazetidine-1- carbonyl) cyclopropyl]-2- methoxy- benzamido}-6- (trifluoromethyl)- 1- benzothiophene- 2- carboxamide | 758.1 | 1H NMR (400 MHz, DMSO-d6) δ ppm 10.89-10.62 (m, 2H), 8.66 (s, 1H), 8.20-8.12 (m, 1H), 8.07 (dd, J = 8.4, 3.8 Hz, 1H), 7.99 (br dd, J = 5.4, 3.2 Hz, 1H), 7.81 (d, J = 8.6 Hz, 1H), 7.64-7.49 (m, 2H), 7.38 (br d, J = 8.3 Hz, 1H), 7.16 (d, J = 8.8 Hz, 1H), 4.61- 4.41 (m, 2H), 4.38- 4.23 (m, 2H), 4.21- 4.09 (m, 1H), 4.07- 4.00 (m, 1H), 3.90 (d, J = 4.6 Hz, 3H), 3.02 (d, J = 3.9 Hz, 3H), 2.37- 2.26 (m, 1H), 1.87- 1.76 (m, 1H), 1.39- 1.32 (m, 1H), 1.28- 1.15 (m, 2H). | 2.34 A Example was prepared from Example 48. |
| 169 | 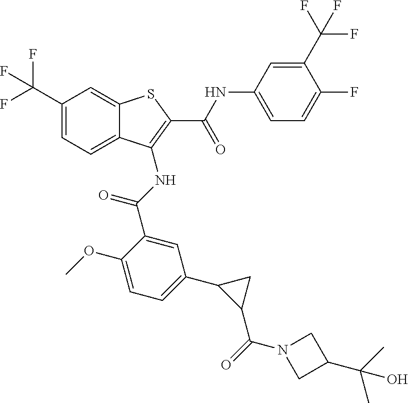 [SMILES: COc1ccc(C2CC2C(=O)N2CC(C(C)(C)O)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-(5- {2-[3-(2- hydroxypropan- 2-yl)azetidine- 1- carbonyl] cyclopropyl}-2- methoxy- benzamido)-6- (trifluoromethyl)- 1- benzothiophene- 2- carboxamide | 738.3 | 1H NMR (400 MHz, DMSO-d6) δ ppm 10.90-10.61 (m, 2H), 8.66 (s, 1H), 8.20-8.11 (m, 1H), 8.08 (d, J = 8.3 Hz, 1H), 8.03-7.89 (m, 1H), 7.81 (d, J = 8.8 Hz, 1H), 7.60-7.45 (m, 2H), 7.41-7.28 (m, 1H), 7.14 (br d, J = 8.6 Hz, 1H), 4.59-4.42 (m, 1H), 4.16-4.07 (m, 1H), 4.06-3.98 (m, 1H), 3.90 (br s, 3H), 3.79-3.69 (m, 2H), 2.54 (br d, J = 8.3 Hz, 1H), 2.31-2.21 (m, 1H), 1.79-1.67 (m, 1H), 1.38-1.27 (m, 1H), 1.20-1.09 (m, 1H), 1.05-0.93 (m, 6H). | 2.42 B Example was prepared from Example 48. |
| 170 | 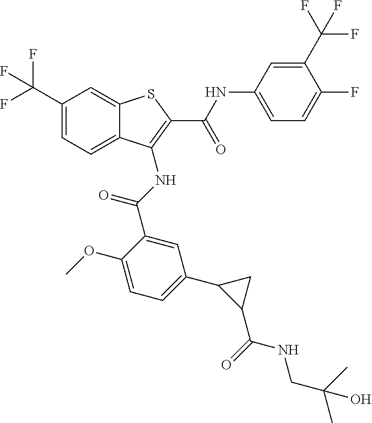 [SMILES: COc1ccc(C2CC2C(=O)NCC(C)(C)O)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-(5- {2-[(2- hydroxy-2- methylpropyl) carbamoyl] cyclopropyl}-2- methoxy- benzamido)-6- (trifluoromethyl)- 1- benzothiophene- 2- carboxamide | 712.2 | 1H NMR (400 MHz, DMSO-d6) δ ppm 10.89-10.62 (m, 2H), 8.66 (s, 1H), 8.20-8.12 (m, 1H), 8.07 (dd, J = 8.4, 3.8 Hz, 1H), 7.99 (br dd, J = 5.4, 3.2 Hz, 1H), 7.81 (d, J = 8.6 Hz, 1H), 7.64-7.49 (m, 2H), 7.38 (br d, J = 8.3 Hz, 1H), 7.16 (d, J = 8.8 Hz, 1H), 4.61- 4.41 (m, 2H), 4.38- 4.23 (m, 2H), 4.21- 4.09 (m, 1H), 4.07- 4.00 (m, 1H), 3.90 (d, J = 4.6 Hz, 3H), 3.02 (d, J = 3.9 Hz, 3H), 2.37- 2.26 (m, 1H), 1.87- 1.76 (m, 1H), 1.39- 1.32 (m, 1H), 1.28- 1.15 (m, 2H). | 2.32 A Example was prepared from Example 48. |
| 171 | 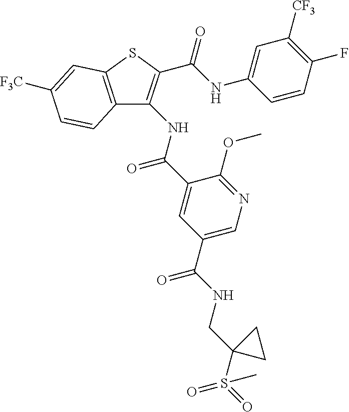 [SMILES: COc1ncc(C(=O)NCC2(S(C)(=O)=O)CC2)cc1C(=O)Nc1c(C(=O)Nc2ccc(F)c(C(F)(F)F)c2)sc2cc(C(F)(F)F)ccc12] | N3-(2-((4- fluoro-3- (trifluoromethyl) phenyl) carbamoyl)-6- (trifluoromethyl) benzo[b] thiophen-3-yl)-2- methoxy-N5- ((1- (methylsulfonyl) cyclopropyl) methyl) pyridine-3,5- dicarboxamide | 733.1 | 1H NMR (400 MHz, DMSO-d6) δ ppm 10.97-10.73 (m, 2H), 8.95-8.86 (m, 1H), 8.84 (d, J = 2.2 Hz, 1H), 8.67 (s, 2H), 8.22- 8.12 (m, 1H), 8.09 (d, J = 8.6 Hz, 1H), 8.03- 7.94 (m, 1H), 7.82 (d, J = 8.8 Hz, 1H), 7.53 (t, J = 9.9 Hz, 1H), 4.06 (s, 4H), 3.84 (br d, J = 5.9 Hz, 2H), 3.06 (s, 3H), 1.31-1.18(m, 2H), 1.18-1.04 (m, 2H) | 2.28 B |
| 172 | 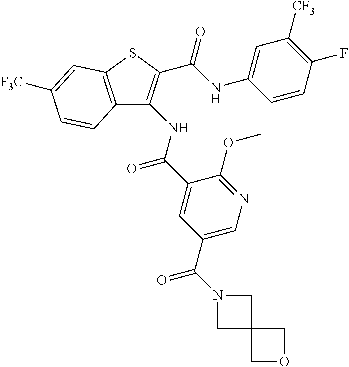 [SMILES: COc1ncc(C(=O)N2CC3(COC3)C2)cc1C(=O)Nc1c(C(=O)Nc2ccc(F)c(C(F)(F)F)c2)sc2cc(C(F)(F)F)ccc12] | N-(2-((4- fluoro-3- (trifluoromethyl) phenyl) carbamoyl)-6- (trifluoromethyl) benzo[b] thiophen-3-yl)-2- methoxy-5-(2- oxa-6- azaspiro[3.3] heptane-6- carbonyl) nicotinamide | 683.2 | 1H NMR (400 MHz, DMSO-d6) δ ppm 10.92-10.66 (m, 2H), 8.64-8.64 (m, 1H), 8.73-8.53 (m, 1H), 8.37 (d, J = 1.7 Hz, 1H), 8.12-8.12 (m, 1H), 8.04-8.04 (m, 1H), 8.23-7.90 (m, 1H), 7.82 (d, J = 8.6 Hz, 1H), 7.54 (t, J = 9.7 Hz, 1H), 4.67 (br d, J = 5.6 Hz, 4H), 4.52 (br s, 2H), 4.23 (br s, 2H), 4.04 (s, 3H) | 2.22 B |
| 173 | 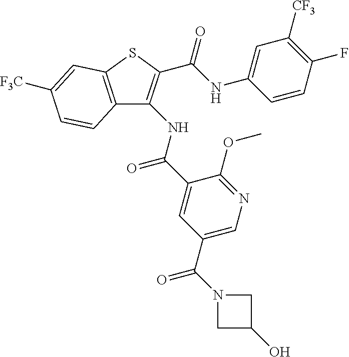 [SMILES: COc1ncc(C(=O)N2CC(O)C2)cc1C(=O)Nc1c(C(=O)Nc2ccc(F)c(C(F)(F)F)c2)sc2cc(C(F)(F)F)ccc12] | N-(2-((4- fluoro-3- (trifluoromethyl) phenyl) carbamoyl)-6- (trifluoromethyl) benzo[b] thiophen-3-yl)-5-(3- hydroxyazetidine- 1-carbonyl)- 2- methoxy- nicotinamide | 657.2 | 1H NMR (400 MHz, DMSO-d6) δ ppm 10.92-10.70 (m, 2H), 8.66 (s, 1H), 8.63 (d, J = 2.0 Hz, 1H), 8.39 (d, J = 2.2 Hz, 1H), 8.20- 8.12 (m, 1H), 8.10 (d, J = 8.3 Hz, 1H), 8.02- 7.93 (m, 1H), 7.82 (d, J = 8.1 Hz, 1H), 7.54 (t, J = 9.7 Hz, 1H), 5.82- 5.73 (m, 1H), 4.56- 4.47 (m, 2H), 4.32- 4.20 (m, 1H), 4.15- 4.06 (m, 1H), 4.03 (s, 3H), 3.85-3.75 (m, 1H) | 2.13 A |
| 174 | 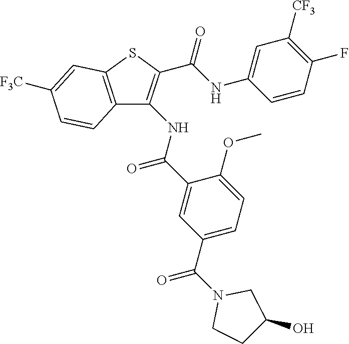 [SMILES: COc1ccc(C(=O)N2CC[C@H](O)C2)cc1C(=O)Nc1c(C(=O)Nc2ccc(F)c(C(F)(F)F)c2)sc2cc(C(F)(F)F)ccc12] | (S)-N-(4- fluoro-3- (trifluoromethyl) phenyl)-3-(5- (3- hydroxy- pyrrolidine- 1- carbonyl)-2- methoxy- benzamido)-6- (trifluoromethyl) benzo[b] thiophene-2- carboxamide | 670.2 | ¹H NMR (400 MHz, DMSO-d₆) δ ppm 11.02-10.64 (m, 1H), 8.64 (s, 1H), 8.16 (br s, 1H), 8.09 (br d, J = 8.3 Hz, 1H), 7.99-7.93 (m, 2H), 7.84-7.70 (m, 2H), 7.54 (t, J = 9.6 Hz, 1H), 7.27 (br d, J = 8.8 Hz, 1H), 5.13- 4.90 (m, 1H), 4.38- 4.18 (m, 1H), 3.97 (br s, 3H), 3.66-3.52 (m, 3H), 2.01-1.71 (m, 2H). | 2.12 B |
| 175 | 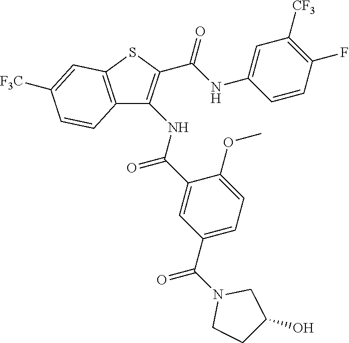 [SMILES: COc1ccc(C(=O)N2CC[C@@H](O)C2)cc1C(=O)Nc1c(C(=O)Nc2ccc(F)c(C(F)(F)F)c2)sc2cc(C(F)(F)F)ccc12] | (R)-N-(4- fluoro-3- (trifluoromethyl) phenyl)-3-(5- (3- hydroxy- pyrrolidine-1- carbonyl)-2- methoxy- benzamido)-6- (trifluoromethyl) benzo[b] thiophene-2- carboxamide | 670.2 | ¹H NMR (400 MHz, DMSO-d₆) δ ppm 11.02-10.61 (m, 1H), 8.65 (s, 1H), 8.16 (br s, 1H), 8.09 (d, J = 8.3 Hz, 1H), 8.02-7.91 (m, 2H), 7.85-7.70 (m, 2H), 7.54 (t, J = 9.5 Hz, 1H), 7.27 (br d, J = 8.6 Hz, 1H), 5.13-4.86 (m, 1H), 4.39-4.15 (m, 1H), 3.97 (br s, 3H), 3.58 (br s, 3H), 2.04-1.68 (m, 2H). | 2.12 B |
| 176 | 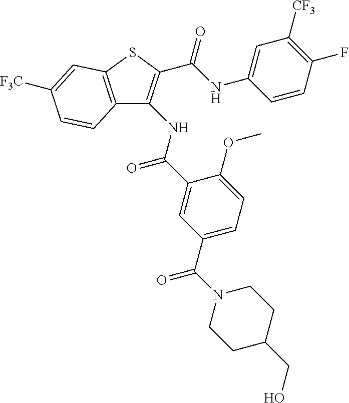 [SMILES: COc1ccc(C(=O)N2CCC(CO)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (4- (hydroxymethyl) piperidine-1- carbonyl)-2- methoxy- benzamido)-6- (trifluoromethyl) benzo[b] thiophene-2- carboxamide | 698.3 | ¹H NMR (400 MHz, DMSO-d₆) δ ppm 11.06-10.68 (m, 1H), 8.64 (s, 1H), 8.18- 8.13 (m, 1H), 8.11- 8.06 (m, 1H), 8.02- 7.92 (m, 1H), 7.85- 7.75 (m, 2H), 7.60- 7.52 (m, 2H), 7.33- 7.23 (m, 1H), 4.58- 4.47 (m, 1H), 3.96 (br s, 3H), 3.51-3.45 (m, 2H), 3.29-3.23 (m, 3H), 1.79-1.54 (m, 3H), 1.16-0.96 (m, 2H). | 2.19 B |
| 177 | 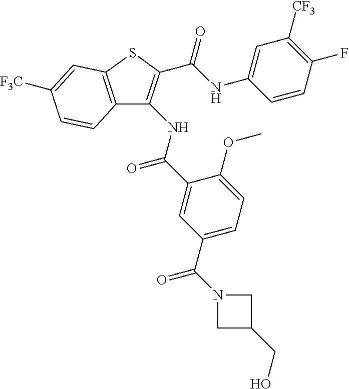 [SMILES: COc1ccc(C(=O)N2CC(CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (3- (hydroxymethyl) azetidine-1- carbonyl)-2- methoxy- benzamido)-6- (trifluoromethyl) benzo[b] thiophene-2- carboxamide | 670.2 | 1H NMR (400 MHz, DMSO-d6) δ ppm 10.90-10.79 (m, 1H), 10.79-10.67 (m, 1H), 8.66 (s, 1H), 8.21- 8.12 (m, 1H), 8.12- 8.03 (m, 2H), 8.03- 7.92 (m, 1H), 7.88- 7.75 (m, 2H), 7.54 (t, J = 9.8 Hz, 1H), 7.29 (d, J = 9.0 Hz, 1H), 4.87- 4.77 (m, 1H), 4.33- 4.24 (m, 1H), 4.07- 3.94 (m, 5H), 3.82- 3.74 (m, 1H), 3.53 (t, J = 5.3 Hz, 2H), 2.76- 2.67 (m, 1H) | 2.12 A |
| 178 | 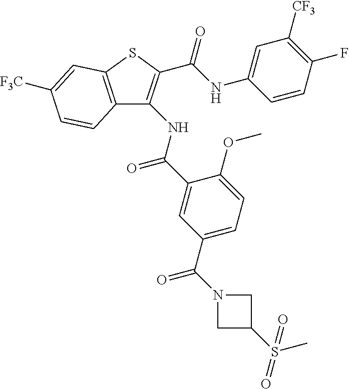 [SMILES: COc1ccc(C(=O)N2CC(S(C)(=O)=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(3- (methylsulfonyl) azetidine-1- carbonyl) benzamido)-6- (trifluoromethyl) benzo[b] thiophene-2- carboxamide | 708.4 | 1H NMR (400 MHz, DMSO-d6) δ ppm 10.95-10.66 (m, 2H), 8.66 (s, 1H), 8.25- 8.15 (m, 1H), 8.15- 8.04 (m, 2H), 7.97 (br dd, J = 5.0, 4.3 Hz, 1H), 7.86 (dd, J = 8.7, 1.8 Hz, 1H), 7.83-7.70 (m, 1H), 7.53 (t, J = 9.8 Hz, 1H), 7.30 (d, J = 8.8 Hz, 1H), 4.71-4.59 (m, 1H), 4.55-4.44 (m, 1H), 4.41-4.29 (m, 2H), 4.29-4.14 (m, 1H), 3.97 (s, 3H), 3.06 (s, 3H) | 2.17 B |
| 179 | 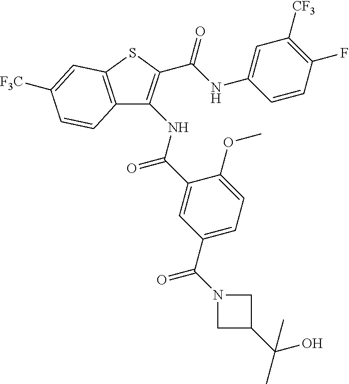 [SMILES: COc1ccc(C(=O)N2CC(C(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (3-(2- hydroxypropan- 2-yl)azetidine- 1-carbonyl)-2- methoxy- benzamido)-6- (trifluoromethyl) benzo[b] thiophene-2- carboxamide | 698.3 | 1H NMR (400 MHz, DMSO-d6) δ ppm 10.92-10.69 (m, 2H), 8.66 (s, 1H), 8.22- 8.13 (m, 1H), 8.13- 8.03 (m, 2H), 8.02- 7.92 (m, 1H), 7.88- 7.73 (m,2H), 7.54 (t, J = 9.9 Hz, 1H), 7.29 (d, J = 9.0 Hz, 1H), 4.55 (s, 1H), 4.25-4.11 (m, 2H), 4.03-3.87 (m, 5H), 2.64-2.54 (m, 1H), 1.02 (br d, J = 16.4 Hz, 6H) | 2.26 A |
| 180 | 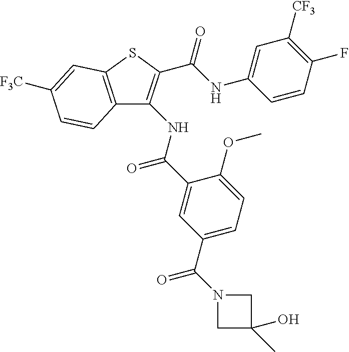 [SMILES: COc1ccc(C(=O)N2CC(C)(O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (3-hydroxy-3- methylazetidine- 1-carbonyl)- 2- methoxy- benzamido)-6- (trifluoromethyl) benzo[b] thiophene-2- carboxamide | 670.2 | 1H NMR (400 MHz, DMSO-d6) δ ppm 10.89-10.65 (m, 2H), 8.66 (s, 1H), 8.21- 8.13 (m, 1H), 8.11- 8.04 (m, 2H), 8.02- 7.94 (m, 1H), 7.87- 7.74 (m, 2H), 7.53 (t, J = 9.8 Hz, 1H), 7.28 (d, J = 8.8 Hz, 1H), 5.67 (s, 1H), 4.16-4.06 (m, 2H), 3.98 (s, 3H), 3.89 (br d, J = 2.7 Hz, 2H), 1.38 (s, 3H) | 2.18 A |
| 181 | 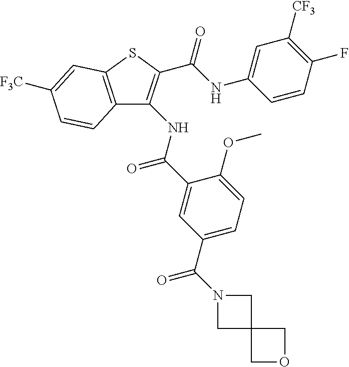 [SMILES: COc1ccc(C(=O)N2CC3(COC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(2- oxa-6- azaspiro[3.3] heptane-6- carbonyl) benzamido)-6- (trifluoromethyl) benzo[b] thiophene-2- carboxamide | 682.2 | 1H NMR (400 MHz, DMSO-d6) δ ppm 10.89-10.67 (m, 2H), 8.66 (s, 1H), 8.15 (dd, J = 6.5, 2.3 Hz, 1H), 8.11-8.03 (m, 2H), 8.03-7.90 (m, 1H), 7.82 (d, J = 9.0 Hz, 2H), 7.54 (t, J = 9.7 Hz, 1H), 7.29 (d, J = 8.8 Hz, 1H), 4.67 (br s, 4H), 4.51- 4.41 (m, 2H), 4.21 (br d, J = 3.4 Hz, 2H), 3.98 (s, 3H) | 2.20 A |
| 182 | 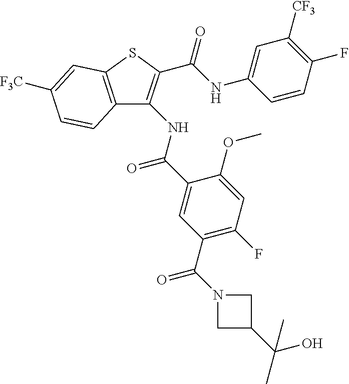 [SMILES: COc1cc(F)c(C(=O)N2CC(C(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(4- fluoro-5-(3-(2- hydroxypropan- 2-yl)azetidine- 1-carbonyl)-2- methoxy- benzamido)-6- (trifluoromethyl) benzo[b] thiophene-2- carboxamide | 716.2 | 1H NMR (400 MHz, DMSO-d6) δ ppm 10.76 (br s, 2H), 8.95- 8.81 (m, 1H), 8.65 (s, 1H), 8.43-8.30 (m, 1H), 8.21-8.11 (m, 1H), 8.00-7.89 (m, 1H), 7.85-7.76 (m, 1H), 7.54 (t, J = 9.8 Hz, 1H), 7.24 (d, J = 12.0 Hz, 1H), 4.57 (br s, 1H), 3.98 (s, 3H), 3.92 (br t, J = 6.1 Hz, 2H), 2.64-2.54 (m, 2H), 2.10 (s, 1H), 1.04 (s, 3H), 0.98 (s, 3H) | 2.28 A |
| 183 | 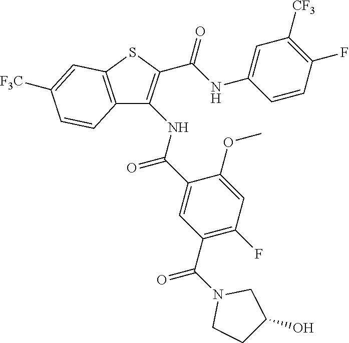 [SMILES: COc1cc(F)c(C(=O)N2CC[C@@H](O)C2)cc1C(=O)Nc1c(C(=O)Nc2ccc(F)c(C(F)(F)F)c2)sc2cc(C(F)(F)F)ccc12] | (R)-N-(4- fluoro-3- (trifluoromethyl) phenyl)-3-(4- fluoro-5-(3- hydroxy- pyrrolidine-1- carbonyl)-2- methoxy- benzamido)-6- (trifluoromethyl) benzo[b] thiophene-2- carboxamide | 688.3 | 1H NMR (400 MHz, DMSO-d6) δ ppm 10.83-10.69 (m, 2H), 8.67 (s, 1H), 8.23- 8.13 (m, 1H), 8.07 (d, J = 8.6 Hz, 1H), 8.01- 7.94 (m, 1H), 7.93- 7.86 (m, 1H), 7.80 (dd, J = 1.2, 8.8 Hz, 1H), 7.54 (t, J = 9.7 Hz, 1H), 7.31-7.20 (m, 1H), 5.07-4.94 (m, 1H), 4.36-4.22 (m, 1H), 3.98 (s, 3H), 3.90 (s, 2H), 3.57-3.50 (m, 1H), 3.44-3.36 (m, 2H), 3.26-3.04 (m, 1H) | 2.15 A |
| 184 | 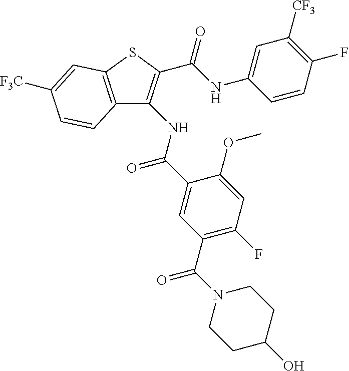 [SMILES: COc1cc(F)c(C(=O)N2CCC(O)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(4- fluoro-5-(4- hydroxypiperidine- 1- carbonyl)-2- methoxy- benzamido)-6- (trifluoromethyl) benzo[b] thiophene-2- carboxamide | 702.1 | 1H NMR (400 MHz, DMSO-d6) δ ppm 10.78 (br s, 1H), 10.74 (br s, 1H), 8.67 (s, 1H), 8.20-8.13 (m, 1H), 8.07 (d, J = 8.6 Hz, 1H), 8.01-7.91 (m, 1H), 7.85-7.74 (m, 2H), 7.54 (t, J = 9.5 Hz, 1H), 7.26 (d, J = 12.0 Hz, 1H), 4.80 (d, J = 3.9 Hz, 1H), 3.97 (s, 3H), 3.78-3.70 (m, 1H), 3.44-3.36 (m, 2H), 3.28-3.22 (m, 1H), 3.15-3.03 (m, 1H), 1.84-1.75 (m, 1H), 1.71-1.61 (m, 1H), 1.43-1.25 (m, 2H) | 2.18 A |
| 185 | 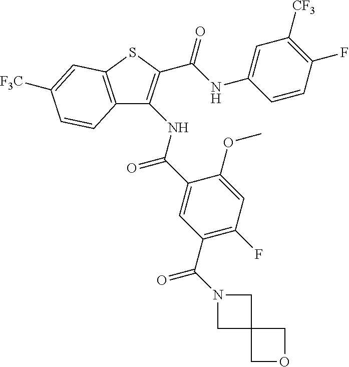 [SMILES: COc1cc(F)c(C(=O)N2CC3(COC3)C2)cc1C(=O)Nc1c(C(=O)Nc2ccc(F)c(C(F)(F)F)c2)sc2cc(C(F)(F)F)ccc12] | 3-(4-fluoro-2- methoxy-5-(2- oxa-6- azaspiro[3.3] heptane-6- carbonyl) benzamido)-N-(4- fluoro-3- (trifluoromethyl) phenyl)-6- (trifluoromethyl) benzo[b] thiophene-2- carboxamide | 700.1 | 1H NMR (400 MHz, DMSO-d6) δ ppm 10.76 (br s, 2H), 8.67 (s, 1H), 8.15 (dd, J = 2.3, 6.5 Hz, 1H), 8.05 (d, J = 8.6 Hz, 1H), 8.02-7.87 (m, 2H), 7.81 (dd, J = 1.5, 8.8 Hz, 1H), 7.54 (t, J = 9.8 Hz, 1H), 7.25 (d, J = 12.2 Hz, 1H), 4.66 (q, J = 6.6 Hz, 4H), 4.02 (s, 3H), 3.99 (s, 4H) | 2.22 A |
| 186 | 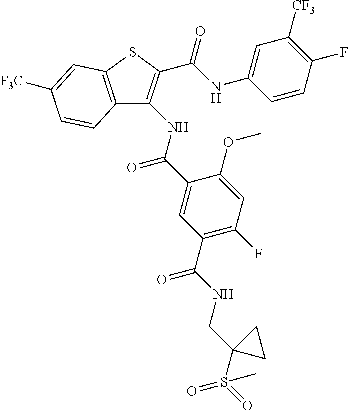 [SMILES: COc1cc(F)c(C(=O)NCC2(S(C)(=O)=O)CC2)cc1C(=O)Nc1c(C(=O)Nc2ccc(F)c(C(F)(F)F)c2)sc2cc(C(F)(F)F)ccc12] | 4-fluoro-N1- (2-((4-fluoro-3- (trifluoromethyl) phenyl) carbamoyl)-6- (trifluoromethyl) benzo[b] thiophen-3-yl)-6- methoxy-N3- ((1- (methylsulfonyl) cyclopropyl) methyl) isophthalamide | 750.2 | 1H NMR (400 MHz, DMSO-d6) δ ppm 10.84-10.72 (m, 2H), 8.67 (d, J = 0.7 Hz, 1H), 8.43 (br d, J = 4.4 Hz, 1H), 8.27-8.12 (m, 2H), 8.05 (d, J = 8.6 Hz, 1H), 8.02- 7.91 (m, 1H), 7.81 (dd, J = 1.7, 8.8 Hz, 1H), 7.54 (t, J = 9.9 Hz, 1H), 7.27 (d, J = 12.7 Hz, 1H), 3.99 (s, 3H), 3.83 (d, J = 5.9 Hz, 3H), 3.08 (s, 3H), 1.31-1.19 (m, 2H), 1.17-1.07 (m, 2H) | 2.25 A |
| 187 | 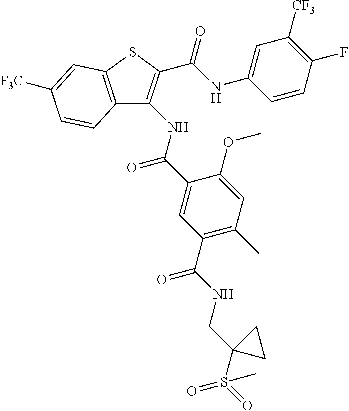 [SMILES: COc1cc(C)c(C(=O)NCC2(S(C)(=O)=O)CC2)cc1C(=O)Nc1c(C(=O)Nc2ccc(F)c(C(F)(F)F)c2)sc2cc(C(F)(F)F)ccc12] | N1-(2-((4- fluoro-3- (trifluoromethyl) phenyl) carbamoyl)-6- (trifluoromethyl) benzo[b] thiophen-3-yl)-6- methoxy-4- methyl-N3-((1- (methylsulfonyl) cyclopropyl) methyl) isophthalamide | 744.3 (M − H)⁻ | 1H NMR (400 MHz, DMSO-d6) δ ppm 10.86-10.80 (m, 1H), 10.79 (br d, J = 0.7 Hz, 1H), 8.68 (s, 1H), 8.51 (t, J = 6.4 Hz, 1H), 8.17 (dd, J = 2.7, 6.8 Hz, 1H), 8.02 (d, J = 8.6 Hz, 1H), 7.98 (br dd, J = 3.5, 9.2 Hz, 1H), 7.91 (s, 1H), 7.85- 7.74 (m, 1H), 7.55 (t, J = 9.8 Hz, 1H), 7.16 (s, 1H), 4.00 (s, 3H), 3.79 (d, J = 5.9 Hz, 2H), 3.07 (s, 3H), 2.45 (s, 3H), 1.29-1.19 (m, 2H), 1.15-1.00 (m, 2H) | 2.27 A |
| 188 | 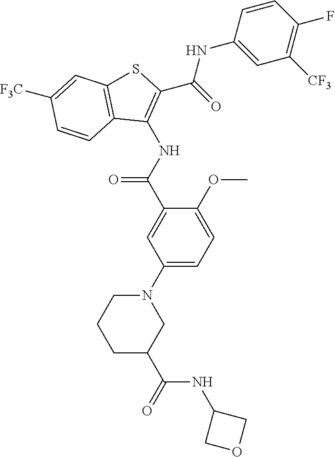 [SMILES: COc1ccc(N2CCCC(C(=O)NC3COC3)C2)cc1C(=O)Nc1c(C(=O)Nc2ccc(F)c(C(F)(F)F)c2)sc2cc(C(F)(F)F)ccc12] | 1-(3-((2-((4- fluoro-3- (trifluoromethyl) phenyl) carbamoyl)-6- (trifluoromethyl) benzo[b] thiophen-3- yl)carbamoyl)- 4- methoxyphenyl)- N-(oxetan-3- yl)piperidine- 3-carboxamide | 739.2 | 1H NMR (400 MHz, DMSO-d6) δ ppm 10.85 (s, 1H), 10.73 (s, 1H), 8.66 (s, 1H), 8.62 (d, J = 6.1 Hz, 1H), 8.18- 8.13 (m, 2H), 8.06 (d, J = 8.8 Hz, 1H), 7.81 (dd, J = 1.5, 8.6 Hz, 1H), 7.54 (t, J = 9.7 Hz, 1H), 7.42 (d, J = 2.4 Hz, 1H), 7.24-7.18 (m, 1H), 7.15-7.11 (m, 1H), 4.82-4.76 (m, 1H), 4.70 (dt, J = 3.9, 6.7 Hz, 1H), 3.91 (s, 3H), 3.32 (s, 2H), 3.37- 3.25 (m, 1H), 3.17 (d, J = 5.4 Hz, 2H), 2.71- 2.63 (m, 1H), 2.50 (td, J = 1.8, 3.7 Hz, 4H), 2.53-2.46 (m, 2H) | 2.33 B Isomers separated by Prep SFC (Method 8) Prep SFC RT = 4.19 min) |
| 189 | 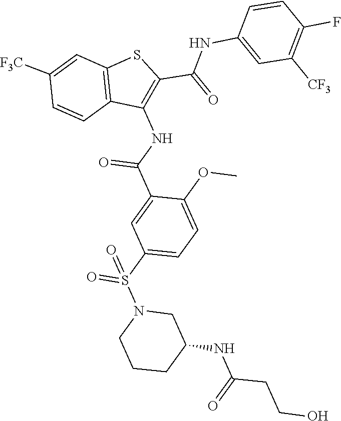 [SMILES: COc1ccc(S(=O)(=O)N2CCC[C@@H](NC(=O)CCO)C2)cc1C(=O)Nc1c(C(=O)Nc2ccc(F)c(C(F)(F)F)c2)sc2cc(C(F)(F)F)ccc12] | (R)-N-(4- fluoro-3- (trifluoromethyl) phenyl)-3-(5- ((3-(3- hydroxy- propanamido) piperidin-1- yl)sulfonyl)-2- methoxy- benzamido)-6- (trifluoromethyl) benzo[b] thiophene-2- carboxamide | 791.2 | 1H NMR (400 MHz, DMSO-d6) δ = 10.10- 10.02 (m, 1H), 9.99- 9.92 (m, 1H), 7.87- 7.81 (m, 1H), 7.40- 7.33 (m, 1H), 7.31- 7.25 (m, 1H), 7.24- 7.20 (m, 1H), 7.17- 7.10 (m, 1H), 7.09- 7.04 (m, 1H), 7.02- 6.94 (m, 2H), 6.74- 6.67 (m, 1H), 6.65- 6.59 (m, 1H), 3.77- 3.68 (m, 1H), 3.21- 3.15 (m, 3H), 2.97- 2.80 (m, 1H), 2.79- 2.71 (m, 2H), 2.64- 2.56 (m, 1H), 2.46- 2.39 (m, 1H), 1.60- 1.47 (m, 1H), 1.41- 1.35 (m, 2H), 1.35- 1.27 (m, 1H), 0.95- 0.76 (m, 2H), 0.71- 0.59 (m, 1H), 0.49- 0.23 (m, 1H). | 2.17 A |
| 190 | 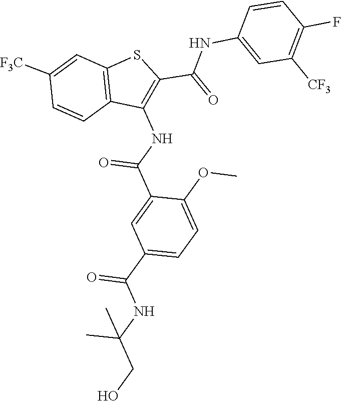 [SMILES: COc1ccc(C(=O)NC(C)(C)CO)cc1C(=O)Nc1c(C(=O)Nc2ccc(F)c(C(F)(F)F)c2)sc2cc(C(F)(F)F)ccc12] | N3-(2-((4- fluoro-3- (trifluoromethyl) phenyl) carbamoyl)-6- (trifluoromethyl) benzo[b] thiophen-3-yl)-N1- (1-hydroxy-2- methylpropan- 2-yl)-4- methoxy- isophthalamide | 672.0 | 1H NMR (400 MHz, DMSO-d6) δ ppm 10.89-10.80 (m, 1H), 10.80-10.66 (m, 1H), 8.67 (s, 1H), 8.29 (d, J = 2.2 Hz, 1H), 8.20- 8.15 (m, 1H), 8.08 (br d, J = 8.3 Hz, 1H), 8.05- 7.94 (m, 2H), 7.85- 7.74 (m, 1H), 7.62- 7.48 (m, 2H), 7.27 (br d, J = 8.6 Hz, 1H), 4.92 (t, J = 6.1 Hz, 1H), 3.98 (s, 3H), 3.50 (br d, J = 5.9 Hz, 2H), 1.31 (s, 6H) | 2.26 A |
| 191 | 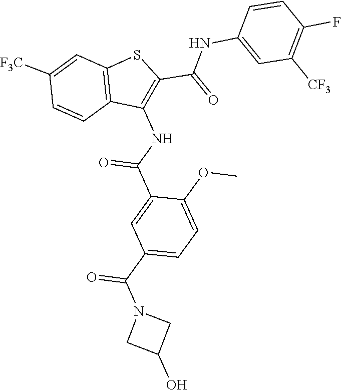 [SMILES: COc1ccc(C(=O)N2CC(O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (3- hydroxyazetidine- 1-carbonyl)- 2- methoxy- benzamido)-6- (trifluoromethyl) benzo[b] thiophene-2- carboxamide | 656.2 | 1H NMR (400 MHz, DMSO-d6) δ ppm 10.82 (s, 1H), 10.74 (s, 1H), 8.66 (s, 1H), 8.16 (dd, J = 6.1, 2.0 Hz, 1H), 8.11-8.03 (m, 2H), 8.02-7.95 (m, 1H), 7.82 (br d, J = 8.8 Hz, 2H), 7.54 (t, J = 9.8 Hz, 1H), 7.28 (d, J = 8.8 Hz, 1H), 5.75 (d, J = 5.9 Hz, 1H), 4.55-4.38 (m, 2H), 4.32-4.20 (m, 1H), 4.07-3.91 (m, 4H), 3.85-3.71 (m, 1H) | 2.12 A |
| 192 | 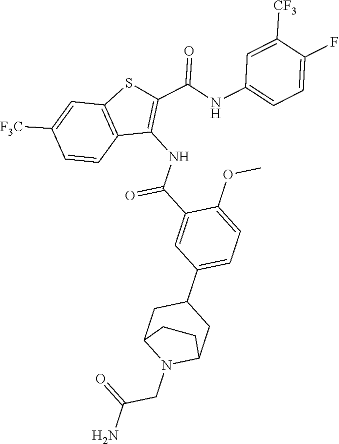 [SMILES: COc1ccc(C2CC3CCC(C2)N3CC(N)=O)cc1C(=O)Nc1c(C(=O)Nc2ccc(F)c(C(F)(F)F)c2)sc2cc(C(F)(F)F)ccc12] | 3-(5-(8-(2- amino-2- oxoethyl)-8- azabicyclo[3.2.1] octan-3-yl)- 2- methoxy- benzamido)-N-(4- fluoro-3- (trifluoromethyl) phenyl)-6- (trifluoromethyl) benzo[b] thiophene-2- carboxamide | 723.1 | 1H NMR (400 MHz, DMSO-d6) δ ppm 10.91-10.81 (m, 1H), 10.77 (s, 1H), 8.69 (s, 1H), 8.22-8.12 (m, 1H), 8.09-7.96 (m, 1H), 7.86-7.77 (m, 2H), 7.73 (br s, 2H), 7.55 (br t, J = 9.4 Hz, 1H), 7.29-7.19 (m, 1H), 4.07-3.90 (m, 5H), 3.78 (br d, J = 5.1 Hz, 1H), 3.70 (br s, 1H),, 3.19 (br s, 1H), 2.41-2.29 (m, 2H), 2.13-2.00 (m, 1H), 1.91 (br s, 2H), 1.84 (br d, J = 11.7 Hz, 1H), 1.56 (br d, J = 8.8 Hz, 1H), 1.23 (s, 1H) | 2.44 A |
| 193 | 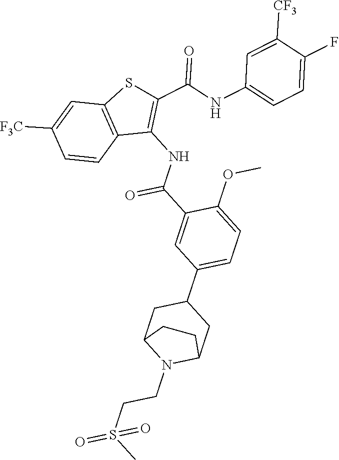 [SMILES: COc1ccc(C2CC3CCC(C2)N3CCS(C)(=O)=O)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(8- (2- (methylsulfonyl) ethyl)-8- azabicyclo[3.2.1] octan-3- yl)benzamido)- 6- (trifluoromethyl) benzo[b] thiophene-2- carboxamide | 772.2 | 1H NMR (400 MHz, DMSO-d6) δ ppm 10.91-10.81 (m, 1H), 10.84 (br s, 1H), 8.64 (s, 1H), 8.13 (dd, J = 2.7, 6.6 Hz, 1H), 8.08 (d, J = 8.6 Hz, 1H), 8.00-7.88 (m, 1H), 7.80 (d, J = 8.6 Hz, 1H), 7.69 (br s, 1H), 7.56-7.41 (m, 2H), 7.13 (br d, J = 8.6 Hz, 1H), 3.92 (br s, 3H), 3.23 (br t, J = 6.6 Hz, 4H), 3.09 (s, 3H), 2.97- 2.86 (m, 1H), 2.63 (t, J = 6.6 Hz, 2H), 2.31- 2.17 (m, 2H), 1.89- 1.78 (m, 2H), 1.55- 1.44 (m, 2H), 1.43- 1.30 (m, 2H). | 2.50 A |
| 194 | 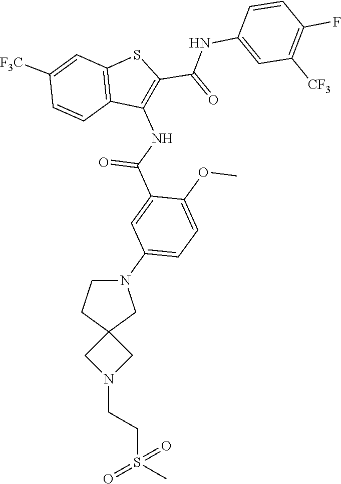 [SMILES: COc1ccc(N2CCC3(CN(CCS(C)(=O)=O)C3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(2- (2- (methylsulfonyl) ethyl)-2,6- diazaspiro[3.4] octan-6- yl)benzamido)- 6- (trifluoromethyl) benzo[b] thiophene-2- carboxamide | 771.2 (M − H)⁻ | 1H NMR (400 MHz, DMSO-d6) δ ppm 10.92-10.85 (m, 1H), 10.77 (s, 1H), 10.22- 10.13 (m, 1H), 8.69 (s, 1H), 8.22-8.13 (m, 1H), 8.07-7.96 (m, 1H), 7.89-7.77 (m, 2H), 7.60-7.49 (m, 1H), 7.32 (br d, J = 9.3 Hz, 1H), 7.21-6.95 (m, 1H), 4.40-4.12 (m, 2H), 4.02-3.93 (m, 3H), 3.81-3.74 (m, 2H), 3.69- 3.46 (m, 3H), 3.11 (s, 3H), 2.59 (br d, J = 1.0 Hz, 2H), 2.42-2.36 (m, 1H), 2.29-2.23 (m, 2H), 2.19-2.13 (m, 1H), 2.02-1.97 (m, 1H | 2.00 B |
| 195 | 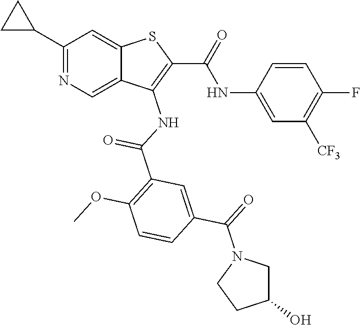 [SMILES: COc1ccc(C(=O)N2CC[C@@H](O)C2)cc1C(=O)Nc1c(C(=O)Nc2ccc(F)c(C(F)(F)F)c2)sc2cc(C3CC3)ncc12] | (R)-6- cyclopropyl-N- (4-fluoro-3- (trifluoromethyl) phenyl)-3-(5- (3- hydroxy- pyrrolidine-1- carbonyl)-2- methoxy- benzamido) thieno[3,2-c] pyridine-2- carboxamide | 643.2 | 1H NMR (400 MHz, DMSO-d6) δ ppm 11.09 (br s, 1H), 10.65 (br s, 1H), 9.00 (s, 1H), 8.19 (br d, J = 4.5 Hz, 1H), 8.08-7.91 (m, 3H), 7.81-7.70 (m, 1H), 7.54 (t, J = 9.5 Hz, 1H), 7.28 (d, J = 9.0 Hz, 1H), 6.53 (s, 1H), 5.07-4.95 (m, 1H), 4.33-4.19 (m, 1H), 3.98 (s, 3H), 3.63- 3.55 (m, 2H), 3.17 (d, J = 5.0 Hz, 1H), 2.26- 2.19 (m, 1H), 1.98- 1.54 (m, 2H), 1.23 (s, 2H), 1.17-1.10 (m, 1H), 1.07-0.93 (m, 1H) | 1.93 A |
| 196 | 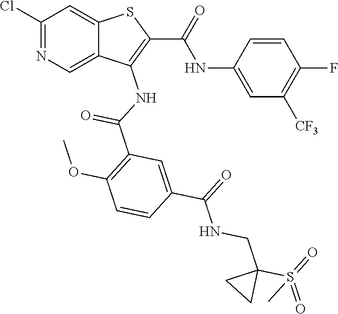 [SMILES: COc1ccc(C(=O)NCC2(S(C)(=O)=O)CC2)cc1C(=O)Nc1c(C(=O)Nc2ccc(F)c(C(F)(F)F)c2)sc2cc(Cl)ncc12] | N3-(6-chloro- 2-((4-fluoro-3- (trifluoromethyl) phenyl) carbamoyl) thieno[3,2-c] pyridin-3- yl)-4-methoxy- N1-((1- (methylsulfonyl) cyclopropyl) methyl) isophthalamide | 700.2 | 1H NMR (400 MHz, DMSO-d6) δ ppm 11.14 (td, J = 1.1, 2.5 Hz, 1H), 10.76 (br dd, J = 0.9, 1.6 Hz, 1H), 9.01 (d, J = 0.7 Hz, 1H), 8.73 (br t, J = 5.9 Hz, 1H), 8.43 (d, J = 2.2 Hz, 1H), 8.39 (s, 1H), 8.17 (dd, J = 2.4, 6.4 Hz, 1H), 8.08 (dd, J = 2.3, 8.7 Hz, 1H), 8.03-7.93 (m, 1H), 7.54 (t, J = 9.9 Hz, 1H), 7.34 (d, J = 8.8 Hz, 1H), 3.99 (s, 3H), 3.84 (br d, J = 6.1 Hz, 3H), 3.06 (s, 3H), 1.29- 1.07 (m, 4H) | 1.98 A |
| 197 | 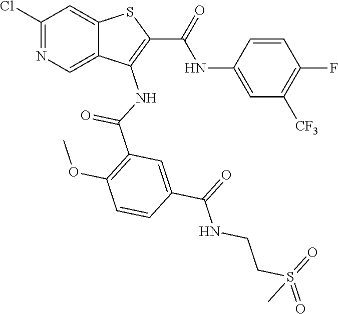 [SMILES: COc1ccc(C(=O)NCCS(C)(=O)=O)cc1C(=O)Nc1c(C(=O)Nc2ccc(F)c(C(F)(F)F)c2)sc2cc(Cl)ncc12] | N3-(6-chloro- 2-((4-fluoro-3- (trifluoromethyl) phenyl)carbamoyl) thieno[3, 2-c]pyridin-3- yl)-4-methoxy- N1-(2- (methylsulfonyl) ethyl) isophthalamide | 674.1 | 1H NMR (400 MHz, DMSO-d6) δ ppm 11.19-11.07 (m, 1H), 10.84-10.71 (m, 1H), 9.01 (d, J = 0.7 Hz, 1H), 8.86-8.72 (m, 1H), 8.43 (d, J = 2.0 Hz, 1H), 8.40 (s, 1H), 8.17 (dd, J = 2.3, 6.2 Hz, 1H), 8.07 (dd, J = 2.3, 8.7 Hz, 1H), 8.02- 7.94 (m, 1H), 7.60- 7.47 (m, 1H), 7.34 (d, J = 8.8 Hz, 1H), 3.98 (s, 3H), 3.68 (q, J = 6.7 Hz, 2H), 3.38 (s, 2H), 3.03 (s, 3H) | 1.87 A |
| 199 | 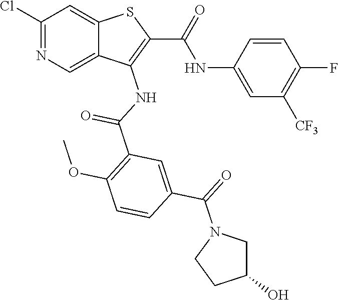 [SMILES: COc1ccc(C(=O)N2CC[C@@H](O)C2)cc1C(=O)Nc1c(C(=O)Nc2ccc(F)c(C(F)(F)F)c2)sc2cc(Cl)ncc12] | (R)-6-chloro- N-(4-fluoro-3- (trifluoromethyl) phenyl)-3-(5- (3- hydroxypyrrolidine- 1- carbonyl)-2- methoxy- benzamido) thieno[3, 2-c]pyridine-2- carboxamide | 638.1 | 1H NMR (400 MHz, DMSO-d6) δ ppm 11.17-11.04 (m, 1H), 10.81 (dt, J = 2.7, 4.6 Hz, 1H), 9.01 (d, J = 0.7 Hz, 1H), 8.39 (d, J = 0.7 Hz, 1H), 8.22-8.13 (m, 1H), 8.09-8.00 (m, 1H), 7.98 (br dd, J = 3.8, 8.2 Hz, 1H), 7.81-7.68 (m, 1H), 7.54 (t, J = 9.9 Hz, 1H), 7.27 (d, J = 8.6 Hz, 1H), 5.05-4.90 (m, 1H), 4.36-4.18 (m, 1H), 3.96 (s, 3H), 3.67- 3.37 (m, 4H), 3.31- 3.20 (m, 1H), 1.99- 1.71 (m, 2H) | 1.83 A |
| 200 | 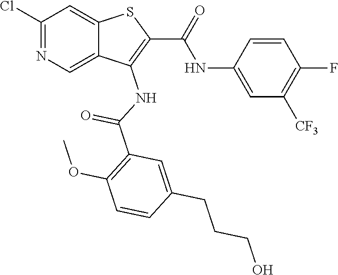 [SMILES: COc1ccc(CCCO)cc1C(=O)Nc1c(C(=O)Nc2ccc(F)c(C(F)(F)F)c2)sc2cc(Cl)ncc12] | 6-chloro-N-(4- fluoro-3- (trifluoromethyl) phenyl)-3-(5- (3- hydroxypropyl)- 2- methoxy- benzamido) thieno[3, 2-c]pyridine-2- carboxamide | 582.3 | 1H NMR (400 MHz, DMSO-d6) δ ppm 11.11 (br dd, J = 2.0, 4.2 Hz, 1H), 10.86- 10.70 (m, 1H), 8.99 (s, 1H), 8.39 (s, 1H), 8.16 (dd, J = 2.4, 6.4 Hz, 1H), 8.04-7.93 (m, 1H), 7.71 (s, 1H), 7.55 (t, J = 9.7 Hz, 1H), 7.40 (dd, J = 1.8, 8.4 Hz, 1H), 7.15 (d, J = 8.6 Hz, 1H), 4.46 (t, J = 5.1 Hz, 1H), 3.92 (s, 3H), 3.41 (br d, J = 5.1 Hz, 2H), 2.61 (brt, J = 7.6 Hz, 2H), 1.75- 1.62 (m, 2H) | 2.11 A |
| 201 | 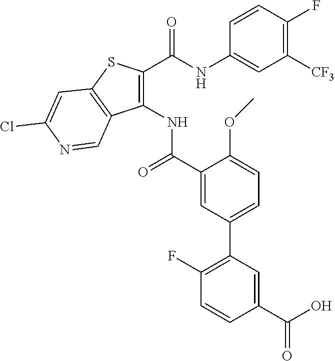 [SMILES: COc1ccc(-c2cc(C(=O)O)ccc2F)cc1C(=O)Nc1c(C(=O)Nc2ccc(F)c(C(F)(F)F)c2)sc2cc(Cl)ncc12] | 3′-((6-chloro-2- ((4-fluoro-3- (trifluoromethyl) phenyl) carbamoyl) thieno[3, 2-c]pyridin-3- yl)carbamoyl)- 6-fluoro-4′- methoxy-[1,1′- biphenyl]-3- carboxylic acid | 662.2 | 1H NMR (400 MHz, DMSO-d6) δ 9.09- 8.98 (m, 1H), 8.42- 8.30 (m, 1H), 8.19- 8.12 (m, 1H), 8.09- 8.05 (m, 2H), 8.03- 7.93 (m, 2H), 7.88- 7.66 (m, 1H), 7.59- 7.46 (m, 1H), 7.44- 7.22 (m, 2H), 4.01- 3.88 (m, 3H) | 2.70 C |
| 202 | 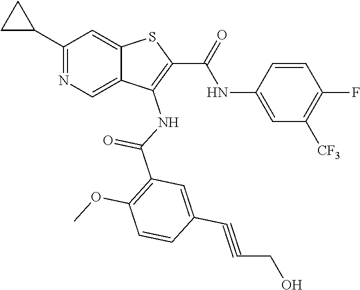 [SMILES: COc1ccc(C#CCO)cc1C(=O)Nc1c(C(=O)Nc2ccc(F)c(C(F)(F)F)c2)sc2cc(C3CC3)ncc12] | 6-cyclopropyl- N-(4-fluoro-3- (trifluoromethyl) phenyl)-3-(5- (3- hydroxyprop- 1-yn-1-yl)-2- methoxy- benzamido) thieno[3, 2-c]pyridine-2- carboxamide | 584.3 | | 2.2 A |
| 203 | 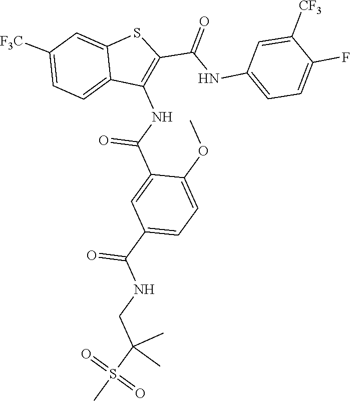 [SMILES: COc1ccc(C(=O)NCC(C)(C)S(C)(=O)=O)cc1C(=O)Nc1c(C(=O)Nc2ccc(F)c(C(F)(F)F)c2)sc2cc(C(F)(F)F)ccc12] | N3-(2-{[4- fluoro-3- (trifluoromethyl) phenyl] carbamoyl}-6- (trifluoromethyl)- 1- benzothiophen- 3-yl)-N1-(2- methanesulfonyl- 2- methylpropyl)- 4- methoxybenzene- 1,3- dicarboxamide | 732.1 | 1H NMR (400 MHz, DMSO-d6) δ ppm 10.91-10.71 (m, 1H), 8.67 (br s, 1H), 8.54 (t, J = 6.8 Hz, 1H), 8.40- 8.33 (m, 1H), 8.21- 8.04 (m, 2H), 7.98 (br d, J = 4.0 Hz, 1H), 7.88-7.78 (m, 1H), 7.58-7.49 (m, 1H), 7.34 (d, J = 8.5 Hz, 1H), 4.05-3.94 (m, 3H), 3.67 (d, J = 6.0 Hz, 2H), 3.00 (s, 3H), 1.30 (s, 6H). | 2.27 A |
| 204 | 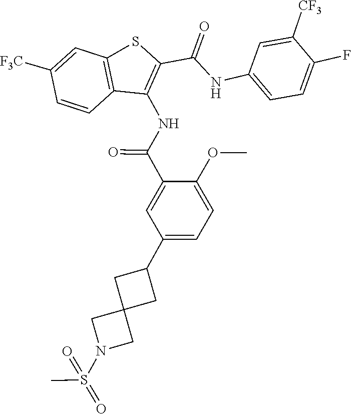 [SMILES: COc1ccc(C2CC3(C2)CN(S(C)(=O)=O)C3)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(2- (methylsulfonyl)- 2- azaspiro[3.3] heptan-6- yl)benzamido)- 6- (trifluoromethyl) benzo[b] thiophene-2- carboxamide | 728.10 | 1H NMR (400 MHz, DMSO-d6) δ ppm 10.82 (s, 1H), 10.74 (s, 1H), 8.66 (s, 1H), 8.14 (dd, J = 2.7, 6.6 Hz, 1H), 8.03-8.02 (m, 1H), 8.11-7.91 (m, 1H), 7.81(dd, J = 1.6, 8.9 Hz, 1H), 7.65 (d, J = 2.2 Hz, 1H), 7.55 (t, J = 9.9 Hz, 1H), 7.42 (dd, J = 2.3, 8.7 Hz, 1H), 7.17 (d, J = 8.6 Hz, 1H), 4.00 (s, 1H), 3.93 (s, 3H), 3.78 (s, 2H), 3.42-3.36 (m, 1H), 3.34-3.29 (m, 1H), 2.97 (s, 3H), 2.60- 2.52 (m, 2H), 2.27- 2.13 (m, 2H) | 2.49 A |
| 205 | 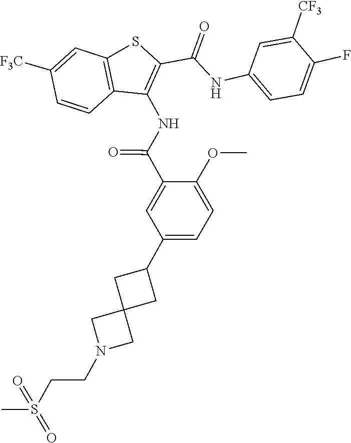 [SMILES: COc1ccc(C2CC3(C2)CN(CCS(C)(=O)=O)C3)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(2- (2- (methylsulfonyl) ethyl)-2- azaspiro[3.3] heptan-6- yl)benzamido)- 6- (trifluoromethyl) benzo[b] thiophene-2- carboxamide | 758.3 | 1H NMR (400 MHz, DMSO-d6) δ ppm 10.80 (s, 1H), 10.73 (s, 1H), 8.66 (s, 1H), 8.13 (dd, J = 2.7, 6.4 Hz, 1H), 8.06 (d, J = 8.6 Hz, 1H), 8.03-7.87 (m, 1H), 7.82 (dd, J = 1.6, 8.9 Hz, 1H), 7.62 (d, J = 2.2 Hz, 1H), 7.55 (t, J = 9.8 Hz, 1H), 7.40 (dd, J = 2.3, 8.7 Hz, 1H), 7.16 (d, J = 8.6 Hz, 1H), 3.91 (s, 3H), 78 (s, 2H), 3.20- 3.06 (m, 4H), 3.02 (s, 3H), 2.85-2.70 (m, 3H), 2.43 (dt, J = 2.4, 8.7 Hz, 2H), 2.18- 2.00 (m, 2H) | 2.09 B |
| 206 | 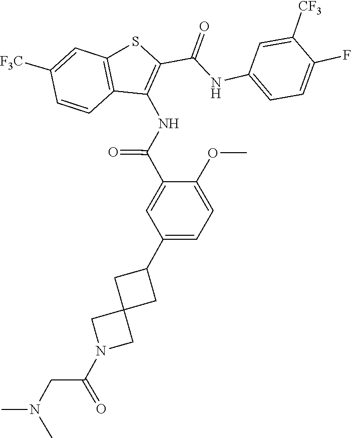 [SMILES: COc1ccc(C2CC3(C2)CN(C(=O)CN(C)C)C3)cc1C(=O)Nc1c(C(=O)Nc2ccc(F)c(C(F)(F)F)c2)sc2cc(C(F)(F)F)ccc12] | 3-(5-(2- (dimethylglycyl)- 2- azaspiro[3.3] heptan-6-yl)-2- methoxy- benzamido)- N-(4- fluoro-3- (trifluoromethyl) phenyl)-6- (trifluoromethyl) benzo[b] thiophene-2- carboxamide | 737.30 | 1H NMR (400 MHz, DMSO-d6) δ ppm 10.88-10.67 (m, 2H), 8.66 (s, 1H), 8.17- 8.10 (m, 1H), 8.06 (d, J = 8.6 Hz, 1H), 8.02- 7.94 (m, 1H), 7.82 (dd, J = 1.6, 8.7 Hz, 1H), 7.63 (d, J = 2.0 Hz, 1H), 7.55 (t, J = 9.8 Hz, 1H), 7.46-7.37 (m, 1H), 7.17 (d, J = 8.6 Hz, 1H), 4.29 (s, 1H), 4.06 (s, 1H), 4.00 (s, 1H), 3.93 (s, 3H), 3.77 (s, 1H), 2.95 (s, 3H), 2.91 (s, 1H), 2.52 (br s, 2H), 2.19 (d, J = 7.6 Hz, 7H) | 2.12 B |
| 207 | 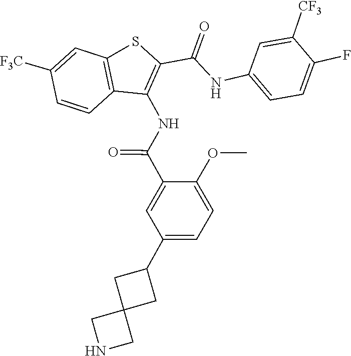 [SMILES: COc1ccc(C2CC3(CNC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(2- azaspiro[3.3] heptan-6- yl)benzamido)- 6- (trifluoromethyl) benzo[b] thiophene-2- carboxamide | 652.20 | 1H NMR (400 MHz, DMSO-d6) δ ppm 10.67 (m, 2H), 8.62- 8.51 (m, 1H), 8.16- 8.02 (m, 2H), 7.95- 7.86 (m, 1H), 7.79- 7.70 (m, 1H), 7.57 (d, J = 2.7 Hz, 1H), 7.52- 7.43 (m,1H), 7.39- 7.30 (m, 1H), 7.13 (d, J = 8.8 Hz, 1H), 3.92- 3.84 (m, 3H), 3.78 (s, 1H), 3.56 (s, 2H), 2.18- 2.09 (m, 4H), 1.88 (s, 1H), 1.23 (s, 2H) | 2.08 B |
| 208 | 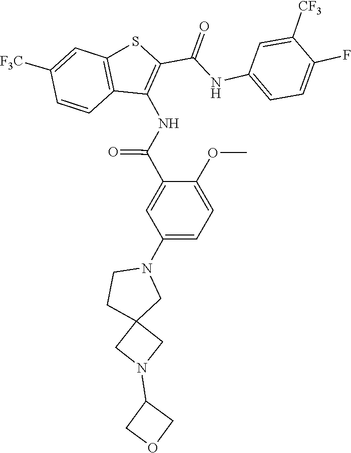 [SMILES: COc1ccc(N2CCC3(C2)CN(C2COC2)C3)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(2- (oxetan-3-yl)- 2,6- diazaspiro[3.4] octan-6- yl)benzamido)- 6- (trifluoromethyl) benzo[b] thiophene-2- carboxamide | 723.20 | 1H NMR (400 MHz, DMSO-d6) δ ppm 10.67 (m, 2H ) 8.67- 8.61 (m, 1H), 8.15- 8.10 (m, 1H), 8.09- 8.04 (m, 1H), 8.02- 7.94 (m, 1H), 7.83- 7.76 (m, 1H), 7.57- 7.49 (m,1H), 7.14- 7.09 (m, 1H), 7.01- 6.93 (m, 1H), 6.78- 6.71 (m, 1H), 4.55 (t, J = 6.6 Hz, 2H), 4.36 (dd, J = 5.4, 6.4 Hz, 2H), 4.13-4.05 (m, 2H), 3.86 (s, 3H), 3.76- 3.66 (m, 2H), 3.19 (s, 3H), 2.17-2.10 (m, 2H), 1.23 (s, 2H) | 2.44 A |
| 209 | 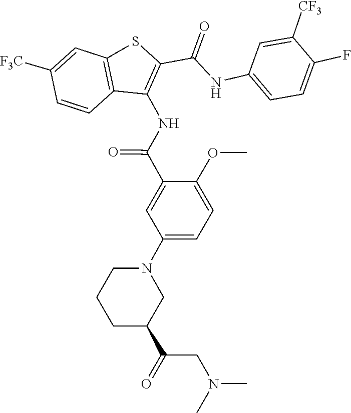 [SMILES: COc1ccc(N2CCC[C@H](C(=O)CN(C)C)C2)cc1C(=O)Nc1c(C(=O)Nc2ccc(F)c(C(F)(F)F)c2)sc2cc(C(F)(F)F)ccc12] | (R)-3-(5-(3-(2- (dimethylamino) acetamido) piperidin-1-yl)-2- methoxy- benzamido)-N-(4- fluoro-3- (trifluoromethyl) phenyl)-6- (trifluoromethyl) benzo[b] thiophene-2- carboxamide | 740.30 | 1H NMR (400 MHz, DMSO-d6) δ ppm 10.91-10.67 (m, 2H), 8.66 (d, J = 0.7 Hz, 1H), 8.16 (dd, J = 2.7, 6.6 Hz, 1H), 8.06 (d, J = 8.6 Hz, 1H), 8.01- 7.92 (m, 1H), 7.81 (dd, J = 1.6, 8.9 Hz, 1H), 7.68 (d, J = 8.3 Hz, 1H), 7.55 (t, J = 9.7 Hz, 1H), 7.39 (d, J = 2.9 Hz, 1H), 7.23- 7.05 (m, 1H), 3.95- 3.82 (m, 4H), 3.28- 3.19 (m, 1H), 2.87 (s, 2H), 2.79-2.61 (m, 2H), 2.19 (s, 6H), 1.81- 1.68 (m, 3H), 1.67- 1.55 (m, 2H), 1.53- 1.40 (m, 1H) | 2.46 A |
| 210 | 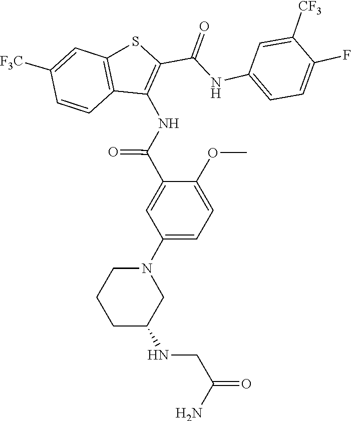 [SMILES: COc1ccc(N2CCC[C@@H](NCC(N)=O)C2)cc1C(=O)Nc1c(C(=O)Nc2ccc(F)c(C(F)(F)F)c2)sc2cc(C(F)(F)F)ccc12] | (S)-3-(5-(3-((2- amino-2- oxoethyl)amino) piperidin-1- yl)-2- methoxy- benzamido)-N-(4- fluoro-3- (trifluoromethyl) phenyl)-6- (trifluoromethyl) benzo[b] thiophene-2- carboxamide | 712.20 | 1H NMR (400 MHz, DMSO-d6) δ ppm 10.93-10.72 (m, 1H), 8.66 (s, 1H), 8.15 (dd, J = 2.3, 6.5 Hz, 1H), 8.09-8.02 (m, 1H), 8.02-7.94 (m, 1H), 7.84-7.76 (m, 1H), 7.54 (t, J = 9.8 Hz, 1H), 7.43-7.35 (m, 1H), 7.34-7.25 (m, 1H), 7.21-7.08 (m, 1H), 7.08-6.95 (m, 3H), 3.92-3.86 (m, 3H), 3.52-3.48 (m, 1H), 3.15-3.07 (m, 2H), 2.64-2.55 (m, 1H), 2.40 (br dd, J = 1.2, 4.2 Hz, 1H), 1.96- 1.83 (m, 2H), 1.81- 1.68 (m, 1H), 1.60- 1.46 (m, 3H), 1.20- 1.05 (m, 1H). | 2.27 A |
| 211 | 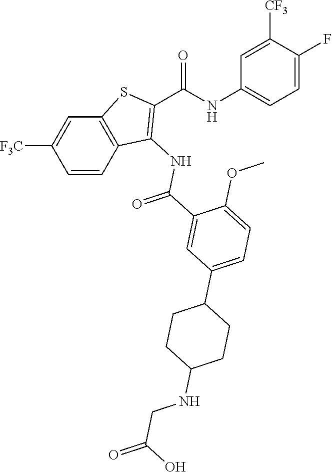 [SMILES: COc1ccc(C2CCC(NCC(=O)O)CC2)cc1C(=O)Nc1c(C(=O)Nc2ccc(F)c(C(F)(F)F)c2)sc2cc(C(F)(F)F)ccc12] | (4-(3-((2-((4- fluoro-3- (trifluoromethyl) phenyl) carbamoyl)-6- (trifluoromethyl) benzo[b] thiophen-3- yl)carbamoyl)- 4- methoxyphenyl) cyclohexyl) glycine | 712.20 | 1H NMR (400 MHz, DMSO-d6) δ ppm 10.77-10.86 (m, 2 H) 8.64-8.69 (m, 1 H) 8.13-8.17 (m, 1 H) 8.04-8.08 (m, 1 H) 7.96-8.03 (m, 1 H) 7.79-7.84 (m, 1 H) 7.63-7.67 (m, 1 H) 7.51-7.59 (m, 1 H) 7.40-7.46 (m, 1 H) 7.12-7.19 (m, 1 H) 4.33 (m, 1H) 3.93 (s, 3 H) 3.27-3.55 (m, 2 H) 3.18 (br d, J = 7.58 Hz, 2 H) 2.50 (dt, J = 3.67, 1.83 Hz, 2 H) 2.06-2.12 (m, 2 H) 1.77-1.85 (m, 2 H) 1.44 (br d, J = 9.05 Hz, 2 H) | 2.04 A Isomers were separated by prep SFC (Method 24) to give the 1st eluting isomer (Prep SFC RT = 4.36 min, 100% de) and Example 211 (Prep SFC RT = 7.11 min.). 91% de Analytical SFC (Method 25). |
| 212 | 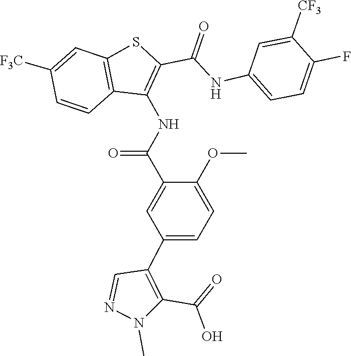 [SMILES: COc1ccc(-c2cnn(C)c2C(=O)O)cc1C(=O)Nc1c(C(=O)Nc2ccc(F)c(C(F)(F)F)c2)sc2cc(C(F)(F)F)ccc12] | 4-(3-((2-((4- fluoro-3- (trifluoromethyl) phenyl) carbamoyl)-6- (trifluoromethyl) benzo[b] thiophen-3- yl)carbamoyl)- 4- methoxyphenyl)- 1-methyl-1H- pyrazole-5- carboxylic acid | 679.20 | 1H NMR (400 MHz, DMSO-d6) δ ppm 12.5-11.9 (m, 1H), 10.87 (br s, 1 H) 10.75 (br s, 1 H) 8.67 (s, 1 H) 8.16-8.20 (m, 1 H) 8.01-8.11 (m, 1 H) 7.93 (s, 2 H) 7.83 (s, 1 H) 7.81 (s, 1 H) 7.50-7.59 (m, 2 H) 7.22 (d, J = 8.53 Hz, 1 H) 3.90 (s, 3 H) ) 2.61 (s, 3 H) | 2.48 C |
| 213 | 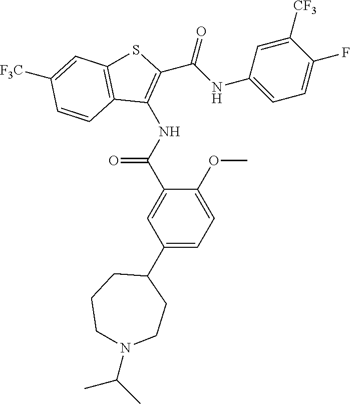 [SMILES: COc1ccc(C2CCCN(C(C)C)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (1- isopropylazepan- 4-yl)-2- methoxy- benzamido)-6- (trifluoromethyl) benzo[b] thiophene-2- carboxamide | 696.30 | 1H NMR (400 MHz, DMSO-d6) δ ppm 10.95-10.63 (m, 2H), 8.68 (s, 1H), 8.16 (dd, J = 2.7, 6.4 Hz, 1H), 8.06 (d, J = 8.6 Hz, 1H), 8.03-7.94 (m, 1H), 7.82 (dd, J = 1.6, 8.7 Hz, 1H), 7.69 (br d, J = 1.5 Hz, 1H), 7.55 (t, J = 9.8 Hz, 1H), 7.43 (br dd, J = 1.2, 8.8 Hz, 1H), 7.18 (br d, J = 8.3 Hz, 1H), 3.95 (s, 3H), 3.13- 2.86 (m, 4H), 2.86- 2.72 (m, 2H), 2.01- 1.60 (m, 6H), 1.22- 0.93 (m, 6H) | 2.24 A |
| 214 | 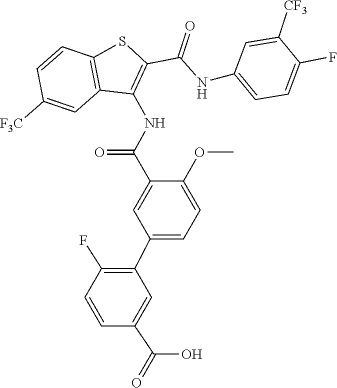 [SMILES: COc1ccc(-c2cc(C(=O)O)ccc2F)cc1C(=O)Nc1c(C(=O)Nc2ccc(F)c(C(F)(F)F)c2)sc2ccc(C(F)(F)F)cc12] | 6-fluoro-3′-((2- ((4-fluoro-3- (trifluoromethyl) phenyl) carbamoyl)-5- (trifluoromethyl) benzo[b] thiophen-3- yl)carbamoyl)- 4′-methoxy- [1,1′-biphenyl]- 3-carboxylic acid | 695.00 | 1H NMR (300 MHz, DMSO-d6) δ ppm 12.2-11.7 (m, 1H), 10.75-11.01 (m, 2 H) 8.34-8.43 (m, 1 H) 8.30-8.40 (m, 1 H) 8.13-8.20 (m, 1 H) 8.02-8.10 (m, 1 H) 8.06 (s, 2 H) 7.76- 8.00 (m, 3 H) 7.75- 7.91 (m, 1 H) 7.36 (d, J = 9.07 Hz, 2 H) 3.97 (s, 3 H) | 3.10 C |
| 215 | 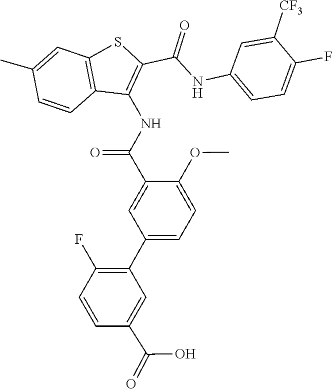 [SMILES: COc1ccc(-c2cc(C(=O)O)ccc2F)cc1C(=O)Nc1c(C(=O)Nc2ccc(F)c(C(F)(F)F)c2)sc2cc(C)ccc12] | 6-fluoro-3′-((2- ((4-fluoro-3- (trifluoromethyl) phenyl) carbamoyl)-6- methylbenzo[b] thiophen-3- yl)carbamoyl)- 4′-methoxy- [1,1′-biphenyl]- 3-carboxylic acid | 641.00 | 1H NMR (400 MHz, DMSO-d6) δ ppm 12.2-11.3 (m, 1H), 10.83-10.87 (m, 1 H) 10.56-10.60 (m, 1 H) 8.37-8.44 (m, 1 H) 8.12-8.19 (m, 1 H) 8.07-8.12 (m, 1 H) 7.98-8.05 (m, 1 H) 7.88-7.94 (m, 2 H) 7.79-7.87 (m, 1 H) 7.69-7.75 (m, 2 H) 7.49-7.59 (m, 1 H) 7.43-7.48 (m, 1 H) 7.35-7.42 (m, 1 H) 3.86 (s, 3 H) 2.53 (dt, J = 3.64, 1.95 Hz, 3 H) | 3.08 C |
| 216 | 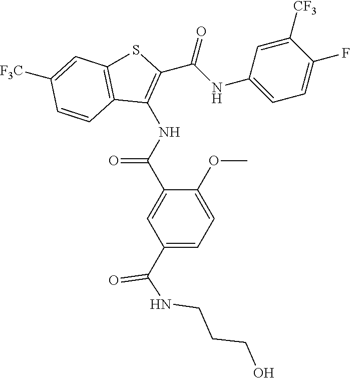 [SMILES: COc1ccc(C(=O)NCCCO)cc1C(=O)Nc1c(C(=O)Nc2ccc(F)c(C(F)(F)F)c2)sc2cc(C(F)(F)F)ccc12] | N3-(2-((4- fluoro-3- (trifluoromethyl) phenyl) carbamoyl)-6- (trifluoromethyl) benzo[b] thiophen-3-yl)-N1- (3- hydroxypropyl)- 4- methoxy- isophthalamide | 658.10 | 1H NMR (400 MHz, DMSO-d6) δ ppm 10.92-10.68 (m, 2H), 8.67 (s, 1H), 8.50 (t, J = 5.6 Hz, 1H), 8.36 (d, J = 2.2 Hz, 1H), 8.16 (dd, J = 2.4, 6.4 Hz, 1H), 8.12-8.02 (m, 2H), 8.02-7.92 (m, 1H), 7.82 (dd, J = 1.6, 8.9 Hz, 1H), 7.54 (t, J = 10.0 Hz, 1H), 7.31 (d, J = 8.6 Hz, 1H), 4.47 (br t, J = 4.8 Hz, 1H), 3.98 (s, 3H), 3.45 (br d, J = 4.9 Hz, 2H), 3.32-3.28 (m, 2H), 1.67 (quin, J = 6.7 Hz, 2H). | 2.12 C |
| 217 | 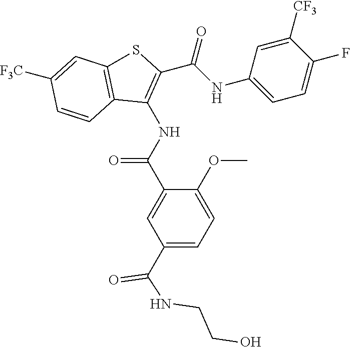 [SMILES: COc1ccc(C(=O)NCCO)cc1C(=O)Nc1c(C(=O)Nc2ccc(F)c(C(F)(F)F)c2)sc2cc(C(F)(F)F)ccc12] | N3-(2-((4- fluoro-3- (trifluoromethyl) phenyl) carbamoyl)-6- (trifluoromethyl) benzo[b] thiophen-3-yl)-N1- (2- hydroxyethyl)- 4- methoxy- isophthalamide | 644.20 | 1H NMR (400 MHz, DMSO-d6) δ ppm 10.91-10.83 (m, 2H), 10.81-10.72 (m, 1H), 8.68 (s, 1H), 8.49 (t, J = 5.6 Hz, 1H), 8.38 (d, J = 2.4 Hz, 1H), 8.17 (dd, J = 2.6, 6.2 Hz, 1H), 8.10-8.04 (m, 1H), 8.00 (td, J = 2.0, 4.5 Hz, 1H), 7.82 (dd, J = 1.7, 8.8 Hz, 1H), 7.54 (t, J = 9.8 Hz, 1H), 7.31 (d, J = 8.8 Hz, 1H), 5.60 (s, 1H) 4.73 (s, 2H), 3.98 (s, 3H), 3.54-3.47 (m, 2H). | 2.09 C |
| 218 | 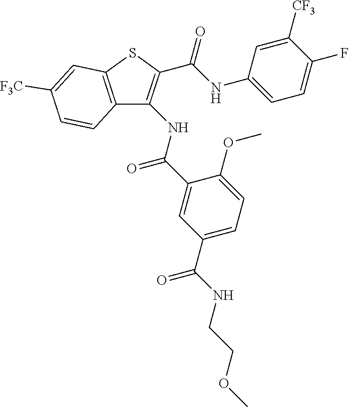 [SMILES: COCCNC(=O)c1ccc(OC)c(C(=O)Nc2c(C(=O)Nc3ccc(F)c(C(F)(F)F)c3)sc3cc(C(F)(F)F)ccc23)c1] | N3-(2-((4- fluoro-3- (trifluoromethyl) phenyl) carbamoyl)-6- (trifluoromethyl) benzo[b] thiophen-3-yl)-4- methoxy-N1- (2- methoxyethyl) isophthalamide | 658.2 | NMR Not clean | 2.27 A |
| 219 | 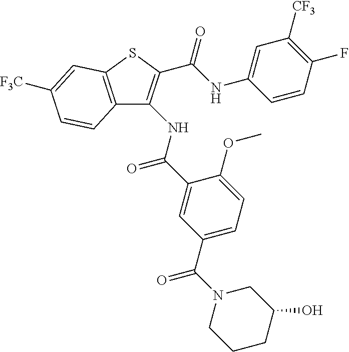 [SMILES: COc1ccc(C(=O)N2CCC[C@@H](O)C2)cc1C(=O)Nc1c(C(=O)Nc2ccc(F)c(C(F)(F)F)c2)sc2cc(C(F)(F)F)ccc12] | (R)-N-(4- fluoro-3- (trifluoromethyl) phenyl)-3-(5- (3- hydroxy- piperidine-1- carbonyl)-2- methoxy- benzamido)-6- (trifluoromethyl) benzo[b] thiophene-2- carboxamide | 684.2 | 1H NMR (400 MHz, DMSO-d6) δ ppm 10.53-10.79 (m, 2 H) 8.66 (s, 1 H) 8.16 (br s, 1 H) 8.08 (d, J = 8.57 Hz, 1 H) 7.96-7.99 (m, 1 H) 7.82 (br d, J = 7.09 Hz, 2 H) 7.50- 7.64 (m, 2 H) 7.28 (d, J = 8.80 Hz, 1 H) 4.81 (s, 1H), (3.97 (s, 3 H) 3.24 (br s, 2 H) 3.17 (br s, 4 H) 1.23 (s, 2 H) | 2.20 A |
| 220 | 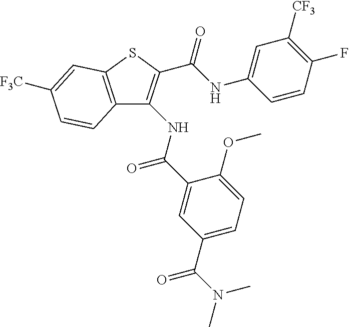 [SMILES: COc1ccc(C(=O)N(C)C)cc1C(=O)Nc1c(C(=O)Nc2ccc(F)c(C(F)(F)F)c2)sc2cc(C(F)(F)F)ccc12] | N3-(2-((4- fluoro-3- (trifluoromethyl) phenyl) carbamoyl)-6- (trifluoromethyl) benzo[b] thiophen-3-yl)-4- methoxy- N1,N1- dimethyl- isophthalamide | 628.2 | 1H NMR (400 MHz, DMSO-d6) δ ppm 10.70-10.84 (m, 2 H) 8.64-8.68 (m, 1 H) 8.11-8.17 (m, 1 H) 8.04-8.11 (m, 2 H) 7.93-8.02 (m, 1 H) 7.78-7.84 (m, 1 H) 7.62-7.67 (m, 1 H) 7.50-7.58 (m, 1 H) 7.26-7.31 (m, 1 H) 3.97 (s, 3 H) 3.50 (s, 3 H) 3.25 (s, 3 H) | 2.29 A |
| 221 | 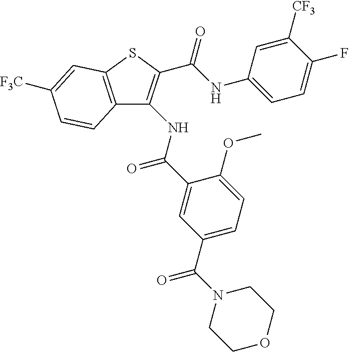 [SMILES: COc1ccc(C(=O)N2CCOCC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5- (morpholine-4- carbonyl) benzamido)-6- (trifluoromethyl) benzo[b] thiophene-2- carboxamide | 670.2 | 1H NMR (400 MHz, DMSO-d6) δ ppm 10.87 (s, 1H), 10.77 (d, J = 0.7 Hz, 1H), 8.65 (s, 1 H) 7.95- 8.17 (m, 3 H) 7.74- 7.85 (m, 2 H) 7.58- 7.65 (m, 1 H) 7.54 (t, J = 9.49 Hz, 1 H) 7.24- 7.32 (m, 1 H) 3.96 (s, 3 H) 3.58 (br s, 2 H) 3.49 (br d, J = 9.78 Hz, 2 H) 3.23 (s, 2 H) 1.23 (s, 2 H) | 2.26 A |
| 222 | 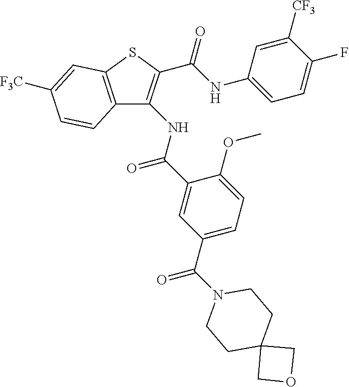 [SMILES: COc1ccc(C(=O)N2CCC3(CC2)COC3)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(2- oxa-7- azaspiro[3.5] nonane-7- carbonyl) benzamido)-6- (trifluoromethyl) benzo[b] thiophene-2- carboxamide | 710.2 | 1H NMR (400 MHz, DMSO-d6) δ ppm 10.93-10.59 (m, 2H), 8.66 (s, 1H), 8.16 (dd, J = 6.4, 2.4 Hz, 1H), 8.08 (d, J = 8.6 Hz, 1H), 8.02-7.93 (m, 1H), 7.87-7.74 (m, 2H), 7.64-7.57 (m, 1H), 7.54 (t, J = 9.8 Hz, 1H), 7.29 (d, J = 8.6 Hz, 1H), 4.32 (s, 4H), 3.98 (s, 3H), 1.76 (br dd, J = 2.9, 2.0 Hz, 4H) | 2.27 A |
| 223 | 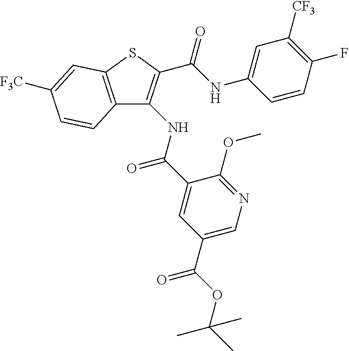 [SMILES: COc1ncc(C(=O)OC(C)(C)C)cc1C(=O)Nc1c(C(=O)Nc2ccc(F)c(C(F)(F)F)c2)sc2cc(C(F)(F)F)ccc12] | tert-butyl 5- ((2-((4-fluoro- 3- (trifluoromethyl) phenyl) carbamoyl)-6- (trifluoromethyl) benzo[b] thiophen-3- yl)carbamoyl)- 6- methoxynicotinate | 658.1 | 1H NMR (400 MHz, DMSO-d6) δ ppm 10.87 (br s, 1H), 10.77 (br d, J = 1.7 Hz, 1H), 8.85 (d, J = 2.4 Hz, 1H), 8.67 (s, 1H), 8.53 (d, J = 2.2 Hz, 1H), 8.19- 8.06 (m,2H), 8.05- 7.93 (m, 1H), 7.82 (d, J = 8.1 Hz, 1H), 7.53 (t, J = 9.9 Hz, 1H), 4.07 (s, 3H), 1.55 (s, 9H) | 2.83 A |
| 224 | 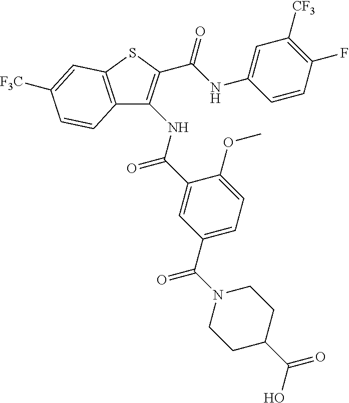 [SMILES: COc1ccc(C(=O)N2CCC(C(=O)O)CC2)cc1C(=O)Nc1c(C(=O)Nc2ccc(F)c(C(F)(F)F)c2)sc2cc(C(F)(F)F)ccc12] | 1-((3-((2-((4- fluoro-3- (trifluoromethyl) phenyl) carbamoyl)-6- (trifluoromethyl) benzo[b] thiophen-3- yl)carbamoyl)- 4- methoxyphenyl) sulfonyl) piperidine-4- carboxylic acid | 746.1 | 1H NMR (400 MHz, DMSO-d6) δ ppm 10.84 (s, 1H), , 10.80- 10.70 (m, 1H) 8.67 (s, 1H), 8.23-8.17 (m, 1H), 8.10 (d, J = 8.3 Hz, 1H), 8.04 (d, J = 2.0 Hz, 1H), 8.00-7.93 (m, 1H), 7.91-7.87 (m, 1H), 7.84 (dd, J = 1.3, 8.9 Hz, 1H), 7.58- 7.50 (m, 1H), 7.48- 7.40 (m, 1H), 4.03- 3.99 (m, 3H), 3.45- 3.40 (m, 2H), 2.41- 2.33 (m, 2H), 2.27- 2.20 (m, 1H), 1.89- 1.81 (m, 2H), 1.59- 1.50 (m, 2H). | 1.91 B |
| 225 | 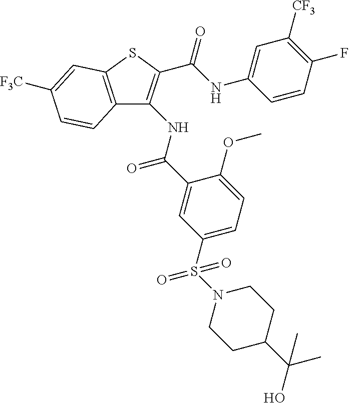 [SMILES: COc1ccc(S(=O)(=O)N2CCC(C(C)(C)O)CC2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- ((4-(2- hydroxypropan- 2-yl)piperidin- 1-yl)sulfonyl)- 2- methoxy- benzamido)-6- (trifluoromethyl) benzo[b] thiophene-2- carboxamide | 762.2 | 1H NMR (400 MHz, DMSO-d6) δ ppm 10.88 (br s, 1H), 10.83- 10.72 (m, 1H), 8.67 (s, 1H), 8.23-8.14 (m, 1H), 8.13-8.07 (m, 1H), 8.07-7.92 (m, 2H), 7.92-7.76 (m, 2H), 7.54 (t, J = 9.7 Hz, 1H), 7.48-7.36 (m, 1H), 4.18-4.10 (m, 1H), 4.01 (s, 3H), 3.70- 3.61 (m, 2H), 2.10- 1.98 (m, 2H), 1.73- 1.62 (m, 2H), 1.32- 1.14 (m, 2H), 1.07- 0.86 (m, 7H). | 2.38 B |
| 226 | 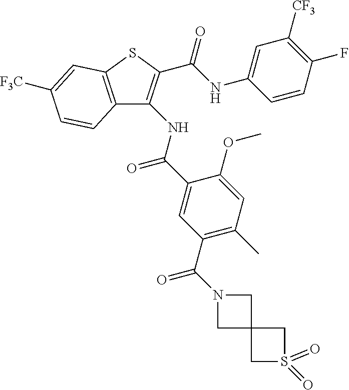 [SMILES: COc1cc(C)c(C(=O)N2CC3(C2)CS(=O)(=O)C3)cc1C(=O)Nc1c(C(=O)Nc2ccc(F)c(C(F)(F)F)c2)sc2cc(C(F)(F)F)ccc12] | 3-(5-(2,2- dioxido-2-thia- 6- azaspiro[3.3] heptane-6- carbonyl)-2- methoxy-4- methylbenzamido)- N-(4- fluoro-3- (trifluoromethyl) phenyl)-6- (trifluoromethyl) benzo[b] thiophene-2- carboxamide | 744.2 | 1H NMR (400 MHz, DMSO-d6) δ ppm 10.82 (s, 1H), 10.76 (s, 1H), 8.67 (s, 1H), 8.18 (dd, J = 2.4, 6.6 Hz, 1H), 8.04 (d, J = 8.8 Hz, 1H), 8.01-7.93 (m, 1H), 7.86-7.73 (m, 2H), 7.55 (t, J = 9.9 Hz, 1H), 7.18 (s, 1H), 4.51- 4.37 (m, 4H), 4.26 (br s, 2H), 4.16 (br s, 2H), 3.98 (s, 3H), 2.41 (s, 3H) | 2.20 B |
| 227 | 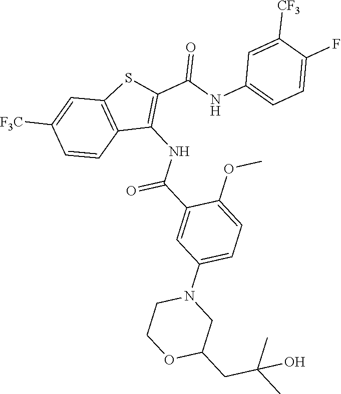 [SMILES: COc1ccc(N2CCOC(CC(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (2-(2-hydroxy- 2- methylpropyl) morpholino)-2- methoxy- benzamido)-6- (trifluoromethyl) benzo[b] thiophene-2- carboxamide | 714.3 | 1H NMR (400 MHz, DMSO-d6) δ ppm 10.88-10.83 (m, 1H), 10.85 (s, 1H), 8.66 (s, 1H), 8.73-8.54 (m, 1H), 8.15 (dd, J = 2.6, 6.5 Hz, 1H), 8.18- 8.11 (m, 1H), 8.08- 7.97 (m, 1H), 7.81 (dd, J = 1.7, 8.7 Hz, 1H), 7.54 (t, J = 9.8 Hz, 1H), 7.39 (s, 1H), 7.17 (s, 1H), 4.22 (s, 1H), 4.30- 4.12 (m, 1H), 3.91 (s, 3H), 3.99-3.85 (m, 1H), 3.67-3.59 (m, 1H), 3.41-3.40 (m, 1H), 3.49-3.39 (m, 1H), 3.32 (s, 1H), 2.69- 2.66 (m, 1H), 2.38- 2.28 (m, 1H), 1.61- 1.49 (m, 1H), 1.15 (s, 6H) | 2.51 A. The isomers were separated by prep SFC (Method 26) to give Example 227 (Prep SFC RT = 7.71 min). 99% ee Analytical SFC (Method 27). |
| 228 | 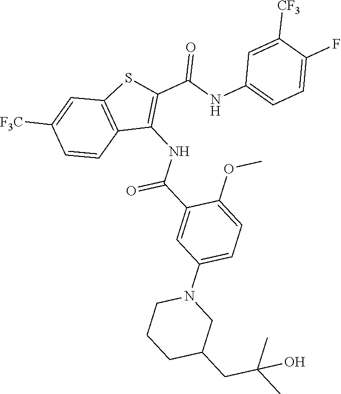 [SMILES: COc1ccc(N2CCCC(CC(C)(C)O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (2-(2-hydroxy- 2- methylpropyl) morpholino)-2- methoxy- benzamido)-6- (trifluoromethyl) benzo[b] thiophene-2- carboxamide | 714.2 | 1H NMR (400 MHz, DMSO-d6) δ ppm 10.88-10.83 (m, 1H), 10.85 (s, 1H), 8.73- 8.54 (m, 1H), 8.66 (s, 1H), 8.15 (dd, J = 2.6, 6.5 Hz, 1H), 8.18- 8.11 (m, 1H), 8.08- 7.97 (m, 1H), 7.81 (dd, J = 1.7, 8.7 Hz, 1H), 7.54 (t, J = 9.8 Hz, 1H), 7.39 (s, 1H), 7.17 (s, 1H), 4.22 (s, 1H), 4.30- 4.12 (m, 1H), 3.91 (s, 3H), 3.99-3.85 (m, 1H), 3.67-3.59 (m, 1H), 3.41-3.40 (m, 1H), 3.37 (br s, 1H), 3.32 (s, 1H), 2.69- 2.66 (m, 1H), 2.38- 2.28 (m, 1H), 1.61- 1.49 (m, 1H), 1.15 (s, 6H) | 2.51 A. The isomers were separated by prep SFC (Method 26) to give Example 228 (Prep SFC RT = 5.13 min). 99% ee Analytical SFC (Method 27). |
| 229 | 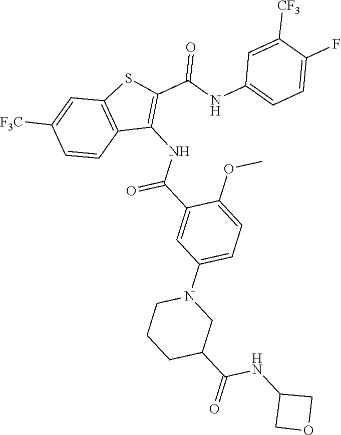 [SMILES: COc1ccc(N2CCCC(C(=O)NC3COC3)C2)cc1C(=O)Nc1c(C(=O)Nc2ccc(F)c(C(F)(F)F)c2)sc2cc(C(F)(F)F)ccc12] | 1-(3-((2-((4- fluoro-3- (trifluoromethyl) phenyl) carbamoyl)-6- (trifluoromethyl) benzo [b] thiophen-3- yl)carbamoyl)- 4- methoxyphenyl)- N-(oxetan-3- yl)piperidine- 3-carboxamide | 739.2 | 1H NMR (400 MHz, DMSO-d6) δ ppm 10.86-10.82 (m, 1H), 10.73 (s, 1H), 8.66 (s, 1H), 8.64-8.60 (m, 1H), 8.19-8.13 (m, 1H), 8.06 (d, J = 8.1 Hz, 1H), 8.01-7.95 (m, 1H), 7.87-7.74 (m, 1H), 7.54 (t, J = 9.8 Hz, 1H), 7.42 (d, J = 2.9 Hz, 1H), 7.24-7.17 (m, 1H), 7.16-7.10 (m, 1H), 4.86-4.76 (m, 1H),4.41 (dt, J = 4.3, 6.2 Hz, 1H), 3.94 (s, 3H), 3.32 (s, 4H), 3.34- 3.26 (m, 1H), 2.50 (td, J = 1.8, 3.7 Hz, 6H), 2.53-2.42 (m, 1H) | 2.33 B The isomers were separated by prep SFC (Method 8) Prep SFC RT = 7.79 min. 99% ee |
| 230 | 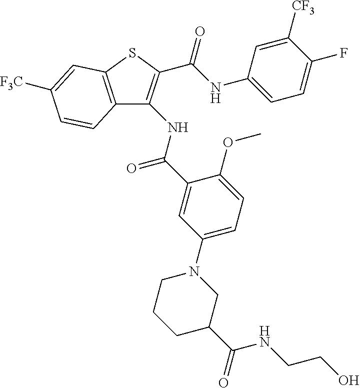 [SMILES: COc1ccc(N2CCCC(C(=O)NCCO)C2)cc1C(=O)Nc1c(C(=O)Nc2ccc(F)c(C(F)(F)F)c2)sc2cc(C(F)(F)F)ccc12] | 1-(3-((2-((4- fluoro-3- (trifluoromethyl) phenyl) carbamoyl)-6- (trifluoromethyl) benzo[b] thiophen-3- yl)carbamoyl)- 4- methoxyphenyl)- N-(2- hydroxyethyl) piperidine-3- carboxamide | 727.2 | 1H NMR (400 MHz, DMSO-d6) δ ppm 10.86 (s, 1H), 10.75 (s, 1H), 8.67 (s, 1H), 8.17 (dd, J = 2.6, 6.7 Hz, 1H), 8.06 (d, J = 8.8 Hz, 1H), 7.98 (td, J = 3.5, 9.0 Hz, 1H), 7.90 (t, J = 5.5 Hz, 1H), 7.81 (dd, J = 1.5, 8.8 Hz, 1H), 7.54 (t, J = 9.8 Hz, 1H), 7.41 (d, J = 3.2 Hz, 1H), 7.25-7.16 (m, 1H), 7.14-7.08 (m, 1H), 4.64 (t, J = 5.5 Hz, 1H), 3.90 (s, 3H), 3.58- 3.52 (m, 1H), 3.47- 3.36 (m, 3H), 3.18- 3.07 (m, 2H), 2.69- 2.65 (m, 1H), 2.60- 2.54 (m, 1H), 2.48- 2.41 (m, 1H), 1.88- 1.78 (m, 1H), 1.78- 1.66 (m, 1H), 1.61- 1.38 (m, 2H) | 2.24 B The isomers were separated by prep SFC (Method 8) Prep SFC RT = 4.88 min.. |
| 231 | 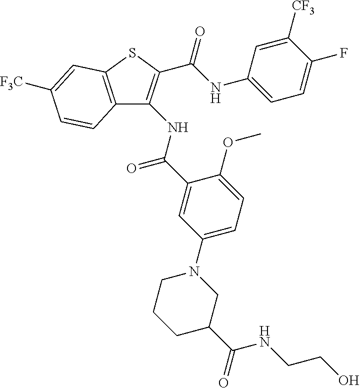 [SMILES: COc1ccc(N2CCCC(C(=O)NCCO)C2)cc1C(=O)Nc1c(C(=O)Nc2ccc(F)c(C(F)(F)F)c2)sc2cc(C(F)(F)F)ccc12] | 1-(3-((2-((4- fluoro-3- (trifluoromethyl) phenyl) carbamoyl)-6- (trifluoromethyl) benzo[b] thiophen-3- yl)carbamoyl)- 4- methoxyphenyl)- N-(2- hydroxyethyl) piperidine-3- carboxamide | 727.2 | 1H NMR (400 MHz, DMSO-d6) δ ppm 10.85 (s, 1H), 10.73 (s, 1H), 8.66 (d, J = 1.0 Hz, 1H), 8.19-8.14 (m, 1H), 8.06 (d, J = 8.1 Hz, 1H), 8.01-7.95 (m, 1H), 7.91-7.85 (m, 1H), 7.81 (dd, J = 1.3, 8.9 Hz, 1H), 7.54 (t, J = 9.7 Hz, 1H), 7.41 (d, J = 2.4 Hz, 1H), 7.23- 7.17 (m, 1H), 7.16- 7.11 (m, 1H), 4.63 (t, J = 5.4 Hz, 1H), 4.12- 4.03 (m, 1H), 3.94 (s, 3H), 3.61-3.50 (m, 2H), 3.48-3.37 (m, 2H), 3.32 (s, 4H), 2.50 (td, J = 1.8, 3.7 Hz, 4H) | 2.24 B The isomers were separated by prep SFC (Method 8) RT = 3.33 min. |
| 232 | 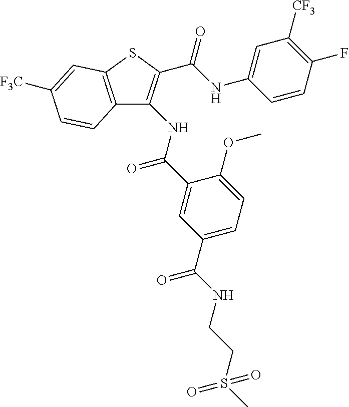 [SMILES: COc1ccc(C(=O)NCCS(C)(=O)=O)cc1C(=O)Nc1c(C(=O)Nc2ccc(F)c(C(F)(F)F)c2)sc2cc(C(F)(F)F)ccc12] | N3-(2-((4- fluoro-3- (trifluoromethyl) phenyl) carbamoyl)-6- (trifluoromethyl) benzo[b] thiophen-3-yl)-4- methoxy-N1- (2- (methylsulfonyl) ethyl) isophthalamide | 714.2 (M − H)⁻ | 1H NMR (400 MHz, DMSO-d6) δ ppm 10.85 (br s, 1H), 10.79- 10.71 (m, 1H), 8.77 (br t, J = 5.6 Hz, 1H), 8.67 (s, 1H), 8.38 (d, J = 2.4 Hz, 1H), 8.16 (dd, J = 2.3, 6.5 Hz, 1H), 8.12-8.03 (m, 2H), 8.02-7.93 (m, 1H), 7.82 (dd, J = 1.7, 8.8 Hz, 1H), 7.54 (t, J = 9.7 Hz, 1H), 7.34 (d, J = 8.8 Hz, 1H), 4.00 (s, 3H), 3.72-3.61 (m, 2H), 3.40-3.35 (m, 2H), 3.02 (s, 3H) | 2.13 A |
| 233 | 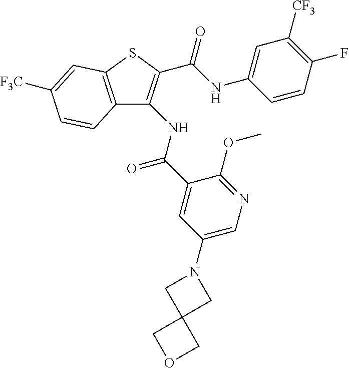 [SMILES: COc1ncc(N2CC3(COC3)C2)cc1C(=O)Nc1c(C(=O)Nc2ccc(F)c(C(F)(F)F)c2)sc2cc(C(F)(F)F)ccc12] | N-(2-((4- fluoro-3- (trifluoromethyl) phenyl) carbamoyl)-6- (trifluoromethyl) benzo[b] thiophen-3-yl)-2- methoxy-5-(2- oxa-6- azaspiro[3.3] heptan-6- yl)nicotinamide | 653.1 | 1H NMR (400 MHz, DMSO-d6) δ ppm 10.91-10.72 (m, 2H), 8.66 (s, 1H), 8.14 (dd, J = 2.4, 6.4 Hz, 1H), 8.05 (d, J = 8.8 Hz, 1H), 8.01-7.94 (m, 1H), 7.80 (dd, J = 1.0, 8.6 Hz, 1H), 7.64 (d, J = 2.7 Hz, 1H), 7.55 (t, J = 10.1 Hz, 1H), 7.38 (d, J = 2.7 Hz, 1H), 4.71 (s, 4H), 3.98 (s, 4H), 3.93 (s, 3H) | 2.43 A |
| 234 | 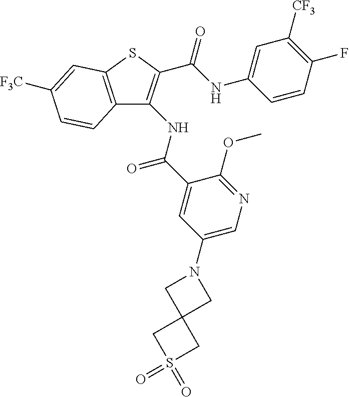 [SMILES: COc1ncc(N2CC3(C2)CS(=O)(=O)C3)cc1C(=O)Nc1c(C(=O)Nc2ccc(F)c(C(F)(F)F)c2)sc2cc(C(F)(F)F)ccc12] | 5-(2,2-dioxido- 2-thia-6- azaspiro[3.3] heptan-6-yl)-N- (2-((4-fluoro-3- (trifluoromethyl) phenyl) carbamoyl)-6- (trifluoromethyl) benzo[b] thiophen-3-yl)-2- methoxy- nicotinamide | 701.2 (M − H)⁻ | 1H NMR (400 MHz, H2O + D2O) δ ppm 10.95-10.71 (m, 2H), 8.67 (s, 1H), 8.21- 8.14 (m, 1H), 8.05 (d, J = 8.8 Hz, 1H), 8.01- 7.94 (m, 1H), 7.80 (dd, J = 1.2, 8.6 Hz, 1H), 7.70 (d, J = 2.9 Hz, 1H), 7.55 (t, J = 9.7 Hz, 1H), 7.43 (d, J = 2.9 Hz, 1H), 4.48 (s, 4H), 4.06 (s, 4H), 3.94 (s, 3H) | 2.36 A |
| 235 | 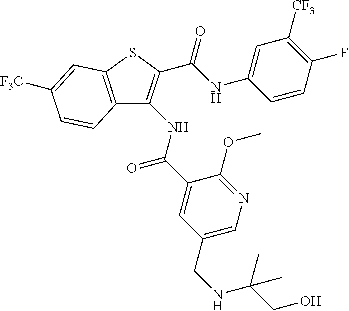 [SMILES: COc1ncc(CNC(C)(C)CO)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (((1-hydroxy- 2- methylpropan- 2- yl)amino) methyl)-2- methoxy- benzamido)-6- (trifluoromethyl) benzo[b] thiophene-2- carboxamide | 658.3 | 1H NMR (400 MHz, DMSO-d6) δ ppm 11.02-10.75 (m, 2H), 8.64 (s, 1H), 8.18- 8.10 (m, 1H), 8.07 (d, J = 8.3 Hz, 1H), 7.96 (td, J = 2.3, 5.4 Hz, 1H), 7.86-7.70 (m, 2H), 7.60-7.41 (m, 2H), 7.23-7.06 (m, 1H), 4.71-4.52 (m, 1H), 3.93 (br s, 3H), 3.62 (br s, 2H), 3.24 (s, 2H), 1.90 (s, 1H), 0.99 (s, 6H) | 2.12 A |
| 236 | 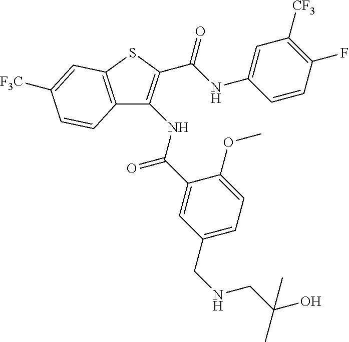 [SMILES: COc1ccc(CNCC(C)(C)O)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (((2-hydroxy- 2- methylpropyl) amino)methyl)- 2- methoxy- benzamido)-6- (trifluoromethyl) benzo[b] thiophene-2- carboxamide | 658.2 | 1H NMR (400 MHz, DMSO-d6) δ ppm 10.90 (s, 1H), 10.82 (s, 1H), 8.77-8.51 (m, 2H), 8.24-8.14 (m, 1H), 8.11 (d, J = 2.0 Hz, 1H), 8.07-7.94 (m, 2H), 7.82 (d, J = 8.6 Hz, 1H), 7.74 (dd, J = 2.0, 8.6 Hz, 1H), 7.56 (t, J = 9.9 Hz, 1H), 7.34 (d, J = 8.8 Hz, 1H), 5.15 (s, 1H), 4.16 (br s, 2H), 4.02 (s, 3H), 2.75 (br s, 2H), 1.15 (s,6H) | 2.24 A |
| 237 | 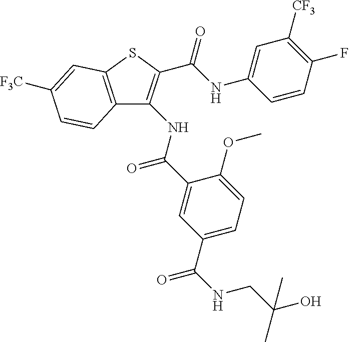 [SMILES: COc1ccc(C(=O)NCC(C)(C)O)cc1C(=O)Nc1c(C(=O)Nc2ccc(F)c(C(F)(F)F)c2)sc2cc(C(F)(F)F)ccc12] | N3-(2-((4- fluoro-3- (trifluoromethyl) phenyl) carbamoyl)-6- (trifluoromethyl) benzo[b] thiophen-3-yl)-N1- (2-hydroxy-2- methylpropyl)- 4- methoxy- isophthalamide | 672.2 | 1H NMR (400 MHz, DMSO-d6) δ ppm 10.85 (s, 1H), 10.74 (s, 1H), 8.67 (s, 1H), 8.37 (d, J = 2.2 Hz, 1H), 8.35-8.25 (m, 1H), 8.16 (dd, J = 2.7, 6.4 Hz, 1H), 8.13-8.04 (m, 2H), 8.03-7.94 (m, 1H), 7.86-7.77 (m, 1H), 7.58-7.47 (m, 1H), 7.31 (d, J = 8.8 Hz, 1H), 4.58 (s, 1H), 3.99 (s, 3H), 3.25 (d, J = 6.1 Hz, 2H), 1.09 (s, 6H) | 2.19 A |
| 238 | 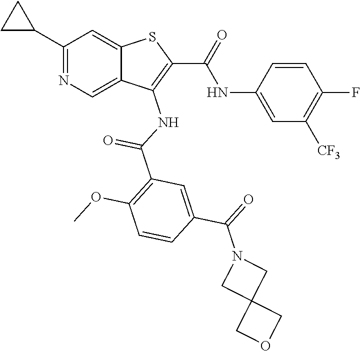 [SMILES: COc1ccc(C(=O)N2CC3(COC3)C2)cc1C(=O)Nc1c(C(=O)Nc2ccc(F)c(C(F)(F)F)c2)sc2cc(C3CC3)ncc12] | 6-cyclopropyl- N-(4-fluoro-3- (trifluoromethyl) phenyl)-3-(2- methoxy-5-(2- oxa-6- azaspiro[3.3] heptane-6- carbonyl) benzamido) thieno[3, 2-c]pyridine-2- carboxamide | 655.2 | 1H NMR (400 MHz, DMSO-d6) δ ppm 11.09 (s, 1H), 10.65 (s, 1H), 9.00 (s, 1H), 8.20- 8.10 (m, 2H), 8.06- 7.98 (m, 1H), 7.83 (dd, J = 8.5, 2.0 Hz, 1H), 7.54 (br t, J = 9.8 Hz, 1H), 7.30 (br d, J = 8.5 Hz, 1H), 7.23- 6.89 (m, 1H), 4.67 (s, 4H), 4.47 (br s, 2H), 4.21 (br s, 2H), 3.99 (s, 3H), 3.17 (s, 2H), 2.07 (s, 1H), 1.31-1.21 (m, 2H) | 2.02 A |
| 239 | 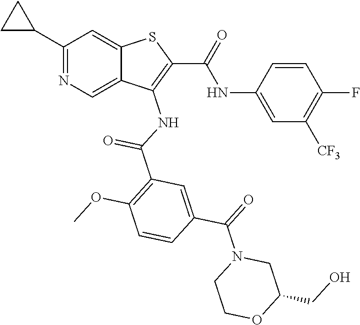 [SMILES: COc1ccc(C(=O)N2CCO[C@@H](CO)C2)cc1C(=O)Nc1c(C(=O)Nc2ccc(F)c(C(F)(F)F)c2)sc2cc(C3CC3)ncc12] | (R)-6-chloro- N-(4-fluoro-3- (trifluoromethyl) phenyl)-3-(5- (2- (hydroxymethyl) morpholine- 4-carbonyl)-2- methoxy- benzamido) thieno[3, 2-c]pyridine-2- carboxamide | 668.2 | 1H NMR (400 MHz, DMSO-d6) δ ppm 11.18-11.07 (m, 1H), 10.87-10.73 (m, 1H), 9.00 (d, J = 0.7 Hz, 1H), 8.39 (s, 1H), 8.17 (dd, J = 2.3, 6.5 Hz, 1H), 8.02-7.95 (m, 1H), 7.91 (d, J = 1.7 Hz, 1H), 7.65 (dd, J = 2.1, 8.4 Hz, 1H), 7.54 (t, J = 9.8 Hz, 1H), 7.29 (d, J = 8.6 Hz, 1H), 4.85-4.72 (m, 1H), 4.40-4.18 (m, 2H), 3.96 (s, 3H), 3.83 (br s, 2H), 3.49- 3.39 (m, 3H), 2.98 (br s, 2H) | 1.83 A |
| 240 | 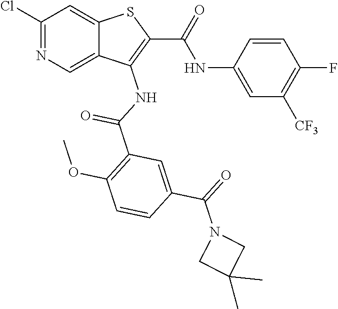 [SMILES: COc1ccc(C(=O)N2CC(C)(C)C2)cc1C(=O)Nc1c(C(=O)Nc2ccc(F)c(C(F)(F)F)c2)sc2cc(Cl)ncc12] | 6-chloro-3-(5- (3,3- dimethyl- azetidine-1- carbonyl)-2- methoxy- benzamido)-N-(4- fluoro-3- (trifluoromethyl) phenyl)thieno [3,2- c]pyridine-2- carboxamide | 636.1 | 1H NMR (400 MHz, DMSO-d6) δ ppm 11.15-10.74 (m, 2H), 9.00 (s, 1H), 8.37 (s, 1H), 8.14 (dd, J = 2.4, 6.4 Hz, 1H), 8.09 (br s, 1H), 8.02-7.92 (m, 1H), 7.84 (dd, J = 2.2, 8.6 Hz, 1H), 7.53 (t, J = 9.8 Hz, 1H), 7.27 (d, J = 8.6 Hz, 1H), 3.96 (br s, 3H), 3.90 (s, 2H), 3.72 (s, 2H), 1.23 (s, 6H) | 2.24 A |
| 241 | 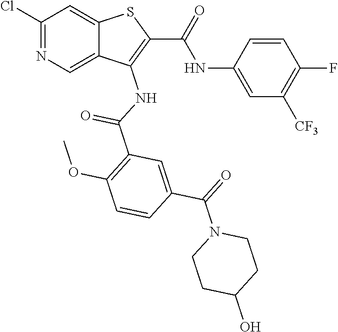 [SMILES: COc1ccc(C(=O)N2CCC(O)CC2)cc1C(=O)Nc1c(C(=O)Nc2ccc(F)c(C(F)(F)F)c2)sc2cc(Cl)ncc12] | 6-chloro-N-(4- fluoro-3- (trifluoromethyl) phenyl)-3-(5- (4- hydroxy- piperidine-1- carbonyl)-2- methoxy- benzamido) thieno[3, 2-c]pyridine-2- carboxamide | 652.2 | 1H NMR (400 MHz, DMSO-d6) δ ppm 11.10 (br dd, J = 4.8, 8.3 Hz, 1H), 10.79 (br d, J = 8.5 Hz, 1H), 9.02 (s, 1H), 8.40 (s, 1H), 8.21-8.14 (m, 1H), 8.00-7.94 (m, 1H), 7.87 (s, 1H), 7.64- 7.51 (m, 2H), 7.29 (br d, J = 9.0 Hz, 1H), 4.79 (d, J = 4.0 Hz, 1H), 3.96 (s, 34H), 3.78-3.59 (m, 3H), 1.79-1.65 (m, 2H), 1.36 (br d, J = 2.5 Hz, 2H), 1.24 (s, 2H) | 1.85 A |
| 242 | 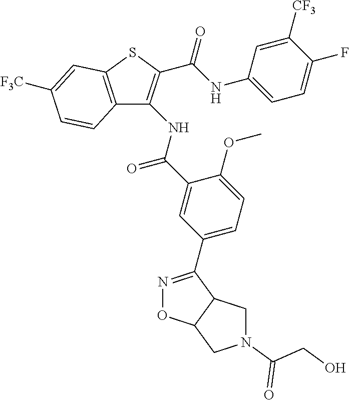 [SMILES: COc1ccc(C2=NOC3CN(C(=O)CO)CC23)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5-(2- hydroxyacetyl)- 3a,5,6,6a- tetrahydro-4H- pyrrolo[3,4- d]isoxazol-3- yl)-2- methoxy- benzamido)-6- (trifluoromethyl) benzo[b] thiophene-2- carboxamide | 725.2 | 1H NMR (400 MHz, DMSO-d6) δ ppm 11.17-10.58 (m, 2H), 8.66-8.57 (m, 1H), 8.19-8.03 (m, 2H), 7.98-7.87 (m, 1H), 7.86-7.74 (m, 3H), 7.56-7.46 (m, 1H), 7.34-7.26 (m, 1H), 5.42-5.27 (m, 1H), 4.77-4.69 (m, 1H), 3.94 (m, 3H), 3.71- 3.53 (m, 2H), 3.32 (s, 2H), 2.50 (td, J = 1.9, 3.6 Hz, 2H) | 2.52 C Isomers separated by Prep SFC (Method 18) to give Example 242 (Prep SFC RT = 6.2 min). 100% ee Analytical SFC (Method 19). |
| 243 | 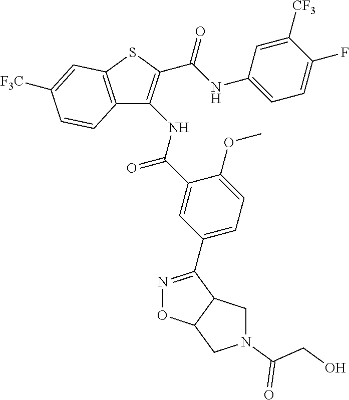 [SMILES: COc1ccc(C2=NOC3CN(C(=O)CO)CC23)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5-(2- hydroxyacetyl)- 3a,5,6,6a- tetrahydro-4H- pyrrolo[3,4- d]isoxazol-3- yl)-2- methoxy- benzamido)-6- (trifluoromethyl) benzo[b] thiophene-2- carboxamide | 725.2 | 1H NMR (400 MHz, DMSO-d6) δ ppm 11.14-10.65 (m, 2H), 8.62 (br s, 2H), 8.19- 8.06 (m, 2H), 7.79 (br d, J = 9.0 Hz, 1H), 7.52 (br s, 2H), 7.32 (br d, J = 9.0 Hz, 2H), 5.56- 5.18 (m, 1H), 4.04 (br s, 1H), 3.92 (m, 3H), 3.92-3.81 (m, 2H), 3.78 (br s, 2H), 3.42 (s, 2H) | 2.52 C Isomers separated by Prep SFC (Method 18) to give Example 243 (Prep SFC RT = 3.0 min). 99.5% ee Analytical SFC (Method 19). |
| 244 | 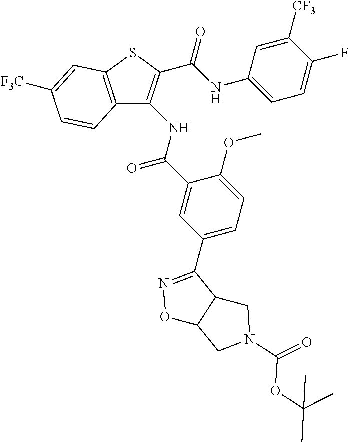 [SMILES: COc1ccc(C2=NOC3CN(C(=O)OC(C)(C)C)CC23)cc1C(=O)Nc1c(C(=O)Nc2ccc(F)c(C(F)(F)F)c2)sc2cc(C(F)(F)F)ccc12] | tert-butyl 3-(3- ((2-((4-fluoro- 3- (trifluoromethyl) phenyl) carbamoyl)-6- (trifluoromethyl) benzo[b] thiophen-3- yl)carbamoyl)- 4- methoxyphenyl)- 3a,4,6,6a- tetrahydro-5H- pyrrolo[3,4- d]isoxazole-5- carboxylate | 765 (M − H)⁻ | 1H NMR (400 MHz, DMSO-d6) δ ppm 10.94-10.67 (m, 2H), 8.36 (s, 1H), 8.22 (d, J = 8.5 Hz, 1H), 7.92 (br d, J = 1.5 Hz, 2H), 7.76- 7.58 (m, 2H), 7.37 (s, 1H), 7.42 (t, j = 8.9, 1H), 7.20 (d, J = 9.0 Hz, 1H), 5.30-5.22 (m, 1H), 4.49-4.41 (m, 1H), 3.85 (s, 3H), 3.78- 3.71 (m, 2H), 3.65- 3.46 (m, 2H), 1.44- 1.28 (m, 9H) | 3.54 C Isomers separated by Prep SFC (Method 28) to give Example 244 (Prep SFC RT = 10.2 min). 100% ee Analytical SFC (Method 29). |
| 245 | 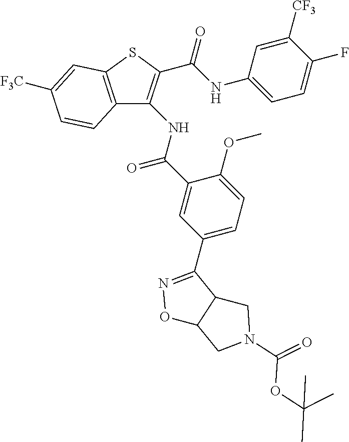 [SMILES: COc1ccc(C2=NOC3CN(C(=O)OC(C)(C)C)CC23)cc1C(=O)Nc1c(C(=O)Nc2ccc(F)c(C(F)(F)F)c2)sc2cc(C(F)(F)F)ccc12] | tert-butyl 3-(3- ((2-((4-fluoro- 3- (trifluoromethyl) phenyl) carbamoyl)-6- (trifluoromethyl) benzo[b] thiophen-3- yl)carbamoyl)- 4- methoxyphenyl)- 3a,4,6,6a- tetrahydro-5H- pyrrolo[3,4- d]isoxazole-5- carboxylate | 765.2 (M − H)⁻ | 1H NMR (400 MHz, DMSO-d6) δ ppm 10.94-10.67 (m, 2H), 8.46 (br s, 1H), 8.19 (d, J = 9.0 Hz, 1H), 7.98 (s, 2H), 7.80 (br s, 1H), 7.72 (br d, J = 9.5 Hz, 1H), 7.69 (br d, J = 8.0 Hz, 1H), 7.42 (br t, J = 9.5 Hz, 1H), 7.25 (d, J = 9.0 Hz, 1H), 5.27 (dd, J = 4.8, 8.8 Hz, 1H), 4.46 (br s, 1H), 3.87 (s, 3H), 3.78 (s, 2H), 3.75 (s, 2H), 1.33 (br s, 9H) | 3.54 C Isomers separated by Prep SFC (Method 28) to give Example 245 (Prep SFC RT = 6.7 min). 99.8% ee Analytical SFC (Method 29). |
| 246 | 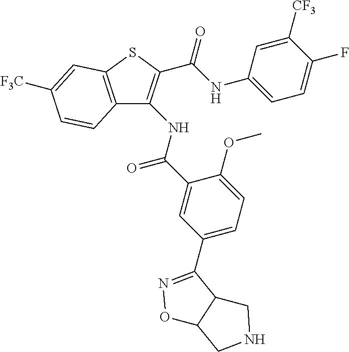 [SMILES: COc1ccc(C2=NOC3CNCC23)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5- (3a,5,6,6a- tetrahydro-4H- pyrrolo[3,4- d]isoxazol-3- yl)benzamido)- 6- (trifluoromethyl) benzo[b] thiophene-2- carboxamide | 667.2 | 1H NMR (400 MHz, DMSO-d6) δ ppm 11.40-10.56 (m, 2H), 8.63-8.58 (m, 1H), 8.12 (br s, 2H), 7.97- 7.89 (m, 1H), 7.79 (br s, 2H), 7.54-7.47 (m, 1H), 7.32-7.26 (m, 2H), 5.24-5.19 (m, 1H), 4.26-4.20 (m, 1H), 3.97-3.91 (m, 3H), 3.20-3.13 (m, 2H), 3.02-2.96 (m, 2H) | 1.75 C Isomers separated by Prep SFC (Method 18) to give Example 246 (Prep SFC RT 6.2 min). 99.6% ee Analytical SFC (Method 19) |
| 247 | 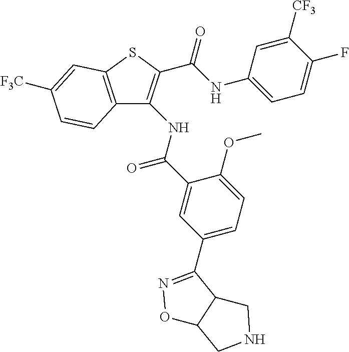 [SMILES: COc1ccc(C2=NOC3CNCC23)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5- (3a,5,6,6a- tetrahydro-4H- pyrrolo[3,4- d]isoxazol-3- yl)benzamido)- 6- (trifluoromethyl) benzo[b] thiophene-2- carboxamide | 667.2 | 1H NMR (400 MHz, DMSO-d6) δ ppm 10.92-10.73 (m, 2H), 8.68-8.55 (m, 1H), 8.18-8.04 (m, 2H), 7.99-7.87 (m, 1H), 7.85-7.73 (m, 2H), 7.58-7.46 (m, 1H), 7.34-7.25 (m, 2H), 5.24-5.18 (m, 1H), 4.27-4.19 (m, 1H), 3.99-3.91 (m, 3H), 3.20-3.13 (m, 2H), 3.03-2.96 (m, 2H) | 1.75 C Isomers separated by Prep SFC (Method 18) to give Example 247 (Prep SFC RT = 4.6 min). 98.1% ee Analytical SFC (Method 19) |
| 248 | 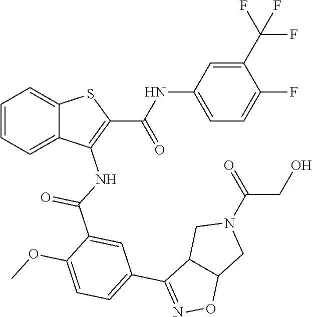 [SMILES: COc1ccc(C2=NOC3CN(C(=O)CO)CC23)cc1C(=O)Nc1c(C(=O)Nc2ccc(F)c(C(F)(F)F)c2)sc2ccccc12] | N-[4-fluoro-3- (trifluoromethyl) phenyl]-3-{5- [5-(2- hydroxyacetyl) 3aH,4H,5H,6H, 6aH- pyrrolo[3,4- d][1,2]oxazol- 3-yl]-2- methoxy- benzamido}-1- benzothiophene- 2- carboxamide | 655.1 (M − H)⁻ | 1H NMR (400 MHz, DMSO-d6) δ ppm 8.14- 7.67 (m, 6H), 7.53- 7.36 (m, 3H), 7.29- 7.21 (m, 1H), 5.41- 5.26 (m, 1H), 4.61- 4.41 (m, 1H), 4.09- 3.92 (m, 2H), 3.92- 3.74 (m, 6H), 3.70- 3.54 (m, 2H). | 2.86 C Isomers separated by Prep SFC (Method 30) to give isomer 1 (Prep SFC RT = 7.52 min, 100% ee) and isomer 2, Example 248 (Prep SFC RT = 10.26 min). 98.3% ee Analytical SFC (Method 31). |
| 249 | 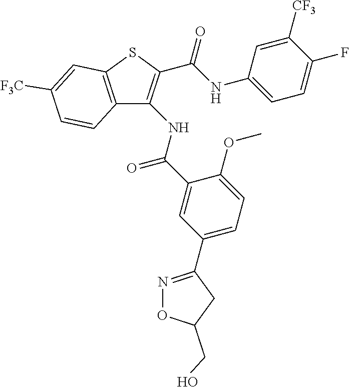 [SMILES: COc1ccc(C2=NOC(CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5- (hydroxymethyl)- 4,5- dihydroisoxazol- 3-yl)-2- methoxy- benzamido)-6- (trifluoromethyl) benzo[b] thiophene-2- carboxamide | 654.0 | ¹H NMR (400 MHz, DMSO-d₆) δ ppm 8.52- 8.45 (m, 1H), 8.18- 8.12 (m, 1H), 8.07- 8.02 (m, 1H), 7.99- 7.94 (m, 1H), 7.87- 7.79 (m, 1H), 7.76- 7.66 (m, 2H), 7.49- 7.39 (m, 1H), 7.26- 7.18 (m, 1H), 4.75- 4.62 (m, 1H), 3.89- 3.82 (m, 3H), 3.52- 3.47 (m, 2H), 1.25- 1.20 (m, 2H). | 2.25 A Isomers separated by Prep SFC (Method 32) to give Example 249 (Prep SFC RT = 10.4 min). Analytical SFC (Method 33). |
| 250 | 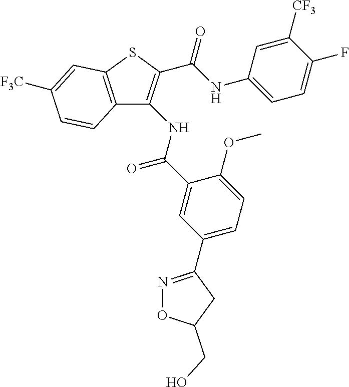 [SMILES: COc1ccc(C2=NOC(CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5- (hydroxymethyl)- 4,5- dihydroisoxazol- 3-yl)-2- methoxy- benzamido)-6- (trifluoromethyl) benzo[b] thiophene-2- carboxamide | 656.2 | ¹H NMR (400 MHz, DMSO-d₆) δ ppm 8.66- 8.59 (m, 1H), 8.17- 8.02 (m, 3H), 7.99- 7.91 (m, 1H), 7.85- 7.75 (m, 2H), 7.56- 7.47 (m, 1H), 7.34- 7.26 (m, 1H), 4.76- 4.65 (m, 1H), 4.00- 3.90 (m, 3H), 3.55- 3.48 (m, 2H), 3.22 3.12 (m, 2H). | 2.38 B Isomers separated by Prep SFC (Method 32) to give Example 250 (Prep SFC RT = 8.7 min) Analytical SFC (Method 33). |
| 251 | 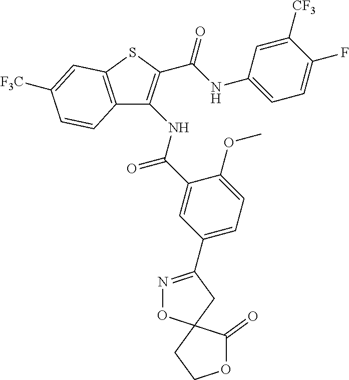 [SMILES: COc1ccc(C2=NOC3(CCOC3=O)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(6- oxo-1,7-dioxa- 2- azaspiro[4.4] non-2-en-3- yl)benzamido)- 6- (trifluoromethyl) benzo[b] thiophene-2- carboxamide | 695.2 | 1H NMR (400 MHz, DMSO-d6) δ ppm 10.88 (br d, J = 2.7 Hz, 1H), 10.79-10.69 (m, 1H), 8.68 (s, 1H), 8.18 (dt, J = 5.9, 1.2 Hz, 1H), 8.13-8.05 (m, 2H), 8.05-7.96 (m, 1H), 7.92-7.81 (m, 2H), 7.59-7.50 (m, 1H), 7.33 (d, J = 8.6 Hz, 1H), 6.52 (s, 1H), 4.63 (t, J = 4.9 Hz, 1H), 4.01- 3.94 (m, 3H), 3.85- 3.57 (m, 1H), 3.56- 3.49 (m, 1H), 2.19- 2.09 (m, 2H) | 2.30 A |
| 253 | 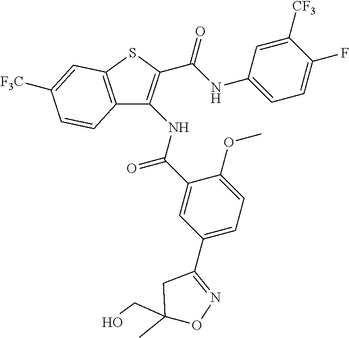 [SMILES: COc1ccc(C2=NOC(C)(CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5- (hydroxymethyl)- 5-methyl- 4,5- dihydroisoxazol- 3-yl)-2- methoxy- benzamido)-6- (trifluoromethyl) benzo[b] thiophene-2- carboxamide | 670.0 | 1H NMR (400 MHz, DMSO-d6) δ ppm 11.13-10.78 (m, 2H), 8.61 (d, J = 0.7 Hz, 1H), 8.18-8.05 (m, 2H), 8.01 (br s, 1H), 7.97- 7.88 (m, 1H), 7.83- 7.70 (m, 2H), 7.51 (t, J = 9.8 Hz, 1H), 7.28 (d, J = 8.3 Hz, 1H), 5.15- 4.99 (m, 1H), 3.94 (br s, 3H), 3.41 (br d, J = 9.8 Hz, 3H), 3.00 (br d, J = 16.4 Hz, 1H), 1.32 (s, 3H) | 2.29 A Isomers separated by Prep SFC (Method 34) to give Example 253 (Prep SFC RT = 10.9 min) Analytical SFC (Method 35). |
| 254 | 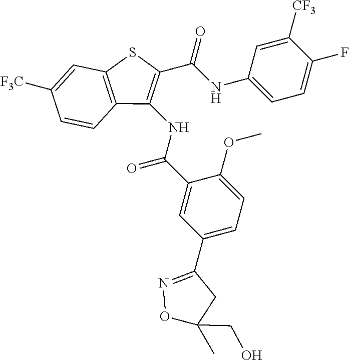 [SMILES: COc1ccc(C2=NOC(C)(CO)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5- (hydroxymethyl)- 5-methyl- 4,5- dihydroisoxazol- 3-yl)-2- methoxy- benzamido)-6- (trifluoromethyl) benzo[b] thiophene-2- carboxamide | 668.2 (M − H)⁻ | 1H NMR (400 MHz, DMSO-d6) δ ppm 10.83 (dt, J = 5.3, 3.9 Hz, 2H), 8.65 (s, 1H), 8.21-8.11 (m, 1H), 8.09 (d, J = 8.6 Hz, 1H), 8.05-8.02 (m, 1H), 8.02-7.91 (m, 1H), 7.87-7.74 (m, 2H), 7.53 (t, J = 9.8 Hz, 1H), 7.30 (d, J = 8.6 Hz, 1H), 5.14-5.01 (m, 1H), 3.96 (s, 3H), 3.41 (br d, J = 10.5 Hz, 2H), 3.00 (br d, J = 16.6 Hz, 2H), 1.32 (s, 3H) | 2.34 A Isomers separated by Prep SFC (Method 34) to give Example 254 (Prep SFC RT = 9.3 min). Analytical SFC (Method 35). |
| 255 | 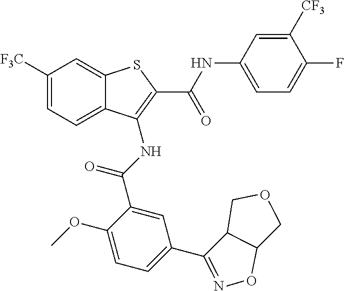 [SMILES: COc1ccc(C2=NOC3COCC23)cc1C(=O)Nc1c(C(=O)Nc2ccc(F)c(C(F)(F)F)c2)sc2cc(C(F)(F)F)ccc12] | 3-(5- {3aH,4H,6H,6aH- furo[3,4- d][1,2]oxazol- 3-yl}-2- methoxy- benzamido)-N-[4- fluoro-3- (trifluoromethyl) phenyl]-6- (trifluoromethyl)- 1- benzothiophene- 2- carboxamide | 668.3 | | 2.42 B The Example was prepared from isomer 2 of the intermediate methyl 2- methoxy-5- (3a,4,6,6a- tetrahydro- furo[3,4- d]isoxazol-3- yl)benzoate [purified by prep SFC (Method 36) to afford isomer 1 (Analytical SFC RT = 1.83 min, >99% ee) and isomer 2 (Analytical SFC RT = 3.83 min, > 99% ee). Analytical SFC (Method 37)]. |
| 256 | 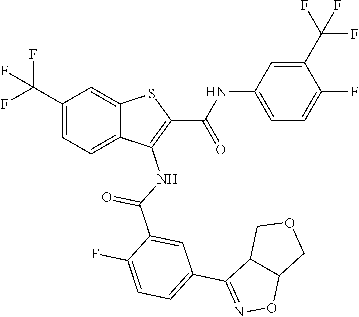 [SMILES: O=C(Nc1c(C(=O)Nc2ccc(F)c(C(F)(F)F)c2)sc2cc(C(F)(F)F)ccc12)c1cc(C2=NOC3COCC23)ccc1F] | 3-(5- {3aH,4H,6H,6aH- furo[3,4- d][1,2]oxazol- 3-yl}-2- fluorobenzamido)- N-[4- fluoro-3- (trifluoromethyl) phenyl]-6- (trifluoromethyl)- 1- benzothiophene- 2- carboxamide | 654.0 (M − H)⁻ | 1H NMR (400 MHz, DMSO-d6) δ ppm 8.55 (br s, 1H), 8.18-8.03 (m, 3H), 7.94-7.80 (m, 2H), 7.78-7.70 (m, 1H), 7.55-7.37 (m, 2H), 5.44-5.32 (m, 1H), 4.58-4.48 (m, 1H), 4.18-4.07 (m, 1H), 3.98-3.90 (m, 1H), 3.80-3.63 (m, 1H), 2.20-2.00 (m, 1H) | 3.59 C Isomers separated by Prep SFC (Method 38) to afford Example 256 (Prep SFC RT = 12.8 min). 100% ee Analytical SFC (Method 39). |
| 257 | 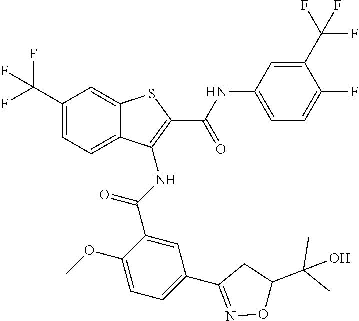 [SMILES: COc1ccc(C2=NOC(C(C)(C)O)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2- hydroxypropan- 2-yl)-4,5- dihydro-1,2- oxazol-3-yl]-2- methoxy- benzamido}-6- (trifluoromethyl)- 1- benzothiophene- 2- carboxamide | 682.3 | 1H NMR (400 MHz, DMSO-d6) δ ppm 10.87 (s, 2H) 8.64 (s, 1H), 8.20-8.12 (m, 1H), 8.08 (d, J = 9.5 Hz, 1H), 7.98 (dddd, J = 1.2, 2.7, 5.9, 7.5 Hz, 1H), 7.80 (d, J = 8.3 Hz, 2H), 7.63-7.44 (m, 1H), 7.41-7.32 (m, 2H), 7.12 (s, 1H), 3.87 (s, 3H), 3.49 (br s, 2H), 3.44-3.39 (m, 2H), 2.87-2.63 (m, 2H), 2.61-2.53 (m, 1H), 1.98-1.82 (m, 1H), 1.58-1.44 (m, 1H) | 3.05 A Isomers separated by Prep SFC (Method 40) to afford Example 257 (Prep SFC RT = 6.9 min). 100% ee Analytical SFC (Method 41). |
| 258 | 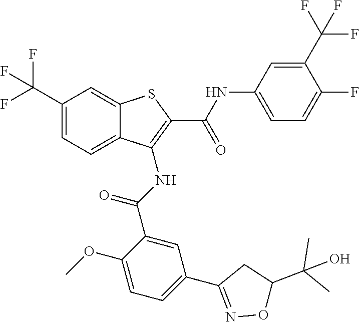 [SMILES: COc1ccc(C2=NOC(C(C)(C)O)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2- hydroxypropan- 2-yl)-4,5- dihydro-1,2- oxazol-3-yl]-2- methoxy- benzamido}-6- (trifluoromethyl)- 1- benzothiophene- 2- carboxamide | 682.3 | 1H NMR (400 MHz, DMSO-d6) δ ppm 10.87 (s, 2H) 8.64 (s, 1H), 8.20-8.12 (m, 1H), 8.08 (d, J = 9.5 Hz, 1H), 7.98 (dd, J = 1.2, 2.7, 5.9, 7.5 Hz, 1H), 7.80 (d, J = 8.3 Hz, 2H), 7.63-7.44 (m, 1H), 7.41-7.32 (m, 2H), 7.12 (s, 1H), 3.87 (s, 3H), 3.49 (br s, 2H), 3.44-3.39 (m, 2H), 2.87-2.63 (m, 2H), 2.61-2.53 (m, 1H), 1.98-1.82 (m, 1H), 1.58-1.44 (m, 1H) | 3.01 A Isomers separated by Prep SFC (Method 40) to afford Example 258 (Prep SFC RT = 9.0 min). 95.4% ee Analytical SFC (Method 41) |
| 259 | 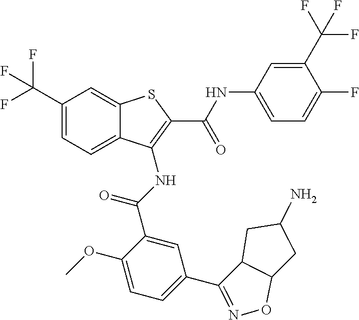 [SMILES: COc1ccc(C2=NOC3CC(N)CC23)cc1C(=O)Nc1c(C(=O)Nc2ccc(F)c(C(F)(F)F)c2)sc2cc(C(F)(F)F)ccc12] | 3-(5-{5-amino- 3aH,4H,5H,6H, 6aH- cyclopenta[d] [1,2]oxazol-3- yl}-2- methoxy- benzamido)-N-[4- fluoro-3- (trifluoromethyl) phenyl]-6- (trifluoromethyl)- 1- benzothiophene- 2- carboxamide | 681.2 | 1H NMR (400 MHz, DMSO-d6) δ ppm 8.64 (s, 1H), 8.18-8.12 (m, 1H), 8.11-8.01 (m, 2H), 8.00-7.91 (m, 1H), 7.87-7.74 (m, 2H), 7.53 (t, J = 9.7 Hz, 1H), 7.31 (d, J = 8.8 Hz, 1H), 5.16-5.09 (m, 1H), 4.28-4.20 (m, 1H), 3.96 (s, 3H), 2.15- 2.07 (m, 1H), 1.94- 1.85 (m, 2H), 1.82- 1.66 (m, 2H). | 2.12 A The Example was prepared from isomer 3 from the Intermediate tert-butyl (3- (3-((2-((4- fluoro-3- (trifluoro- methyl)phenyl) carbamoyl)-6- (trifluoromethyl) benzo[b] thiophen-3- yl)carbamoyl)- 4- methoxyphenyl)- 3a,5,6,6a- tetrahydro- 4H- cyclopenta[d] isoxazol-5- yl)carbamate, which was purified by SFC (See Methods 46) |
| 260 | 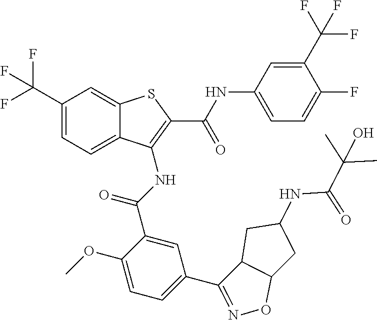 [SMILES: COc1ccc(C2=NOC3CC(NC(=O)C(C)(C)O)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2-hydroxy- 2- methyl- propanamido)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 765.2 (M − H)⁻ | 1H NMR (400 MHz, DMSO-d6) δ ppm 10.90-10.82 (m, 1H), 10.77-10.69 (m, 1H), 8.66 (s, 1H), 8.23-8.15 (m, 1H), 8.10 (dd, J = 3.1, 5.3 Hz, 2H), 8.01- 7.92 (m, 1H), 7.86 (dd, J = 2.2, 8.6 Hz, 1H), 7.82 (dd, J = 1.0, 8.8 Hz, 1H), 7.69 (d, J = 8.3 Hz, 1H), 7.53 (t, J = 9.8 Hz, 1H), 7.32 (d, J = 8.8 Hz, 1H), 5.33 (s, 1H), 5.13 (dd, J = 4.9, 9.0 Hz, 1H), 4.29-4.22 (m, 1H), 3.96 (s, 4H), 2.13-2.04 (m, 1H), 2.00-1.86 (m, 3H), 1.21 (s, 3H), 1.17 (s, 3H). | 2.32 A The Example was prepared from Example 259. |
| 261 | 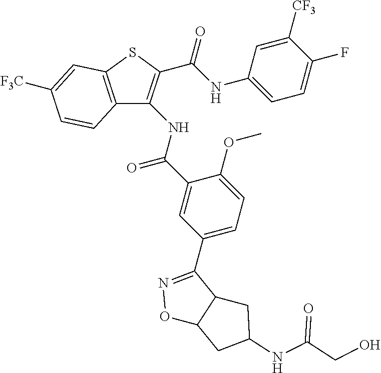 [SMILES: COc1ccc(C2=NOC3CC(NC(=O)CO)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2- hydroxy- acetamido)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 737.1 (M − H)⁻ | 1H NMR (400 MHz, DMSO-d6) δ ppm 10.87 (br s, 1H), 10.73 (br s, 1H), 8.66 (s, 1H), 8.21-8.15 (m, 1H), 8.13-8.05 (m, 2H), 8.01-7.94 (m, 1H), 7.89-7.76 (m, 3H), 7.53 (t, J = 9.9 Hz, 1H), 7.32 (d, J = 9.0 Hz, 1H), 5.43 (t, J = 5.7 Hz, 1H), 5.13 (dd, J = 4.8, 9.2 Hz, 1H), 4.30-4.21 (m, 1H), 4.12-4.01 (m, 1H), 3.96 (s, 3H), 3.79- 3.70 (m, 2H), 2.15- 2.07 (m, 1H), 2.00- 1.86 (m, 3H). | 2.21 A The Example was prepared from Example 259. |
| 262 | 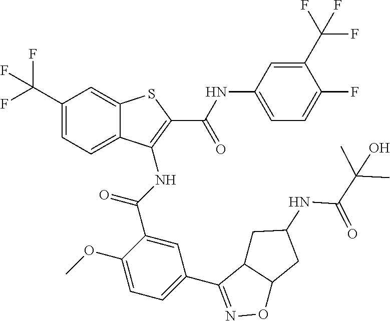 [SMILES: COc1ccc(C2=NOC3CC(NC(=O)C(C)(C)O)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2-hydroxy- 2- methyl- propanamido)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 767.2 | 1H NMR (400 MHz, DMSO-d6) δ ppm 10.84 (s, 1H), 10.73 (s, 1H), 8.66 (s, 1H), 8.23- 8.15 (m, 1H), 8.10 (d, J = 2.0 Hz, 1H), 8.06 (d, J = 8.6 Hz, 1H), 8.01-7.93 (m, 1H), 7.86 (dd, J = 2.1, 8.7 Hz, 1H), 7.84-7.76 (m, 1H), 7.54 (t, J = 10.0 Hz, 1H), 7.41 (d, J = 7.6 Hz, 1H), 7.32 (d, J = 9.0 Hz, 1H), 5.32 (s, 1H), 5.24-5.15 (m, 1H), 4.22-4.07 (m, 2H), 3.98 (s, 3H), 2.29- 2.14 (m, 2H), 2.04- 1.96 (m, 1H), 1.81- 1.69 (m, 1H), 1.16 (s, 3H), 1.00 (s, 3H). | 2.33 A The Example was prepared from isomer 1 from the Intermediate tert-butyl (3- (3-((2-((4- fluoro-3- (trifluoromethyl) phenyl) carbamoyl)-6- (trifluoromethyl) benzo[b] thiophen-3- yl)carbamoyl)- 4- methoxyphenyl)- 3a,5,6,6a- tetrahydro- 4H- cyclopenta[d] isoxazol-5- yl)carbamate, which was purified by SFC (See Methods 46) |
| 263 | 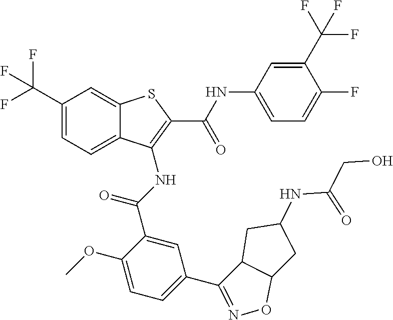 [SMILES: COc1ccc(C2=NOC3CC(NC(=O)CO)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2- hydroxyacetamido)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 737.2 | 1H NMR (400 MHz, DMSO-d6) δ ppm 10.86 (s, 1H), 10.74 (s, 1H), 8.67 (s, 1H), 8.18 (dd, J = 1.7, 6.6 Hz, 1H), 8.13-8.04 (m, 2H), 8.01-7.92 (m, 1H), 7.89-7.75 (m, 2H), 7.54 (t, J = 9.5 Hz, 1H), 7.51-7.41 (m, 1H), 7.32 (d, J = 8.8 Hz, 1H), 5.45 (t, J = 5.5 Hz, 1H), 5.18 (ddd, J = 3.8, 6.2, 9.4 Hz, 1H), 4.27-4.19 (m, 1H), 4.18-4.10 (m, 1H), 3.75-3.59 (m, 2H), 2.35-2.22 (m, 2H), 2.00-1.89 (m, 1H), 1.78-1.65 (m, 1H), 1.29 1.18 (m, 1H). | 2.24 A The Example was prepared from isomer 1 from the Intermediate tert-butyl (3- (3-((2-((4- fluoro-3- (trifluoromethyl) phenyl) carbamoyl)-6- (trifluoromethyl) benzo[b] thiophen-3- yl)carbamoyl)- 4- methoxyphenyl)- 3a,5,6,6a- tetrahydro- 4H- cyclopenta[d] isoxazol-5- yl)carbamate, which was purified by SFC (See Methods 46) |
| 264 | 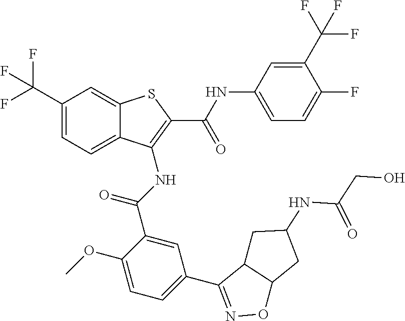 [SMILES: COc1ccc(C2=NOC3CC(NC(=O)CO)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2- hydroxyacetamido)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 739.2 | 1H NMR (400 MHz, DMSO-d6) δ ppm 10.86 (s, 1H), 10.74 (s, 1H), 8.66 (s, 1H), 8.18 (dd, J = 2.4, 6.6 Hz, 1H), 8.12-8.02 (m, 2H), 8.00-7.93 (m, 1H), 7.84 (ddd, J = 2.0, 8.7, 13.1 Hz, 2H), 7.60- 7.43 (m, 2H), 7.32 (d, J = 8.8 Hz, 1H), 5.45 (s, 1H), 5.18 (ddd, J = 3.4, 6.1, 9.8 Hz, 1H), 4.28- 4.19 (m, 1H), 4.14 (ddd, J = 4.5, 9.6, 14.0 Hz, 1H), 3.98 (s, 2H), 3.91-3.80 (m, 1H), 3.68 (dd, J = 5.6, 7.8 Hz, 2H), 2.36-2.23 (m, 2H), 2.00-1.90 (m, 1H), 1.77-1.65 (m, 1H), 1.23 (s, 1H) | 2.23 A The Example was prepared from isomer 2 from the Intermediate tert-butyl (3- (3-((2-((4- fluoro-3- (trifluoromethyl) phenyl) carbamoyl)-6- (trifluoromethyl) benzo[b] thiophen-3- yl)carbamoyl)- 4- methoxyphenyl)- 3a,5,6,6a- tetrahydro- 4H- cyclopenta[d] isoxazol-5- yl)carbamate, which was purified by SFC (See Methods 46) |
| 265 | 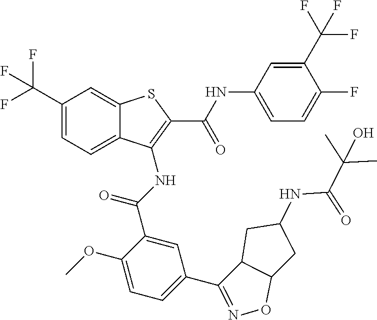 [SMILES: COc1ccc(C2=NOC3CC(NC(=O)C(C)(C)O)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2-hydroxy- 2- methyl- propanamido)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 767.2 | 1H NMR (400 MHz, DMSO-d6) δ ppm 10.84 (s, 1H), 10.73 (s, 1H), 8.67 (s, 1H), 8.18 (br d, J = 6.8 Hz, 1H), 8.11 (d, J = 2.2 Hz, 1H), 8.06 (d, J = 8.6 Hz, 1H), 8.01-7.93 (m, 1H), 7.86 (dd, J = 2.2, 8.8 Hz, 1H), 7.82 (dd, J = 1.2, 8.6 Hz, 1H), 7.60-7.47 (m, 1H), 7.41 (d, J = 7.8 Hz, 1H), 7.32 (d, J = 9.0 Hz, 1H), 5.31 (s, 1H), 5.21 (br d, J = 6.6 Hz, 1H), 4.22-4.05 (m, 2H), 3.98 (s, 3H), 2.28-2.17 (m, 2H), 2.04-1.95 (m, 1H), 1.80-1.70 (m, 1H), 1.16 (s, 3H), 1.01 (s, 3H). | 2.32 A The Example was prepared from isomer 2 from the Intermediate tert-butyl (3- (3-((2-((4- fluoro-3- (trifluoromethyl) phenyl) carbamoyl)-6- (trifluoromethyl) benzo[b] thiophen-3- yl)carbamoyl)- 4- methoxyphenyl)- 3a,5,6,6a- tetrahydro- 4H- cyclopenta[d] isoxazol-5- yl)carbamate, which was purified by SFC (See Methods 46) |
| 266 | 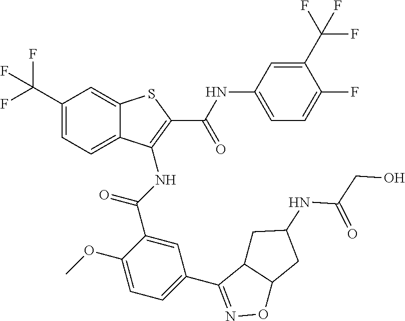 [SMILES: COc1ccc(C2=NOC3CC(NC(=O)CO)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2- hydroxyacetamido)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 737.2 | 1H NMR (400 MHz, DMSO-d6) δ ppm 10.87 (s, 1H), 10.74 (s, 1H), 8.66 (s, 1H), 8.18 (dd, J = 2.6, 6.2 Hz, 1H), 8.13-8.04 (m, 2H), 8.01-7.92 (m, 1H), 7.88-7.75 (m, 3H), 7.53 (t, J = 9.7 Hz, 1H), 7.32 (d, J = 8.8 Hz, 1H), 5.44 (t, J = 5.6 Hz, 1H), 5.13 (dd, J = 5.0, 9.2 Hz, 1H), 4.30- 4.22 (m, 1H), 4.11- 4.01 (m, 1H), 3.96 (s, 3H), 3.76 (dd, J = 2.2, 5.6 Hz, 2H), 2.16-2.05 (m, 1H), 2.01-1.83 (m, 3H). | 2.23 A The Example was prepared from isomer 4 from the Intermediate tert-butyl (3- (3-((2-((4- fluoro-3- (trifluoromethyl) phenyl) carbamoyl)-6- (trifluoromethyl) benzo[b] thiophen-3- yl)carbamoyl)- 4- methoxyphenyl)- 3a,5,6,6a- tetrahydro- 4H- cyclopenta[d] isoxazol-5- yl)carbamate, which was purified by SFC (See Methods 46) |
| 267 | 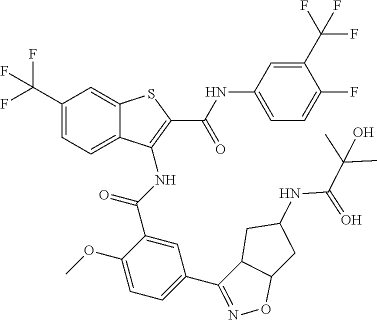 [SMILES: COc1ccc(C2=NOC3CC(NC(=O)C(C)(C)O)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5-(2-hydroxy- 2- methyl- propanamido)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 765.2 (M − H)⁻ | 1H NMR (400 MHz, DMSO-d6) δ ppm 10.87 (s, 1H), 10.74 (s, 1H), 8.66 (s, 1H), 8.19 (dd, J = 2.1, 6.5 Hz, 1H), 8.13-8.04 (m, 2H), 7.97 (ddd, J = 2.9, 5.3, 6.7 Hz, 1H), 7.84 (ddd, J = 1.6, 9.0, 15.6 Hz, 2H), 7.69 (d, J = 8.3 Hz, 1H), 7.53 (t, J = 9.8 Hz, 1H), 7.32 (d, J = 8.8 Hz, 1H), 5.33 (s, 1H), 5.13 (br dd, J = 4.2, 9.3 Hz, 1H), 4.29- 4.19 (m, 1H), 3.96 (s, 4H), 2.14-1.82 (m, 4H), 1.27-1.08 (m, 6H). | 2.33 A The Example was prepared from isomer 4 from the Intermediate tert-butyl (3- (3-((2-((4- fluoro-3- (trifluoromethyl) phenyl) carbamoyl)-6- (trifluoromethyl) benzo[b] thiophen-3- yl)carbamoyl)- 4- methoxyphenyl)- 3a,5,6,6a- tetrahydro- 4H- cyclopenta[d] isoxazol-5- yl)carbamate, which was purified by SFC (See Methods 46) |
| 268 | 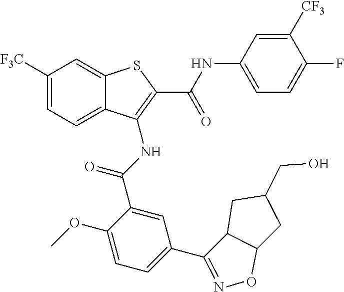 [SMILES: COc1ccc(C2=NOC3CC(CO)CC23)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [5- (hydroxymethyl)- 3aH,4H,5H,6H, 6aH- cyclopenta[d] [1,2]oxazol-3- yl]-2- methoxy- benzamido}-6- (trifluoromethyl)- 1- benzothiophene- 2- carboxamide | 696.1 | 1H NMR (400 MHz, DMSO-d6) δ ppm 10.85-10.74 (m, 2H), 8.65 (s, 1H), 8.17-8.16 (d, 1H), 8.12-8.09 (m, 2H), 8.08 (s, 1H), 7.85- 7.81 (m, 2H), 7.56- 7.51 (m, 1H), 7.33- 7.30 (d, 1H), 5.14- 5.12 (t, 1H), 4.49 (s, 1H), 4.21-4.16 (m, 1H), 3.97 (s, 3H), 3.51- 3.41 (m, 1H), 2.08- 1.89 (m, 3H), 1.87- 1.77 (m, 1H), 1.69- 1.51 (m, 2H) | 2.39 A The example was prepared from isomer 3 of the intermediate methyl 5-(5- (hydroxymethyl)- 3a,5,6,6a- tetrahydro- 4H- cyclopenta[d] isoxazol-3- yl)-2- methoxy- benzoate which was purified by prep SFC purification (See Methods 47) |
| 269 | 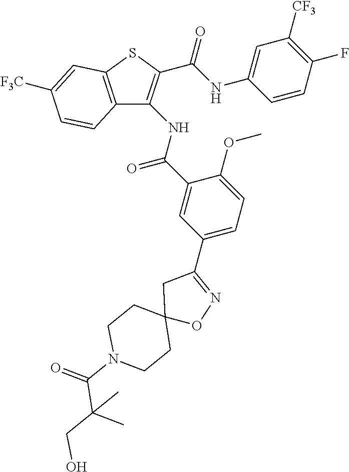 [SMILES: COc1ccc(C2=NOC3(CCN(C(=O)C(C)(C)CO)CC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (8-(3-hydroxy- 2,2- dimethyl- propanoyl)-1-oxa- 2,8- diazaspiro[4.5] dec-2-en-3-yl)- 2- methoxy- benzamido)-6- (trifluoromethyl) benzo[b] thiophene-2- carboxamide | 795.4 | 1H NMR (400 MHz, DMSO-d6) δ ppm 10.94-10.67 (m, 2H), 8.67 (s, 1H), 8.24-8.11 (m, 1H), 8.11-8.03 (m, 2H), 8.03-7.94 (m, 1H), 7.83 (br t, J = 10.5 Hz, 2H), 7.54 (br t, J = 9.5 Hz, 1H), 7.33 (br d, J = 9.0 Hz, 1H), 4.58- 4.48 (m, 1H), 3.99 (s, 3H), 3.75-3.63 (m, 2H), 3.63-3.50 (m, 2H), 3.43 (br d, J = 4.9 Hz, 2H), 3.23 (br s, 2H), 1.74 (br s, 4H), 1.17 (s, 6H) | 2.39 A |
| 270 | 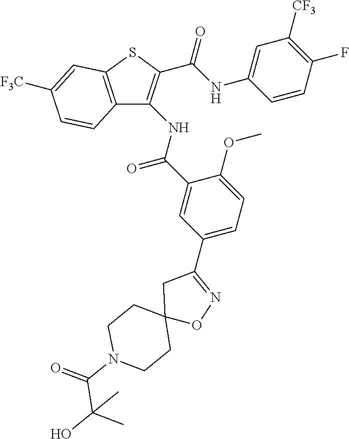 [SMILES: COc1ccc(C2=NOC3(CCN(C(=O)C(C)(C)O)CC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (8-(2-hydroxy- 2- methylpropanoyl)- 1-oxa-2,8- diazaspiro[4.5] dec-2-en-3-yl)- 2- methoxy- benzamido)-6- (trifluoromethyl) benzo[b] thiophene-2- carboxamide | 781.2 | 1H NMR (400 MHz, DMSO-d6) δ ppm 10.92-10.68 (m, 2H), 8.67 (s, 1H), 8.15 (dd, J = 6.4, 2.7 Hz, 1H), 8.11-8.03 (m, 2H), 8.03-7.93 (m, 1H), 7.88-7.76 (m, 2H), 7.54 (t, J = 9.7 Hz, 1H), 7.33 (d, J = 8.3 Hz, 1H), 5.43 (s, 2H), 3.99 (s, 3H), 3.24 (s, 4H), 1.84- 1.64 (m, 5H), 1.33 (s, 6H) | 2.36 A |
| 271 | 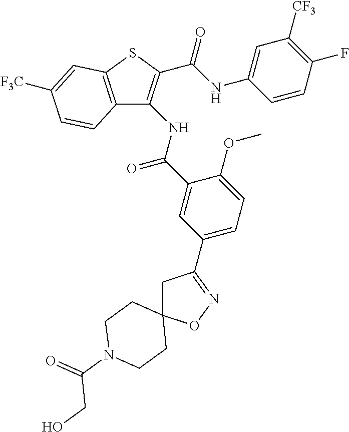 [SMILES: COc1ccc(C2=NOC3(CCN(C(=O)CO)CC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (8-(2- hydroxyacetyl)- 1-oxa-2,8- diazaspiro[4.5] dec-2-en-3-yl)- 2- methoxy- benzamido)-6- (trifluoromethyl) benzo[b] thiophene-2- carboxamide | 753.2 | 1H NMR (400 MHz, DMSO-d6) δ ppm 10.90-10.65 (m, 2H), 8.67 (s, 1H), 8.19-8.12 (m, 1H), 8.12-8.03 (m, 2H), 8.03-7.93 (m, 1H), 7.90-7.77 (m, 2H), 7.59-7.49 (m, 1H), 7.38-7.25 (m, 1H), 4.59-4.50 (m, 1H), 4.15-4.08 (m, 2H), 3.99 (s, 3H), 3.90 (s, 1H), 3.72-3.62 (m, 1H), 3.55-3.40 (m, 3H), 3.24 (s, 2H), 1.83- 1.60 (m, 4H) | 2.28 A |
| 272 | 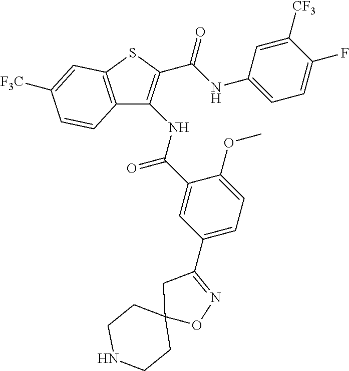 [SMILES: COc1ccc(C2=NOC3(CCNCC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(1- oxa-2,8- diazaspiro[4.5] dec-2-en-3- yl)benzamido)- 6- (trifluoromethyl) benzo[b] thiophene-2- carboxamide | 695.0 | 1H NMR (400 MHz, DMSO-d6) δ ppm 10.87-10.62 (m, 2H), 8.63 (s, 1H), 8.17-8.06 (m, 2H), 8.05-8.00 (m, 1H), 7.97 (td, J = 1.7, 8.3 Hz, 2H), 7.85-7.73 (m, 2H), 7.51 (t, J = 10.1 Hz,1H), 7.30 (d, J = 9.0 Hz, 1H), 3.95 (br s, 3H), 3.17 (s, 2H), 2.98- 2.89 (m, 2H), 2.80- 2.70 (m, 1H), 1.89 (s, 2H), 1.70 (br t, J = 5.0 Hz, 3H) | 2.13 A |
| 273 | 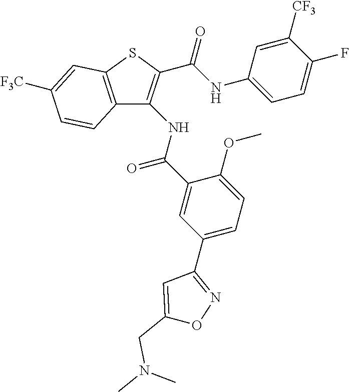 [SMILES: COc1ccc(-c2cc(CN(C)C)on2)cc1C(=O)Nc1c(C(=O)Nc2ccc(F)c(C(F)(F)F)c2)sc2cc(C(F)(F)F)ccc12] | 3-(5-(5- ((dimethylamino) methyl) isoxazol-3-yl)-2- methoxy- benzamido)-N-(4- fluoro-3- (trifluoromethyl) phenyl)-6- (trifluoromethyl) benzo[b] thiophene-2- carboxamide | 681.2 | 1H NMR (400 MHz, DMSO-d6) δ ppm 10.89 (s, 1H), 10.77 (s, 1H), 8.68 (s, 1H), 8.32 (d, J = 2.0 Hz, 1H), 8.15 (dd, J = 2.3, 6.5 Hz, 1H), 8.12-8.05 (m, 2H), 8.05-7.95 (m, 1H), 7.82 (d, J = 8.3 Hz, 1H), 7.54 (t, J = 9.8 Hz, 1H), 7.41 (d, J = 8.8 Hz, 1H), 7.24 (s, 1H), 4.49-4.34 (m, 1H), 4.02 (s, 3H), 2.93 (br dd, J = 6.7, 12.6 Hz, 1H), 2.72 (s, 4H), 1.23 (s, 1H), 1.16 (t, J = 7.3 Hz, 1H) | 2.46 A |
| 274 | 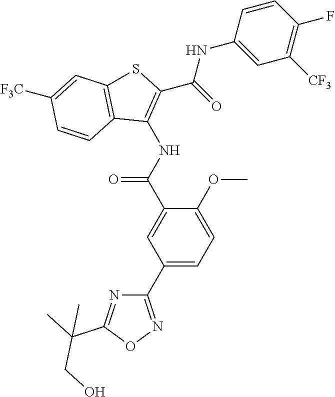 [SMILES: COc1ccc(-c2noc(C(C)(C)CO)n2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5-(1-hydroxy- 2- methylpropan- 2-yl)-1,2,4- oxadiazol-3- yl)-2- methoxy- benzamido)-6- (trifluoromethyl) benzo[b] thiophene-2- carboxamide | 695.2 (M − H)⁻ | 1H NMR (400 MHz, DMSO-d6) δ ppm 10.91 (s, 1H), 10.78 (s, 1H), 8.68 (s, 1H), 8.44 (d, J = 2.0 Hz, 1H), 8.21- 8.14 (m, 2H), 8.10 (d, J = 8.3 Hz, 1H), 8.04- 7.96 (m, 1H), 7.83 (br d, J = 7.8 Hz, 1H), 7.53 (br t, J = 9.9 Hz, 1H), 7.43 (d, J = 8.8 Hz, 1H), 5.13 (t, J = 5.5 Hz, 1H), 4.03 (s, 3H), 3.65-3.57 (m, 2H), 1.38 (s, 6H) | 2.49 A |
| 275 | 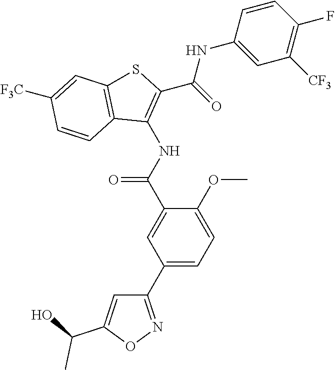 [SMILES: COc1ccc(-c2cc([C@@H](C)O)on2)cc1C(=O)Nc1c(C(=O)Nc2ccc(F)c(C(F)(F)F)c2)sc2cc(C(F)(F)F)ccc12] | (R)-N-(4- fluoro-3- (trifluoromethyl) phenyl)-3-(5- (5-(1- hydroxyethyl) isoxazol-3-yl)- 2- methoxy- benzamido)-6- (trifluoromethyl) benzo[b] thiophene-2- carboxamide | 669.2 | 1H NMR (400 MHz, DMSO-d6) δ ppm 10.91-10.86 (m, 1H), 10.80-10.72 (m, 1H), 8.68 (s, 1H), 8.29 (d, J = 2.2 Hz, 1H), 8.16 (dd, J = 2.4, 6.8 Hz, 1H), 8.11 (d, J = 8.3 Hz, 1H), 8.08-7.98 (m, 2H), 7.83 (dd, J = 1.7, 8.6 Hz, 1H), 7.54 (t, J = 9.8 Hz, 1H), 7.37 (d, J = 8.8 Hz, 1H), 6.88 (s, 1H), 5.79 (d, J = 5.4 Hz, 1H), 4.94-4.83 (m, 1H), 3.98 (s, 3H), 1.46 (d, J = 6.6 Hz, 3H) | 2.38 B |
| 276 | 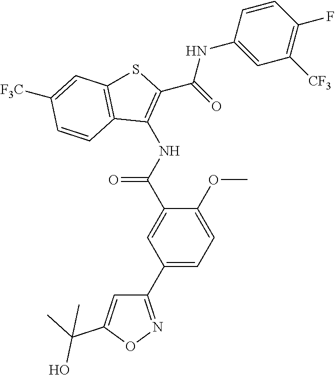 [SMILES: COc1ccc(-c2cc(C(C)(C)O)on2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5-(2- hydroxypropan- 2-yl)isoxazol- 3-yl)-2- methoxy- benzamido)-6- (trifluoromethyl) benzo[b] thiophene-2- carboxamide | 682.2 | 1H NMR (400 MHz, DMSO-d6) δ ppm 10.92-10.84 (m, 1H), 10.77 (br d, J = 3.2 Hz, 1H), 8.67 (s, 1H), 8.29 (d, J = 2.2 Hz, 1H), 8.16 (dd, J = 2.2, 6.6 Hz, 1H), 8.11 (d, J = 8.6 Hz, 1H), 8.07-7.98 (m, 2H), 7.82 (dd, J = 1.1, 8.7 Hz, 1H), 7.53 (t, J = 9.8 Hz, 1H), 7.37 (d, J = 8.8 Hz, 1H), 6.84 (s, 1H), 5.67 (s, 1H), 3.98 (s, 3H), 1.52 (s, 6H) | 2.44 A |
| 277 | 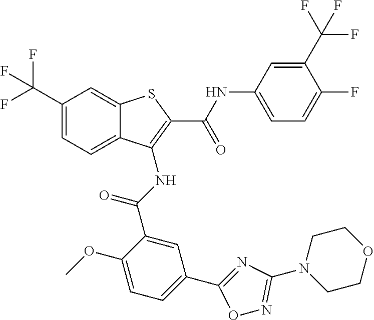 [SMILES: COc1ccc(-c2nc(N3CCOCC3)no2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{2- methoxy-5-[3- (morpholin-4- yl)-1,2,4- oxadiazol-5- yl]benzamido}- 6- (trifluoromethyl)- 1- benzothiophene- 2- carboxamide | 710.2 | 1H NMR (400 MHz, DMSO-d6) δ ppm 10.95-10.86 (m, 1H), 10.83-10.74 (m, 1H), 8.67 (s, 1H), 8.41 (d, J = 2.2 Hz, 1H), 8.25- 8.14 (m, 2H), 8.10 (d, J = 8.6 Hz, 1H), 8.04- 7.93 (m, 1H), 7.87- 7.78 (m, 1H), 7.54 (t, J = 9.9 Hz, 1H), 7.45 (d, J = 9.0 Hz, 1H), 4.03 (s, 3H), 3.75-3.67 (m, 4H), 3.41-3.37 (m, 4H). | 2.66 A |
| 278 | 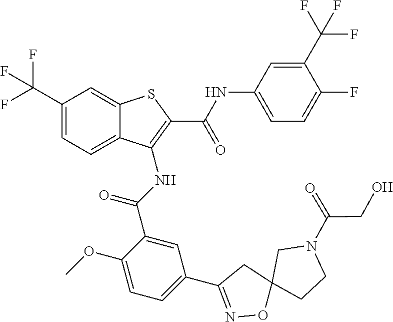 [SMILES: COc1ccc(C2=NOC3(CCN(C(=O)CO)C3)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [7-(2- hydroxyacetyl)- 1-oxa-2,7- diazaspiro[4.4] non-2-en-3-yl]- 2- methoxy- benzamido}-6- (trifluoromethyl)- 1- benzothiophene- 2- carboxamide | 739.3 | 1H NMR (400 MHz, DMSO-d6) δ ppm 10.99-10.66 (m, 1H), 8.67 (s, 1H), 8.21-8.12 (m, 1H), 8.12-8.04 (m, 2H), 8.03-7.96 (m, 1H), 7.83 (ddd, J = 1.8, 8.6, 13.8 Hz, 2H), 7.60- 7.48 (m, 1H), 7.34 (d, J = 9.0 Hz, 1H), 4.66- 4.55 (m, 1H), 4.05 (br d, J = 5.4 Hz, 1H), 4.02- 3.93 (m, 4H), 3.73- 3.41 (m, 7H), 2.28- 2.05 (m, 2H). | 2.27 A |
| 279 | 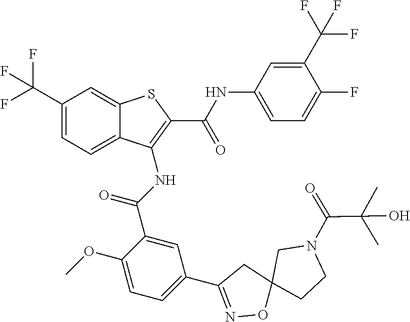 [SMILES: COc1ccc(C2=NOC3(CCN(C(=O)C(C)(C)O)C3)C2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [7-(2-hydroxy- 2- methylpropanoyl)- 1-oxa-2,7- diazaspiro[4.4] non-2-en-3-yl]- 2- methoxy- benzamido}-6- (trifluoromethyl)- 1- benzothiophene- 2- carboxamide | 767.2 | 1H NMR (400 MHz, DMSO-d6) δ ppm 8.56 (br s, 1H), 8.13 (d, J = 9.0 Hz, 1H), 8.10-8.04 (m, 1H), 8.02 (br s, 1H), 7.96-7.86 (m, 1H), 7.83-7.67 (m, 2H), 7.48 (t, J = 9.9 Hz, 1H), 7.28 (br d, J = 8.6 Hz, 1H), 5.29-5.12 (m, 1H), 4.25-4.10 (m, 1H), 3.92 (br s, 3H), 3.81 (br d, J = 12.7 Hz, 1H), 3.66-3.41 (m, 5H), 2.21-1.97 (m, 2H), 1.89 (s, 1H), 1.35- 1.21 (m, 7H). | 2.23 A |
| 280 | 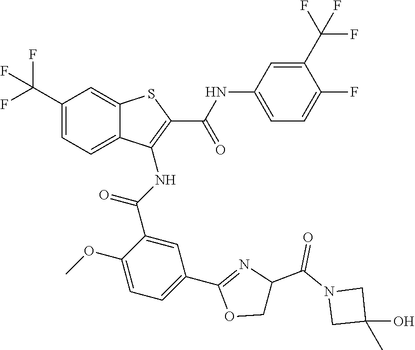 [SMILES: COc1ccc(C2=NC(C(=O)N3CC(C)(O)C3)CO2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-{5- [4-(3-hydroxy- 3- methylazetidine- 1-carbonyl)- 4,5-dihydro- 1,3-oxazol-2- yl]-2- methoxy- benzamido}-6- (trifluoromethyl)- 1- benzothiophene- 2- carboxamide | 739.2 | 1H NMR (400 MHz, DMSO-d6) δ ppm 10.88 (s, 1H), 10.76 (s, 1H), 8.68 (s, 1H), 8.26- 8.15 (m, 1H), 8.15- 8.05 (m, 2H), 8.05- 7.93 (m, 1H), 7.89 (td, J = 2.6, 8.6 Hz, 1H), 7.83 (dd, J = 1.1, 8.7 Hz, 1H), 7.55 (t, J = 9.8 Hz, 1H), 7.33 (d, J = 8.8 Hz, 1H), 5.71 (d, J = 7.8 Hz, 1H), 5.32-5.19 (m, 1H), 4.25-4.06 (m, 2H), 3.98 (s, 3H), 3.84- 3.73 (m, 2H), 3.70- 3.56 (m, 2H), 1.40 (s, 3H). | 2.28 A |
| 281 | 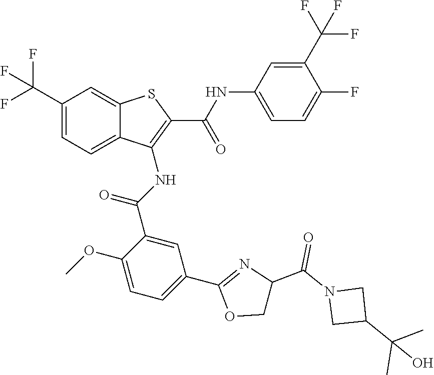 [SMILES: COc1ccc(C2=NC(C(=O)N3CC(C(C)(C)O)C3)CO2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-(5- {4-[3-(2- hydroxypropan- 2-yl)azetidine- 1-carbonyl]- 4,5-dihydro- 1,3-oxazol-2- yl}-2- methoxy- benzamido)-6- (trifluoromethyl)- 1- benzothiophene- 2- carboxamide | 767.3 | 1H NMR (400 MHz, DMSO-d6) δ ppm 10.87 (s, 1H), 10.75 (s, 1H), 8.67 (s, 1H), 8.24- 8.15 (m, 1H), 8.14- 8.06 (m, 2H), 8.03- 7.95 (m, 1H), 7.88 (br d, J = 9.0 Hz, 1H), 7.82 (dd, J = 1.3, 8.7 Hz, 1H), 7.54 (t, J = 9.8 Hz, 1H), 7.32 (d, J = 8.8 Hz, 1H), 5.22 (ddd, J = 3.8, 7.4, 11.1 Hz, 1H), 4.56 (d, J = 4.6 Hz, 1H), 4.27- 4.15 (m, 2H), 3.97 (s, 3H), 3.82 (br d, J = 7.6 Hz, 2H), 3.66-3.51 (m, 2H), 2.65-2.56 (m, 1H). | 2.23 A |
| 282 | 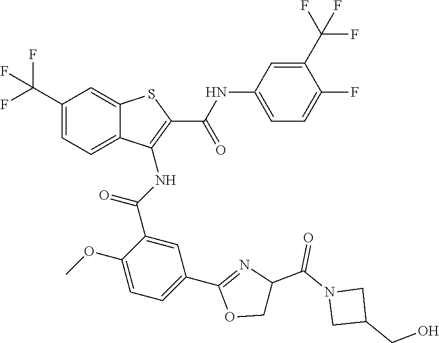 [SMILES: COc1ccc(C2=NC(C(=O)N3CC(CO)C3)CO2)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-(5- {4-[3- (hydroxymethyl) azetidine-1- carbonyl]-4,5- dihydro-1,3- oxazol-2-yl}-2- methoxy- benzamido)-6- (trifluoromethyl)- 1- benzothiophene- 2- carboxamide | 739.3 | 1H NMR (400 MHz, DMSO-d6) δ ppm 10.88 (s, 1H), 10.76 (s, 1H), 8.68 (s, 1H), 8.19 (br d, J = 4.4 Hz, 1H), 8.15-8.05 (m, 2H), 8.00 (br dd, J = 3.4, 8.3Hz, 1H), 7.92-7.85 (m, 1H), 7.83 (dd, J = 1.0, 8.8 Hz, 1H), 7.55 (t, J = 9.8 Hz, 1H), 7.33 (d, J = 8.8 Hz, 1H), 5.29-5.18 (m, 1H), 4.83 (dt, J = 3.7, 5.3Hz, 1H), 4.38-4.22 (m, 1H), 4.08-3.99 (m, 1H), 3.97 (s, 3H), 3.95- 3.87 (m, 1H), 3.71- 3.50 (m, 5H), 2.72 (br d, J = 4.6 Hz, 1H). | 2.23 A |
| 283 | 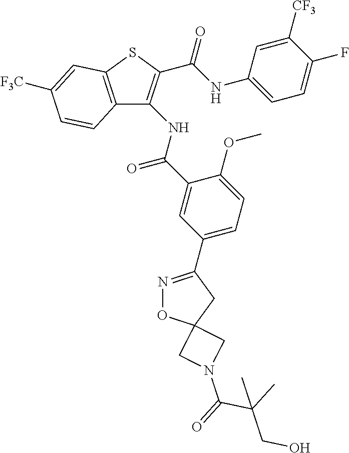 [SMILES: COc1ccc(C2=NOC3(C2)CN(C(=O)C(C)(C)CO)C3)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (2-(3-hydroxy- 2,2- dimethyl- propanoyl)-5-oxa- 2,6- diazaspiro[3.4] oct-6-en-7-yl)- 2- methoxy- benzamido)-6- (trifluoromethyl) benzo[b] thiophene-2- carboxamide | 765.2 (M − H)⁻ | 1H NMR (400 MHz, DMSO-d6) δ ppm 10.85 (br dd, J = 4.4, 3.2 Hz, 1H), 10.78-10.69 (m, 1H), 8.67 (s, 1H), 8.20-8.13 (m, 1H), 8.11-8.04 (m, 2H), 8.04-7.93 (m, 1H), 7.88-7.76 (m, 2H), 7.54 (t, J = 10.0 Hz, 1H), 7.35 (d, J = 8.6 Hz, 1H), 4.79 (t, J = 5.3 Hz, 2H), 4.64-4.50 (m, 2H), 3.99 (s, 4H), 3.76 (s, 2H), 3.36 (d, J = 5.4 Hz, 2H), 1.06 (s, 6H) | 2.30 A |
| 284 | 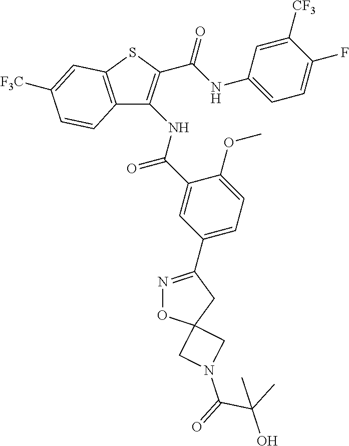 [SMILES: COc1ccc(C2=NOC3(C2)CN(C(=O)C(C)(C)O)C3)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (2-(2-hydroxy- 2- methylpropanoyl)- 5-oxa-2,6- diazaspiro[3.4] oct-6-en-7-yl)- 2- methoxy- benzamido)-6- (trifluoromethyl) benzo[b] thiophene-2- carboxamide | 751.2 (M − H)⁻ | 1H NMR (400 MHz, DMSO-d6) δ ppm 10.94-10.67 (m, 2H), 8.67 (s, 1H), 8.22-8.11 (m, 1H), 8.11-8.04 (m, 2H), 8.04-7.93 (m, 1H), 7.88-7.75 (m, 2H), 7.54 (t, J = 9.9 Hz, 1H), 7.35 (d, J = 8.6 Hz, 1H), 5.17 (s, 1H), 4.72-4.63 (m, 1H), 4.60-4.51 (m, 1H), 4.20-4.13 (m, 1H), 4.07-4.01 (m, 1H), 4.01-3.91 (m, 3H), 3.76 (s, 2H), 1.27 (br d, J = 4.6 Hz, 6H) | 2.26 A |
| 285 | 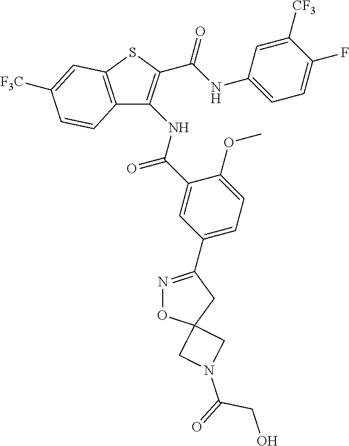 [SMILES: COc1ccc(C2=NOC3(C2)CN(C(=O)CO)C3)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (2-(2- hydroxyacetyl)- 5-oxa-2,6- diazaspiro[3.4] oct-6-en-7-yl)- 2- methoxy- benzamido)-6- (trifluoromethyl) benzo[b] thiophene-2- carboxamide | 723.2 | 1H NMR (400 MHz, DMSO-d6) δ ppm 10.90-10.82 (m, 1H), 10.75 (br dd, J = 3.7, 2.4 Hz, 1H), 8.67 (s, 1H), 8.15 (dd, J = 6.2, 2.1 Hz, 1H), 8.11-8.04 (m, 2H), 8.00 (br dd, J = 8.4, 3.8 Hz, 1H), 7.89-7.74 (m, 2H), 7.54 (t, J = 10.0 Hz, 1H), 7.35 (d, J = 8.8 Hz, 1H), 5.02 (t, J = 6.0 Hz, 1H), 4.53-4.45 (m,1H), 4.45-4.34 (m, 1H), 4.25-4.15 (m, 1H), 4.13-4.05 (m, 1H), 3.99 (s, 3H), 3.95 (d, J = 5.4 Hz, 2H), 3.77 (s, 1H) | 2.18 A |
| 286 | 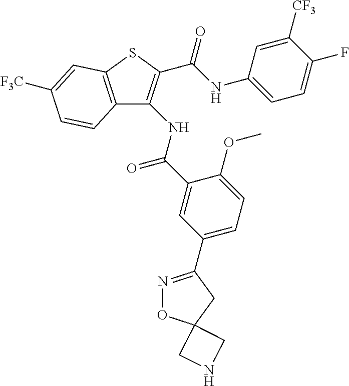 [SMILES: COc1ccc(C2=NOC3(CNC3)C2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(2- methoxy-5-(5- oxa-2,6- diazaspiro[3.4] oct-6-en-7- yl)benzamido)- 6- (trifluoromethyl) benzo[b] thiophene-2- carboxamide | 667.2 | 1H NMR (400 MHz, DMSO-d6) δ ppm 10.88 (br d, J = 2.7 Hz, 1H), 10.79-10.69 (m, 1H) 8.65 (s, 1H), 8.18- 8.11 (m, 1H), 8.11- 8.02 (m, 2H), 8.02- 7.92 (m, 1H), 7.88- 7.76 (m, 2H), 7.53 (t, J = 9.3 Hz, 2H), 7.31 (br d, J = 9.3 Hz, 1H), 3.97 (br s, 3H), 3.83 (br d, J = 8.3 Hz, 2H), 3.71- 3.64 (m, 3H), 1.90 (s, 1H) | 2.18 A |
| 287 | 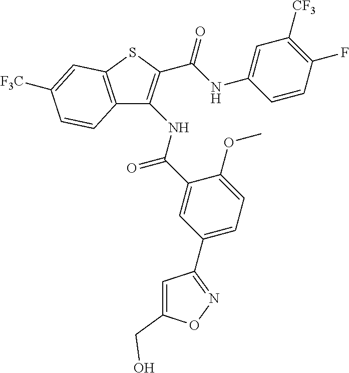 [SMILES: COc1ccc(-c2cc(CO)on2)cc1C(=O)Nc1c(C(=O)Nc2ccc(F)c(C(F)(F)F)c2)sc2cc(C(F)(F)F)ccc12] | N-(4-fluoro-3- (trifluoromethyl) phenyl)-3-(5- (5- (hydroxymethyl) isoxazol-3- yl)-2- methoxy- benzamido)-6- (trifluoromethyl) benzo[b] thiophene-2- carboxamide | 654.2 | 1H NMR (400 MHz, DMSO-d6) δ ppm 10.89 (br d, J = 3.7 Hz, 1H), 9.90 (s, 2H), 8.69- 8.43 (m, 2H), 8.38- 7.97 (m, 2H), 7.86- 7.68 (m, 3H), 7.59- 7.33 (m,1H), 6.91 (s, 1H), 5.75-5.67 (m, 1H), 4.61 (br d, J = 5.4 Hz, 2H), 3.98 (s, 3H) | 2.31 A |
| 288 | 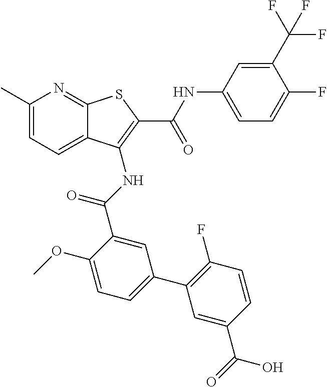 [SMILES: COc1ccc(-c2cc(C(=O)O)ccc2F)cc1C(=O)Nc1c(C(=O)Nc2ccc(F)c(C(F)(F)F)c2)sc2nc(C)ccc12] | 6-fluoro-3′-[(2- {[4-fluoro-3- (trifluoromethyl) phenyl] carbamoyl}-6- methylthieno[2, 3-b]pyridin-3- yl)carbamoyl]- 4′-methoxy- [1,1′-biphenyl]- 3-carboxylic acid | 642.0 | 1H NMR (300MHz, DMSO-d6) δ = 13.21 (s, 1H), 11.31-10.96 (m, 1H), 10.72-10.53 (m, 1H), 8.26-8.21 (m, 1H), 8.21-8.15 (m, 1H), 8.11-8.05 (m, 2H), 8.04-7.94 (m, 2H), 7.85-7.78 (m, 1H), 7.57-7.48 (m, 1H), 7.47-7.35 (m, 3H), 4.15-3.90 (m, 3H), 2.68-2.64 (m, 3H). | 2.33 C |
| 289 | 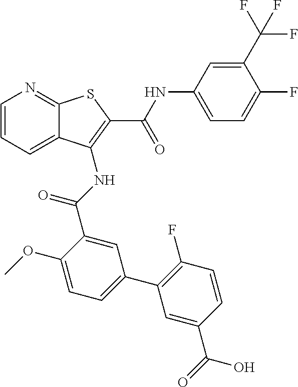 [SMILES: COc1ccc(-c2cc(C(=O)O)ccc2F)cc1C(=O)Nc1c(C(=O)Nc2ccc(F)c(C(F)(F)F)c2)sc2ncccc12] | 6-fluoro-3′-[(2- {[4-fluoro-3- (trifluoromethyl) phenyl] carbamoyl} thieno[2, 3-b]pyridin-3- yl)carbamoyl]- 4′-methoxy- [1,1′-biphenyl]- 3-carboxylic acid | 626.0 (M − H)⁻ | 1H NMR (400 MHz, DMSO-d6) δ ppm 13.42-12.96 (m, 1H), 11.11 (s, 1H), 10.73 (s, 1H), 8.76 (dd, J = 2.0, 4.5 Hz, 1H), 8.35 (dd, J = 1.5, 8.0 Hz, 1H), 8.18 (br d, J = 6.5 Hz, 1H), 8.10-7.97 (m, 4H), 7.84-7.71 (m, 1H), 7.61-7.36 (m, 4H), 4.01 (s, 3H) | 2.37 C |
| 290 | 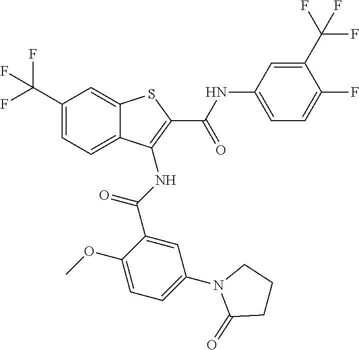 [SMILES: COc1ccc(N2CCCC2=O)cc1C(=O)Nc1c(C(=O)Nc2ccc(F)c(C(F)(F)F)c2)sc2cc(C(F)(F)F)ccc12] | N-[4-fluoro-3- (trifluoromethyl) phenyl]-3-[2- methoxy-5-(2- oxopyrrolidin- 1- yl)benzamido]- 6- (trifluoromethyl)- 1- benzothiophene- 2- carboxamide | 640.0 | 1H NMR (500 MHz, DMSO-d6) δ ppm 10.85-10.78 (m, 1H), 8.62 (s, 1H), 8.11 (br d, J = 4.6 Hz, 1H), 8.06- 8.01 (m, 2H), 7.99- 7.93 (m, 1H), 7.81 (br d, J = 8.6 Hz, 2H), 7.52 (t, J = 9.6 Hz, 1H), 7.30- 7.18 (m, 1H), 3.95 (s, 3H), 3.78 (br t, J = 7.2 Hz, 2H), 2.55 (s, 1H), 2.47 (br t, J = 8.1 Hz, 2H), 2.11-1.98 (m, 2H). | 2.53 A |

What is claimed is:

1. A compound of Formula (I): 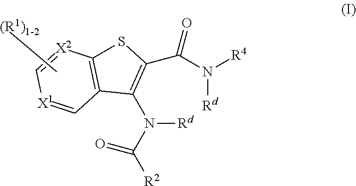
or a pharmaceutically acceptable salt thereof, wherein:
X¹ and X² are each N or CR¹; provided that X¹ and X² are not both N;
R¹ is H, halo, C₁₋₄ alkyl substituted with 0-5 halo, or C₃₋₆ cycloalkyl;
R² is phenyl substituted with 1-3 R³ and 1 R⁵, or a 5 to 6-membered heteroaryl comprising 1-4 heteroatoms selected from O, S(═O)ₚ, N, and NR^{2a} and substituted with 0-3 R³ and 0-1 R⁵;
R^{2a} is H or C₁₋₃ alkyl substituted with 0-2 halo or —OH;
R³ is halo, CN, OH, C₁₋₄ alkyl, or —OC₁₋₄ alkyl substituted with 0-5 halo, OH, —OC₁₋₄ alkyl, aryl, or heterocyclyl;
R⁴ is C₁₋₆ alkyl substituted with 0-5 halo, CN, OH, or OC₁₋₃ alkyl, —(CR^{d}R^{d})₀₋₁—C₃₋₁₀-cycloalkyl substituted with 0-2 R^{4a} or 0-2 R^{4b}, phenyl substituted with 0-2 R^{4a} or 0-2 R^{4b}, —(CR^{d}R^{d})ₙ-3 to 12-membered heterocyclyl comprising 1-4 heteroatoms selected from O, S(═O)ₚ, N, NH, and NC₁₋₃ alkyl, and substituted with 0-2 R^{4a} or 0-2 R^{4b};
R^{4a} or R^{4b} is halo, CN, or C₁₋₄ alkyl substituted with 0-5 halo, OH, or —OC₁₋₄ alkyl substituted with 0-5 halo;
R⁵ is —NR^{5a}R^{5a}, —(CH₂)₁₋₂—NR^{5b}R^{5b}, —C(═O)NR^{5b}R^{5b}, —S(═O)ₚNR^{5b}R^{5b}, C₃₋₆ alkyl substituted with 0-2 OH, C₂-s alkenyl substituted with 0-3 R⁶ and 0-2 R⁷, C₂₋₈ alkynyl substituted with 0-3 R⁶ and 0-2 R⁷, C₃₋₁₂ carbocyclyl substituted with 0-3 R⁶ and 0-2 R⁷, or a 3- to 12-membered heterocyclyl comprising 1-4 heteroatoms selected from O, S(═O)ₚ, N and NR¹⁰ and substituted with 0-3 R⁶ and 0-1 R⁷;
R^{5a} and R^{5a} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R^{5b} is H or C₁₋₆ alkyl substituted with 0-3 R⁶ and 0-2 R⁷; or R^{5b} and R^{5b} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R⁶ is halo, CN, ═O, —OH, —OC₁₋₄ alkyl, or C₁₋₄ alkyl substituted with 0-2 halo or OH;
R⁷ is C₁₋₆ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, —C(═O)NR^{a}S(═O)ₚR^{e}, C₃₋₆ carbocyclyl substituted with 0-5 R^{e}, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NR^{a}R^{a}, —C(═O)NR^{a}OR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —NR^{a}S(═O)ₚNR^{a}R^{a}, —OC(═O)NR^{a}R^{a}, —OC(═O)NR^{a}OR^{b}, —S(═O)ₚNR^{a}R^{a}, —S(═O)ₚR^{e}, or —OP(═O)(OH)₂, —(CH₂)ₙ—C₃₋₆ carbocyclyl substituted with 0-3 R^{e}, or —(CH₂)ₙ-heterocyclyl comprising 1-4 heteroatoms selected from O, S(═O)ₚ, and N, and substituted with 0-3 R^{e};
R¹⁰ is H, C₁₋₄ alkyl substituted with 0-2 R¹¹, —C(═O)R^{b}, —C(═O)OR^{b}, —C(═O)NR^{a}R^{a}, —C(═O)C(═O)OR^{b}, —S(═O)ₚR^{e}, C₃₋₆ carbocyclyl substituted with 0-5 R^{e}, or a 4- to 6-membered heterocyclyl comprising 1-4 heteroatoms selected from O, S(═O)ₚ, N and NR¹² and substituted with 0-5 R^{e};
R¹¹ is —OR^{b}, —C(═O)OR^{b}, —C(═O)NR^{a}R^{a}, —S(═O)ₚR^{e}, or aryl;
R¹² is H, C₁₋₄ alkyl, or aryl;
R^{a} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₆ alkyl substituted with 0-5 R^{e}, C₂₋₆ alkenyl substituted with 0-5 R, C₂₋₆ alkynyl substituted with 0-5 R^{e}, C₃₋₆ carbocyclyl, or heterocyclyl;
R^{d} is H or C₁₋₄ alkyl;
R^{e} is halo, CN, NO₂, ═O, C₁₋₆ alkyl substituted with 0-5 R⁹, C₂₋₆ alkenyl substituted with 0-5 R⁹, C₂₋₆ alkynyl substituted with 0-5 R^{g}, —(CH₂)ₙ-carbocyclyl substituted with 0-5 R^{g}, —(CH₂)ₙ-heterocyclyl substituted with 0-5 R⁹, —(CH₂)ₙOR^{f}, —C(═O)OR^{f}, —C(═O)NR^{f}R^{f}, —NRC(═O)R^{f}, —S(═O)ₚR^{f}, —S(═O)ₚNR^{f}R^{f}, —NR^{f}S(═O)ₚR^{f}, —NR^{f}C(═O)OR^{f}, —OC(═O)NR^{f}R^{f}, or —(CH₂)ₙNR^{f}R^{f};
R^{f} is H, C₁₋₆ alkyl substituted with 0-1 —OC₁₋₄ alkyl, C₃₋₆ cycloalkyl, aryl, or heterocyclyl; or R^{f} and R^{f} together with the nitrogen atom to which they are both attached form a heterocyclyl;
R^{g} is halo, CN, OH, S(═O)₂C₁₋₃ alkyl, C₁₋₆ alkyl, C₃₋₆ cycloalkyl, or aryl;
n is zero, 1, 2, or 3; and
p is zero, 1, or 2.

2. The compound of claim 1, having Formula (II): 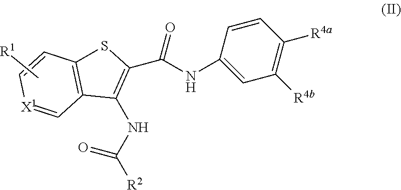
or a pharmaceutically acceptable salt thereof, wherein:
X¹ is N or CR¹;
R¹ is H, halo or C₁₋₃ alkyl substituted with 0-4 halo;
R² is 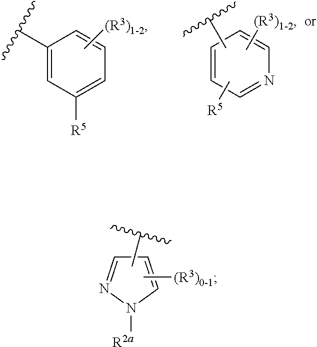
R^{2a} is C₁₋₃ alkyl substituted with 0-1 —OH;
R³ is halo, C₁₋₄ alkyl, or —OC₁₋₄ alkyl substituted with 0-4 halo;
R^{4a} is halo;
R^{4b} is C₁₋₄ alkyl substituted with 0-4 halo;
R⁵ is —NR^{5a}R^{5a}, —C(═O)NR^{5b}R^{5b}, C₂₋₆ alkenyl substituted with 0-3 R⁶ and 0-2 R⁷, C₂₋₆ alkynyl substituted with 0-3 R⁶ and 0-2 R⁷, C₃₋₆ cycloalkyl substituted with 0-3 R⁶ and 0-2 R⁷, phenyl substituted with 0-3 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R^{5a} and R^{5a} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R^{5b} is H or C₁₋₅ alkyl substituted with 0-1 R⁶ and 0-1 R⁷; or R^{5b} and R^{5b} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R⁶ is halo, CN, ═O, —OH, —OC₁₋₃ alkyl, or C₁₋₃ alkyl substituted with 0-2 halo or OH;
R⁷ is C₁₋₅ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(═O)ₚNR^{a}R^{a}, —OC(═O)NR^{a}R^{a}, —OC(═O)NR^{a}OR^{b}, —S(═O)ₚNR^{a}R^{a}, —S(═O)ₚR^{c}, or —OP(═O)(OH)₂;
R¹⁰ is H, C₁₋₄ alkyl substituted with 0-2 R¹¹, —C(═O)R^{b}, —C(═O)OR^{b}, —C(═O)NR^{a}R^{a}, —C(═O)C(═O)OR^{b}, —S(═O)R^{e} C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OR^{b}, —C(═O)NR^{a}R^{a}, or —S(═O)₂R^{c};
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R, C₃₋₆ carbocyclyl, or heterocyclyl;
R^{d} is H or C₁₋₃ alkyl;
R^{e} is halo, CN, ═O, C₁₋₆ alkyl substituted with 0-5 R⁹, C₂₋₆ alkenyl substituted with 0-5 R⁹, C₂₋₆ alkynyl substituted with 0-5 R^{g}, —(CH₂)ₙ—C₃₋₆ cycloalkyl, —(CH₂)ₙ—C₆ aryl, —(CH₂)ₙ-heterocyclyl, —(CH₂)ⁿOR^{f}, —C(═O)OR^{f}, or —S(═O)ₚR^{f};
R^{f} is H or C₁₋₃ alkyl substituted with 0-1 —OC₁₋₄ alkyl;
R^{g} is halo, CN, OH, C₁₋₆ alkyl, C₃₋₆ cycloalkyl, or aryl;
n is zero, 1, 2, or 3; and
p is zero, 1, or 2.

3. The compound of claim 2, having Formula (III): 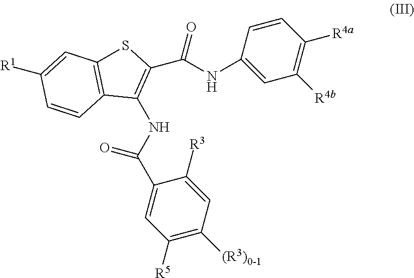
or a pharmaceutically acceptable salt thereof, wherein:
R¹ is C₁₋₃ alkyl substituted with 0-3 halo;
R³ is halo, C₁₋₃ alkyl or —OC₁₋₄ alkyl;
R^{4a} is halo;
R^{4b} is C₁₋₃ alkyl substituted with 0-4 F;
R⁵ is —NR^{5a}R^{5a}, —C(═O)NR^{5b}R^{5b}, C₂₋₆ alkenyl substituted with 0-2 R⁶ and 0-2 R⁷, C₂₋₆ alkynyl substituted with 0-2 R⁶ and 0-2 R⁷, C₃₋₆ cycloalkyl substituted with 0-2 R⁶ and 0-2 R⁷, phenyl substituted with 0-2 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R^{5a} and R^{5a} together with the nitrogen atom to which they are both attached form a heterocyclyl comprising additional 0-5 heteroatoms selected from O, S(═O)ₚ, N, and NR¹⁰, and substituted with 0-2 R⁶ and 0-2 R⁷;
R^{5b} is H or C₁₋₅ alkyl substituted with 0-1 R⁶ and 0-1 R⁷; or R^{5b} and R^{5b} together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R⁶ is halo, CN, ═O, —OH, —OC₁₋₃ alkyl, or C₁₋₃ alkyl;
R⁷ is C₁₋₅ alkyl substituted with 0-1 R⁸ and 0-1 R⁹, —OR^{b}, —NR^{a}R^{a}, —NR^{a}C(═O)R^{b}, —NR^{a}C(═O)OR, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, —C(═O)NHOR^{b}, or C₁₋₃ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(═O)ₚNR^{a}R^{a}, —OC(═O)NR^{a}R^{a}, —OC(═O)NR^{a}OR^{b}, —S(═O)ₚNR^{a}R^{a}, or —S(═O)ₚR^{c};
R¹⁰ is H, C₁₋₄ alkyl substituted with 0-2 R¹¹, —C(═O)R^{b}, —C(═O)OR^{b}, —C(═O)C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OR^{b}, —C(═O)NR^{a}R^{a}, or —S(═O)₂C₁₋₄ alkyl;
R¹² is H or C₁₋₃ alkyl;
R^{a} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₄ alkyl substituted with 0-4 R^{e}, C₂₋₄ alkenyl substituted with 0-4 R^{e}, C₂₋₄ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e} or C₃₋₆ carbocyclyl;
R^{d} is H or C₁₋₂ alkyl;
R^{e} is halo, CN, ═O, C₁₋₅ alkyl substituted with 0-5 R^{g}, —(CH₂)ₙ—C₃₋₆ cycloalkyl, —(CH₂)ₙ-heterocyclyl, —(CH₂)ₙ—C₆ aryl, —(CH₂)ₙ-heteroaryl, —(CH₂)ₙOR^{f}, —C(═O)OR^{f}, or —S(═O)ₚR^{f};
R^{f} is H or C₁₋₃ alkyl;
R^{g} is halo, CN, OH, C₁₋₅ alkyl, or C₃₋₆ cycloalkyl; and
n is zero, 1, or 2.

4. The compound of claim 3, or a pharmaceutically acceptable salt thereof, wherein:
R¹ is C₁₋₂ alkyl substituted with 0-3 halo;
R³ is —OC₁₋₃ alkyl;
R^{4a} is halo;
R^{4b} is C₁₋₂ alkyl substituted with 0-4 F;
R⁵ is 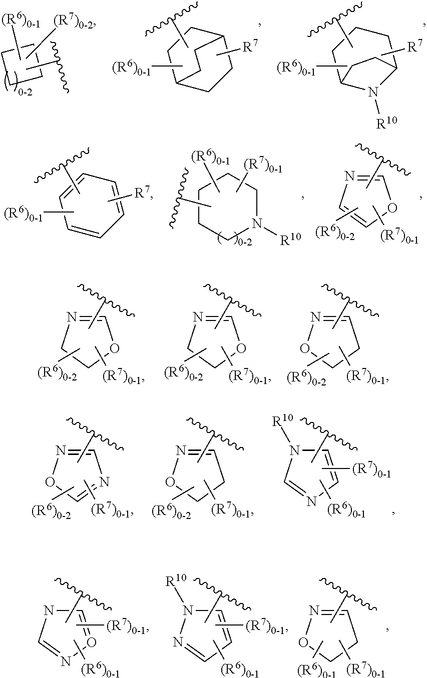 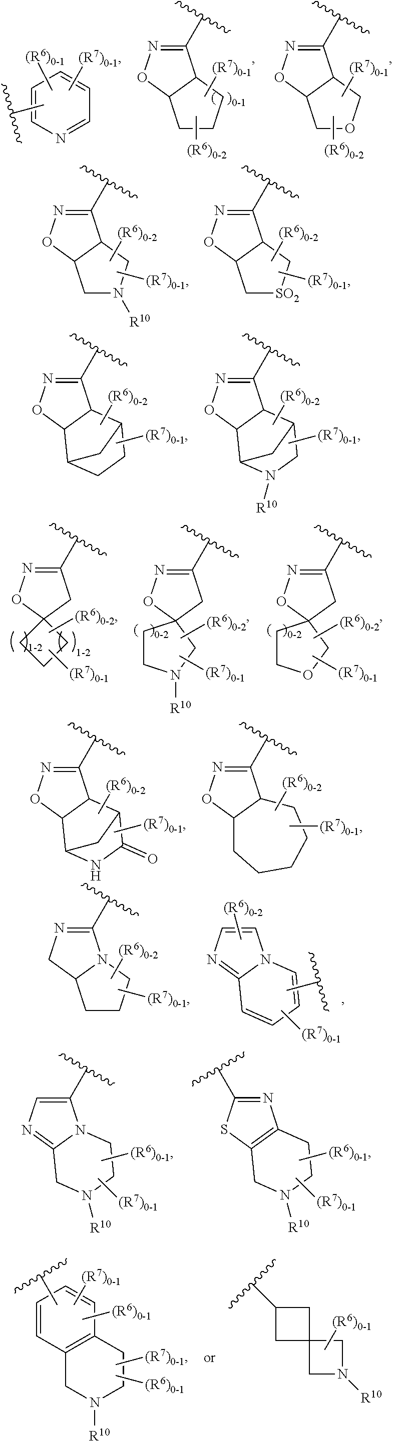
R⁶ is halo, ═O, —OH, or —OC₁₋₂ alkyl, or C₁₋₂ alkyl;
R⁷ is C₁₋₅ alkyl substituted with 0-1 R⁸ and 0-1 R⁹, —NR^{a}R^{a}, —NR^{a}C(═O)R^{b}, —NR^{a}C(═O)OR, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —S(═O)ₚR^{c}, —S(═O)ₚNR^{a}R^{a}, or C₃₋₆ cycloalkyl substituted with 0-2 R^{e};
R⁸ is halo, —C(═O)OR^{b} or C₁₋₃ alkyl substituted with 0-3 halo;
R⁹ is —OR^{b}, —NR^{a}R^{a}, —NHC(═O)R^{b}, —NHC(═O)OR^{b}, —NHS(═O)ₚR^{e}, —NHS(═O)ₚNR^{a}R^{a}, —OC(═O)NR^{a}R^{a}, —OC(═O)NR^{a}OR^{b}, —S(═O)ₚNR^{a}R^{a}, or —S(═O)ₚR^{c};
R¹⁰ is H, C₁₋₃ alkyl substituted with 0-2 R¹¹, —C(═O)R^{b}, —C(═O)OR^{b}, —C(═O)C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N, and NR¹², and substituted with 0-4 R^{e};
R¹¹ is —OH, —C(═O)OH, —C(═O)OC₁₋₄ alkyl, or —C(═O)NR^{a}R^{a};
R¹² is H or C₁₋₂ alkyl;
R^{a} is H, C₁₋₄ alkyl substituted with 0-4 R^{e}, —(CH₂)₀₋₁-phenyl substituted with 0-4 R^{e}, C₃₋₆ cycloalkyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₃ alkyl substituted with 0-4 R^{e}, C₂₋₃ alkenyl substituted with 0-4 R^{e}, C₂₋₃ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{e} is C₁₋₄ alkyl;
R^{e} is halo, CN, ═O, C₁₋₅ alkyl substituted with 0-5 R^{g}, —(CH₂)ₙ—C₃₋₆ cycloalkyl, —(CH₂)ₙ-heterocyclyl, —(CH₂)ₙ—C₆ aryl, —(CH₂)ₙ-heteroaryl, —(CH₂)ₙOR^{f}, —C(═O)OR^{f}, or —S(═O)ₚR^{f};
R^{f} is H or C₁₋₂ alkyl; and
R^{g} is halo, CN, OH, or C₁₋₅ alkyl.

5. The compound of claim 4, having Formula (IV): 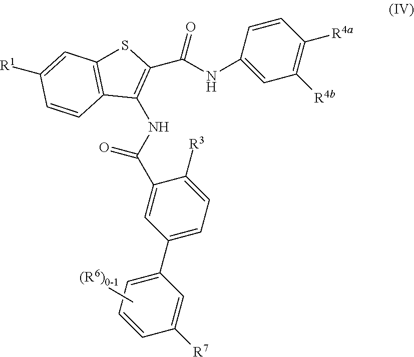
or a pharmaceutically acceptable salt thereof, wherein:
R¹ is C₁₋₂ alkyl substituted with 0-3 halo;
R³ is —OC₁₋₃ alkyl;
R^{4a} is halo;
R⁴ is C₁₋₂ alkyl substituted with 0-3 halo;
R⁶ is halo or C₁₋₂ alkyl;
R⁷ is C₁₋₂ alkyl substituted with 0-1 R⁸ and 0-1 R⁹, —NR^{a}C(═O)OR^{b}, —NR^{a}S(═O)ₚR^{c}, —C(═O)R^{b}, —C(═O)OR^{b}, —C(═O)NR^{a}R^{a}, —S(═O)ₚR^{c}, —S(═O)ₚNR^{a}R^{a}, or C₃₋₆ cycloalkyl substituted with 0-2 R^{e};
R⁸ is halo, —C(═O)OR^{b} or C₁₋₂ alkyl substituted with 0-3 halo;
R⁹ is —OR^{b}, —NR^{a}R^{a}, —NHC(═O)R^{b}, —NHC(═O)OR^{b}, —NHS(═O)ₚR^{e}, —NHS(═O)ₚNR^{a}R^{a}, —OC(═O)NR^{a}R^{a}, —S(═O)ₚNR^{a}R^{a}, or —S(═O)ₚR^{c};
R^{a} is H, C₁₋₃ alkyl substituted with 0-3 R^{e}, —(CH₂)₀₋₁-phenyl substituted with 0-3 R^{e}, C₃₋₆ cycloalkyl substituted with 0-3 R^{e} or heterocyclyl substituted with 0-3 R^{e}; or R^{a} and R^{a} together with the nitrogen atom to which they are both attached form a heterocyclyl substituted with 0-3 R^{e};
R^{b} is H, C₁₋₃ alkyl substituted with 0-3 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₃ alkyl;
R^{e} is halo, CN, ═O, C₁₋₄ alkyl substituted with 0-5 R^{g}, —(CH₂)ₙ—C₃₋₆ cycloalkyl, —(CH₂)ₙ-heterocyclyl, —(CH₂)ₙ—C₆ aryl, —(CH₂)ₙ-heteroaryl, —(CH₂)ₙOR^{f}, or —C(═O)OR^{f};
R^{f} is H or C₁₋₂ alkyl; and
R^{g} is halo, CN, OH, C₁₋₄ alkyl.

6. The compound of claim 5, having Formula (V): 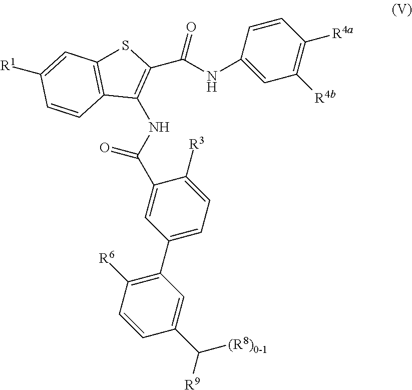
or a pharmaceutically acceptable salt thereof, wherein:
R¹ is CF₃;
R³ is —OC₁₋₂ alkyl;
R^{4a} is F;
R^{4b} is CF₃;
R⁶ is halo;
R⁸ is —C(═O)OR^{b} or —CF₃;
R⁹ is —OR^{b}, —NR^{a}R^{a}, —NHC(═O)R^{b}, —NHS(═O)ₚR^{c}, —OC(═O)NR^{a}R^{a}, or —S(═O)₂R^{e};
R^{a} is H, C₁₋₃ alkyl, —(CH₂)₀₋₁-phenyl substituted with 0-2 R^{e}, or C₃₋₆ cycloalkyl; or R^{a} and R^{a} together with the nitrogen atom to which they are both attached form a heterocyclyl substituted with 0-3 R^{e};
R^{b} is H, C₁₋₃ alkyl substituted with 0-2 R^{e}, C₃₋₆ cycloalkyl, or heterocyclyl;
R^{c} is C₁₋₃ alkyl;
R^{e} is —OR; and
R^{f} is H or C₁₋₂ alkyl.

7. The compound of claim 4, or a pharmaceutically acceptable salt thereof, wherein:
R¹ is CF₃;
R³ is —OCH₃;
R^{4a} is F;
R^{4b} is CF₃;
R⁵ is 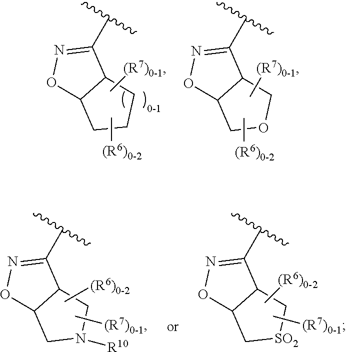
R⁶ is halo, —OH, —OC₁₋₂ alkyl, or C₁₋₂ alkyl;
R⁷ is C₁₋₅ alkyl substituted with 0-1 R⁸ and 0-1 R⁹, —NR^{a}R^{a}, or —NR^{a}C(═O)R^{b};
R⁸ is —C(═O)OR;
R⁹ is OH;
R¹⁰ is H, C₁₋₃ alkyl substituted with 0-2 R¹¹, —C(═O)R^{b}, —C(═O)OR^{b}, —C(═O)NR^{a}R^{a}, or a 4- to 6-membered heterocyclyl comprising 1-4 heteroatoms selected from O, S(═O)ₚ, N and NR¹², and substituted with 0-4 R^{e};
R¹¹ is —OH, —C(═O)OH, or C(═O)NR^{a}R^{a};
R¹² is H and C₁₋₂ alkyl;
R^{a} is H or C₁₋₃ alkyl;
R^{b} is H or C₁₋₃ alkyl substituted with 0-1 R^{e}; and
R^{e} is OH.

8. The compound of claim 7, or a pharmaceutically acceptable salt thereof, wherein:
R⁵ is 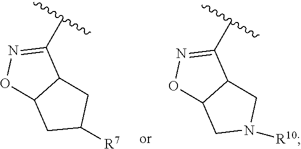
R⁷ is C₁₋₄ alkyl substituted with 0-1 OH;
R¹⁰ is —C(═O)R^{b};
R^{b} is H or C₁₋₃ alkyl substituted with 0-1 R^{e}; and
R^{e} is OH.

9. The compound of claim 4, or a pharmaceutically acceptable salt thereof, wherein:
R¹ is CF₃;
R³ is —OCH₃;
R^{4a} is F;
R^{4b} is CF₃;
R⁵ is 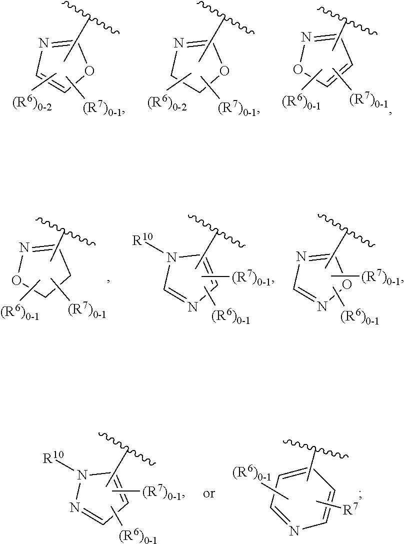
R⁶ is halo, —OH, or C₁₋₂ alkyl;
R⁷ is —NR^{a}R^{a}, —C(═O)NR^{a}R^{a}, or —S(═O)₂NR^{a}R^{a};
R¹⁰ is H, C₁₋₄ alkyl substituted with 0-1 R¹¹, or —C(═O)R^{b};
R¹¹ is —OH or —C(═O)OH;
R^{a} is H or C₁₋₃ alkyl; or R^{a} and R^{a} together with the nitrogen atom to which they are both attached form a heterocyclyl;
R^{b} is H or C₁₋₃ alkyl;
R^{c} is C₁₋₃ alkyl or —(CH₂)₀₋₁OR^{f}; and
R^{f} is H or C₁₋₂ alkyl.

10. The compound of claim 9, or a pharmaceutically acceptable salt thereof, wherein:
R⁵ is 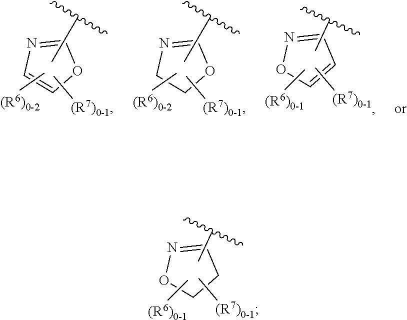
R⁶ is halo, —OH, or C₁₋₂ alkyl;
R⁷ is —NR^{a}R^{a}, —C(═O)NR^{a}R^{a}, or —S(═O)₂NR^{a}R^{a};
R^{a} is H or C₁₋₃ alkyl; or R^{a} and R^{a} together with the nitrogen atom to which they are both attached form a heterocyclyl substituted with 0-2 R^{e};
R^{e} is —CH₂OR^{f} and
R^{f} is H or C₁₋₂ alkyl.

11. The compound of claim 3, or a pharmaceutically acceptable salt thereof, wherein:
R¹ is CF₃;
R³ is —OCH₃;
R^{4a} is F;
R^{4b} is CF₃;
R⁵ is —C(═O)NR^{5b}R^{5b};
R^{5b} is H or C₁₋₅ alkyl substituted with 0-1 R⁶ and 0-1 R⁷; or R^{5b} and R^{5b} together with the nitrogen atom to which they are both attached form a heterocyclyl selected from 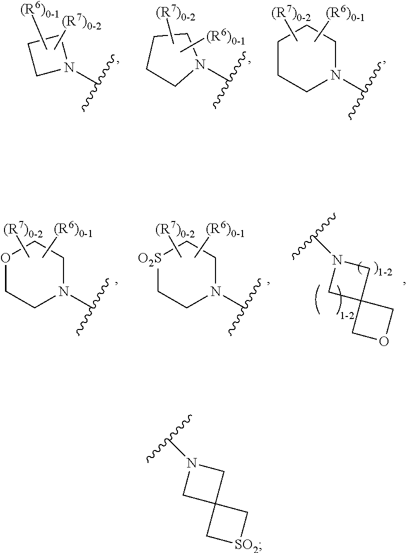
R⁶ is halo, —OH, or C₁₋₃ alkyl;
R⁷ is —S(═O)₂C₁₋₃ alkyl or C₃₋₆ cycloalkyl substituted with 0-2 R^{e};
R^{a} is H or C₁₋₃ alkyl;
R^{b} is H or C₁₋₃ alkyl; and
R^{e} is —S(═O)₂C₁₋₃ alkyl.

12. The compound of claim 11, or a pharmaceutically acceptable salt thereof, wherein:
R^{5b} is H or C₁₋₄ alkyl substituted with 0-1 R⁶ and 0-1 R⁷;
R⁶ is halo, —OH, or C₁₋₄ alkyl substituted with 0-1 OH;
R⁷ is —S(═O)₂C₁₋₂ alkyl or C₃₋₆ cycloalkyl substituted with 0-2 R^{e}; and
R^{e} is —S(═O)₂C₁₋₃ alkyl.

13. The compound of claim 11, or a pharmaceutically acceptable salt thereof, wherein:
R^{5a} and R^{5a} together with the nitrogen atom to which they are both attached form a heterocyclyl selected from 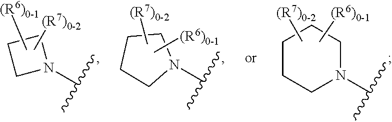
R⁶ is halo, —OH, or C₁₋₃ alkyl;
R⁷ is —S(═O)₂C₁₋₃ alkyl or C₃₋₆ cycloalkyl substituted with 0-2 R^{e};
R^{a} is H or C₁₋₃ alkyl;
R^{b} is H or C₁₋₃ alkyl; and
R^{e} is —S(═O)₂C₁₋₃ alkyl.

14. The compound of claim 4, or a pharmaceutically acceptable salt thereof, wherein:
R¹ is CF₃;
R³ is —OCH₃;
R^{4a} is F;
R^{4b} is CF₃;
R⁵ is 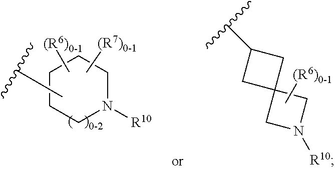
R⁶ is halo, ═O, —OH, —OC₁₋₂ alkyl, or C₁₋₂ alkyl;
R⁷ is C₁₋₂ alkyl substituted with 0-1 R⁸ and 0-1 R⁹, —NR^{a}R^{a}, —NR^{a}C(═O)R^{b}, —NR^{a}C(═O)OR^{b}, or —C(═O)OR^{b};
R⁸ is —C(═O)OR^{b}, —C(═O)NHR^{a}, —C(═O)NHOR^{b}, or C₁₋₂ alkyl substituted with 0-3 halo or OH;
R⁹ is —NR^{a}C(═O)R^{b};
R¹⁰ is H, C₁₋₃ alkyl substituted with 0-2 R¹¹, —C(═O)R^{b}, —C(═O)OR^{b}, —C(═O)C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, —C(═O)OC₁₋₄ alkyl, or C(═O)NR^{a}R^{a};
R¹² is H or C₁₋₂ alkyl;
R^{a} is H or C₁₋₃ alkyl;
R^{b} is H, C₁₋₃ alkyl substituted with 0-2 R^{e}, C₃₋₆ cycloalkyl substituted with 0-2 R^{e}, or heterocyclyl substituted with 0-2 R^{e};
R^{e} is C₁₋₃ alkyl, OH, or —NR^{f}R^{f}; and
R^{f} is H or C₁₋₃ alkyl.

15. The compound of claim 14, or a pharmaceutically acceptable salt thereof, wherein:
R⁵ is 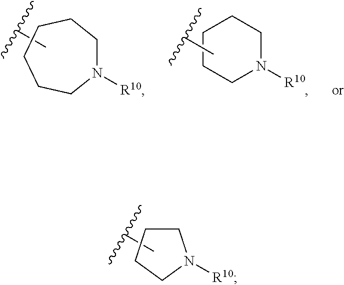
R¹⁰ is H, C₁₋₃ alkyl substituted with 0-2 R¹¹, —C(═O)R^{b}, —C(═O)OR^{b}, —C(═O)C(═O)OR^{b}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, —C(═O)OC₁₋₄ alkyl, or C(═O)NR^{a}R^{a};
R¹² is H or C₁₋₂ alkyl;
R^{a} is H or C₁₋₃ alkyl;
R^{b} is H, or C₁₋₃ alkyl substituted with 0-1 R^{e};
R^{e} is C₁₋₃ alkyl, OH, NR^{f}R^{f}; and
R^{f} is H or C₁₋₃ alkyl.

16. The compound of claim 3, or a pharmaceutically acceptable salt thereof, wherein:
R¹ is CF₃;
R³ is —OCH₃;
R^{4a} is F;
R^{4b} is CF₃;
R⁵ is —NR^{5a}R^{5a};
R^{5a} and R^{5a} together with the nitrogen atom to which they are both attached form a heterocyclyl selected from 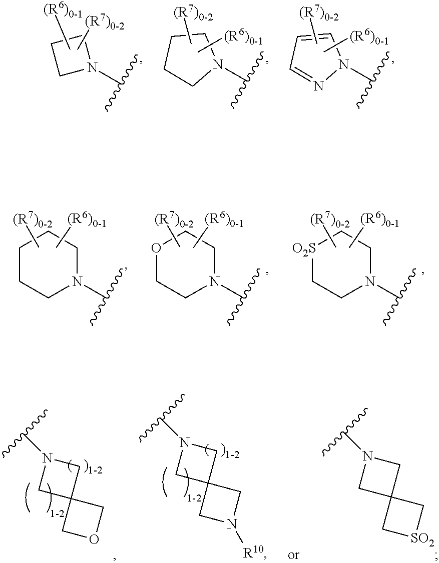
R⁶ is halo, —OH, or C₁₋₃ alkyl;
R⁷ is —S(═O)₂C₁₋₃ alkyl or C₃₋₆ cycloalkyl substituted with 0-2 R^{e};
R¹⁰ is H, C₁₋₄ alkyl substituted with 0-1R¹¹, or —C(═O)R^{b};
R¹¹ is —OH or —C(═O)OH;
R^{a} is H or C₁₋₃ alkyl;
R^{b} is H or C₁₋₃ alkyl; and
R^{e} is —S(═O)₂C₁₋₃ alkyl.

17. The compound of claim 4, or a pharmaceutically acceptable salt thereof, wherein:
R⁵ is 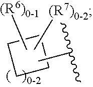
R⁶ is —OH, or —OC₁₋₂ alkyl, or C₁₋₂ alkyl;
R⁷ is C₁₋₂ alkyl substituted with 0-1 R⁸ and 0-1 R⁹, —NR^{a}R^{a}, —C(═O)R^{b}, —C(═O)OR^{b}, or —C(═O)NR^{a}R^{a};
R⁸ is halo;
R⁹ is —OR;
R^{a} is H, C₁₋₃ alkyl, C₃₋₆ cycloalkyl, or heterocyclyl; or R^{a} and R^{a} together with the nitrogen atom to which they are both attached form a heterocyclyl substituted with 0-3 R^{e};
R^{b} is H, C₁₋₃ alkyl substituted with 0-1 R^{e}, or heterocyclyl;
R^{e} is —OR; and
R^{f} is H or C₁₋₂ alkyl.

18. The compound of claim 3, or a pharmaceutically acceptable salt thereof, wherein:
R¹ is CF₃;
R³ is —OC₁₋₂ alkyl;
R^{4a} is F;
R^{4b} is CF₃;
R⁵ is —C(═O)NR^{5b}R^{5b}, 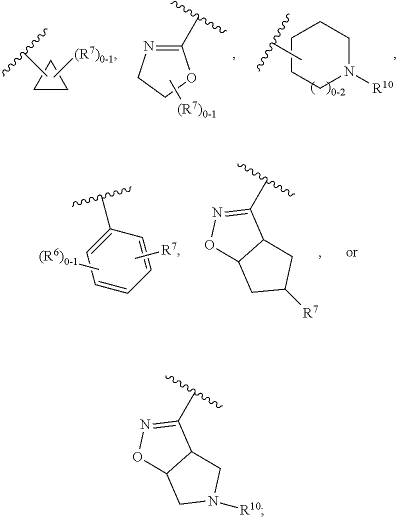
R^{5b} is H or C₁₋₄ alkyl substituted with 0-1 R⁶ and 0-1 R⁷; or R^{5b} and R^{5b} together with the nitrogen atom to which they are both attached form 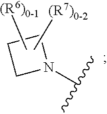
R⁶ is halo, —OH, or C₁₋₃ alkyl;
R⁷ is C₁₋₃ alkyl substituted with 0-1 R⁸ and 0-1 R⁹, —C(═O)NR^{a}R^{a}, —C(═O)OR^{b}, —NR^{a}C(═O)R^{b}, —S(═O)₂NR^{a}R^{a}, —S(═O)₂C₁₋₃ alkyl, or C₃₋₆ cycloalkyl substituted with 0-2 R^{e};
R⁸ is halo, —C(═O)OR^{b}, or C₁₋₃ alkyl substituted with 0-3 halo;
R⁹ is —OH, —NR^{a}R^{a}, —NR^{a}C(═O)R^{b}, NR^{a}S(═O)_{P}C₁₋₄ alkyl, or —OC(═O)NR^{a}R^{a};
R¹⁰ is H, C₁₋₄ alkyl substituted with 0-2 R¹¹, —C(═O)R^{b}, —C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-4 R^{e};
R¹¹ is —OH, —C(═O)OH, —C(═O)NR^{a}R^{a}, or —S(═O)₂C₁₋₄ alkyl;
R¹² is H or C₁₋₃ alkyl;
R^{a} is H, C₁₋₃ alkyl, —(CH₂)₀₋₁—C₃₋₆ cycloalkyl, or —(CH₂)₀₋₁-heterocyclyl; or R^{a} and R^{a} together with the nitrogen atom to which they are both attached form a 5- or 6-membered heterocyclyl substituted with 0-2 R^{e};
R^{b} is H, C₁₋₃ alkyl substituted with 0-4 R^{e}, or heterocyclyl;
R^{e} is C₁₋₃ alkyl, —(CH₂)₀₋₁OR^{f}, or —S(═O)₂C₁₋₃ alkyl; and
R^{f} is H or C₁₋₃ alkyl.

19. A pharmaceutical composition comprising a compound of claim 1, or a pharmaceutically acceptable salt thereof, and a pharmaceutically acceptable carrier.

20. A method for treating a disease associated with relaxin comprising administering a therapeutically effective amount of the pharmaceutical composition of claim 19 to a patient in need thereof.

21. The method of claim 20 wherein the disease is selected from the group consisting of angina pectoris, unstable angina, myocardial infarction, heart failure, acute coronary disease, acute heart failure, chronic heart failure, and cardiac iatrogenic damage.

22. The method of claim 21 wherein the disease is heart failure.
